(19) United States
(12) Patent Application Publication
(10) Pub. No.: US 2026/0108527 A1
(43) Pub. Date: Apr. 23, 2026

(54) POLO LIKE KINASE 4 INHIBITORS

(71) Applicant: ORIC Pharmaceuticals, Inc., South San Francisco, CA (US)

(72) Inventors: Chudi NDUBAKU, Montréal, Québec (CA); Jared Thomas MOORE, San Rafael, CA (US); Paul Anthony GIBBONS, San Francisco, CA (US); F. Anthony ROMERO, Redwood City, CA (US); Daniel Gordon Michael SHORE, San Mateo, CA (US); Joanne TAN, San Mateo, CA (US); Rakesh Harsukhlal VEKARIYA, Santa Clara, CA (US)

(21) Appl. No.: 19/117,563

(22) PCT Filed: Oct. 2, 2023
(86) PCT No.: PCT/US2023/075669

§ 371 Date: Apr. 1, 2025

Related U.S. Application Data
(60) Provisional application No. 63/378,142, filed on Oct. 3, 2022.
(60) Provisional application No. 63/383,773, filed on Nov. 15, 2022.

(51) Int. Cl. A61K 31/5377; A61K 31/416; A61K 31/4178; A61K 31/4196; A61K 31/422; A61K 31/427; A61K 31/4439; A61K 31/4709; A61K 31/497; A61K 31/506; C07B 59/00; C07D 401/14; C07D 403/04; C07D 403/14; C07D 405/14; C07D 409/14; C07D 413/14; C07D 417/14

(52) U.S. Cl. CPC A61K 31/5377; A61K 31/416; A61K 31/4178; A61K 31/4196; A61K 31/422; A61K 31/427; A61K 31/4439; A61K 31/4709; A61K 31/497; A61K 31/506; C07B 59/002; C07D 401/14; C07D 403/04; C07D 403/14; C07D 405/14; C07D 409/14; C07D 413/14; C07D 417/14; C07B 2200/05

(57) ABSTRACT

Disclosed herein are compounds of Formula (IV), or pharmaceutically acceptable salts thereof, that are inhibitors of Polo Like Kinase 4 (PLK4). Also disclosed herein are pharmaceutical compositions comprising the compounds of Formula (IV), or pharmaceutically acceptable salts thereof, and one or more pharmaceutically acceptable excipients. Further disclosed herein are methods of treating cancer in a subject in need thereof, comprising administering to the subject an amount of a compound of Formula (IV), or a pharmaceutically acceptable salt thereof.

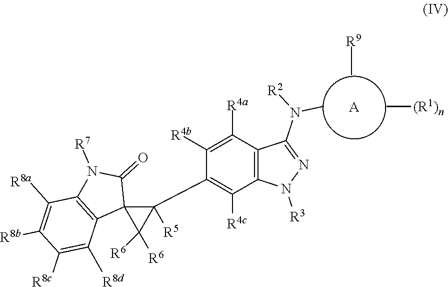

### CROSS-REFERENCE

This application claims the benefit of U.S. Provisional Application Ser. No. 63/378,142 filed Oct. 3, 2022 and U.S. Provisional Application Ser. No. 63/383,773 filed Nov. 15, 2022; which are hereby incorporated by reference in their entirety.

### BACKGROUND OF THE INVENTION

The Polo-like kinases (PLKs) are a family of serine/threonine kinases that play a critical role in cell cycle regulation and cellular responses under stress (Helmke et al. 2016; Zitouni et al. 2014). Mammalian cells express five PLK family members (PLK1-5). All PLKs share a similar structure, with an N-terminal kinase catalytic domain and C-terminal Polo-box domains (PBDs) (Archambault et al. 2015). Polo-like kinase 4 (PLK4), also known as SAK, is a regulator of centriole duplication (Habedanck et al. 2005; Kleylein-Sohn et al. 2007). In proliferating tissues, PLK4 is expressed as a low-abundance enzyme under normal conditions and is required for centriole biogenesis via phosphorylation and interaction with centriolar proteins (Habedanck et al. 2005; Maniswami et al. 2018). Overexpression of PLK4 results in centriole amplification and further genomic instability and tumorigenesis (Holland et al. 2010). Aberrant PLK4 expression has been reported to be involved in several common human cancers (Marina and Saavedra 2014; Shinmura et al. 2014). Thus, strong evidence supports the critical role of PLK4 in carcinogenesis and therapeutic invention. Thus, there is a need for compounds that inhibit PLK4 in subjects having cancer for the treatment of those cancers.

### SUMMARY OF THE INVENTION

In one embodiment is provided a compound of Formula (IV), or a pharmaceutically acceptable salt, solvate, or stereoisomer thereof

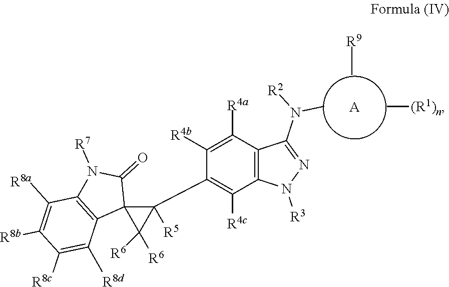

wherein:
Ring A is C₆-C₁₀aryl or heteroaryl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, —P(O)(R^{a})₂, —P(O)₂(R^{a})₂, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; or two R^{1b} on the same atom are taken together to form an oxo;
n is 0, 1, 2, 3, 4, 5, 6, 7, or 8;
R² is hydrogen, C₁-C₆alkyl, C₁-C₆haloalkyl, or C₁-C₆deuteroalkyl;
R³ is hydrogen, C₁-C₆alkyl, C₁-C₆haloalkyl, or C₁-C₆deuteroalkyl;
each of R^{4a}, R^{4b}, and R^{4c} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R⁶ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁷ is hydrogen, C₁-C₆alkyl, C₁-C₆haloalkyl, C₁-C₆deuteroalkyl, C₁-C₆hydroxyalkyl, or C₁-C₆aminoalkyl;
each of R^{8a}, R^{8b}, R^{8c}, and R^{8d} is independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
R⁹ is heteroaryl optionally substituted with one or more R^{1a}, or oxetanyl substituted with one or more R^{1a};
each R^{a} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆alkoxy, C₁-C₆aminoalkyl, C₁-C₆alkylamino, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
provided the compound of Formula (IV) is not

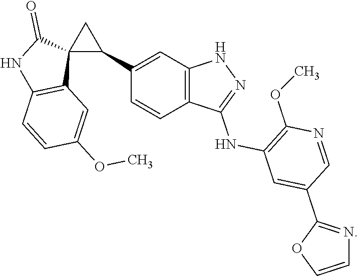 [SMILES: COc1ccc2c(c1)[C@]1(C[C@H]1c1ccc3c(Nc4cc(-c5ncco5)cnc4OC)n[nH]c3c1)C(=O)N2]

In another embodiment is provided a compound of Formula (IVa):

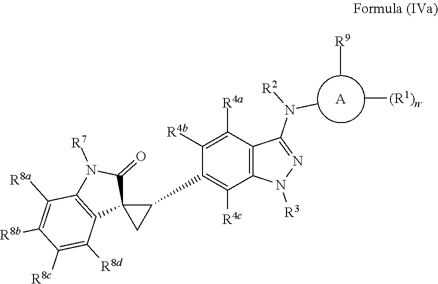

In another embodiment is provided a compound of Formula (IVb):

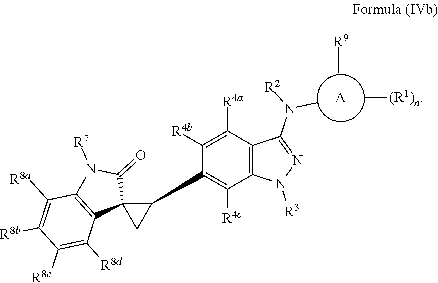

In another embodiment is provided a compound of Formula (V), or a pharmaceutically acceptable salt, solvate, or stereoisomer thereof

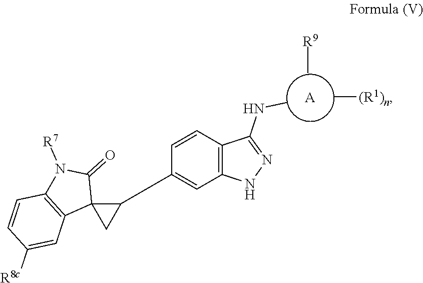

wherein:
Ring A is C₆-C₁₀aryl or heteroaryl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, —P(O)(R^{a})₂, —P(O)₂(R^{a})₂, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3,4, 5, 6, 7, or 8;
R⁷ is hydrogen, C₁-C₆alkyl, C₁-C₆haloalkyl, C₁-C₆deuteroalkyl, C₁-C₆hydroxyalkyl, or C₁-C₆aminoalkyl;
R^{8c} is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
R⁹ is heteroaryl optionally substituted with one or more R^{1a}, or oxetanyl substituted with one or more R^{1a};
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆alkoxy, C₁-C₆aminoalkyl, C₁-C₆alkylamino, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
provided the compound of Formula (V) is not

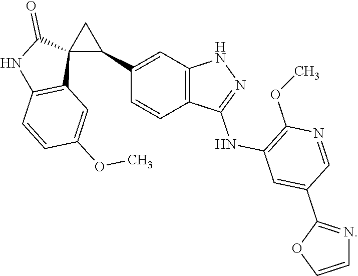 [SMILES: COc1ccc2c(c1)[C@]1(C[C@H]1c1ccc3c(Nc4cc(-c5ncco5)cnc4OC)n[nH]c3c1)C(=O)N2]

In another embodiment is provided a compound of Formula (Va), or a pharmaceutically acceptable salt, solvate, or stereoisomer thereof:

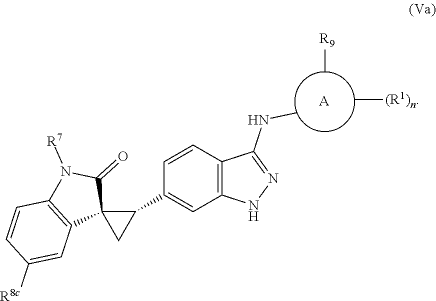

In another embodiment is provided a compound of Formula (Vb), or a pharmaceutically acceptable salt, solvate, or stereoisomer thereof:

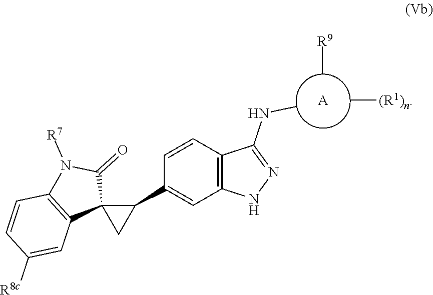

In another embodiment is provided a pharmaceutical composition comprising a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, and one or more pharmaceutically acceptable excipients.

Also provided herein are methods of treating cancer in a subject in need thereof, comprising administering to the subject a therapeutically effective amount of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof. In other embodiments are provided such methods of treating cancer, wherein the cancer in the subject expresses polo-like kinase 4 (PLK4). In a further embodiment are provided such methods of treatment wherein the cancer in the subject exhibits an overexpression of the E3 ubiquitin-protein ligase (TRIM37). In other embodiments are provided such methods of treating cancer, wherein the cancer in the subject exhibits an overexpression of the gene that encodes the tripartite motif-containing protein 37 (TRIM37). In other embodiments are provided such methods of treating cancer, wherein the cancer in the subject has been determined to overexpress the gene that encodes the tripartite motif-containing protein 37 (TRIM37) prior to administration of the compound to the subject.

In other embodiments are provided methods of treating cancer in a subject, comprising:
a. obtaining a biological sample of the cancer from the subject;
b. determining whether the biological sample of the cancer overexpresses the gene that encodes the tripartite motif-containing protein 37 (TRIM37); and administering to the subject a therapeutically effective amount of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, if the biological sample of the cancer is determined to overexpress the gene that encodes the tripartite motif-containing protein 37 (TRIM37).

In other embodiments are provided methods of inhibiting polo-like kinase 4 (PLK4) in a subject in need thereof, comprising administering to the subject a therapeutically effective amount of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof. In some embodiments, the subject has cancer.

In other embodiments are provided compounds of Formula (I), Formula (Ia), Formula (Ib), Formula (IT), Formula (III), Formula (IV), Formula (IVa), Formula (IVb), Formula (V), Formula (Va), or Formula (Vb), or a pharmaceutically acceptable salt, solvate, or stereoisomer thereof, for use in a method of treating cancer in a subject in need thereof. In some embodiments, the cancer in the subject expresses polo-like kinase 4 (PLK4). In some embodiments, the cancer in the subject has been determined to express polo-like kinase 4 (PLK4) prior to administering the compound to the subject. In some embodiments, the cancer in the subject exhibits an overexpression of the gene that encodes the tripartite motif-containing protein 37 (TRIM37).

Further provided herein are uses of a compound of Formula (I), Formula (Ia), Formula (Ib), Formula (IT), Formula (III), Formula (IV), Formula (IVa), Formula (IVb), Formula (V), Formula (Va), or Formula (Vb), or a pharmaceutically acceptable salt, solvate, or stereoisomer thereof, in the manufacture of a medicament for the treatment of cancer in a subject in need thereof. In some embodiments, the cancer in the subject expresses polo-like kinase 4 (PLK4). In some embodiments, the cancer in the subject exhibits an overexpression of the E3 ubiquitin-protein ligase (TRIM37) protein. In some embodiments, the cancer in the subject exhibits an amplification of the gene that encodes the tripartite motif-containing protein 37 (TRIM37).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

As used in the specification and appended claims, unless specified to the contrary, the following terms have the meaning indicated below.

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 10% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Administering” when used in conjunction with a therapeutic means to administer a therapeutic systemically or locally, as directly into or onto a target tissue, or to administer a therapeutic to a subject whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a subject by, e.g., oral administration whereby the therapeutic reaches the target tissue or cells. “Administering” a composition may be accomplished by injection, topical administration, and oral administration or by other methods alone or in combination with other known techniques.

The term “C₂-C₆alkenyl” as used herein, means an alkyl moiety comprising 2 to 6 carbon atoms having at least one carbon-carbon double bond. The carbon-carbon double bond in such a group may be anywhere along the 2 to 6 carbon atom chain that will result in a stable compound. Examples of such groups include, but are not limited to, ethenyl, propenyl, butenyl, allyl, and pentenyl. The alkenyl may be in either the cis or trans conformation about the double bond(s), and should be understood to include both isomers. Examples of alkenyls include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl.

The term “C₁-C₆alkyl,” as used herein, refers to a straight or branched chain hydrocarbon monoradical, which may be fully saturated or unsaturated, having from one to six carbon atoms. Examples of saturated hydrocarbon monoradical include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “C₂-C₆alkynyl,” as used herein, means an alkyl moiety comprising from 2 to 6 carbon atoms and having at least one carbon-carbon triple bond. The carbon-carbon triple bond in such a group may be anywhere along the 2 to 6 carbon chain that will result in a stable compound. Examples of such groups include, but are not limited to, ethyne, propyne, 1-butyne, 2-butyne, 1-pentyne, 2-pentyne, 1-hexyne, 2-hexyne, and 3-hexyne, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated.

The term “C₆-C₁₀aryl,” as used herein, refers to a radical derived from a hydrocarbon ring system comprising hydrogen, 6 to 10 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl.

The term “C₁-C₆ aminoalkyl,” as used herein, refers to a C₁-C₆alkyl radical, as defined above, that is substituted with one or more amino groups. The amino groups in such C₁-C₆ aminoalkyl groups may be unsubstituted, mono-substituted, or disubstituted. Examples of C₁-C₆ aminoalkyl groups include, but are not limited to, —CH₂NH₂, —CH₂N(H)CH₃, —CH₂N(CH₃)₂, and the like.

The term “C₃-C₁₀cycloalkyl” refers to a partially or fully saturated, monocyclic, or polycyclic carbocyclic ring comprising from 3 to 10 carbon atoms, which may include fused (when fused with an aryl or a heteroaryl ring, the cycloalkyl is bonded through a non-aromatic ring atom) or bridged ring systems. In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl

The term “C₁-C₆deuteroalkyl,” as used herein, means a C₁-C₆alkyl group as defined herein wherein one or more hydrogen atoms in the C₁-C₆alkyl group is replaced with a deuterium atom.

The term “C₁-C₆ haloalkyl,” as used herein, refers to a C₁-C₆alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

The term “C₁-C₆ hydroxyalkyl,” as used herein, refers to a C₁-C₆alkyl radical, as defined above, that is substituted with one or more hydroxy groups.

The term “animal” as used herein includes, but is not limited to, humans and non-human vertebrates such as wild, domestic and farm animals. As used herein, the terms “subject,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the subject is a primate. In certain embodiments, the primate or subject is a human.

In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

The term “Aurora kinase A,” or “AurA,” as used herein, means the human protein known to those of ordinary skill in the art as Aurora kinase A, and that is encoded by the AURKA gene.

The term “Aurora kinase B,” or “AurB,” as used herein, means the human protein known to those of ordinary skill in the art as Aurora kinase B, and that is encoded by the AURKB gene.

A “cyano” group refers to a —CN group.

The term “halo” or “halogen,” as used herein, refers to bromo, chloro, fluoro or iodo. In some embodiments, halogen is fluoro or chloro. In some embodiments, halogen is fluoro.

The term “heterocycloalkyl,” as used herein, refers to a 3- to 24-membered partially or fully saturated ring radical comprising 2 to 23 carbon atoms and from one to 8 heteroatoms selected from boron, nitrogen, oxygen, phosphorous, and sulfur. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring).

The term “C₁-C₆heteroalkyl,” as used herein, means an alkyl group in which one or more skeletal atoms of the alkyl are selected from an atom other than carbon, e.g., boron, oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl.

The term “heteroaryl,” as used herein refers to a 5- to 14-membered ring system radical comprising hydrogen atoms, one to thirteen carbon atoms, one to six heteroatoms selected from boron, nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl).

By “pharmaceutically acceptable,” as used herein, is meant the carrier, diluent or excipient must be compatible with the other ingredients of the formulation and not deleterious to the recipient thereof.

The term “pharmaceutical composition” means a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “pharmaceutically acceptable salt,” as used herein, means a salt of a compound of the present invention that retains the biological effectiveness of the free acids and bases of the specified derivative and that is not biologically or otherwise undesirable.

The term “PLK4,” as used herein, means the human protein known to those of ordinary skill in the art as polo-like kinase 4, and that is encoded by the PLK4 gene

The term “oxo,” as used herein, refers to a carbonyl moiety such that alkyl substituted by oxo refers to a ketone group.

The term “solvate,” as used herein, means a molecular complex between compounds of the present invention and solvent molecules. Examples of solvates include, but are not limited to, compounds of the invention in combination water, isopropanol, ethanol, methanol, dimethylsulfoxide (DMSO), ethyl acetate, acetic acid, ethanolamine, or mixtures thereof. The term “hydrate” can be used when said solvent is water. It is specifically contemplated that in the present invention one solvent molecule can be associated with one molecule of the compounds of the present invention, such as a hydrate. Furthermore, it is specifically contemplated that in the present invention, more than one solvent molecule may be associated with one molecule of the compounds of the present invention, such as a dihydrate. Additionally, it is specifically contemplated that in the present invention less than one solvent molecule may be associated with one molecule of the compounds of the present invention, such as a hemihydrate. Furthermore, solvates of the present invention are contemplated as solvates of compounds of the present invention that retain the biological effectiveness of the non-hydrate form of the compounds.

Where a compound of the invention contains an alkenyl group, geometric cis/trans (or Z/E) isomers are possible. Where the compound contains, for example, a keto or oxime group or an aromatic moiety, tautomeric isomerism (‘tautomerism’) can occur. Examples of tautomerism include keto and enol tautomers. A single compound may exhibit more than one type of isomerism. Included within the scope of the invention are all stereoisomers, geometric isomers, and tautomeric forms of the inventive compounds, including compounds exhibiting more than one type of isomerism, and mixtures of one or more thereof. Cis/trans isomers may be separated by conventional techniques well known to those skilled in the art, for example, chromatography and fractional crystallization.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. The terms “racemic” or “racemic mixture,” as used herein, refer to a 1:1 mixture of enantiomers of a particular compound. A mixture of racemates in which one racemate is present in a greater amount than the other racemate in such mixture may be described as “enantiomerically enriched.” The term “diastereomers,” on the other hand, refers to the relationship between a pair of stereoisomers that comprise two or more asymmetric centers and are not mirror images of one another. Designations that are conventional in the art may be used to describe stereoisomers of compounds, or the stereochemistry of a particular asymmetric carbon atom, of the compounds disclosed herein, or mixtures thereof. For example, a single racemate or stereocenter of a compound, may be described as of the (+), the (−), the (R)-, or the (S) configuration. A mixture of racemates may be described by use of the (+) symbol.

The compounds of the present invention may have asymmetric carbon atoms. The carbon-carbon bonds of the compounds of the present invention may be depicted herein using a solid line ( [symbol] ), a solid wedge ( [symbol] ) or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the present invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomeric conglomerates may be separated by conventional techniques known to those skilled in the art. See, e.g. “Stereochemistry of Organic Compounds” by E L Eliel (Wiley, New York, 1994), the disclosure of which is incorporated herein by reference in its entirety.

The term “substituted,” as used herein, means that the specified group or moiety bears one or more substituents. The term “unsubstituted,” means that the specified group bears no substituents. The term “optionally substituted” means that the specified group is unsubstituted or substituted by one or more substituents. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆aryl group, also called “phenyl” herein, is substituted with one additional substituent, one of ordinary skill in the art would understand that such a group has 4 open positions left on carbon atoms of the C₆aryl group (6 initial positions, minus one to which the remainder of the compound of the present invention is bonded, minus an additional substituent, to leave 4). In such cases, the remaining 4 carbon atoms are each bound to one hydrogen atom to fill their valencies. Similarly, if a C₆aryl group in the present compounds is said to be “disubstituted,” one of ordinary skill in the art would understand it to mean that the C₆aryl group has 3 carbon atoms remaining that are unsubstituted. Those three unsubstituted carbon atoms are each bound to one hydrogen atom to fill their valencies.

In accordance with a convention used in the art, the symbol

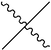

is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g.,

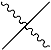

represents a methyl group,

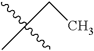 [SMILES: *CC]

represents an ethyl group, and

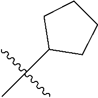 [SMILES: *C1CCCC1]

represents a cyclopentyl group, etc.

If a group, as for example, (R¹)ₙ is depicted as “floating” Ring A in the formula:

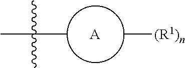

then, unless otherwise defined, the substituent R¹ may reside on any atom of the ring system, assuming replacement of a depicted, implied, or expressly defined hydrogen from one of the ring atoms, so long as a stable structure is formed. A ring system A may be, for example, but not limited to aryl, heteroaryl, cycloalkyl, cycloheteroalkyl, spirocyclyl or a fused ring system.

If a group “R” is depicted as “floating” on a ring system A as shown above, and Ring A contains saturated carbons, then “n” can be more than one, assuming each replaces a currently depicted, implied, or expressly defined hydrogen the ring A; then, unless otherwise defined, where the resulting structure is stable, two R¹ groups may reside on the same carbon. For example, when R¹ is a methyl group, there can exist a germinal dimethyl on a carbon of the ring A. In another example, two R¹ groups on the same carbon, including that carbon, may form a ring, thus creating a spirocyclic ring (a “spirocyclyl group”). It is to be understood that in the compounds of Formula (I), Formula (Ia), Formula (Ib), Formula (II), Formula (III), Formula (IV), Formula (IVa), Formula (IVb), Formula (V), Formula (Va), or Formula (Vb) if n is less than the number of substitutable atoms on Ring A, the other substitutable positions on Ring A are bonded to a hydrogen atom.

As used herein, the term “therapeutic” means an agent utilized to treat, combat, ameliorate, prevent, or improve an unwanted condition or disease of a subject.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “TRIM37,” as used herein, means the human protein known those of ordinary skill in the art as tripartite motif-containing protein 37, an E3 ubiquitin ligase that is encoded by the TRIM37 gene.

The term “CFI-400495” means the compound having the Chemical Abstract Service Registry No. 1338806-73-7, and the structure shown below. The preparation of the compound is described in PCT Application Publication No. WO 2011/123946 and is commercially available for purchase.

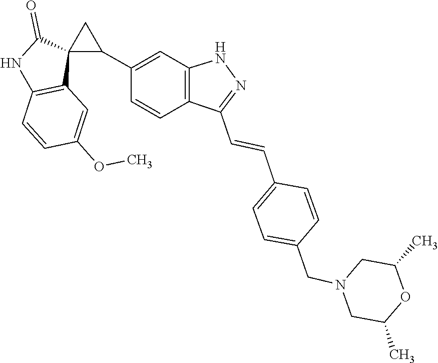

### PLK4 Inhibitor Compounds

Provided herein are compounds of Formula (I), or a pharmaceutically acceptable salt, solvate, or stereoisomer thereof:

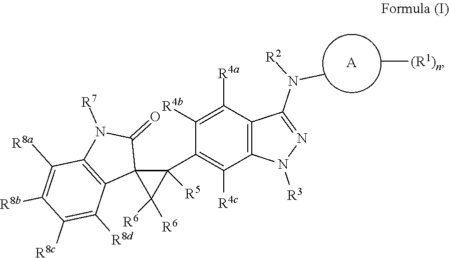

wherein:
Ring A is C₅-C₁₀aryl, heteroaryl, C₃-C₁₀cycloalkyl, or heterocycloalkyl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, —P(O)(R^{a})₂, —P(O)₂(R^{a})₂, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3, 4, 5, 6, 7, or 8;
R² is hydrogen, C₁-C₆alkyl, C₁-C₆haloalkyl, or C₁-C₆deuteroalkyl;
R³ is hydrogen, C₁-C₆alkyl, C₁-C₆haloalkyl, or C₁-C₆deuteroalkyl;
each of R^{4a}, R^{4b}, and R^{4c} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R⁶ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁷ is hydrogen, C₁-C₆alkyl, C₁-C₆haloalkyl, C₁-C₆deuteroalkyl, C₁-C₆hydroxyalkyl, or C₁-C₆aminoalkyl; each of R^{8a}, R^{8b}, R^{8c}, and R^{8d} is independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆alkoxy, C₁-C₆aminoalkyl, C₁-C₆alkylamino, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

Also provided herein are compounds of Formula (Ia), or a pharmaceutically acceptable salt, solvate, or stereoisomer thereof:

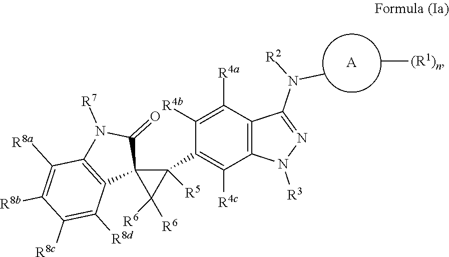

wherein:
Ring A is C₆-C₁₀aryl, heteroaryl, C₃-C₁₀cycloalkyl, or heterocycloalkyl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3, 4, 5, 6, 7, or 8;
R² is hydrogen, C₁-C₆alkyl, C₁-C₆haloalkyl, or C₁-C₆deuteroalkyl;
R³ is hydrogen, C₁-C₆alkyl, C₁-C₆haloalkyl, or C₁-C₆deuteroalkyl;
each of R^{4a}, R^{4b}, and R^{4c} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R⁶ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁷ is hydrogen, C₁-C₆alkyl, C₁-C₆haloalkyl, C₁-C₆deuteroalkyl, C₁-C₆hydroxyalkyl, or C₁-C₆aminoalkyl;
each of R^{8a}, R^{8b}, R^{8c}, and R^{8d} is independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

Further provided herein are compounds of Formula (Ib), or a pharmaceutically acceptable salt, solvate, or stereoisomer thereof:

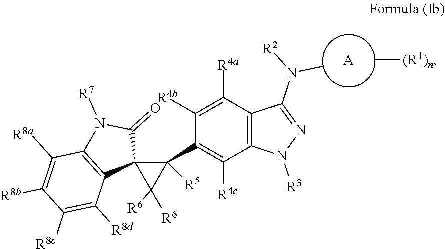

wherein:
Ring A is C₆-C₁₀aryl, heteroaryl, C₃-C₁₀cycloalkyl, or heterocycloalkyl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3, 4, 5, 6, 7, or 8;
R² is hydrogen, C₁-C₆alkyl, C₁-C₆haloalkyl, or C₁-C₆deuteroalkyl;
R³ is hydrogen, C₁-C₆alkyl, C₁-C₆haloalkyl, or C₁-C₆deuteroalkyl;
each of R^{4a}, R^{4b}, and R^{4c} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R⁶ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁷ is hydrogen, C₁-C₆alkyl, C₁-C₆haloalkyl, C₁-C₆deuteroalkyl, C₁-C₆hydroxyalkyl, or C₁-C₆aminoalkyl; each of R^{8a}, R^{8b}, R^{8c}, and R^{8d} is independently hydrogen, deuterium, halogen, —CN, —NO₂, —OH, —OR^{a}, —OC(═O)R^{a}, —OC(═O)OR, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

Also provided are compounds of Formula (II), or a pharmaceutically acceptable salt, solvate, or stereoisomer thereof:

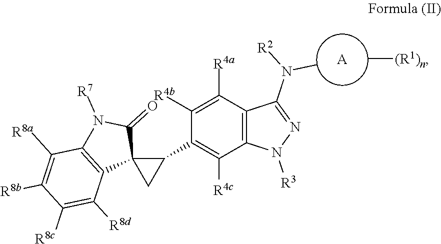

wherein:
Ring A is C₆-C₁₀aryl or heteroaryl, C₃-C₁₀cycloalkyl, and heterocycloalkyl;
each R¹ is independently halogen, —CN, —OH, —OR^{a}, C₁-C₆alkyl, C₁-C₆haloalkyl, —OC₁-C₆haloalkyl, C₁-C₆hydroxyalkyl, C₁-C₆aminoalkyl, C₁-C₆heteroalkyl, C₃-C₁₀cycloalkyl, heterocycloalkyl, C₆-C₁₀aryl, or heteroaryl; wherein each of the C₁-C₆alkyl, C₃-C₁₀cycloalkyl, heterocycloalkyl, C₆-C₁₀aryl, and heteroaryl is optionally and independently substituted with one or more R^{1a};
each R^{1a} is independently deuterium, halogen, —CN, —NO₂, —OH, —OR^{a}, —OC(═O)R^{a}, —OC(═O)OR, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
n is 1, 2, 3, 4, 5, 6, 7, or 8;
R² is hydrogen or C₁-C₆alkyl;
R³ is hydrogen or C₁-C₆alkyl;
R^{4a}, R^{4b}, and R^{4c} are each independently hydrogen, deuterium, or halogen;
R⁷ is hydrogen or C₁-C₆alkyl;
each of R^{8a}, R^{8b}, R^{8c}, and R^{8d} is independently hydrogen, deuterium, halogen, or —OR^{a};
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

In another embodiment are provided compounds of Formula (III), or a pharmaceutically acceptable salt, solvate, or stereoisomer thereof:

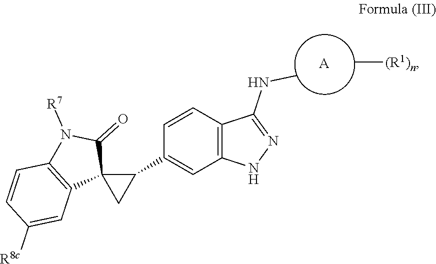

wherein:
Ring A is heteroaryl;
each R¹ is independently halogen, —CN, —OH, —OR^{a}, C₁-C₆alkyl, C₁-C₆haloalkyl, —OC₁-C₆haloalkyl, C₃-C₁₀cycloalkyl, or heterocycloalkyl; wherein each of the C₁-C₆alkyl, C₃-C₁₀cycloalkyl, and heterocycloalkyl is optionally and independently substituted with one or more R^{1a};
each R^{1a} is independently deuterium, —OH, —OR, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
n is 1, 2, 3, 4, 5, 6, 7, or 8;
R⁷ is hydrogen or C₁-C₆alkyl;
R^{8c} is halogen or —OR^{a}; and
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

Further provided herein are compounds of Formula (IV), or a pharmaceutically acceptable salt, solvate, or stereoisomer thereof

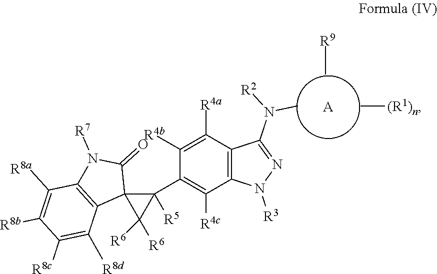

wherein:
Ring A is C₆-C₁₀aryl or heteroaryl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, —P(O)(R^{a})₂, —P(O)₂(R^{a})₂, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3, 4, 5, 6, 7, or 8;
R² is hydrogen, C₁-C₆alkyl, C₁-C₆haloalkyl, or C₁-C₆deuteroalkyl;
R³ is hydrogen, C₁-C₆alkyl, C₁-C₆haloalkyl, or C₁-C₆deuteroalkyl;
each of R^{4a}, R^{4b}, and R^{4c} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R⁶ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁷ is hydrogen, C₁-C₆alkyl, C₁-C₆haloalkyl, C₁-C₆deuteroalkyl, C₁-C₆hydroxyalkyl, or C₁-C₆aminoalkyl; each of R^{8a}, R^{8b}, R^{8c}, and R^{8d} is independently hydrogen, deuterium, halogen, —CN, —NO₂, —OH, —OR^{a}, —OC(═O)R^{a}, —OC(═O)OR, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
R⁹ is heteroaryl optionally substituted with one or more R^{1a}, or oxetanyl substituted with one or more R^{1a};
each R^{a} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆alkoxy, C₁-C₆aminoalkyl, C₁-C₆alkylamino, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
provided the compound of Formula (IV) is not

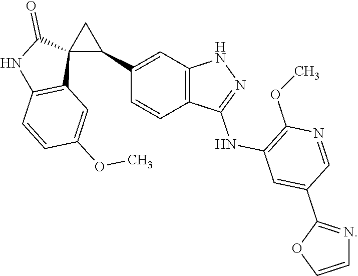 [SMILES: COc1ccc2c(c1)[C@]1(C[C@H]1c1ccc3c(Nc4cc(-c5ncco5)cnc4OC)n[nH]c3c1)C(=O)N2]

In some embodiments are compounds of Formula (IV), or a pharmaceutically acceptable salt, solvate, or stereoisomer thereof, wherein Ring A is C₆-C₁₀aryl. In some embodiments are compounds of Formula (IV), or a pharmaceutically acceptable salt, solvate, or stereoisomer thereof, wherein Ring A is phenyl. In some embodiments are compounds of Formula (IV), or a pharmaceutically acceptable salt, solvate, or stereoisomer thereof, wherein Ring A is heteroaryl. In some embodiments are compounds of Formula (IV), or a pharmaceutically acceptable salt, solvate, or stereoisomer thereof, wherein Ring A is furanyl, pyrrolyl, thiophenyl, oxazolyl, imidazolyl, thiazolyl, pyrazolyl, isoxazolyl, isothiazolyl, triazolyl, pyridinyl, pyrazinyl, pyrimidinyl, or pyridazinyl. In some embodiments are compounds of Formula (IV), or a pharmaceutically acceptable salt, solvate, or stereoisomer thereof, wherein Ring A is pyrazolyl, pyridinyl, pyrazinyl, pyrimidinyl, or pyridazinyl. In some embodiments are compounds of Formula (IV), or a pharmaceutically acceptable salt, solvate, or stereoisomer thereof, wherein Ring A is pyrazolyl, pyridinyl, pyrazinyl, or pyrimidinyl. In some embodiments are compounds of Formula (IV), or a pharmaceutically acceptable salt, solvate, or stereoisomer thereof, wherein Ring A is pyrazolyl. In some embodiments are compounds of Formula (IV), or a pharmaceutically acceptable salt, solvate, or stereoisomer thereof, wherein Ring A is 1-pyrazolyl, 3-pyrazolyl, 4-pyrazolyl, or 5-pyrazolyl. In some embodiments are compounds of Formula (IV), or a pharmaceutically acceptable salt, solvate, or stereoisomer thereof, wherein Ring A is 1-pyrazolyl. In some embodiments are compounds of Formula (IV), or a pharmaceutically acceptable salt, solvate, or stereoisomer thereof, wherein Ring A is 3-pyrazolyl. In some embodiments are compounds of Formula (IV), or a pharmaceutically acceptable salt, solvate, or stereoisomer thereof, wherein Ring A is 4-pyrazolyl. In some embodiments are compounds of Formula (IV), or a pharmaceutically acceptable salt, solvate, or stereoisomer thereof, wherein Ring A is 5-pyrazolyl. In some embodiments are compounds of Formula (IV), or a pharmaceutically acceptable salt, solvate, or stereoisomer thereof, wherein Ring A is pyridinyl. In some embodiments are compounds of Formula (IV), or a pharmaceutically acceptable salt, solvate, or stereoisomer thereof, wherein Ring A is 2-pyridinyl, 3-pyridinyl, 4-pyridinyl, 5-pyridinyl, or 6-pyridinyl. In some embodiments are compounds of Formula (IV), or a pharmaceutically acceptable salt, solvate, or stereoisomer thereof, wherein Ring A is 2-pyridinyl. In some embodiments are compounds of Formula (IV), or a pharmaceutically acceptable salt, solvate, or stereoisomer thereof, wherein Ring A is 3-pyridinyl. In some embodiments are compounds of Formula (IV), or a pharmaceutically acceptable salt, solvate, or stereoisomer thereof, wherein Ring A is 4-pyridinyl. In some embodiments are compounds of Formula (IV), or a pharmaceutically acceptable salt, solvate, or stereoisomer thereof, wherein Ring A is 5-pyridinyl. In some embodiments are compounds of Formula (IV), or a pharmaceutically acceptable salt, solvate, or stereoisomer thereof, wherein Ring A is 6-pyridinyl. In some embodiments are compounds of Formula (IV), or a pharmaceutically acceptable salt, solvate, or stereoisomer thereof, wherein Ring A is pyrazinyl. In some embodiments are compounds of Formula (IV), or a pharmaceutically acceptable salt, solvate, or stereoisomer thereof, wherein Ring A is 2-pyrazinyl, 3-pyrazinyl, 5-pyrazinyl, or 6-pyrazinyl. In some embodiments are compounds of Formula (IV), or a pharmaceutically acceptable salt, solvate, or stereoisomer thereof, wherein Ring A is 2-pyrazinyl. In some embodiments are compounds of Formula (IV), or a pharmaceutically acceptable salt, solvate, or stereoisomer thereof, wherein Ring A is 3-pyrazinyl. In some embodiments are compounds of Formula (IV), or a pharmaceutically acceptable salt, solvate, or stereoisomer thereof, wherein Ring A is 5-pyrazinyl. In some embodiments are compounds of Formula (IV), or a pharmaceutically acceptable salt, solvate, or stereoisomer thereof, wherein Ring A is 6-pyrazinyl. In some embodiments are compounds of Formula (IV), or a pharmaceutically acceptable salt, solvate, or stereoisomer thereof, wherein Ring A is pyrimidinyl. In some embodiments are compounds of Formula (IV), or a pharmaceutically acceptable salt, solvate, or stereoisomer thereof, wherein Ring A is 2-pyrimidinyl, 4-pyrimidinyl, 5-pyrimidinyl, or 6-pyrimidinyl. In some embodiments are compounds of Formula (IV), or a pharmaceutically acceptable salt, solvate, or stereoisomer thereof, wherein Ring A is 2-pyrimidinyl. In some embodiments are compounds of Formula (IV), or a pharmaceutically acceptable salt, solvate, or stereoisomer thereof, wherein Ring A is 4-pyrimidinyl. In some embodiments are compounds of Formula (IV), or a pharmaceutically acceptable salt, solvate, or stereoisomer thereof, wherein Ring A is 5-pyrimidinyl. In some embodiments are compounds of Formula (IV), or a pharmaceutically acceptable salt, solvate, or stereoisomer thereof, wherein Ring A is 6-pyrimidinyl. In some embodiments are compounds of Formula (IV), or a pharmaceutically acceptable salt, solvate, or stereoisomer thereof, wherein Ring A is pyridazinyl. In some embodiments are compounds of Formula (IV), or a pharmaceutically acceptable salt, solvate, or stereoisomer thereof, wherein Ring A is 3-pyridazinyl, 4-pyridazinyl, 5-pyridazinyl, or 6-pyridazinyl. In some embodiments are compounds of Formula (IV), or a pharmaceutically acceptable salt, solvate, or stereoisomer thereof, wherein Ring A is 3-pyridazinyl. In some embodiments are compounds of Formula (IV), or a pharmaceutically acceptable salt, solvate, or stereoisomer thereof, wherein Ring A is 4-pyridazinyl. In some embodiments are compounds of Formula (IV), or a pharmaceutically acceptable salt, solvate, or stereoisomer thereof, wherein Ring A is 5-pyridazinyl. In some embodiments are compounds of Formula (IV), or a pharmaceutically acceptable salt, solvate, or stereoisomer thereof, wherein Ring A is 6-pyridazinyl. In some embodiments are compounds of Formula (IV), or a pharmaceutically acceptable salt, solvate, or stereoisomer thereof, wherein each R¹ is independently methyl, ethyl, trifluoromethyl, methoxy, ethoxy, methanesulfonyl, ethanesulfonyl, acetyl, or dimethylamino. In some embodiments are compounds of Formula (IV), or a pharmaceutically acceptable salt, solvate, or stereoisomer thereof, wherein n is 1, 2, or 3. In some embodiments are compounds of Formula (IV), or a pharmaceutically acceptable salt, solvate, or stereoisomer thereof, wherein n is 1. In some embodiments are compounds of Formula (IV), or a pharmaceutically acceptable salt, solvate, or stereoisomer thereof, wherein n is 2. In some embodiments are compounds of Formula (IV), or a pharmaceutically acceptable salt, solvate, or stereoisomer thereof, wherein n is 3. In some embodiments are compounds of Formula (IV), or a pharmaceutically acceptable salt, solvate, or stereoisomer thereof, wherein R² is hydrogen. In some embodiments are compounds of Formula (IV), or a pharmaceutically acceptable salt, solvate, or stereoisomer thereof, wherein R³ is hydrogen. In some embodiments are compounds of Formula (IV), or a pharmaceutically acceptable salt, solvate, or stereoisomer thereof, wherein R^{4a}, R^{4b}, and R^{4c} are independently hydrogen or halogen. In some embodiments are compounds of Formula (IV), or a pharmaceutically acceptable salt, solvate, or stereoisomer thereof, wherein R^{4a} is halogen and R^{4b} and R^{4c} are hydrogen. In some embodiments are compounds of Formula (IV), or a pharmaceutically acceptable salt, solvate, or stereoisomer thereof, wherein R^{4a} and R^{4c} are hydrogen and R^{4b} is halogen. In some embodiments are compounds of Formula (IV), or a pharmaceutically acceptable salt, solvate, or stereoisomer thereof, wherein R^{4a} and R^{4b} are hydrogen and R^{4c} is halogen. In some embodiments are compounds of Formula (IV), or a pharmaceutically acceptable salt, solvate, or stereoisomer thereof, wherein R^{4a} and R^{4b} are halogen and R^{4c} is hydrogen. In some embodiments are compounds of Formula (IV), or a pharmaceutically acceptable salt, solvate, or stereoisomer thereof, wherein R^{4a} and R^{4c} are halogen and R^{4b} is hydrogen. In some embodiments are compounds of Formula (IV), or a pharmaceutically acceptable salt, solvate, or stereoisomer thereof, wherein R^{4a}, R^{4b} and R^{4c} are halogen. In some embodiments are compounds of Formula (IV), or a pharmaceutically acceptable salt, solvate, or stereoisomer thereof, wherein R^{4a}, R^{4b}, and R^{4c} are hydrogen. In some embodiments are compounds of Formula (IV), or a pharmaceutically acceptable salt, solvate, or stereoisomer thereof, wherein R⁵ is hydrogen. In some embodiments are compounds of Formula (IV), or a pharmaceutically acceptable salt, solvate, or stereoisomer thereof, wherein each R⁶ is hydrogen. In some embodiments are compounds of Formula (IV), or a pharmaceutically acceptable salt, solvate, or stereoisomer thereof, wherein R⁷ is hydrogen or C₁-C₆alkyl. In some embodiments are compounds of Formula (IV), or a pharmaceutically acceptable salt, solvate, or stereoisomer thereof, wherein R⁶ is hydrogen. In some embodiments are compounds of Formula (IV), or a pharmaceutically acceptable salt, solvate, or stereoisomer thereof, wherein R⁷ is C₁-C₆alkyl. In some embodiments are compounds of Formula (IV), or a pharmaceutically acceptable salt, solvate, or stereoisomer thereof, wherein each of R^{8a}, R^{8b}, R^{8c}, and R^{8d} is independently hydrogen, halogen, or —OR^{a}. In some embodiments are compounds of Formula (IV), or a pharmaceutically acceptable salt, solvate, or stereoisomer thereof, wherein each of R^{a}, R^{8b}, and R^{8c} are hydrogen and R^{8c} is hydrogen, halogen, or —OR^{a}. In some embodiments are compounds of Formula (IV), or a pharmaceutically acceptable salt, solvate, or stereoisomer thereof, wherein R¹ is halogen or —OR^{a}. In some embodiments are compounds of Formula (IV), or a pharmaceutically acceptable salt, solvate, or stereoisomer thereof, wherein R^{8c} is halogen. In some embodiments are compounds of Formula (IV), or a pharmaceutically acceptable salt, solvate, or stereoisomer thereof, wherein R^{8c} is fluoro, chloro, bromo, or iodo. In some embodiments are compounds of Formula (IV), or a pharmaceutically acceptable salt, solvate, or stereoisomer thereof, wherein R^{8c} is —OR^{a}. In some embodiments are compounds of Formula (IV), or a pharmaceutically acceptable salt, solvate, or stereoisomer thereof, wherein R^{a} is C₁-C₆alkyl. In some embodiments are compounds of Formula (IV), or a pharmaceutically acceptable salt, solvate, or stereoisomer thereof, wherein R^{a} is —CH₃. In some embodiments are compounds of Formula (IV), or a pharmaceutically acceptable salt, solvate, or stereoisomer thereof, wherein R⁹ is furanyl, pyrrolyl, thiophenyl, oxazolyl, imidazolyl, thiazolyl, pyrazolyl, isoxazolyl, isothiazolyl, triazolyl, pyridinyl, pyrazinyl, pyrimidinyl, pyridazinyl, or tetrazolyl, each of which is optionally substituted with one or more R^{1a}. In some embodiments are compounds of Formula (IV), or a pharmaceutically acceptable salt, solvate, or stereoisomer thereof, wherein R⁹ is furanyl optionally substituted with one or more R^{1a}. In some embodiments are compounds of Formula (IV), or a pharmaceutically acceptable salt, solvate, or stereoisomer thereof, wherein R⁹ is pyrrolyl optionally substituted with one or more R^{1a}. In some embodiments are compounds of Formula (IV), or a pharmaceutically acceptable salt, solvate, or stereoisomer thereof, wherein R⁹ is thiophenyl optionally substituted with one or more R^{1a}. In some embodiments are compounds of Formula (IV), or a pharmaceutically acceptable salt, solvate, or stereoisomer thereof, wherein R⁹ is oxazolyl optionally substituted with one or more R^{1a}. In some embodiments are compounds of Formula (IV), or a pharmaceutically acceptable salt, solvate, or stereoisomer thereof, wherein R⁹ is imidazolyl optionally substituted with one or more R^{1a}. In some embodiments are compounds of Formula (IV), or a pharmaceutically acceptable salt, solvate, or stereoisomer thereof, wherein R⁹ is thiazolyl optionally substituted with one or more R^{1a}. In some embodiments are compounds of Formula (IV), or a pharmaceutically acceptable salt, solvate, or stereoisomer thereof, wherein R⁹ is pyrazolyl optionally substituted with one or more R^{1a}. In some embodiments are compounds of Formula (IV), or a pharmaceutically acceptable salt, solvate, or stereoisomer thereof, wherein R⁹ is isoxazolyl optionally substituted with one or more R^{1a}. In some embodiments are compounds of Formula (IV), or a pharmaceutically acceptable salt, solvate, or stereoisomer thereof, wherein R⁹ is isothiazolyl optionally substituted with one or more R^{1a}. In some embodiments are compounds of Formula (IV), or a pharmaceutically acceptable salt, solvate, or stereoisomer thereof, wherein R⁹ is triazolyl optionally substituted with one or more R^{1a}. In some embodiments are compounds of Formula (IV), or a pharmaceutically acceptable salt, solvate, or stereoisomer thereof, wherein R⁹ is pyridinyl optionally substituted with one or more R^{1a}. In some embodiments are compounds of Formula (IV), or a pharmaceutically acceptable salt, solvate, or stereoisomer thereof, wherein R⁹ is pyrazinyl optionally substituted with one or more R^{1a}. In some embodiments are compounds of Formula (IV), or a pharmaceutically acceptable salt, solvate, or stereoisomer thereof, wherein R⁹ is pyrimidinyl optionally substituted with one or more R^{1a}. In some embodiments are compounds of Formula (IV), or a pharmaceutically acceptable salt, solvate, or stereoisomer thereof, wherein R⁹ is pyridazinyl optionally substituted with one or more R^{1a}. In some embodiments are compounds of Formula (IV), or a pharmaceutically acceptable salt, solvate, or stereoisomer thereof, wherein R⁹ is tetrazolyl optionally substituted with one or more R^{1a}. In some embodiments are compounds of Formula (IV), or a pharmaceutically acceptable salt, solvate, or stereoisomer thereof, wherein R⁹ is oxetanyl substituted with one or more R^{1a}.

In some embodiments are compounds of Formula (IV), or a pharmaceutically acceptable salt, solvate, or stereoisomer thereof, wherein the compound of Formula (IV) has the structure of Formula (IVa):

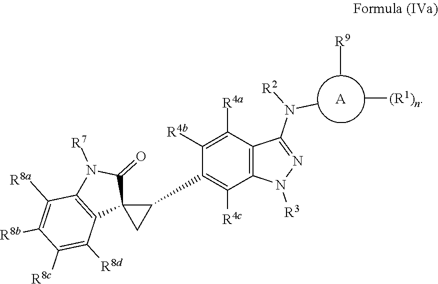

In some embodiments are compounds of Formula (IV), or a pharmaceutically acceptable salt, solvate, or stereoisomer thereof, wherein the compound of Formula (IV) has the structure of Formula (IVb):

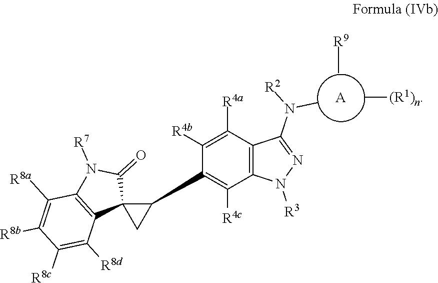

In another embodiment are provided compounds of Formula (V), or a pharmaceutically acceptable salt, solvate, or stereoisomer thereof,

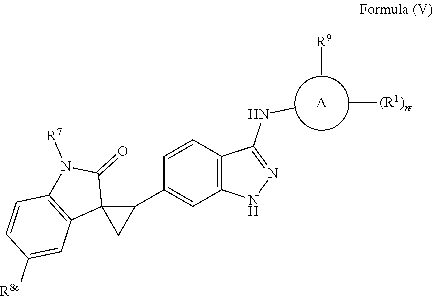

wherein:
Ring A is C₆-C₁₀aryl or heteroaryl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, —P(O)(R^{a})₂, —P(O)₂(R^{a})₂, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3, 4, 5, 6, 7, or 8;
R⁷ is hydrogen, C₁-C₆alkyl, C₁-C₆haloalkyl, C₁-C₆deuteroalkyl, C₁-C₆hydroxyalkyl, or C₁-C₆aminoalkyl;
R^{8c} is hydrogen, deuterium, halogen, —CN, —NO₂, —OH, —OR^{a}, —OC(═O)R^{a}, —OC(═O)OR^{a}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
R⁹ is heteroaryl optionally substituted with one or more R^{1a}, or oxetanyl substituted with one or more R^{1a};
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆alkoxy, C₁-C₆aminoalkyl, C₁-C₆alkylamino, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
provided the compound of Formula (V) is not

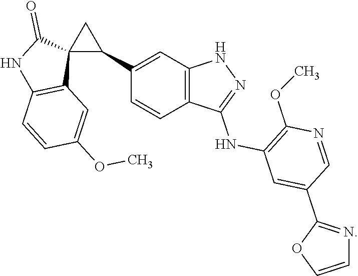 [SMILES: COc1ccc2c(c1)[C@]1(C[C@H]1c1ccc3c(Nc4cc(-c5ncco5)cnc4OC)n[nH]c3c1)C(=O)N2]

In some embodiments are compounds of Formula (V), or a pharmaceutically acceptable salt, solvate, or stereoisomer thereof, wherein Ring A is C₆-C₁₀aryl. In some embodiments are compounds of Formula (V), or a pharmaceutically acceptable salt, solvate, or stereoisomer thereof, wherein Ring A is phenyl. In some embodiments are compounds of Formula (V), or a pharmaceutically acceptable salt, solvate, or stereoisomer thereof, wherein Ring A is heteroaryl. In some embodiments are compounds of Formula (V), or a pharmaceutically acceptable salt, solvate, or stereoisomer thereof, wherein Ring A is pyrazolyl, pyridinyl, pyrazinyl, pyrimidinyl, or pyridazinyl. In some embodiments are compounds of Formula (V), or a pharmaceutically acceptable salt, solvate, or stereoisomer thereof, wherein Ring A is pyrazolyl. In some embodiments are compounds of Formula (V), or a pharmaceutically acceptable salt, solvate, or stereoisomer thereof, wherein Ring A is 1-pyrazolyl, 3-pyrazolyl, 4-pyrazolyl, or 5-pyrazolyl. In some embodiments are compounds of Formula (V), or a pharmaceutically acceptable salt, solvate, or stereoisomer thereof, wherein Ring A is 1-pyrazolyl. In some embodiments are compounds of Formula (V), or a pharmaceutically acceptable salt, solvate, or stereoisomer thereof, wherein Ring A is 3-pyrazolyl. In some embodiments are compounds of Formula (V), or a pharmaceutically acceptable salt, solvate, or stereoisomer thereof, wherein Ring A is 4-pyrazolyl. In some embodiments are compounds of Formula (V), or a pharmaceutically acceptable salt, solvate, or stereoisomer thereof, wherein Ring A is 5-pyrazolyl. In some embodiments are compounds of Formula (V), or a pharmaceutically acceptable salt, solvate, or stereoisomer thereof, wherein Ring A is pyridinyl. In some embodiments are compounds of Formula (V), or a pharmaceutically acceptable salt, solvate, or stereoisomer thereof, wherein Ring A is 2-pyridinyl, 3-pyridinyl, 4-pyridinyl, 5-pyridinyl, or 6-pyridinyl. In some embodiments are compounds of Formula (V), or a pharmaceutically acceptable salt, solvate, or stereoisomer thereof, wherein Ring A is 2-pyridinyl. In some embodiments are compounds of Formula (V), or a pharmaceutically acceptable salt, solvate, or stereoisomer thereof, wherein Ring A is 3-pyridinyl. In some embodiments are compounds of Formula (V), or a pharmaceutically acceptable salt, solvate, or stereoisomer thereof, wherein Ring A is 4-pyridinyl. In some embodiments are compounds of Formula (V), or a pharmaceutically acceptable salt, solvate, or stereoisomer thereof, wherein Ring A is 5-pyridinyl. In some embodiments are compounds of Formula (V), or a pharmaceutically acceptable salt, solvate, or stereoisomer thereof, wherein Ring A is 6-pyridinyl. In some embodiments are compounds of Formula (V), or a pharmaceutically acceptable salt, solvate, or stereoisomer thereof, wherein Ring A is pyrazinyl. In some embodiments are compounds of Formula (V), or a pharmaceutically acceptable salt, solvate, or stereoisomer thereof, wherein Ring A is 2-pyrazinyl, 3-pyrazinyl, 5-pyrazinyl, or 6-pyrazinyl. In some embodiments are compounds of Formula (V), or a pharmaceutically acceptable salt, solvate, or stereoisomer thereof, wherein Ring A is 2-pyrazinyl. In some embodiments are compounds of Formula (V), or a pharmaceutically acceptable salt, solvate, or stereoisomer thereof, wherein Ring A is 3-pyrazinyl. In some embodiments are compounds of Formula (V), or a pharmaceutically acceptable salt, solvate, or stereoisomer thereof, wherein Ring A is 5-pyrazinyl. In some embodiments are compounds of Formula (V), or a pharmaceutically acceptable salt, solvate, or stereoisomer thereof, wherein Ring A is 6-pyrazinyl. In some embodiments are compounds of Formula (V), or a pharmaceutically acceptable salt, solvate, or stereoisomer thereof, wherein Ring A is pyrimidinyl. In some embodiments are compounds of Formula (V), or a pharmaceutically acceptable salt, solvate, or stereoisomer thereof, wherein Ring A is 2-pyrimidinyl, 4-pyrimidinyl, 5-pyrimidinyl, or 6-pyrimidinyl. In some embodiments are compounds of Formula (V), or a pharmaceutically acceptable salt, solvate, or stereoisomer thereof, wherein Ring A is 2-pyrimidinyl. In some embodiments are compounds of Formula (V), or a pharmaceutically acceptable salt, solvate, or stereoisomer thereof, wherein Ring A is 4-pyrimidinyl. In some embodiments are compounds of Formula (V), or a pharmaceutically acceptable salt, solvate, or stereoisomer thereof, wherein Ring A is 5-pyrimidinyl. In some embodiments are compounds of Formula (V), or a pharmaceutically acceptable salt, solvate, or stereoisomer thereof, wherein Ring A is 6-pyrimidinyl. In some embodiments are compounds of Formula (V), or a pharmaceutically acceptable salt, solvate, or stereoisomer thereof, wherein Ring A is pyridazinyl. In some embodiments are compounds of Formula (V), or a pharmaceutically acceptable salt, solvate, or stereoisomer thereof, wherein Ring A is 3-pyridazinyl, 4-pyridazinyl, 5-pyridazinyl, or 6-pyridazinyl. In some embodiments are compounds of Formula (V), or a pharmaceutically acceptable salt, solvate, or stereoisomer thereof, wherein Ring A is 3-pyridazinyl. In some embodiments are compounds of Formula (V), or a pharmaceutically acceptable salt, solvate, or stereoisomer thereof, wherein Ring A is 4-pyridazinyl. In some embodiments are compounds of Formula (V), or a pharmaceutically acceptable salt, solvate, or stereoisomer thereof, wherein Ring A is 5-pyridazinyl. In some embodiments are compounds of Formula (V), or a pharmaceutically acceptable salt, solvate, or stereoisomer thereof, wherein Ring A is 6-pyridazinyl. In some embodiments are compounds of Formula (V), or a pharmaceutically acceptable salt, solvate, or stereoisomer thereof, wherein R⁹ is furanyl, pyrrolyl, thiophenyl, oxazolyl, imidazolyl, thiazolyl, pyrazolyl, isoxazolyl, isothiazolyl, triazolyl, pyridinyl, pyrazinyl, pyrimidinyl, pyridazinyl, or tetrazolyl, each of which is optionally substituted with one or more R^{1a}. In some embodiments are compounds of Formula (V), or a pharmaceutically acceptable salt, solvate, or stereoisomer thereof, wherein R⁹ is furanyl optionally substituted with one or more R^{1a}. In some embodiments are compounds of Formula (V), or a pharmaceutically acceptable salt, solvate, or stereoisomer thereof, wherein R⁹ is pyrrolyl optionally substituted with one or more R^{1a}. In some embodiments are compounds of Formula (V), or a pharmaceutically acceptable salt, solvate, or stereoisomer thereof, wherein R⁹ is thiophenyl optionally substituted with one or more R^{1a}. In some embodiments are compounds of Formula (V), or a pharmaceutically acceptable salt, solvate, or stereoisomer thereof, wherein R⁹ is oxazolyl optionally substituted with one or more R^{1a}. In some embodiments are compounds of Formula (V), or a pharmaceutically acceptable salt, solvate, or stereoisomer thereof, wherein R⁹ is imidazolyl optionally substituted with one or more R^{1a}. In some embodiments are compounds of Formula (V), or a pharmaceutically acceptable salt, solvate, or stereoisomer thereof, wherein R⁹ is thiazolyl optionally substituted with one or more R^{1a}. In some embodiments are compounds of Formula (V), or a pharmaceutically acceptable salt, solvate, or stereoisomer thereof, wherein R⁹ is pyrazolyl optionally substituted with one or more R^{1a}. In some embodiments are compounds of Formula (V), or a pharmaceutically acceptable salt, solvate, or stereoisomer thereof, wherein R⁹ is isoxazolyl optionally substituted with one or more R^{1a}. In some embodiments are compounds of Formula (V), or a pharmaceutically acceptable salt, solvate, or stereoisomer thereof, wherein R⁹ is isothiazolyl optionally substituted with one or more R^{1a}. In some embodiments are compounds of Formula (V), or a pharmaceutically acceptable salt, solvate, or stereoisomer thereof, wherein R⁹ is triazolyl optionally substituted with one or more R^{1a}. In some embodiments are compounds of Formula (V), or a pharmaceutically acceptable salt, solvate, or stereoisomer thereof, wherein R⁹ is pyridinyl optionally substituted with one or more R^{1a}. In some embodiments are compounds of Formula (V), or a pharmaceutically acceptable salt, solvate, or stereoisomer thereof, wherein R⁹ is pyrazinyl optionally substituted with one or more R^{1a}. In some embodiments are compounds of Formula (V), or a pharmaceutically acceptable salt, solvate, or stereoisomer thereof, wherein R⁹ is pyrimidinyl optionally substituted with one or more R^{1a}. In some embodiments are compounds of Formula (V), or a pharmaceutically acceptable salt, solvate, or stereoisomer thereof, wherein R⁹ is pyridazinyl optionally substituted with one or more R^{1a}. In some embodiments are compounds of Formula (V), or a pharmaceutically acceptable salt, solvate, or stereoisomer thereof, wherein R⁹ is tetrazolyl optionally substituted with one or more R^{1a}. In some embodiments are compounds of Formula (V), or a pharmaceutically acceptable salt, solvate, or stereoisomer thereof, wherein R⁹ is oxetanyl substituted with one or more R^{1a}. In some embodiments are compounds of Formula (V), or a pharmaceutically acceptable salt, solvate, or stereoisomer thereof, wherein R⁷ is hydrogen. In some embodiments are compounds of Formula (V), or a pharmaceutically acceptable salt, solvate, or stereoisomer thereof, wherein R^{8c} is halogen or —OR^{a}. In some embodiments are compounds of Formula (V), or a pharmaceutically acceptable salt, solvate, or stereoisomer thereof, wherein R^{8c} is halogen. In some embodiments are compounds of Formula (V), or a pharmaceutically acceptable salt, solvate, or stereoisomer thereof, wherein R^{8c} is fluoro, chloro, bromo, or iodo. In some embodiments are compounds of Formula (V), or a pharmaceutically acceptable salt, solvate, or stereoisomer thereof, wherein R^{8c} is —OR^{a}. In some embodiments are compounds of Formula (V), or a pharmaceutically acceptable salt, solvate, or stereoisomer thereof, wherein R^{a} is C₁-C₆alkyl. In some embodiments are compounds of Formula (V), or a pharmaceutically acceptable salt, solvate, or stereoisomer thereof, wherein R^{a} is —CH₃. In some embodiments are compounds of Formula (V), or a pharmaceutically acceptable salt, solvate, or stereoisomer thereof, wherein n is 0, 1, or 2. In some embodiments are compounds of Formula (V), or a pharmaceutically acceptable salt, solvate, or stereoisomer thereof, wherein n is 0. In some embodiments are compounds of Formula (V), or a pharmaceutically acceptable salt, solvate, or stereoisomer thereof, wherein n is 1. In some embodiments are compounds of Formula (V), or a pharmaceutically acceptable salt, solvate, or stereoisomer thereof, wherein n is 2. In some embodiments are compounds of Formula (V), or a pharmaceutically acceptable salt, solvate, or stereoisomer thereof, wherein each R¹ is independently methyl, ethyl, trifluoromethyl, methoxy, ethoxy, methanesulfonyl, ethanesulfonyl, acetyl, or dimethylamino.

In some embodiments are compounds of Formula (V), or a pharmaceutically acceptable salt, solvate, or stereoisomer thereof, wherein the compound of Formula (V) has the structure of Formula (Va):

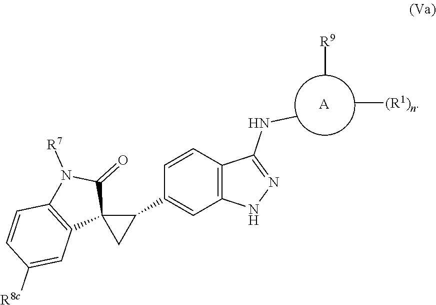

In some embodiments are compounds of Formula (V), or a pharmaceutically acceptable salt, solvate, or stereoisomer thereof, wherein the compound of Formula (V) has the structure of Formula (Vb):

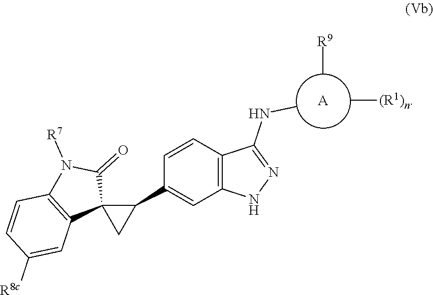

Also provided herein is a compound, or a pharmaceutically acceptable salt, solvate, or stereoisomer thereof, selected from the group consisting of 6-methoxy-5-({6-[(1R,2S)-5′-methoxy-2′-oxo-1′,2′-dihydrospiro[cyclopropane-1,3′-indol]-2-yl]-1H-indazol-3-yl}amino)-2,3-dihydro-1H-1-benzothiophene-1,1-dione; (1R,2S)-2-{3-[4-(methanesulfonyl)-2-methoxyanilino]-1H-indazol-6-yl}-5′-methoxyspiro[cyclopropane-1,3′-indol]-2′(1′H)-one; (1R,2S)-2-[3-(4-acetyl-2-methoxyanilino)-1H-indazol-6-yl]-5′-methoxyspiro[cyclopropane-1,3′-indol]-2′(1′H)-one; (1R,2S)-5′-methoxy-2-(3-{[3-methoxy-6-(1,3-oxazol-2-yl)pyridin-2-yl]amino}-1H-indazol-6-yl)spiro[cyclopropane-1,3′-indol]-2′(1′H)-one; (1R,2S)-2-{3-[4-(ethanesulfonyl)-2-methoxyanilino]-1H-indazol-6-yl}-5′-methoxyspiro[cyclopropane-1,3′-indol]-2′(1′H)-one; 6-methoxy-5-({6-[(1R,2S)-5′-methoxy-2′-oxo-1′,2′-dihydrospiro[cyclopropane-1,3′-indol]-2-yl]-1H-indazol-3-yl}amino)-1H-1-benzothiophene-1,1-dione; (1R,2S)-2-{3-[2-ethoxy-4-(pyrazin-2-yl)anilino]-1H-indazol-6-yl}-5′-methoxyspiro[cyclopropane-1,3′-indol]-2′(1′H)-one; (1R,2S)-2-{3-[(3-ethoxyquinolin-2-yl)amino]-1H-indazol-6-yl}-5′-methoxyspiro[cyclopropane-1,3′-indol]-2′(1′H)-one; (1R,2S)-2-(3-{4-[3-(dimethylamino)oxetan-3-yl]-2-methoxyanilino}-1H-indazol-6-yl)-5′-methoxyspiro[cyclopropane-1,3′-indol]-2′(1′H)-one; (1R,2S)-2-{3-[2-ethoxy-4-(1H-1,2,4-triazol-1-yl)anilino]-1H-indazol-6-yl}-5′-methoxyspiro[cyclopropane-1,3′-indol]-2′(1′H)-one; (1R,2S)-5′-methoxy-2-{3-[2-methoxy-5-(1-methyl-JH-pyrazol-4-yl)anilino]-1H-indazol-6-yl}spiro[cyclopropane-1,3′-indol]-2′(1′H)-one; 7-ethoxy-6-((6-((1R,2S)-5′-methoxy-2′-oxospiro[cyclopropane-1,3′-indolin]-2-yl)-1H-indazol-3-yl)amino)quinoline 1-oxide; (1R,2S)-5′-methoxy-2-{3-[2-methoxy-5-(1,3-oxazol-2-yl)anilino]-1H-indazol-6-yl}spiro[cyclopropane-1,3′-indol]-2′(1′H)-one; (1R,2S)-5′-methoxy-2-{3-[2-methoxy-5-(1,3-thiazol-2-yl)anilino]-1H-indazol-6-yl}spiro[cyclopropane-1,3′-indol]-2′(1′H)-one; (1R,2S)-5′-methoxy-2-{3-[2-methoxy-5-(1,3-thiazol-4-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-4-yl)anilino]-1H-indazol-6-yl}spiro[cyclopropane-1,3′-indol]-2′(1′H)-one; (1R,2S)-5′-methoxy-2-{3-[2-methoxy-4-(2-methyl-2H-tetrazol-5-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-5-yl)anilino]-1H-indazol-6-yl}spiro[cyclopropane-1,3′-indol]-2′(1′H)-one; (1R,2S)-5′-methoxy-2-(3-{[3-methoxy-6-(1,3-oxazol-2-yl)pyrazin-2-yl]amino}-1H-indazol-6-yl)spiro[cyclopropane-1,3′-indol]-2′(1′H)-one; (1R,2S)-2-(3-{[2-(dimethylamino)-5-methoxypyrimidin-4-yl]amino}-1H-indazol-6-yl)-5′-methoxyspiro[cyclopropane-1,3′-indol]-2′(1′H)-one; (1R,2S)-5′-methoxy-2-{3-[2-methoxy-5-(2-methyl-2H-tetrazol-5-yl)anilino]-1H-indazol-6-yl}spiro[cyclopropane-1,3′-indol]-2′(1′H)-one; and (1R,2S)-2-(3-{5-[3-(dimethylamino)oxetan-3-yl]-2-methoxyanilino}-1H-indazol-6-yl)-5′-methoxyspiro[cyclopropane-1,3′-indol]-2′(1′H)-one.

Also provided herein is a compound, or a pharmaceutically acceptable salt, solvate, or stereoisomer thereof, selected from the group consisting of 6-methoxy-5-({6-[(1R,2S)-5′-methoxy-2′-oxo-1′,2′-dihydrospiro[cyclopropane-1,3′-indol]-2-yl]-1H-indazol-3-yl}amino)-2,3-dihydro-1H-1-benzothiophene-1,1-dione; (1R,2S)-2-{3-[4-(methanesulfonyl)-2-methoxyanilino]-1H-indazol-6-yl}-5′-methoxyspiro[cyclopropane-1,3′-indol]-2′(1′H)-one; (1R,2S)-2-[3-(4-acetyl-2-methoxyanilino)-1H-indazol-6-yl]-5′-methoxyspiro[cyclopropane-1,3′-indol]-2′(1′H)-one; (1R,2S)-2-{3-[4-(ethanesulfonyl)-2-methoxyanilino]-1H-indazol-6-yl}-5′-methoxyspiro[cyclopropane-1,3′-indol]-2′(1′H)-one; 6-methoxy-5-({6-[(1R,2S)-5′-methoxy-2′-oxo-1′,2′-dihydrospiro[cyclopropane-1,3′-indol]-2-yl]-1H-indazol-3-yl}amino)-1H-1-benzothiophene-1,1-dione; (1R,2S)-2-{3-[(3-ethoxyquinolin-2-yl)amino]-1H-indazol-6-yl}-5′-methoxyspiro[cyclopropane-1,3′-indol]-2′(1′H)-one; 7-ethoxy-6-((6-((1R,2S)-5′-methoxy-2′-oxospiro[cyclopropane-1,3′-indolin]-2-yl)-1H-indazol-3-yl)amino)quinoline 1-oxide; (1R,2S)-2-(3-{[2-(dimethylamino)-5-methoxypyrimidin-4-yl]amino}-1H-indazol-6-yl)-5′-methoxyspiro[cyclopropane-1,3′-indol]-2′(1′H)-one; and (1R,2S)-5′-methoxy-2-{3-[2-methoxy-5-(2-methyl-2H-tetrazol-5-yl)anilino]-1H-indazol-6-yl}spiro[cyclopropane-1,3′-indol]-2′(1′H)-one.

Also provided herein is a compound, or a pharmaceutically acceptable salt, solvate, or stereoisomer thereof, selected from the group consisting of (1R,2S)-5′-methoxy-2-(3-{[3-methoxy-6-(1,3-oxazol-2-yl)pyridin-2-yl]amino}-1H-indazol-6-yl)spiro[cyclopropane-1,3′-indol]-2′(1′H)-one; (1R,2S)-2-{3-[2-ethoxy-4-(pyrazin-2-yl)anilino]-1H-indazol-6-yl}-5′-methoxyspiro[cyclopropane-1,3′-indol]-2′(1′H)-one; (1R,2S)-2-(3-{4-[3-(dimethylamino)oxetan-3-yl]-2-methoxyanilino}-1H-indazol-6-yl)-5′-methoxyspiro[cyclopropane-1,3′-indol]-2′(1′H)-one; (1R,2S)-2-{3-[2-ethoxy-4-(1H-1,2,4-triazol-1-yl)anilino]-1H-indazol-6-yl}-5′-methoxyspiro[cyclopropane-1,3′-indol]-2′(1′H)-one; (1R,2S)-5′-methoxy-2-{3-[2-methoxy-5-(1-methyl-1H-pyrazol-4-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-2-yl)anilino]-1H-indazol-6-yl}spiro[cyclopropane-1,3′-indol]-2′(1′H)-one; (1R,2S)-5′-methoxy-2-{3-[2-methoxy-5-(1,3-thiazol-2-yl)anilino]-1H-indazol-6-yl}spiro[cyclopropane-1,3′-indol]-2′(1′H)-one; (1R,2S)-5′-methoxy-2-{3-[2-methoxy-5-(1,3-thiazol-4-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-4-yl)anilino]-1H-indazol-6-yl}spiro[cyclopropane-1,3′-indol]-2′(1′H)-one; (1R,2S)-5′-methoxy-2-{3-[2-methoxy-4-(2-methyl-2H-tetrazol-5-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-5-yl)anilino]-1H-indazol-6-yl}spiro[cyclopropane-1,3′-indol]-2′(1′H)-one; (1R,2S)-5′-methoxy-2-(3-{[3-methoxy-6-(1,3-oxazol-2-yl)pyrazin-2-yl]amino}-1H-indazol-6-yl)spiro[cyclopropane-1,3′-indol]-2′(1′H)-one; and (1R,2S)-2-(3-{5-[3-(dimethylamino)oxetan-3-yl]-2-methoxyanilino}-1H-indazol-6-yl)-5′-methoxyspiro[cyclopropane-1,3′-indol]-2′(1′H)-one.

Also provided herein is a compound, or a pharmaceutically acceptable salt, solvate, or stereoisomer thereof, selected from the group consisting of (1R,2S)-2-{3-[2-ethoxy-4-(methanesulfonyl)anilino]-1H-indazol-6-yl}-5‘-methoxy-l’-methylspiro[cyclopropane-1,3′-indol]-2′(1′H)-one; (1R,2S)-5′-methoxy-2-(3-{[3-methoxy-6-(1-methyl-1H-pyrazol-4-yl)pyrazin-2-yl]amino}-1H-indazol-6-yl)spiro[cyclopropane-1,3′-indol]-2′(1′H)-one; (1R,2S)-5′-methoxy-2-{3-[(5-methoxy[2,5′-bipyrimidin]-4-yl)amino]-1H-indazol-6-yl}spiro[cyclopropane-1,3′-indol]-2′(1′H)-one; (1R,2S)-5′-methoxy-2-(3-{[2-methoxy-5-(1-methyl-1H-pyrazol-4-yl)pyridin-3-yl]amino}-1H-indazol-6-yl)spiro[cyclopropane-1,3′-indol]-2′(1′H)-one; (1R,2S)-5′-methoxy-2-(3-{[5-methoxy-2-(1-methyl-1H-pyrazol-4-yl)pyridin-4-yl]amino}-1H-indazol-6-yl)spiro[cyclopropane-1,3′-indol]-2′(1′H)-one; (1R,2S)-2-(3-{[3-ethoxy-6-(1,3-thiazol-2-yl)pyridin-2-yl]amino}-1H-indazol-6-yl)-5′-methoxyspiro[cyclopropane-1,3′-indol]-2′(1′H)-one; (1R,2S)-2-{3-[2-ethoxy-4-(1-methyl-1H-imidazol-4-yl)anilino]-1H-indazol-6-yl}-5′-methoxyspiro[cyclopropane-1,3′-indol]-2′(1′H)-one; (1R,2S)-5′-methoxy-2-{3-[2-methoxy-5-(1,2-thiazol-3-yl)anilino]-1H-indazol-6-yl}spiro[cyclopropane-1,3′-indol]-2′(1′H)-one; (1R,2S)-5′-methoxy-2-(3-{[5-methoxy-2-(1-methyl-1H-pyrazol-4-yl)pyrimidin-4-yl]amino}-1H-indazol-6-yl)spiro[cyclopropane-1,3′-indol]-2′(1′H)-one; (1R,2S)-5′-methoxy-2-{3-[2-methoxy-5-(1,2-oxazol-3-yl)anilino]-1H-indazol-6-yl}spiro[cyclopropane-1,3′-indol]-2′(1′H)-one; (1R,2S)-2-{3-[2-ethoxy-5-(1,3-thiazol-2-yl)anilino]-1H-indazol-6-yl}-5′-methoxyspiro[cyclopropane-1,3′-indol]-2′(1′H)-one; (1R,2S)-2-(3-{[5-ethoxy-2-(1,3-thiazol-2-yl)pyridin-4-yl]amino}-1H-indazol-6-yl)-5′-methoxyspiro[cyclopropane-1,3′-indol]-2′(1′H)-one; (1R,2S)-2-(3-{[3-ethoxy-6-(1,3-thiazol-2-yl)pyrazin-2-yl]amino}-1H-indazol-6-yl)-5′-methoxyspiro[cyclopropane-1,3′-indol]-2′(1′H)-one; (1R,2S)-5′-methoxy-2-{3-[2-methoxy-5-(3-methoxy-1-methyl-1H-pyrazol-4-yl)anilino]-1H-indazol-6-yl}spiro[cyclopropane-1,3′-indol]-2′(1′H)-one; (1R,2S)-2-{3-[2-ethoxy-5-(1-methyl-1H-pyrazol-4-yl)anilino]-1H-indazol-6-yl}-5′-methoxyspiro[cyclopropane-1,3′-indol]-2′(1′H)-one; (1R,2S)-2-(3-{[3-ethoxy-5-(1H-1,2,4-triazol-1-yl)pyridin-2-yl]amino}-1H-indazol-6-yl)-5′-methoxyspiro[cyclopropane-1,3′-indol]-2′(1′H)-one; (1R,2S)-2-(3-{5-[1-(2,2-difluoroethyl)-1H-pyrazol-4-yl]-2-methoxyanilino}-1H-indazol-6-yl)-5′-methoxyspiro[cyclopropane-1,3′-indol]-2′(1′H)-one; (1R,2S)-5′-methoxy-2-(3-{[5-methoxy-2-(1,3-oxazol-5-yl)pyrimidin-4-yl]amino}-1H-indazol-6-yl)spiro[cyclopropane-1,3′-indol]-2′(1′H)-one; (1R,2S)-2-(3-{[2-ethoxy-5-(1,3-thiazol-2-yl)pyridin-3-yl]amino}-1H-indazol-6-yl)-5′-methoxyspiro[cyclopropane-1,3′-indol]-2′(1′H)-one; (1R,2S)-5′-methoxy-2-(3-{[2-methoxy-5-(1,3-oxazol-4-yl)pyridin-3-yl]amino}-1H-indazol-6-yl)spiro[cyclopropane-1,3′-indol]-2′(1′H)-one; (1R,2S)-2-{3-[(5-ethoxy-2-methylpyrimidin-4-yl)amino]-1H-indazol-6-yl}-5′-[(2H3)methyloxy]spiro[cyclopropane-1,3′-indol]-2′(1′H)-one; (1R,2S)-2-(3-{[5-ethoxy-2-(1,3-thiazol-2-yl)pyrimidin-4-yl]amino}-1H-indazol-6-yl)-5′-methoxyspiro[cyclopropane-1,3′-indol]-2′(1′H)-one; (1R,2S)-2-(3-((5-ethoxy-2-(3-hydroxy-3-methylbut-1-yn-1-yl)pyrimidin-4-yl)amino)-1H-indazol-6-yl)-5′-methoxyspiro[cyclopropane-1,3′-indolin]-2′-one; (1R,2S)-2-(3-((5-(1-(2,2-difluoroethyl)-1H-pyrazol-4-yl)-2-methoxypyridin-3-yl)amino)-1H-indazol-6-yl)-5′-methoxyspiro[cyclopropane-1,3′-indolin]-2′-one; (1R,2S)-2-(3-((6-(1-(2,2-difluoroethyl)-1H-pyrazol-4-yl)-3-methoxypyridin-2-yl)amino)-1H-indazol-6-yl)-5′-methoxyspiro[cyclopropane-1,3′-indolin]-2′-one; (1R,2S)-2-(3-((6-(1-(2,2-difluoroethyl)-1H-pyrazol-4-yl)-3-methoxypyrazin-2-yl)amino)-TH-indazol-6-yl)-5′-methoxyspiro[cyclopropane-1,3′-indolin]-2′-one; (1R)-2-(3-((2-ethoxy-5-(oxazol-4-yl)pyridin-3-yl)amino)-1H-indazol-6-yl)-5′-methoxyspiro[cyclopropane-1,3′-indolin]-2′-one; (1R,2S)-2-(3-((5-ethoxy-2-ethynylpyrimidin-4-yl)amino)-1H-indazol-6-yl)-5′-methoxyspiro[cyclopropane-1,3′-indolin]-2′-one; (1R,2S)-2-(3-((2-ethoxy-5-(1H-imidazol-1-yl)pyridin-3-yl)amino)-1H-indazol-6-yl)-5′-methoxyspiro[cyclopropane-1,3′-indolin]-2′-one; (1R,2S)-5′-methoxy-2-(3-((1-methyl-1H-1,2,4-triazol-5-yl)amino)-1H-indazol-6-yl)spiro[cyclopropane-1,3′-indolin]-2′-one; (1R,2S)-2-(3-{[6-(1H-imidazol-1-yl)-3-methoxypyrazin-2-yl]amino}-1H-indazol-6-yl)-5′-methoxyspiro[cyclopropane-1,3′-indol]-2′(1′H)-one; (1R,2S)-5′-methoxy-2-(3-{[3-methoxy-6-(1H-pyrazol-1-yl)pyrazin-2-yl]amino}-1H-indazol-6-yl)spiro[cyclopropane-1,3′-indol]-2′(1′H)-one; 1R,2S)-5′-methoxy-2-(3-{[5-methoxy-2-(1,3-oxazol-4-yl)pyrimidin-4-yl]amino}-1H-indazol-6-yl)spiro[cyclopropane-1,3′-indol]-2′(1′H)-one; 2-[4-({6-[(1R,2S)-5′-methoxy-2′-oxo-1′,2′-dihydrospiro[cyclopropane-1,3′-indol]-2-yl]-1H-indazol-3-yl}amino)-5-methyl-1H-pyrazol-1-yl]-2-methylpropanenitrile; (1R,2S)-5′-methoxy-2-(3-{[3-methyl-1-(trifluoromethyl)-1H-pyrazol-5-yl]amino}-1H-indazol-6-yl)spiro[cyclopropane-1,3-indol]-2′(1′H)-one; (1R,2S)-5′-methoxy-2-(3-{[2-methoxy-5-(morpholin-4-yl)pyridin-3-yl]amino}-1H-indazol-6-yl)spiro[cyclopropane-1,3′-indol]-2′(1′H)-one; and 5-({6-[(1R,2S)-5′-methoxy-2′-oxo-1′,2′-dihydrospiro[cyclopropane-1,3′-indol]-2-yl]-1H-indazol-3-yl}amino)-1-methyl-1H-pyrazole-3-carbonitrile.

Further provided herein are compounds selected from those set forth in Table 1A.

TABLE 1A

| Example | | |
|---|---|---|
| No. | Structure | Name |
| 1 | 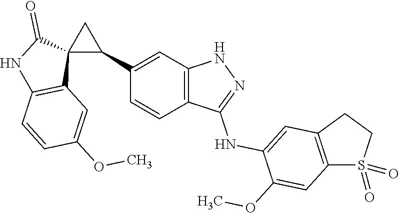 [SMILES: COc1ccc2c(c1)[C@]1(C[C@H]1c1ccc3c(Nc4cc5c(cc4OC)S(=O)(=O)CC5)n[nH]c3c1)C(=O)N2] | 6-methoxy-5-({6-[(1R,2S)-5′- methoxy-2′-oxo-1′,2′- dihydrospiro[cyclopropane-1,3′- indol]-2-yl]-1H-indazol-3- yl}amino)-2,3-dihydro-1H-1- benzothiophene-1,1-dione |
| 2 | 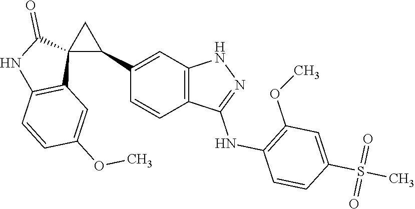 [SMILES: COc1ccc2c(c1)[C@]1(C[C@H]1c1ccc3c(Nc4ccc(S(C)(=O)=O)cc4OC)n[nH]c3c1)C(=O)N2] | (1R,2S)-2-{3-[4- (methanesulfonyl)-2- methoxyanilino]-1H-indazol-6- yl}-5′- methoxyspiro[cyclopropane-1,3′- indol]-2′(1′H)-one |
| 3 | 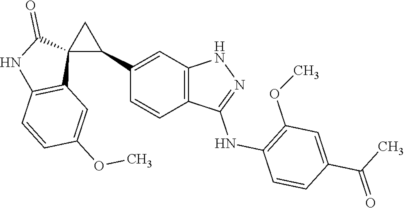 [SMILES: COc1ccc2c(c1)[C@]1(C[C@H]1c1ccc3c(Nc4ccc(C(C)=O)cc4OC)n[nH]c3c1)C(=O)N2] | (1R,2S)-2-[3-(4-acetyl-2- methoxyanilino)-1H-indazol-6- yl]-5′- methoxyspiro[cyclopropane-1,3′- indol]-2′(1′H)-one |
| 4 | 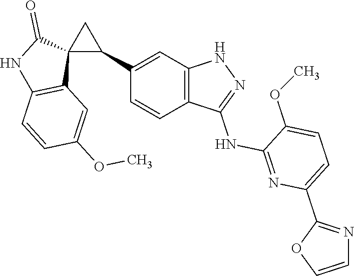 [SMILES: COc1ccc2c(c1)[C@]1(C[C@H]1c1ccc3c(Nc4nc(-c5ncco5)ccc4OC)n[nH]c3c1)C(=O)N2] | (1R,2S)-5′-methoxy-2-(3-{[3- methoxy-6-(1,3-oxazol-2- yl)pyridin-2-yl]amino}-1H- indazol-6-yl)spiro[cyclopropane- 1,3′-indol]-2′(1′H)-one |
| 5 | 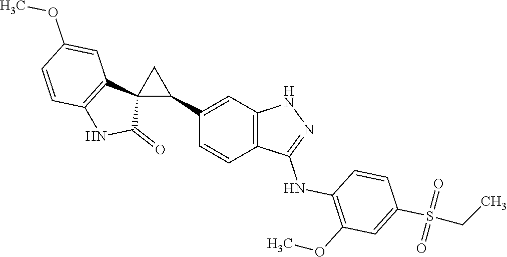 [SMILES: CCS(=O)(=O)c1ccc(Nc2n[nH]c3cc([C@@H]4C[C@@]45C(=O)Nc4ccc(OC)cc45)ccc23)c(OC)c1] | (1R,2S)-2-{3-[4- (ethanesulfonyl)-2- methoxyanilino]-1H-indazol-6- yl}-5′- methoxyspiro[cyclopropane-1,3′- indol]-2′(1′H)-one |
| 6 | 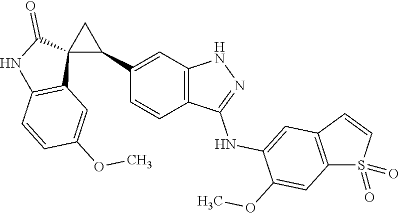 [SMILES: COc1ccc2c(c1)[C@]1(C[C@H]1c1ccc3c(Nc4cc5c(cc4OC)S(=O)(=O)C=C5)n[nH]c3c1)C(=O)N2] | 6-methoxy-5-({6-[(1R,2S)-5′- methoxy-2′-oxo-1′,2′- dihydrospiro[cyclopropane-1,3′- indol]-2-yl]-1H-indazol-3- yl}amino)-1H-1- benzothiophene-1,1-dione |
| 7 | 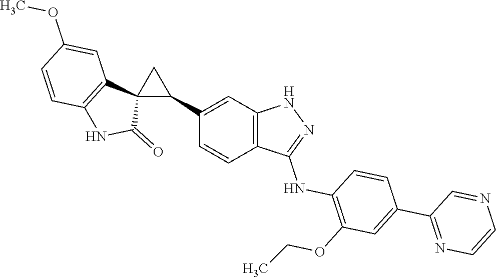 [SMILES: CCOc1cc(-c2cnccn2)ccc1Nc1n[nH]c2cc([C@@H]3C[C@@]34C(=O)Nc3ccc(OC)cc34)ccc12] | (1R,2S)-2-{3-[2-ethoxy-4- (pyrazin-2-yl)anilino]-1H- indazol-6-yl}-5′- methoxyspiro[cyclopropane-1,3′- indol]-2′(1′H)-one |
| 8 | 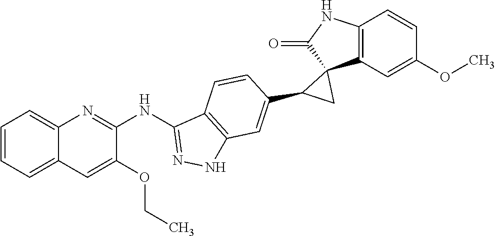 [SMILES: CCOc1cc2ccccc2nc1Nc1n[nH]c2cc([C@@H]3C[C@@]34C(=O)Nc3ccc(OC)cc34)ccc12] | (1R,2S)-2-{3-[(3- ethoxyquinolin-2-yl)amino]-1H- indazol-6-yl}-5′- methoxyspiro[cyclopropane-1,3′- indol]-2′(1′H)-one |
| 9 | 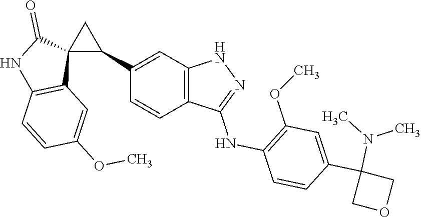 [SMILES: COc1ccc2c(c1)[C@]1(C[C@H]1c1ccc3c(Nc4ccc(C5(N(C)C)COC5)cc4OC)n[nH]c3c1)C(=O)N2] | (1R,2S)-2-(3-{4-[3- (dimethylamino)oxetan-3-yl]-2- methoxyanilino}-1H-indazol-6- yl)-5′- methoxyspiro[cyclopropane-1,3′- indol]-2′(1′H)-one |
| 10 | 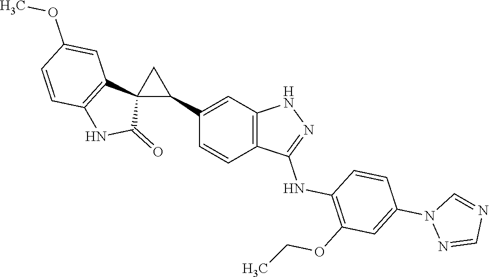 [SMILES: CCOc1cc(-n2cncn2)ccc1Nc1n[nH]c2cc([C@@H]3C[C@@]34C(=O)Nc3ccc(OC)cc34)ccc12] | (1R,2S)-2-{3-[2-ethoxy-4-(1H- 1,2,4-triazol-1-yl)anilino]-1H- indazol-6-yl}-5′- methoxyspiro[cyclopropane-1,3′- indol]-2′(1′H)-one |
| 11 | 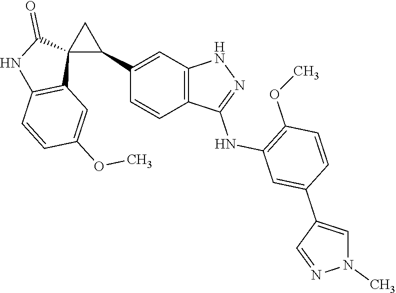 [SMILES: COc1ccc2c(c1)[C@]1(C[C@H]1c1ccc3c(Nc4cc(-c5cnn(C)c5)ccc4OC)n[nH]c3c1)C(=O)N2] | (1R,2S)-5′-methoxy-2-{3-[2- methoxy-5-(1-methyl-1H- pyrazol-4-yl)anilino]-1H- indazol-6-yl}spiro[cyclopropane- 1,3′-indol]-2′(1′H)-one |
| 12 | 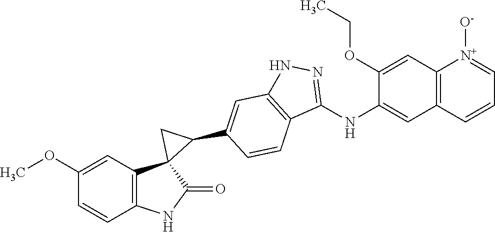 [SMILES: CCOc1cc2c(ccc[n+]2[O-])cc1Nc1n[nH]c2cc([C@@H]3C[C@@]34C(=O)Nc3ccc(OC)cc34)ccc12] | 7-ethoxy-6-((6-((1R,2S)-5′- methoxy-2′- oxospiro[cyclopropane-1,3′- indolin]-2-yl)-1H-indazol-3- yl)amino)quinoline 1-oxide |
| 13 | 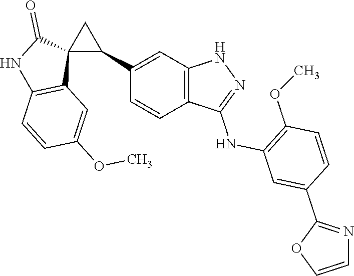 [SMILES: COc1ccc2c(c1)[C@]1(C[C@H]1c1ccc3c(Nc4cc(-c5ncco5)ccc4OC)n[nH]c3c1)C(=O)N2] | (1R,2S)-5′-methoxy-2-{3-[2- methoxy-5-(1,3-oxazol-2- yl)anilino]-1H-indazol-6- yl}spiro[cyclopropane-1,3′- indol]-2′(1′H)-one |
| 14 | 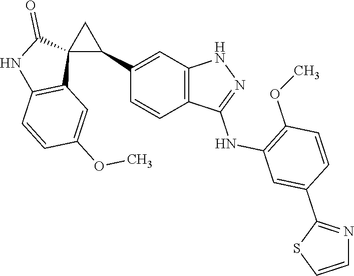 [SMILES: COc1ccc2c(c1)[C@]1(C[C@H]1c1ccc3c(Nc4cc(-c5nccs5)ccc4OC)n[nH]c3c1)C(=O)N2] | (1R,2S)-5′-methoxy-2-{3-[2- methoxy-5-(1,3-thiazol-2- yl)anilino]-1H-indazol-6- yl}spiro[cyclopropane-1,3′- indol]-2′(1′H)-one |
| 15 | 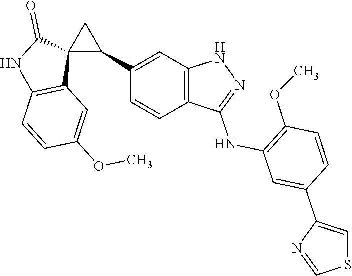 [SMILES: COc1ccc2c(c1)[C@]1(C[C@H]1c1ccc3c(Nc4cc(-c5cscn5)ccc4OC)n[nH]c3c1)C(=O)N2] | (1R,2S)-5′-methoxy-2-{3-[2- methoxy-5-(1,3-thiazol-4- yl)anilino]-1H-indazol-6- yl}spiro[cyclopropane-1,3′- indol]-2′(1′H)-one |
| 16 | 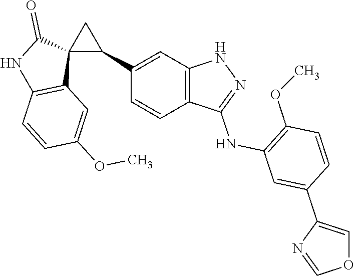 [SMILES: COc1ccc2c(c1)[C@]1(C[C@H]1c1ccc3c(Nc4cc(-c5cocn5)ccc4OC)n[nH]c3c1)C(=O)N2] | (1R,2S)-5′-methoxy-2-{3-[2- methoxy-5-(1,3-oxazol-4- yl)anilino]-1H-indazol-6- yl}spiro[cyclopropane-1,3′- indol]-2′(1′H)-one |
| 17 | 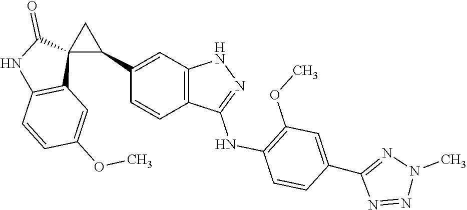 [SMILES: COc1ccc2c(c1)[C@]1(C[C@H]1c1ccc3c(Nc4ccc(-c5nnn(C)n5)cc4OC)n[nH]c3c1)C(=O)N2] | (1R,2S)-5′-methoxy-2-{3-[2- methoxy-4-(2-methyl-2H- tetrazol-5-yl)anilino]-1H- indazol-6-yl}spiro[cyclopropane- 1,3′-indol]-2′(1′H)-one |
| 18 | 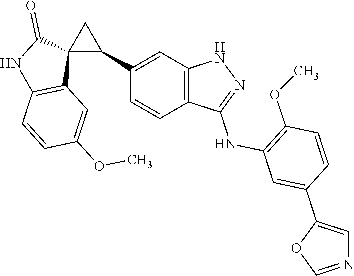 [SMILES: COc1ccc2c(c1)[C@]1(C[C@H]1c1ccc3c(Nc4cc(-c5cnco5)ccc4OC)n[nH]c3c1)C(=O)N2] | (1R,2S)-5′-methoxy-2-{3-[2- methoxy-5-(1,3-oxazol-5- yl)anilino]-1H-indazol-6- yl}spiro[cyclopropane-1,3′- indol]-2′(1′H)-one |
| 19 | 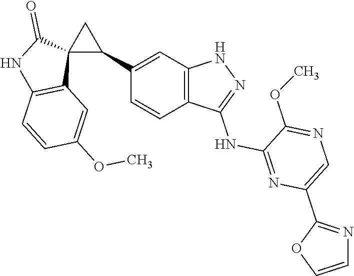 [SMILES: COc1ccc2c(c1)[C@]1(C[C@H]1c1ccc3c(Nc4nc(-c5ncco5)cnc4OC)n[nH]c3c1)C(=O)N2] | (1R,2S)-5′-methoxy-2-(3-{[3- methoxy-6-(1,3-oxazol-2- yl)pyrazin-2-yl]amino}-1H- indazol-6-yl)spiro[cyclopropane- 1,3′-indol]-2′(1′H)-one |
| 20 | 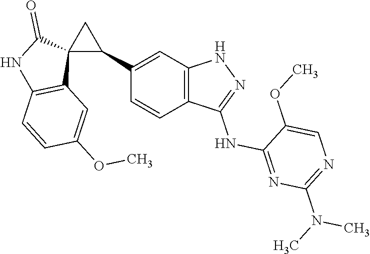 [SMILES: COc1ccc2c(c1)[C@]1(C[C@H]1c1ccc3c(Nc4nc(N(C)C)ncc4OC)n[nH]c3c1)C(=O)N2] | (1R,2S)-2-(3-{[2- (dimethylamino)-5- methoxypyrimidin-4-yl]amino}- 1H-indazol-6-yl)-5′- methoxyspiro[cyclopropane-1,3′- indol]-2′(1′H)-one |
| 21 | 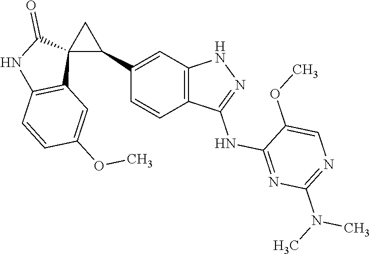 [SMILES: COc1ccc2c(c1)[C@]1(C[C@H]1c1ccc3c(Nc4nc(N(C)C)ncc4OC)n[nH]c3c1)C(=O)N2] | (1R,2S)-5′-methoxy-2-{3-[2- methoxy-5-(2-methyl-2H- tetrazol-5-yl)anilino]-1H- indazol-6-yl}spiro[cyclopropane- 1,3′-indol]-2′(1′H)-one |
| 22 | 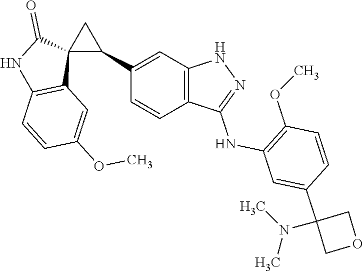 [SMILES: COc1ccc2c(c1)[C@]1(C[C@H]1c1ccc3c(Nc4cc(C5(N(C)C)COC5)ccc4OC)n[nH]c3c1)C(=O)N2] | (1R,2S)-2-(3-{5-[3- (dimethylamino)oxetan-3-yl]-2- methoxyanilino}-1H-indazol-6- yl)-5′- methoxyspiro[cyclopropane-1,3′- indol]-2′(1′H)-one |
| 23 | 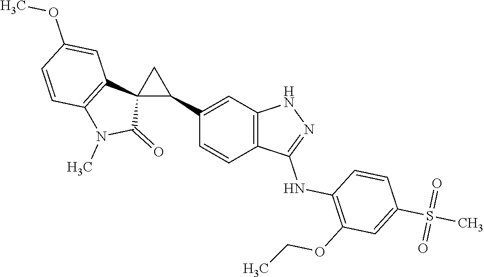 | (1R,2S)-2-{3-[2-ethoxy-4- (methanesulfonyl)anilino]-1H- indazol-6-yl}-5′-methoxy-1′- methylspiro[cyclopropane-1,3′- indol]-2′(1′H)-one |
| 24 | 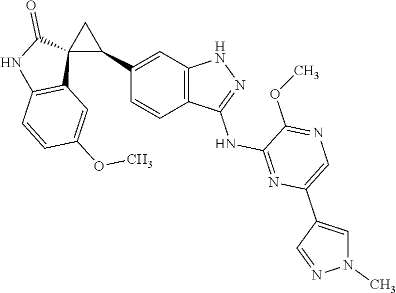 [SMILES: COc1ccc2c(c1)[C@]1(C[C@H]1c1ccc3c(Nc4nc(-c5cnn(C)c5)cnc4OC)n[nH]c3c1)C(=O)N2] | (1R,2S)-5′-methoxy-2-(3-{[3- methoxy-6-(1-methyl-1H- pyrazol-4-yl)pyrazin-2- yl]amino}-1H-indazol-6- yl)spiro[cyclopropane-1,3′- indol]-2′(1′H)-one |
| 25 | 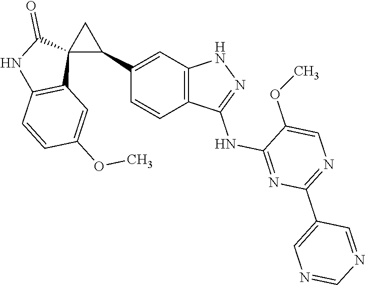 [SMILES: COc1ccc2c(c1)[C@]1(C[C@H]1c1ccc3c(Nc4nc(-c5cncnc5)ncc4OC)n[nH]c3c1)C(=O)N2] | (1R,2S)-5′-methoxy-2-{3-[(5- methoxy[2,5′-bipyrimidin]-4- yl)amino]-1H-indazol-6- yl}spiro[cyclopropane-1,3′- indol]-2′(1′H)-one |
| 26 | 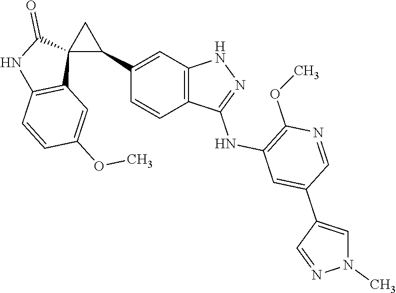 [SMILES: COc1ccc2c(c1)[C@]1(C[C@H]1c1ccc3c(Nc4cc(-c5cnn(C)c5)cnc4OC)n[nH]c3c1)C(=O)N2] | (1R,2S)-5′-methoxy-2-(3-{[2- methoxy-5-(1-methyl-1H- pyrazol-4-yl)pyridin-3- yl]amino}-1H-indazol-6- yl)spiro[cyclopropane-1,3′- indol]-2′(1′H)-one |
| 27 | 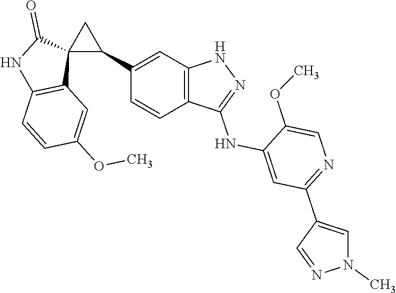 [SMILES: COc1ccc2c(c1)[C@]1(C[C@H]1c1ccc3c(Nc4cc(-c5cnn(C)c5)ncc4OC)n[nH]c3c1)C(=O)N2] | (1R,2S)-5′-methoxy-2-(3-{[5- methoxy-2-(1-methyl-1H- pyrazol-4-yl)pyridin-4- yl]amino}-1H-indazol-6- yl)spiro[cyclopropane-1,3′- indol]-2′(1′H)-one |
| 28 | 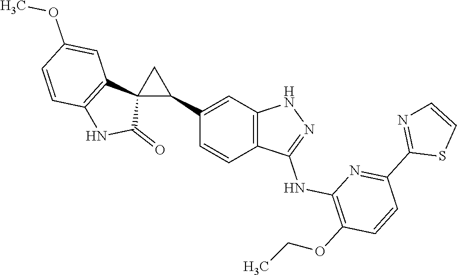 [SMILES: CCOc1ccc(-c2nccs2)nc1Nc1n[nH]c2cc([C@@H]3C[C@@]34C(=O)Nc3ccc(OC)cc34)ccc12] | (1R,2S)-2-(3-{[3-ethoxy-6-(1,3- thiazol-2-yl)pyridin-2- yl]amino}-1H-indazol-6-yl)-5′- methoxyspiro[cyclopropane-1,3′- indol]-2′(1′H)-one |
| 29 | 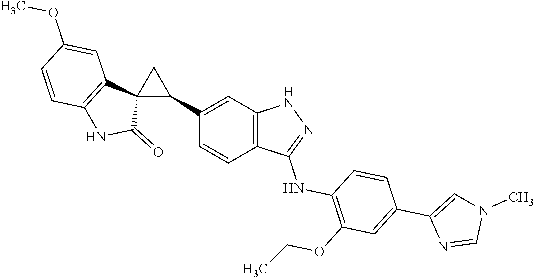 | (1R,2S)-2-{3-[2-ethoxy-4-(1- methyl-1H-imidazol-4- yl)anilino]-1H-indazol-6-yl}-5′- methoxyspiro[cyclopropane-1,3′- indol]-2′(1′H)-one |
| 30 | 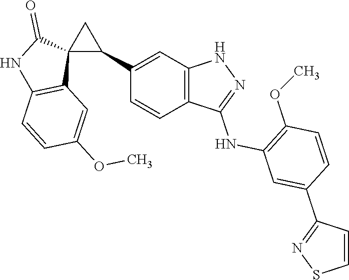 [SMILES: COc1ccc2c(c1)[C@]1(C[C@H]1c1ccc3c(Nc4cc(-c5ccsn5)ccc4OC)n[nH]c3c1)C(=O)N2] | (1R,2S)-5′-methoxy-2-{3-[2- methoxy-5-(1,2-thiazol-3- yl)anilino]-1H-indazol-6- yl}spiro[cyclopropane-1,3′- indol]-2′(1′H)-one |
| 31 | 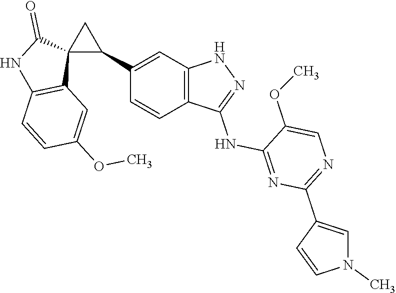 [SMILES: COc1ccc2c(c1)[C@]1(C[C@H]1c1ccc3c(Nc4nc(-c5ccn(C)c5)ncc4OC)n[nH]c3c1)C(=O)N2] | (1R,2S)-5′-methoxy-2-(3-{[5- methoxy-2-(1-methyl-1H- pyrazol-4-yl)pyrimidin-4- yl]amino}-1H-indazol-6- yl)spiro[cyclopropane-1,3′- indol]-2′(1′H)-one |
| 32 | 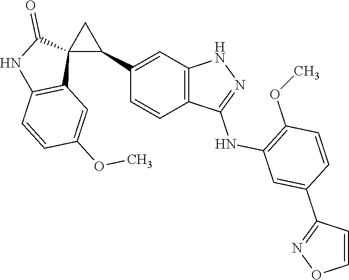 [SMILES: COc1ccc2c(c1)[C@]1(C[C@H]1c1ccc3c(Nc4cc(-c5ccon5)ccc4OC)n[nH]c3c1)C(=O)N2] | (1R,2S)-5′-methoxy-2-{3-[2- methoxy-5-(1,2-oxazol-3- yl)anilino]-1H-indazol-6- yl}spiro[cyclopropane-1,3′- indol]-2′(1′H)-one |
| 33 | 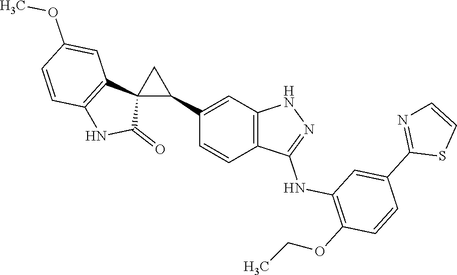 [SMILES: CCOc1ccc(-c2nccs2)cc1Nc1n[nH]c2cc([C@@H]3C[C@@]34C(=O)Nc3ccc(OC)cc34)ccc12] | (1R,2S)-2-{3-[2-ethoxy-5-(1,3- thiazol-2-yl)anilino]-1H-indazol- 6-yl}-5′- methoxyspiro[cyclopropane-1,3′- indol]-2′(1′H)-one |
| 34 | 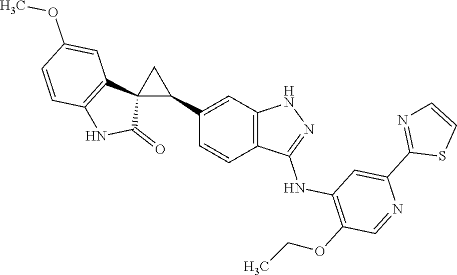 [SMILES: CCOc1cnc(-c2nccs2)cc1Nc1n[nH]c2cc([C@@H]3C[C@@]34C(=O)Nc3ccc(OC)cc34)ccc12] | (1R,2S)-2-(3-{[5-ethoxy-2-(1,3- thiazol-2-yl)pyridin-4- yl]amino}-1H-indazol-6-yl)-5′- methoxyspiro[cyclopropane-1,3′- indol]-2′(1′H)-one |
| 35 | 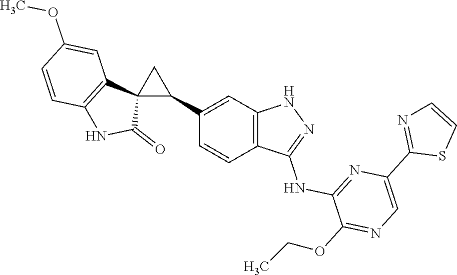 [SMILES: CCOc1ncc(-c2nccs2)nc1Nc1n[nH]c2cc([C@@H]3C[C@@]34C(=O)Nc3ccc(OC)cc34)ccc12] | (1R,2S)-2-(3-{[3-ethoxy-6-(1,3- thiazol-2-yl)pyrazin-2- yl]amino}-1H-indazol-6-yl)-5′- methoxyspiro[cyclopropane-1,3′- indol]-2′(1′H)-one |
| 36 | 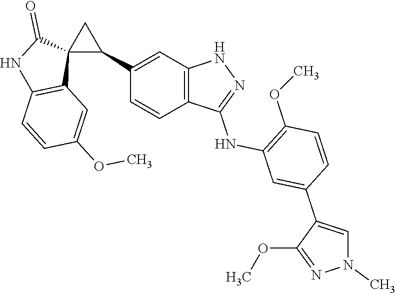 [SMILES: COc1ccc2c(c1)[C@]1(C[C@H]1c1ccc3c(Nc4cc(-c5cn(C)nc5OC)ccc4OC)n[nH]c3c1)C(=O)N2] | (1R,2S)-5′-methoxy-2-{3-[2- methoxy-5-(3-methoxy-1- methyl-1H-pyrazol-4-yl)anilino]- 1H-indazol-6- yl}spiro[cyclopropane-1,3′- indol]-2′(1′H)-one |
| 37 | 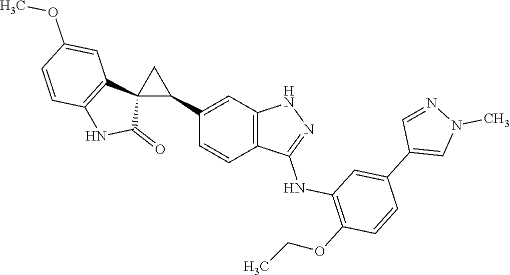 | (1R,2S)-2-{3-[2-ethoxy-5-(1- methyl-1H-pyrazol-4-yl)anilino]- 1H-indazol-6-yl}-5′- methoxyspiro[cyclopropane-1,3′- indol]-2′(1′H)-one |
| 38 | 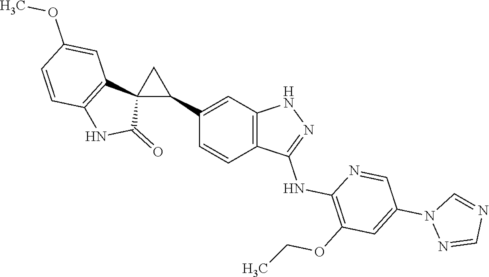 [SMILES: CCOc1cc(-n2cncn2)cnc1Nc1n[nH]c2cc([C@@H]3C[C@@]34C(=O)Nc3ccc(OC)cc34)ccc12] | (1R,2S)-2-(3-{[3-ethoxy-5-(1H- 1,2,4-triazol-1-yl)pyridin-2- yl]amino}-1H-indazol-6-yl)-5′- methoxyspiro[cyclopropane-1,3′- indol]-2′(1′H)-one |
| 39 | 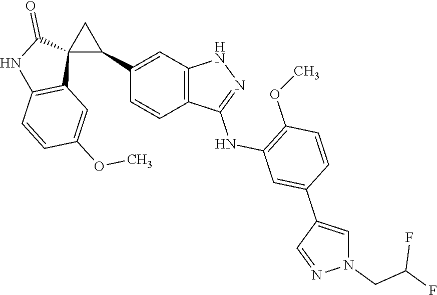 [SMILES: COc1ccc2c(c1)[C@]1(C[C@H]1c1ccc3c(Nc4cc(-c5cnn(CC(F)F)c5)ccc4OC)n[nH]c3c1)C(=O)N2] | (1R,2S)-2-(3-{5-[1-(2,2- difluoroethyl)-1H-pyrazol-4-yl]- 2-methoxyanilino}-1H-indazol- 6-yl)-5′- methoxyspiro[cyclopropane-1,3′- indol]-2′(1′H)-one |
| 40 | 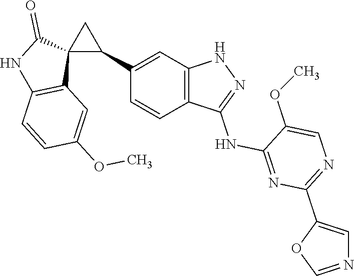 [SMILES: COc1ccc2c(c1)[C@]1(C[C@H]1c1ccc3c(Nc4nc(-c5cnco5)ncc4OC)n[nH]c3c1)C(=O)N2] | (1R,2S)-5′-methoxy-2-(3-{[5- methoxy-2-(1,3-oxazol-5- yl)pyrimidin-4-yl]amino}-1H- indazol-6-yl)spiro[cyclopropane- 1,3′-indol]-2′(1′H)-one |
| 41 | 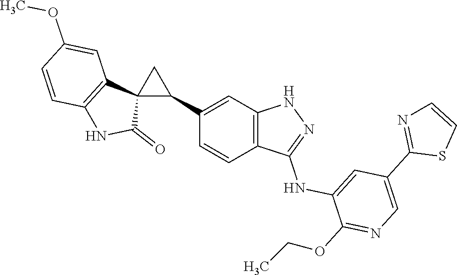 [SMILES: CCOc1ncc(-c2nccs2)cc1Nc1n[nH]c2cc([C@@H]3C[C@@]34C(=O)Nc3ccc(OC)cc34)ccc12] | (1R,2S)-2-(3-{[2-ethoxy-5-(1,3- thiazol-2-yl)pyridin-3- yl]amino}-1H-indazol-6-yl)-5′- methoxyspiro[cyclopropane-1,3′- indol]-2′(1′H)-one |
| 42 | 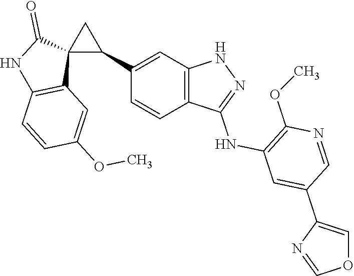 [SMILES: COc1ccc2c(c1)[C@]1(C[C@H]1c1ccc3c(Nc4cc(-c5cocn5)cnc4OC)n[nH]c3c1)C(=O)N2] | (1R,2S)-5′-methoxy-2-(3-{[2- methoxy-5-(1,3-oxazol-4- yl)pyridin-3-yl]amino}-1H- indazol-6-yl)spiro[cyclopropane- 1,3′-indol]-2′(1′H)-one |
| 43 | 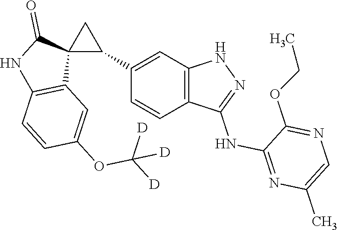 [SMILES: [2H]C([2H])([2H])Oc1ccc2c(c1)[C@@]1(C[C@@H]1c1ccc3c(Nc4nc(C)cnc4OCC)n[nH]c3c1)C(=O)N2] | (1R,2S)-2-{3-[(5-ethoxy-2- methylpyrimidin-4-yl)amino]- 1H-indazol-6-yl}-5′- [(2H3)methyloxy] spiro[cyclopropane- 1,3′-indol]-2′(1′H)-one |
| 44 | 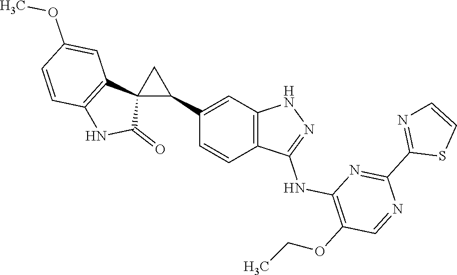 [SMILES: CCOc1cnc(-c2nccs2)nc1Nc1n[nH]c2cc([C@@H]3C[C@@]34C(=O)Nc3ccc(OC)cc34)ccc12] | (1R,2S)-2-(3-{[5-ethoxy-2-(1,3- thiazol-2-yl)pyrimidin-4- yl]amino}-1H-indazol-6-yl)-5′- methoxyspiro[cyclopropane-1,3′- indol]-2′(1′H)-one |
| 45 | 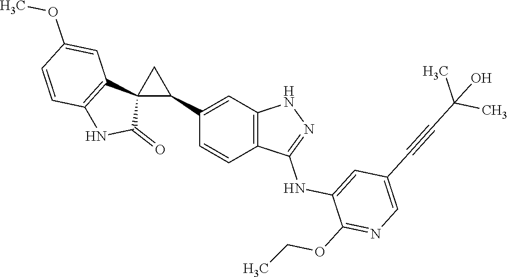 [SMILES: CCOc1ncc(C#CC(C)(C)O)cc1Nc1n[nH]c2cc([C@@H]3C[C@@]34C(=O)Nc3ccc(OC)cc34)ccc12] | (1R,2S)-2-(3-((5-ethoxy-2-(3- hydroxy-3-methylbut-1-yn-1- yl)pyrimidin-4-yl)amino)-1H- indazol-6-yl)-5′- methoxyspiro[cyclopropane-1,3′- indolin]-2′-one |
| 46 | 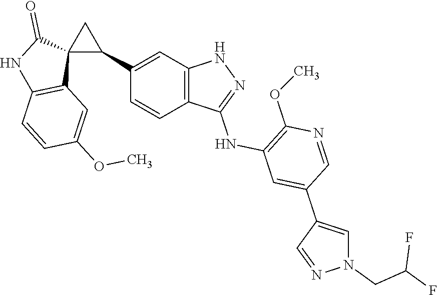 [SMILES: COc1ccc2c(c1)[C@]1(C[C@H]1c1ccc3c(Nc4cc(-c5cnn(CC(F)F)c5)cnc4OC)n[nH]c3c1)C(=O)N2] | (1R,2S)-2-(3-((5-(1-(2,2- difluoroethyl)-1H-pyrazol-4-yl)- 2-methoxypyridin-3-yl)amino)- 1H-indazol-6-yl)-5′- methoxyspiro[cyclopropane-1,3′- indolin]-2′-one |
| 47 | 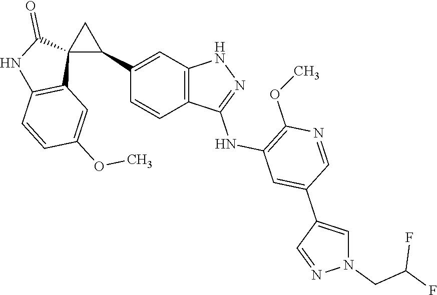 [SMILES: COc1ccc2c(c1)[C@]1(C[C@H]1c1ccc3c(Nc4cc(-c5cnn(CC(F)F)c5)cnc4OC)n[nH]c3c1)C(=O)N2] | (1R,2S)-2-(3-((6-(1-(2,2- difluoroethyl)-1H-pyrazol-4-yl)- 3-methoxypyridin-2-yl)amino)- 1H-indazol-6-yl)-5′- methoxyspiro[cyclopropane-1,3′- indolin]-2′-one |
| 48 | 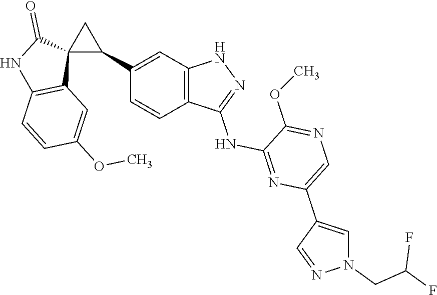 [SMILES: COc1ccc2c(c1)[C@]1(C[C@H]1c1ccc3c(Nc4nc(-c5cnn(CC(F)F)c5)cnc4OC)n[nH]c3c1)C(=O)N2] | (1R,2S)-2-(3-((6-(1-(2,2- difluoroethyl)-1H-pyrazol-4-yl)- 3-methoxypyrazin-2-yl)amino)- 1H-indazol-6-yl)-5′- methoxyspiro[cyclopropane-1,3′- indolin]-2′-one |
| 49 | 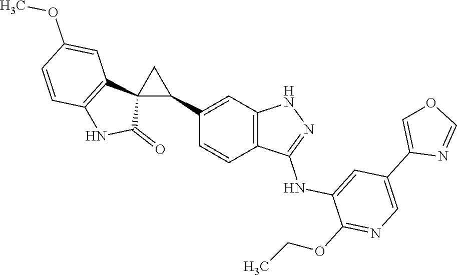 [SMILES: CCOc1ncc(-c2cocn2)cc1Nc1n[nH]c2cc([C@@H]3C[C@@]34C(=O)Nc3ccc(OC)cc34)ccc12] | (1R)-2-(3-((2-ethoxy-5-(oxazol- 4-yl)pyridin-3-yl)amino)-1H- indazol-6-yl)-5′- methoxyspiro[cyclopropane-1,3′- indolin]-2′-one |
| 50 | 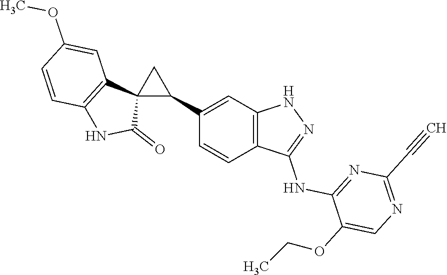 [SMILES: C#Cc1ncc(OCC)c(Nc2n[nH]c3cc([C@@H]4C[C@@]45C(=O)Nc4ccc(OC)cc45)ccc23)n1] | (1R,2S)-2-(3-((5-ethoxy-2- ethynylpyrimidin-4-yl)amino)- 1H-indazol-6-yl)-5′- methoxyspiro[cyclopropane-1,3′- indolin]-2′-one |
| 51 | 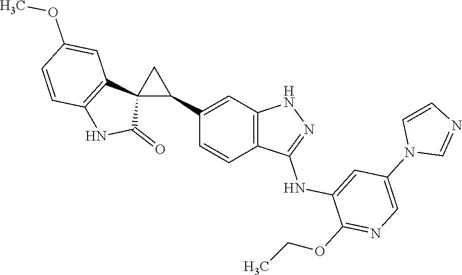 [SMILES: CCOc1ncc(-n2ccnc2)cc1Nc1n[nH]c2cc([C@@H]3C[C@@]34C(=O)Nc3ccc(OC)cc34)ccc12] | (1R,2S)-2-(3-((2-ethoxy-5-(1H- imidazol-1-yl)pyridin-3- yl)amino)-1H-indazol-6-yl)-5′- methoxyspiro[cyclopropane-1,3′- indolin]-2′-one |
| 52 | 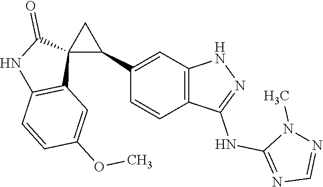 [SMILES: COc1ccc2c(c1)[C@]1(C[C@H]1c1ccc3c(Nc4ncnn4C)n[nH]c3c1)C(=O)N2] | (1R,2S)-5′-methoxy-2-(3-((1- methyl-1H-1,2,4-triazol-5- yl)amino)-1H-indazol-6- yl)spiro[cyclopropane-1,3′- indolin]-2′-one |
| 53 | 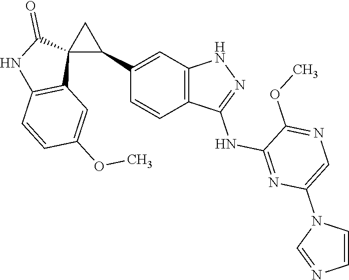 [SMILES: COc1ccc2c(c1)[C@]1(C[C@H]1c1ccc3c(Nc4nc(-n5ccnc5)cnc4OC)n[nH]c3c1)C(=O)N2] | (1R,2S)-2-(3-{[6-(1H-imidazol- 1-yl)-3-methoxypyrazin-2- yl]amino}-1H-indazol-6-yl)-5′- methoxyspiro[cyclopropane-1,3′- indol]-2′(1′H)-one |
| 54 | 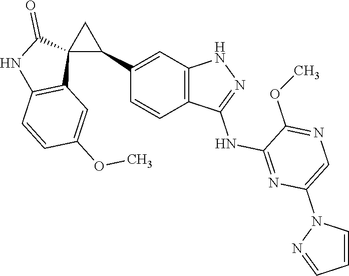 [SMILES: COc1ccc2c(c1)[C@]1(C[C@H]1c1ccc3c(Nc4nc(-n5cccn5)cnc4OC)n[nH]c3c1)C(=O)N2] | (1R,2S)-5′-methoxy-2-(3-{[3- methoxy-6-(1H-pyrazol-1- yl)pyrazin-2-yl]amino}-1H- indazol-6-yl)spiro[cyclopropane- 1,3′-indol]-2′(1′H)-one |
| 55 | 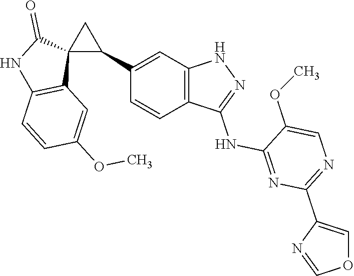 [SMILES: COc1ccc2c(c1)[C@]1(C[C@H]1c1ccc3c(Nc4nc(-c5cocn5)ncc4OC)n[nH]c3c1)C(=O)N2] | (1R,2S)-5′-methoxy-2-(3-{[5- methoxy-2-(1,3-oxazol-4- yl)pyrimidin-4-yl]amino}-1H- indazol-6-yl)spiro[cyclopropane- 1,3′-indol]-2′(1′H)-one |
| 56 | 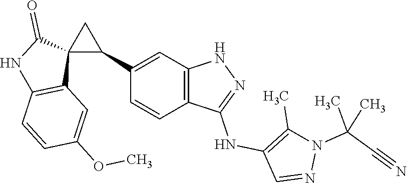 [SMILES: COc1ccc2c(c1)[C@]1(C[C@H]1c1ccc3c(Nc4cnn(C(C)(C)C#N)c4C)n[nH]c3c1)C(=O)N2] | 2-[4-({6-[(1R,2S)-5′-methoxy-2′- oxo-1′,2′- dihydrospiro[cyclopropane-1,3′- indol]-2-yl]-1H-indazol-3- yl}amino)-5-methyl-1H-pyrazol- 1-yl]-2-methylpropanenitrile |
| 57 | 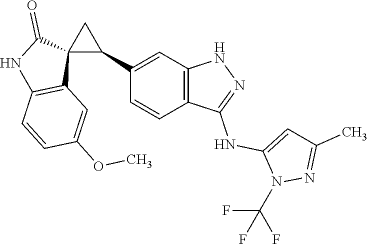 [SMILES: COc1ccc2c(c1)[C@]1(C[C@H]1c1ccc3c(Nc4cc(C)nn4C(F)(F)F)n[nH]c3c1)C(=O)N2] | (1R,2S)-5′-methoxy-2-(3-{[3- methyl-1-(trifluoromethyl)-1H- pyrazol-5-yl]amino}-1H-indazol- 6-yl)spiro[cyclopropane-1,3′- indol]-2′(1′H)-one |
| 58 | 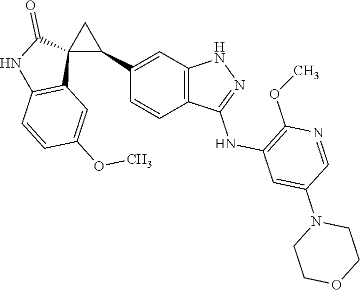 [SMILES: COc1ccc2c(c1)[C@]1(C[C@H]1c1ccc3c(Nc4cc(N5CCOCC5)cnc4OC)n[nH]c3c1)C(=O)N2] | (1R,2S)-5′-methoxy-2-(3-{[2- methoxy-5-(morpholin-4- yl)pyridin-3-yl]amino}-1H- indazol-6-yl)spiro[cyclopropane- 1,3′-indol]-2′(1′H)-one |
| 59 | 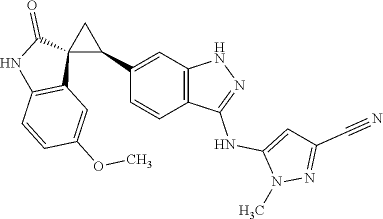 [SMILES: COc1ccc2c(c1)[C@]1(C[C@H]1c1ccc3c(Nc4cc(C#N)nn4C)n[nH]c3c1)C(=O)N2] | 5-({6-[(1R,2S)-5′-methoxy-2′- oxo-1′,2′- dihydrospiro[cyclopropane-1,3′- indol]-2-yl]-1H-indazol-3- yl}amino)-1-methyl-1H- pyrazole-3-carbonitrile |

Further provided herein are compounds of the formula:

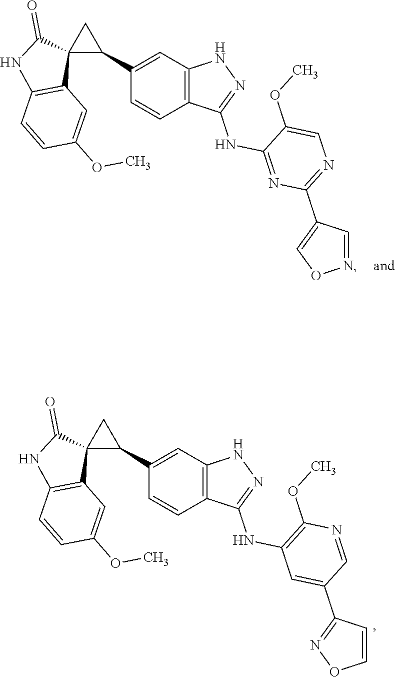 [SMILES: COc1ccc2c(c1)[C@]1(C[C@H]1c1ccc3c(Nc4nc(-c5cnoc5)ncc4OC)n[nH]c3c1)C(=O)N2 ; "and" ; COc1ccc2c(c1)[C@]1(C[C@H]1c1ccc3c(Nc4cc(-c5ccon5)cnc4OC)n[nH]c3c1)C(=O)N2]

or a pharmaceutically acceptable salt thereof.

Further provided herein are pharmaceutical compositions comprising an amount of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, and one or more pharmaceutically acceptable excipients.

### Methods of Treatment

Further provided herein are methods of treating cancer in a subject in need thereof, comprising administering to the subject a therapeutically effective amount of compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, or a pharmaceutical composition disclosed herein comprising a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof. Provided herein are such methods of treating cancer in a subject, wherein the cancer in the subject is a solid tumor. In some embodiments, the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments, the cancer in the subject expresses polo-like kinase 4 (PLK4). In some embodiments, the cancer in the subject has been determined to express polo-like kinase 4 (PLK4) prior to administering to the subject a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof. In some embodiments, the cancer in the subject exhibits an overexpression of the E3 ubiquitin-protein ligase (TRIM37) protein. In some embodiments, the cancer in the subject exhibits an overexpression of the gene that encodes the tripartite motif-containing protein 37 (TRIM37). In some embodiments, the cancer in the subject exhibits an amplification of the gene that encodes the tripartite motif-containing protein 37 (TRIM37).

Further provided herein are methods of treating cancer in a subject in need thereof, comprising administering to the subject a therapeutically effective amount of compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, wherein the cancer in the subject has been determined to overexpress the gene that encodes the tripartite motif-containing protein 37 (TRIM37) prior to administration of the compound to the subject.

Further provided herein are methods of treating cancer in a subject in need thereof, wherein the cancer in the subject has been determined to overexpress the gene that encodes the tripartite motif-containing protein 37 (TRIM37), comprising administering to the subject a therapeutically effective amount of compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof.

Further provided herein are methods of treating cancer in a subject, comprising:
a. obtaining a biological sample of the cancer from the subject;
b. determining whether the biological sample of the cancer overexpresses the gene that encodes the tripartite motif-containing protein 37 (TRIM37); and
c. administering to the subject a therapeutically effective amount of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, if the biological sample of the cancer is determined to overexpress the gene that encodes the tripartite motif-containing protein 37 (TRIM37).

Further provided herein are methods of treating cancer in a subject described herein, wherein the cancer is neuroblastoma or breast cancer. Also provided herein are methods of treating cancer in a subject described herein, wherein the cancer is neuroblastoma. Also provided herein are methods of treating cancer in a subject described herein, wherein the cancer is breast cancer.

Further provided herein are methods of treating cancer in a subject described herein, wherein a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered to the subject with one or more additional therapeutic agents. In some embodiments, the one or more additional therapeutic agents is selected from one or more mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, and immuno-oncology agents.

Further provided herein are methods of inhibiting polo-like kinase 4 (PLK4) in a subject in need thereof, comprising administering to the subject a therapeutically effective amount of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, or a pharmaceutical composition comprising a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof.

Further provided herein are methods of inhibiting polo-like kinase 4 (PLK4) in a subject having cancer, comprising administering to the subject a therapeutically effective amount of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, or a pharmaceutical composition comprising a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, wherein the cancer in the subject has been determined to express polo-like kinase 4 (PLK4) prior to administering the compound or the pharmaceutical composition to the subject.

Further provided herein are methods of treating cancer in a subject in need thereof, comprising administering to the subject a therapeutically effective amount of compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, wherein the cancer in the subject is acute myeloid leukemia, myelodysplastic syndromes, chronic myelomonocytic leukemia, triple negative breast cancer, advanced breast cancer, metastatic breast cancer, or prostate cancer. In some embodiments, the cancer in the subject is acute myeloid leukemia. In some embodiments, the cancer in the subject is myelodysplastic syndromes. In some embodiments, the cancer in the subject is chronic myelomonocytic leukemia. In some embodiments, the cancer in the subject is triple negative breast cancer. In some embodiments, the cancer in the subject is advanced breast cancer. In some embodiments, the cancer in the subject is metastatic breast cancer. In some embodiments, the cancer in the subject is prostate cancer.

Further provided herein are compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, or pharmaceutical compositions comprising a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, for use in methods of treating cancer in a subject in need thereof. In some embodiments are provided such compounds or pharmaceutical compositions for such use, wherein the cancer in the subject is a solid tumor. In some embodiments are provided such compounds or pharmaceutical compositions for such use, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments are provided such compounds or pharmaceutical compositions for such use, wherein the cancer in the subject expresses polo-like kinase 4 (PLK4). In some embodiments are provided such compounds or pharmaceutical compositions for such use, wherein the cancer in the subject has been determined to express polo-like kinase 4 (PLK4) prior to administering the compound or the pharmaceutical composition to the subject. In some embodiments are provided such compounds or pharmaceutical compositions for such use, wherein the cancer in the subject exhibits an overexpression of the E3 ubiquitin-protein ligase (TRIM37) protein. In some embodiments are provided such compounds or pharmaceutical compositions for such use, wherein the cancer in the subject exhibits an overexpression of the gene that encodes the tripartite motif-containing protein 37 (TRIM37). In some embodiments are provided such compounds or pharmaceutical compositions for such use, wherein the cancer in the subject exhibits an amplification of the gene that encodes the tripartite motif-containing protein 37 (TRIM37). In some embodiments are provided such compounds or pharmaceutical compositions for such use, wherein the cancer in the subject has been determined to overexpress the gene that encodes the tripartite motif-containing protein 37 (TRIM37) prior to administration of the compound or the pharmaceutical composition to the subject. In some embodiments are provided such compounds or pharmaceutical compositions for such use, wherein the cancer is neuroblastoma or breast cancer. In some embodiments are provided such compounds or pharmaceutical compositions for such use, wherein the cancer is neuroblastoma. In some embodiments are provided such compounds or pharmaceutical compositions for such use, wherein the cancer is breast cancer.

Further provided herein are compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, or pharmaceutical compositions comprising a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, for use in methods of treating cancer in a subject in need thereof wherein the cancer in the subject is acute myeloid leukemia, myelodysplastic syndromes, chronic myelomonocytic leukemia, triple negative breast cancer, advanced breast cancer, metastatic breast cancer, or prostate cancer. In some embodiments, the cancer in the subject is acute myeloid leukemia. In some embodiments, the cancer in the subject is myelodysplastic syndromes. In some embodiments, the cancer in the subject is chronic myelomonocytic leukemia. In some embodiments, the cancer in the subject is triple negative breast cancer. In some embodiments, the cancer in the subject is advanced breast cancer. In some embodiments, the cancer in the subject is metastatic breast cancer. In some embodiments, the cancer in the subject is prostate cancer.

Further provided herein are compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, or pharmaceutical compositions comprising a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, for use in methods of inhibiting polo-like kinase 4 (PLK4) in a subject having cancer.

Further provided herein are uses of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, in the manufacture of a medicament for the treatment of cancer in a subject in need thereof. In some embodiments are provided such uses, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments the cancer in the subject expresses polo-like kinase 4 (PLK4). In some embodiments the cancer in the subject has been determined to express polo-like kinase 4 (PLK4) prior to administering the compound to the subject. In some embodiments the cancer in the subject exhibits an overexpression of the E3 ubiquitin-protein ligase (TRIM37) protein. In some embodiments the cancer in the subject exhibits an overexpression of the gene that encodes the tripartite motif-containing protein 37 (TRIM37). In some embodiments the cancer in the subject exhibits an amplification of the gene that encodes the tripartite motif-containing protein 37 (TRIM37). In some embodiments the cancer in the subject has been determined to overexpress the gene that encodes the tripartite motif-containing protein 37 (TRIM37) prior to administration of the compound to the subject. In some embodiments the cancer is neuroblastoma or breast cancer. In some embodiments the cancer is neuroblastoma. In some embodiments the cancer is breast cancer.

Further provided herein are uses of a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, in the manufacture of a medicament for the treatment of cancer in a subject in need thereof, wherein the cancer in the subject is acute myeloid leukemia, myelodysplastic syndromes, chronic myelomonocytic leukemia, triple negative breast cancer, advanced breast cancer, metastatic breast cancer, or prostate cancer. In some embodiments, the cancer in the subject is acute myeloid leukemia. In some embodiments, the cancer in the subject is myelodysplastic syndromes. In some embodiments, the cancer in the subject is chronic myelomonocytic leukemia. In some embodiments, the cancer in the subject is triple negative breast cancer. In some embodiments, the cancer in the subject is advanced breast cancer. In some embodiments, the cancer in the subject is metastatic breast cancer. In some embodiments, the cancer in the subject is prostate cancer.

In some embodiments, compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is used in combination with one or more additional anti-cancer agents. In some embodiments, the anti-cancer agent is mitoxantrone, estramustine, etoposide, vinblastine, carboplatin, vinorelbine, paclitaxel, daunomycin, darubicin, epirubicin, docetaxel, cabazitaxel, or doxorubicin. In some embodiments, the anti-cancer agent is paclitaxel, daunomycin, darubicin, epirubicin, docetaxel, cabazitaxel, or doxorubicin. In certain embodiments, the anti-cancer agent is docetaxel.

In some embodiments, one or more additional anti-cancer agents may include, without limitations, surgery, radiation, or chemotherapy. The chemotherapy may be an androgen receptor antagonist, a mitotic inhibitor, an antimetabolite, a platinum-based agent. Examples of androgen receptor antagonist include, without limitations, apalutamide, flutamide, nilutamide, bicalutamide, or enzalutamide. Examples of mitotic inhibitors include, without limitations, a taxane (e.g. paclitaxel, docetaxel, paclitaxel, docetaxel, cabazitaxel, tesetaxel, or nab-paclitaxel) or a vinca alkaloid (e.g., vinblastine, vincristine, vindesine, or vinorelbine). Examples of antimetabolites include, without limitations, 5-Fluorouracil, 6-mercaptopurine, capecitabine, cytarabine, floxuridine, fludarabine, gemcitabine, hydroxycarbamide, methotrexate, pemetrexed, or phototrexate. Examples of platinum-based agents include, without limitations, cisplatin, carboplatin, dicycloplatin, eptaplatin, lobaplatin, miriplatin, nedaplatin, oxaliplatin, picoplatin, satraplatin, or triplatin tetranitrate. The additional anti-cancer therapy may comprise an anti-PDL1 agent, an anti-PD1 agent or an anti CTLA-4 agent. The anti-PD-L1 agent may comprise atezolizumab, avelumab, durvalumab, MPDL3280A (RG7446), MDX-1105 (BMS-936559) or BMS-935559, MSB0010718C, and MEDI4736. The anti-PD1 agent may comprise pembrolizumab, nivolumab, cemiplimab, partalizumab (PDROO1), camrelizumab (SHR1210), sintilimab (IBI308), tislelizumab (BGB-A317), toripalimab (JS 001), dostarlimab (TSR-042, WBP-285), INCMGA00012 (MGA012), AMP-224, or AMP-514 (MEDI0680). The anti-CTLA agent may comprise ipilimumab, or tremelimumab.

Methods of Treatment in Conjunction with Biomarkers

Disclosed herein, in some embodiments, are methods of detecting the presence, absence, or level, of a biomarker. Such biomarkers may comprise genetic alterations in the gene encoding for certain proteins such as tripartite motif-containing protein 37 (TRIM37). The presence, absence, or level, of such biomarkers may be measured in a biological sample obtained from a subject, such as a sample of a solid tumor, such as a prostate cancer, or from a sample of a relevant biological fluid, such as a blood sample. In some instances, the methods of detection disclosed herein are useful for predicting a therapeutic response to a therapy described herein (e.g., a PLK4 inhibitor) in, monitor the treatment using the therapy of, and treating with the therapy, a proliferative disease or condition described herein in a subject. In some embodiments, the presence, or an absence, and/or a level of expression of the one or more biomarkers is detected in the sample obtained from a subject by analyzing the genetic material in the sample. In some embodiments, the genetic material is obtained from blood, serum, plasma, sweat, hair, tears, urine, and other techniques known by one of skill in the art. In some embodiments the sample comprises circulating tumor RNA (ctRNA). In some embodiments the sample comprises peripheral blood mononuclear cells (PBMCs). In some cases, the genetic material is obtained from a tumor biopsy or liquid biopsy. In some embodiments, a tumor biopsy comprises a formalin-fixed paraffin embedded biopsy, a fresh frozen biopsy, a fresh biopsy, or a frozen biopsy. In some embodiments, a liquid biopsy comprises PBMCs, circulating tumor RNA, plasma cell-free RNA, or circulating tumor cells (CTCs). Tumor biopsies can undergo additional analytic processing for sample dissociation, cell sorting, and enrichment of cell populations of interest.

In some embodiments, methods of detecting a presence, absence, or level of a biomarker in the sample obtained from the subject involve detecting a nucleic acid sequence. In some cases, the nucleic acid sequence comprises deoxyribonucleic acid (DNA), such as in the case of detecting complementary DNA (cDNA) of an mRNA transcript. In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Disclosed herein, in some embodiments, the biomarker is detected by subjecting a sample obtained from the subject to a nucleic acid-based detection assay. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, microarray, or sequencing. In some embodiments, the sequencing technique comprises next generation sequencing. In some embodiments, the methods involve a hybridization assay such as fluorogenic qPCR (e.g., TaqMan™, SYBR green, SYBR green I, SYBR green II, SYBR gold, ethidium bromide, methylene blue, Pyronin Y, DAPI, acridine orange, Blue View or phycoerythrin), which involves a nucleic acid amplification reaction with a specific primer pair, and hybridization of the amplified nucleic acid probes comprising a detectable moiety or molecule that is specific to a target nucleic acid sequence. In some instances, a number of amplification cycles for detecting a target nucleic acid in a qPCR assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles. In some instances, hybridization may occur at standard hybridization temperatures, e.g., between about 35° C. and about 65° C. in a standard PCR buffer.

An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more gene products described herein. In some instances, the nucleic acid probe specific to a biomarker comprises a nucleic acid probe sequence sufficiently complementary to the polynucleotide sequence of the biomarker. In some instances, the biomarker comprises a transcribed polynucleotide sequence (e.g., RNA, cDNA). In some embodiments, the nucleic acid probe can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the target nucleic acid sequence. In some embodiments, the target nucleic acid sequence is immobilized on a solid surface and contacted with a probe, for example by running the isolated target nucleic acid sequence on an agarose gel and transferring the target nucleic acid sequence from the gel to a membrane, such as nitrocellulose. In some embodiments, the probe(s) are immobilized on a solid surface, for example, in an Affymetrix gene chip array, and the probe(s) are contacted with the target nucleic acid sequence.

In some embodiments, the term “probe” with regards to nucleic acids, refers to any nucleic acid molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate, and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some embodiments, detecting the one or more biomarkers, such as gene products in a predictive response signature (PRS), comprises sequencing genetic material obtained from a sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modem sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Disclosed herein are methods comprising: (a) providing a sample obtained from a subject with a proliferative disease or condition (e.g., cancer); (b) assaying to detect in the sample obtained from the subject a presence or absence of the relevant biomarker; and (c) detecting the presence or absence of the biomarker in the sample using the methods described herein. In some cases, a hybridization assay, such as those described herein, is used to detect the biomarker in the sample. Exemplary probe sequences that are hybridizable to a target nucleic acid sequence (e.g., one or more genes in the biomarker, such as the PRS) comprise at least 10, but no more than 100 contiguous nucleotides comprising the relevant sequence. In some cases, RNA sequencing (RNAseq) is used to detect the one or more biomarkers.

Detection of the relevant biomarker, in some cases, involves amplification of the subject's nucleic acid by the polymerase chain reaction (PCR). In some embodiments, the PCR assay involves use of a pair of primers capable of amplifying at least about 10 contiguous nucleobases within a nucleic acid sequence, thereby amplifying the one or more gene products in the biomarker. In fluorogenic quantitative PCR, quantitation is based on amount of fluorescence signals (TaqMan and SYBR green). In some embodiments, the nucleic acid probe is conjugated to a detectable molecule. The detectable molecule may be a fluorophore. The nucleic acid probe may also be conjugated to a quencher.

In some embodiments, the assay for detecting the presence or absence of a relevant biomarker comprises reverse-transcribing the relevant mRNA molecule to produce a corresponding complementary DNA (cDNA) molecule). In some embodiments, the assay further comprises contacting the cDNA molecule with a nucleic acid probe comprising a nucleic acid sequence that is complementary to a nucleic acid sequence of the cDNA molecule. In some embodiments, the assay comprises detecting a double-stranded hybridization product between the nucleic acid probe and the cDNA molecule. In some embodiments, the hybridization product is further amplified using a pair of primers. In some embodiments, the primers comprises a first primer with a nucleic acid sequence comprising at least 10 but not more than 50 contiguous nucleic acids within a relevant nucleic acid sequence that binds to a top strand of the double-stranded hybridization product; and a second primer with a nucleic acid sequence comprising at least 10 but not more than 50 contiguous nucleic acids within a nucleic acid sequence that is reverse complement to the relevant nucleic acid sequence that binds to a bottom strand of the double-stranded hybridization product.

Disclosed herein, in some embodiments, are methods comprising preparing a complementary DNA (cDNA) library. In some embodiments, the cDNA library is sequenced using suitable sequence methodologies disclosed herein. In some embodiments, the cDNA library is labeled, a plurality of nucleic acid probes is generated, and fixed to an immobile surface (such as a microarray). In some embodiments, the plurality of nucleic acid probes is capable of hybridizing to at least about 10 contiguous nucleotides of the two or more genes in a sample obtained from the subject. In some embodiments, detecting the presence of or absence of a biomarker includes detecting a high or a low level of expression of the two or more genes as compared to a reference level.

Disclosed herein, in some embodiments, genetic material is extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

Circulating Tumor DNA (ctDNA) and RNA (ctRNA)

In some aspects, circulating tumor DNA (ctDNA) is used to assess the presence of certain DNA molecules and circulating tumor RNA (ctRNA) is used to assess the expression levels of RNA molecules, shed by the tumor into the blood stream.

In some embodiments, detection of ctDNA or ctRNA is useful, for example, for detecting and diagnosing a tumor. Because tumor DNA and RNA has acquired multiple genetic mutations, leading to tumor development, ctDNA and ctRNA are not an exact match to the individual's DNA and RNA, respectively. Finding DNA and RNA with genetic differences aids in tumor detection. Diagnosing the type of tumor using ctDNA or ctRNA can reduce the need for getting a sample of the tumor tissue (tumor biopsy), which can be challenging when a tumor is difficult to access, such as a tumor in the brain or lung.

In some embodiments, a decrease in the quantity of ctDNA or ctRNA suggests the solid tumor is shrinking and treatment with a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt thereof is effective. In some embodiments, a lack of ctDNA or ctRNA in the bloodstream indicates that the cancer has not returned after treatment with a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt thereof.

Described herein are methods of assessing genetic alterations by ctDNA or ctRNA genomic profiling. In some embodiments, the genomic profiling is performed after each treatment cycle with a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt thereof. In some embodiments, the gene mutations indicate that the cancer is becoming resistant to the treatment with a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt thereof. In some embodiments, the lack of gene mutations indicate that the cancer is not becoming resistant to the treatment with a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt thereof.

The compounds of Formula (I), Formula (Ia), Formula (Ib), Formula (II), Formula (III), Formula (IV), Formula (IVa), Formula (IVb), Formula (V), Formula (Va), or Formula (Vb) may be administered as prodrugs. Thus certain derivatives of the compounds, which may have little or no pharmacological activity themselves can, when administered to a mammal, be converted into a compound having the desired activity, for example, by hydrolytic cleavage. Such derivatives are referred to as “prodrugs.” Prodrugs can, for example, be produced by replacing appropriate functionalities present in the compound of Formula (I), Formula (Ia), Formula (Ib), Formula (II), Formula (III), Formula (IV), Formula (IVa), Formula (IVb), Formula (V), Formula (Va), or Formula (Vb) with certain moieties known to those skilled in the art. See, e.g. “Pro-drugs as Novel Delivery Systems”, Vol. 14, ACS Symposium Series (T Higuchi and W Stella) and “Bioreversible Carriers in Drug Design”, Pergamon Press, 1987 (ed. E B Roche, American Pharmaceutical Association), the disclosures of which are incorporated herein by reference in their entireties. Some examples of such prodrugs include: an ester moiety in the place of a carboxylic acid functional group; an ether moiety or an amide moiety in place of an alcohol functional group; and an amide moiety in place of a primary or secondary amino functional group. Examples of replacement groups are known to those of skill in the art. See, e.g. “Design of Prodrugs” by H Bundgaard (Elsevier, 1985), the disclosure of which is incorporated herein by reference in its entirety.

Salts of the present invention can be prepared according to methods known to those of skill in the art. Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edisylate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, gamma.-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

The compounds of Formula (I), Formula (Ia), Formula (Ib), Formula (II), Formula (III), Formula (IV), Formula (IVa), Formula (IVb), Formula (V), Formula (Va), or Formula (Vb) that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

The compounds of Formula (I), Formula (Ia), Formula (Ib), Formula (II), Formula (III), Formula (IV), Formula (IVa), Formula (IVb), Formula (V), Formula (Va), or Formula (Vb) that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the compound of Formula (I), Formula (Ia), Formula (Ib), Formula (II), Formula (III), Formula (IV), Formula (IVa), Formula (IVb), Formula (V), Formula (Va), or Formula (Vb) is a base, the desired salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of Formula (I), Formula (Ia), Formula (Ib), Formula (II), Formula (III), Formula (IV), Formula (IVa), Formula (IVb), Formula (V), Formula (Va), or Formula (Vb) is an acid, the desired salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum, and lithium.

If the compound of Formula (I), Formula (Ia), Formula (Ib), Formula (II), Formula (III), Formula (IV), Formula (IVa), Formula (IVb), Formula (V), Formula (Va), or Formula (Vb) is a solid, it is understood by those skilled in the art that the compounds or salts thereof may exist in different crystal or polymorphic forms, all of which are intended to be within the scope of the present invention and specified formulas.

Also provided herein are isotopically-labeled compounds of Formula (I), Formula (Ia), Formula (Ib), Formula (II), Formula (III), Formula (IV), Formula (IVa), Formula (IVb), Formula (V), Formula (Va), or Formula (Vb), wherein one or more atoms is replaced by an atom having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labeled compounds of the invention, for example, those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula (I), Formula (Ia), Formula (Ib), Formula (II), Formula (III), Formula (IV), Formula (IVa), Formula (IVb), Formula (V), Formula (Va), or Formula (Vb) can generally be prepared by conventional techniques known to those skilled in the art or by processes analogous to those described herein, using an appropriate isotopically-labeled reagent in place of the non-labeled reagent otherwise employed.

In one aspect, the compositions of compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, described herein are used for the treatment of cancer in a subject. In one embodiment, such compositions are in the form of suitable dosage forms. Suitable dosage forms include, for example, liquids, suspensions, powders for reconstitution, tablets, pills, sachets, or capsules of hard or soft gelatin (See, e.g., Remington: The Science and Practice of Pharmacy (Gennaro, 21st Ed. Mack Pub. Co., Easton, PA (2005)).

The compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, may be formulated into pharmaceutical compositions as described below in any pharmaceutical form recognizable to the skilled artisan as being suitable. Pharmaceutical compositions of the invention comprise a therapeutically effective amount of at least one compound of the present invention and an inert, pharmaceutically acceptable carrier or diluent.

The pharmaceutical carriers employed may be either solid or liquid. Exemplary solid carriers are lactose, sucrose, tale, gelatin, agar, pectin, acacia, magnesium stearate, stearic acid, and the like. Exemplary liquid carriers are syrup, peanut oil, olive oil, water, and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol™, Gelucire™ or the like, or formulator, such as CMC (carboxy-methylcellulose), PG (propyleneglycol), or PEG (polyethyleneglycol), may be added. Gelucire™, a semi-solid vehicle that protects active ingredients from light, moisture, and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, a salt of a compound of the present invention may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, a compound of the present invention is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present invention may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the invention to be formulated as tablets, pills, dragees, capsules, liquids, gels, syrups, slurries, suspensions, and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g. sterile pyrogen-free water, before use.

In addition to the formulations described above, the compounds of the present invention may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide (DMSO) also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire™, Capryol™, Labrafil™, Labrasol™, Lauroglycol™, Plurol™, Peceol™ Transcutol™ and the like may be used.

Further, the pharmaceutical composition may be incorporated into a skin patch for delivery of the drug directly onto the skin.

It will be appreciated that the actual dosages of the agents of this invention will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations of the present invention may contain a compound of the present invention, or a salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg.

Additionally, the pharmaceutically acceptable formulations of the present invention may contain a compound of the present invention, or a salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds of the present invention, or salts or solvates thereof, may be administered to a mammal suffering from abnormal cell growth, such as a human, either alone or as part of a pharmaceutically acceptable formulation, once a day, twice a day, three times a day, or four times a day, or even more frequently.

Those of ordinary skill in the art will understand that with respect to the compounds Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, the particular pharmaceutical formulation, the dosage, and the number of doses given per day to a mammal requiring such treatment, are all choices within the knowledge of one of ordinary skill in the art and can be determined without undue experimentation.

Dosages of compositions described herein can be determined by any suitable method. Maximum tolerated doses (MTD) and maximum response doses (MRD) for the compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, can be determined via established animal and human experimental protocols as well as in the examples described herein. For example, toxicity and therapeutic efficacy of the compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of the compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, comprising a formulation that corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered in an amount between about 10 mg to 500 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, or a pharmaceutically acceptable salt thereof) is administered in an amount between about 100 mg to about 400 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof is administered in an amount between about 150 mg to about 350 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof is administered in an amount between about 150 mg to about 300 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered in an amount between about 160 mg to about 300 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered in an amount of about 160 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered in an amount of about 200 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered in an amount of about 240 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered in an amount of about 280 mg per day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered in an amount of about 320 mg per day.

In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the subject. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the subject.

In certain embodiments wherein the subject's condition does not improve, upon the doctor's discretion the administration of a composition described herein are administered chronically, that is, for an extended period of time, including throughout the duration of the subject's life in order to ameliorate or otherwise control or limit the symptoms of the subject's disease. In other embodiments, administration of a composition continues until complete or partial response of a disease.

In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered to a subject in need thereof once a day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, is administered to a subject in need thereof twice a day. In some embodiments, a compound of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof is administered to a subject in need thereof three times a day.

In some instances, the methods described herein comprise administering the compositions and formulations comprising the compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, in combination with one or more additional therapeutic agents, to the subject or subject in need thereof in multiple cycles repeated on a regular schedule with periods of rest in between each cycle. For example, in some instances, treatment given for one week followed by three weeks of rest is one treatment cycle.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is 56 days. In some embodiments, a treatment cycle lasts one, two, three, or four weeks. In some embodiments, a treatment cycle lasts three weeks. In some embodiments, a treatment cycle lasts four weeks. The number of treatment doses scheduled within each cycle also varies depending on the drugs being given.

### Kits and Articles of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded, or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### Methods of Preparation

Compounds of Formula (I), Formula (Ia), Formula (Ib), Formula (II), Formula (III), Formula (IV), Formula (IVa), Formula (IVb), Formula (V), Formula (Va), or Formula (Vb), or a pharmaceutically acceptable salt, solvate, or stereoisomer thereof, may be prepared using the reaction routes and synthetic schemes described below, employing the techniques available in the art using starting materials that are readily available. The preparation of certain embodiments of the present invention is described in detail in the following examples, but those of ordinary skill in the art will recognize that the preparations described may be readily adapted to prepare other embodiments of the present invention. For example, the synthesis of non-exemplified compounds according to the invention may be performed by modifications apparent to those skilled in the art, e.g. by appropriately protecting interfering groups, by changing to other suitable reagents known in the art, or by making routine modifications of reaction conditions. Alternatively, other reactions referred to herein or known in the art will be recognized as having adaptability for preparing other compounds of the invention.

Compounds of Formula (I), Formula (Ia), Formula (Ib), Formula (II), Formula (III), Formula (IV), Formula (IVa), Formula (IVb), Formula (V), Formula (Va), or Formula (Vb) may be prepared from Compounds of Formula (VI), wherein R², R³, R^{4a}, R^{4b}, R^{4c}, R⁵, R⁶, R⁷, R^{8a}, R^{8b}, R^{8c}, R^{8d}, and R⁹ are as defined herein, by allowing the compounds to react with compounds of Formula (VII), wherein A, R¹, R⁹, and n are as defined herein, and wherein LG is a leaving group. LG that may be used include halogens, such as chloro, bromo, and iodo. The reaction of the compounds of Formula (VI) with compounds of Formula (VII) may be conducted using methods known to those of ordinary skill in the art. For example, the reaction of the compounds of Formula (VI) with compounds of Formula (VII) may be conducted in aprotic solvents, such as acetonitrile, DMF, and the like, protic solvents, such as water or alcohols, mixtures of protic and aprotic solvents, such as mixtures of acetonitrile and water, at temperatures in the range from 25° C. to 200° C., and in the presence of an acid or a base. Compounds of Formula (VI) may be prepared by methods disclosed herein and/or by methods known to those of ordinary skill in the art.

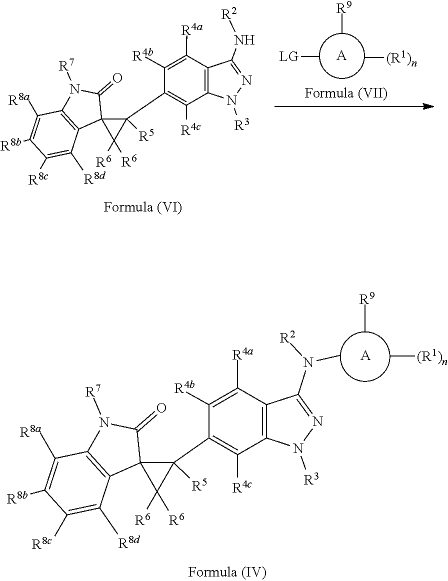

Alternatively, compounds of Formula (IV) may be prepared by allowing compounds of Formula (VIII), wherein R³, R^{4a}, R^{4b}, R^{4c}, R⁵, R⁶, R⁷, R^{8a}, R^{8b}, R^{8c}, R^{8d}, and R⁹ are as defined herein, and Hal is a halogen, such as bromo or iodo, by allowing the compounds to react with compounds of Formula (IX), wherein A, R¹, R², R⁹ and n are as defined herein. Such reactions may be performed in the presence of a catalytic amount of a palladium-containing compound, such as palladium(0) bis(dibenzylideneacetone) (also known as Pd(dba)₂), a phosphate ligand, such as 4,5-bis(diphenylphosphino)-9,9-dimethylxanthene (also known as Xantphos), a base, and in an aprotic solvent. The base may be selected from an organic base, such as a tertiary amine, for example triethyl amine, or an inorganic base, for example cesium carbonate. The aprotic solvent may be, for example, toluene. The reactions of the compounds of Formula (VIII) with the compounds of Formula (IX) may be conducted at temperatures in the range from 25° C. to 200° C., for example such reactions may be conducted in toluene at a temperature of 100° C. The compounds of Formula (IX) are commercially available, or may be prepared by methods known to those having ordinary skill in the art, or by methods similar to those set forth herein.

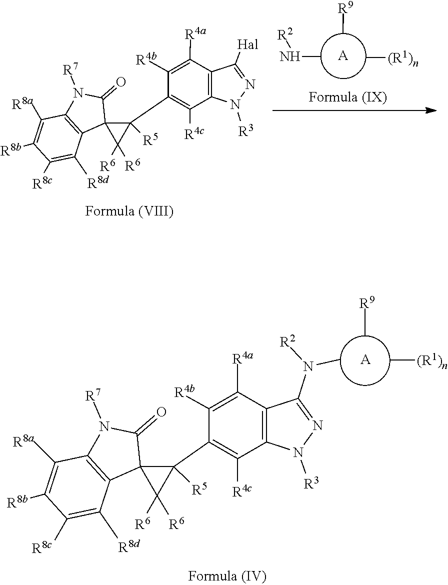

Compounds of Formula (VIII) may be prepared by methods known to those having ordinary skill in the art. For example, the compound (1R,2S)-2-(3-bromo-1H-indazol-6-yl)-5′-methoxyspiro[cyclopropane-1,3′-indolin]-2′-one may be prepared according to the scheme set forth below. Similarly, the compound tert-butyl (1R,2S)-2-(1-(tert-butoxycarbonyl)-3-iodo-1H-indazol-6-yl)-5′-methoxy-2′-oxospiro[cyclopropane-1,3′-indoline]-1′-carboxylate may be prepared according to the scheme set forth below. Other compounds of Formula (VIII) may be prepared by methods known to those of skill in the art by modifications apparent to those skilled in the art, e.g. by using different starting materials, appropriately protecting interfering groups, by changing to other suitable reagents known in the art, or by making routine modifications of reaction conditions.

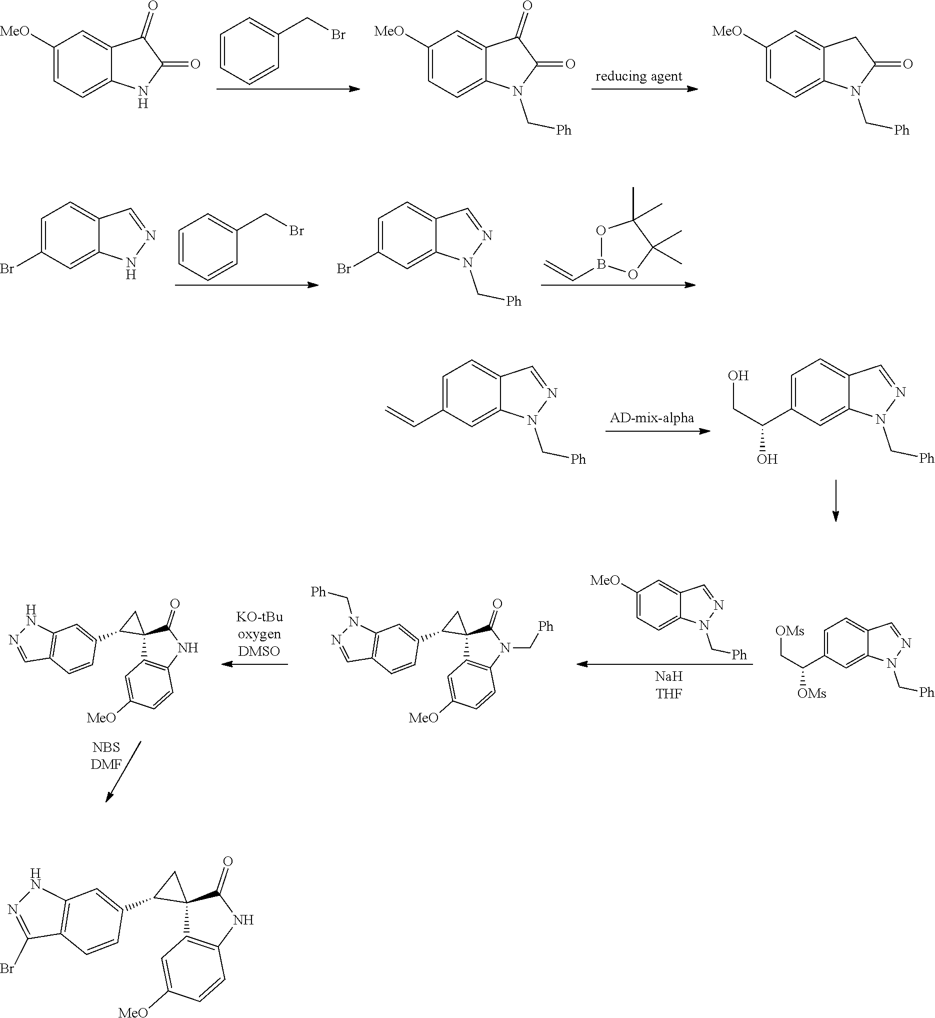

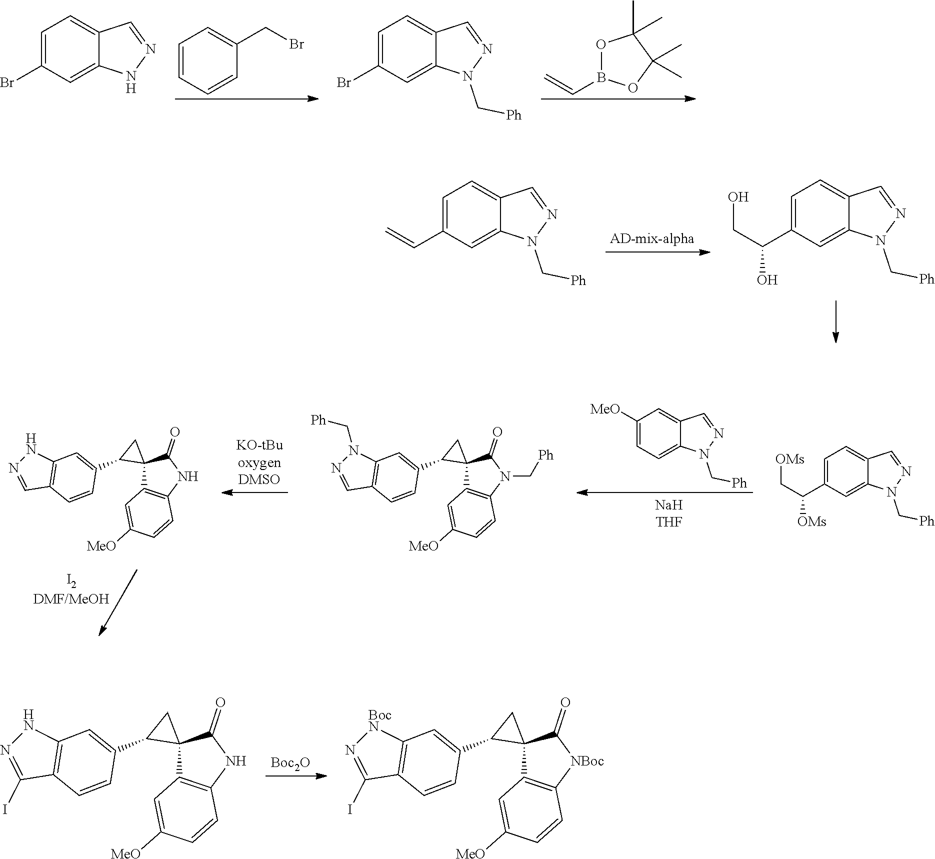

Similarly, (1R,2S)-2-(3-Iodo-1H-indazol-6-yl)-5′-methoxyspiro[cyclopropane-1,3′-indolin]-2′-one may be prepared by allowing (1R,2S)-2-(1H-indazol-6-yl)-5′-methoxyspiro[cyclopropane-1,3′-indolin]-2′-one to react with iodine in DMF and methanol in the presence of potassium carbonate as set forth below.

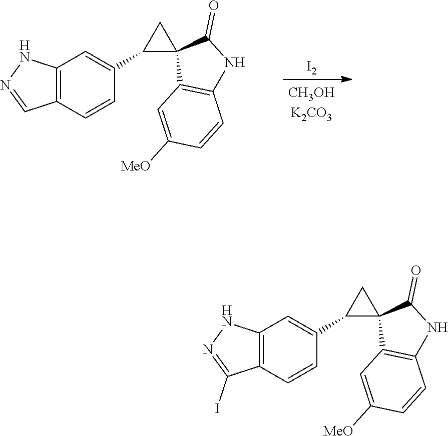 [SMILES: COc1ccc2c(c1)[C@]1(C[C@H]1c1ccc3cn[nH]c3c1)C(=O)N2 ; "I2" ; COc1ccc2c(c1)[C@]1(C[C@H]1c1ccc3c(I)n[nH]c3c1)C(=O)N2]

Compounds of Formula (VI) may be prepared by methods known to those having ordinary skill in the art. For example, tert-butyl (1R,2S)-2-(3-amino-1-(tert-butoxycarbonyl)-1H-indazol-6-yl)-5′-methoxy-2′-oxospiro[cyclopropane-1,3′-indoline]-1′-carboxylate may be prepared from (1R,2S)-2-(3-bromo-1H-indazol-6-yl)-5′-methoxyspiro[cyclopropane-1,3′-indolin]-2′-one as set forth below.

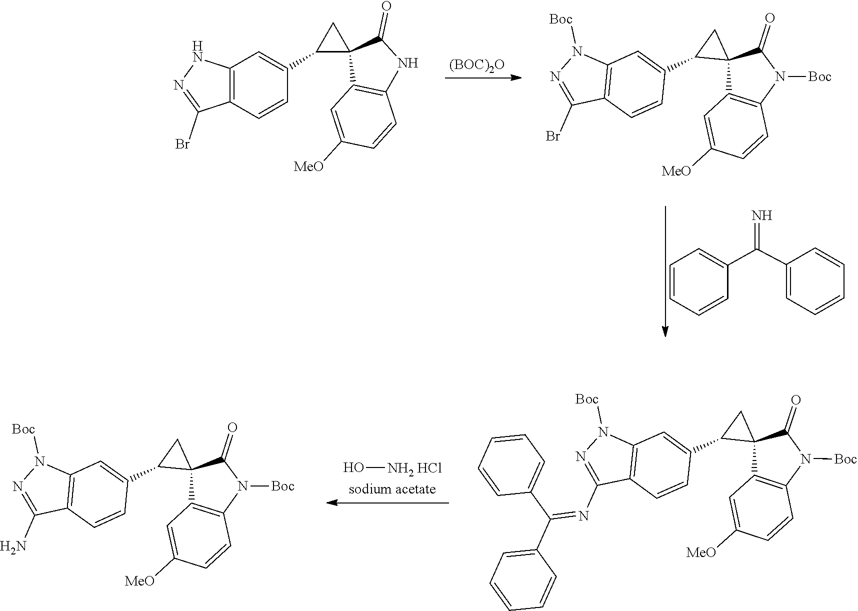

Compounds such as tert-butyl (1R,2S)-2-(3-amino-1-(tert-butoxycarbonyl)-1H-indazol-6-yl)-5′-methoxy-2′-oxospiro[cyclopropane-1,3′-indoline]-1′-carboxylate may be allowed to react with compounds of Formula (VI) as described herein, followed by deprotection of the Boc groups using an acid, such as trifluoroacetic acid, to provide compounds of the present disclosure. For example, tert-butyl (1R,2S)-2-(3-amino-1-(tert-butoxycarbonyl)-1H-indazol-6-yl)-5′-methoxy-2′-oxospiro[cyclopropane-1,3′-indoline]-1′-carboxylate may be allowed to react with 4-chloro-5-methoxypyrimidine to afford (1R,2S)-5′-methoxy-2-(3-((5-methoxypyrimidin-4-yl)amino)-1H-indazol-6-yl)spiro[cyclopropane-1,3′-indolin]-2′-one.

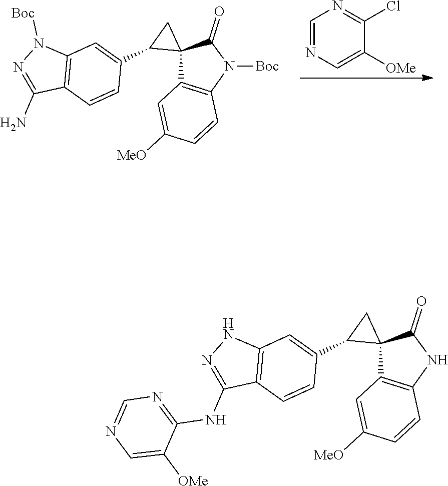 [SMILES: COc1ccc2c(c1)[C@]1(C[C@H]1c1ccc3c(N)nn(C(=O)OC(C)(C)C)c3c1)C(=O)N2C(=O)OC(C)(C)C ; COc1cncnc1Cl ; COc1ccc2c(c1)[C@]1(C[C@H]1c1ccc3c(Nc4ncncc4OC)n[nH]c3c1)C(=O)N2]

In the following preparations and Examples, “Ac” means acetyl, “ACN” and “MeCN” mean acetonitrile, “Me” means methyl, “Et” means ethyl, “Ph” means phenyl, “BOC”, “Boc” or “boc” means N-tert-butoxycarbonyl, “DCM” (CH₂Cl₂) means methylene chloride, “DIPEA” or “DIEA” means diisopropyl ethyl amine, “DMA” means N,N-dimethylacetamide, “DMAP” means 4-(dimethylamino)pyridine, “DMF” means N—N-dimethyl formamide, “DMSO” means dimethylsulfoxide, “DPPP” means 1,3-bis(diphenylphosphino)propane, “HOAc” means acetic acid, “IPA” means isopropyl alcohol, “min” means minute, “NMP” means 1-methyl 2-pyrrolidinone, “TEA” means triethyl amine, “TFA” means trifluoroacetic acid, “DCM” means dichloromethane, “EtOAc” and “EA” mean ethyl acetate, “MgSO₄” means magnesium sulphate, “Na₂SO₄” means sodium sulphate, “MeOH” means methanol, “Et₂O” means diethyl ether, “EtOH” means ethanol, “H₂O” means water, “HCl” means hydrochloric acid, “K₂CO₃” means potassium carbonate, “THF” means tetrahydrofuran, “DBU” means 1,8-diazabicyclo[5.4.0]undec-7-ene, “LiHMDS” or “LHMDS” means lithium hexamethyldisilazide, “TBME” or “MTBE” means tert-butyl methyl ether, “LDA” means lithium diisopropylamide, “N” means Normal, “M” means molar, “mL” means milliliter, “mmol” means millimoles, “pmol” means micromoles, “eq.” means equivalent, “° C.” means degrees Celsius, “Pa” means pascals, “it” or “RT” means room temperature, “h” means hours, “satd.” means saturated, “aq” means aqueous, “anhyd.” or “anh.” means anhydrous, “MBTE” means methyl tert-butyl ether, “PE” means petroleum ether, and “TBSCl” means tert-butyldimethylsily chloride.

### EXAMPLES

### Intermediate 1. (1R,2S)-2-(3-Amino-1H-indazol-6-yl)-5′-methoxyspiro[cyclopropane-1,3′-indolin]-2′-one

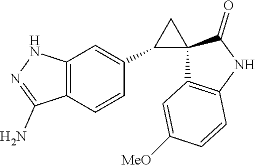 [SMILES: COc1ccc2c(c1)[C@]1(C[C@H]1c1ccc3c(N)n[nH]c3c1)C(=O)N2]

### Step A. (E)-3-(3-Fluoro-4-isocyanobenzylidene)-5-methoxyindolin-2-one

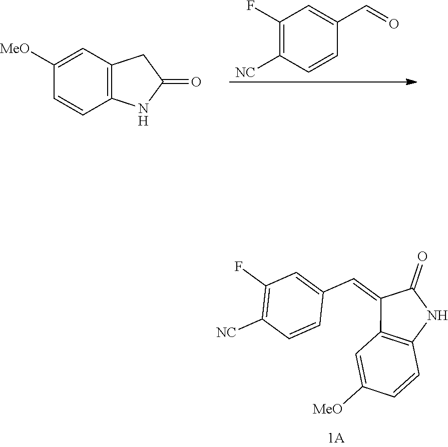 [SMILES: COc1ccc2c(c1)CC(=O)N2 ; N#Cc1ccc(C=O)cc1F ; COc1ccc2c(c1)/C(=C\c1ccc(C#N)c(F)c1)C(=O)N2]

A round bottom flask was charged with 5-methoxyoxindole (5.00 g, 30.6 mmol), 4-cyano-3-fluorobenzaldehyde (4.57 g, 30.6 mmol), piperidine (835 μL, 8.40 mmol) and ethanol (120 mL). The reaction was refluxed for 4 h and was stirred for 16 h at rt. The reaction was cooled to 0° C. and the resulting precipitate was collected by filtration and dried to give the title compound (5.10 g, 57%) as a dark red solid. m/z (ESI, +ve ion)=295.0 [M+H]⁺.

### Step B. racemic-2-Fluoro-4-((1R,2S)-5′-methoxy-2′-oxospiro[cyclopropane-1,3′-indolin]-2-yl)benzonitrile

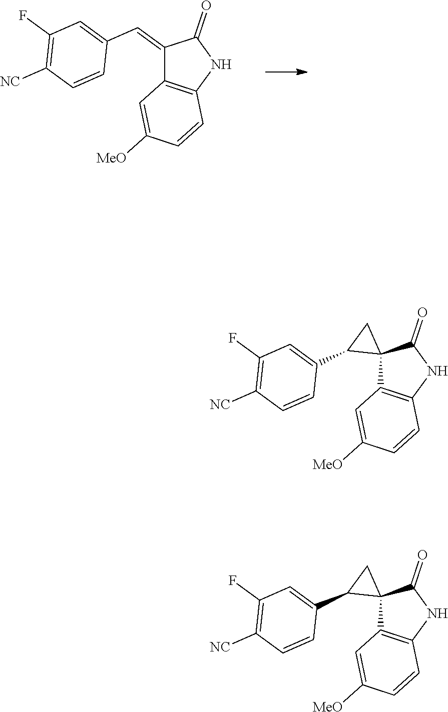 [SMILES: COc1ccc2c(c1)/C(=C\c1ccc(C#N)c(F)c1)C(=O)N2 ; COc1ccc2c(c1)[C@]1(C[C@H]1c1ccc(C#N)c(F)c1)C(=O)N2 ; COc1ccc2c(c1)[C@]1(C[C@@H]1c1ccc(C#N)c(F)c1)C(=O)N2]

To a solution of trimethylsulfoxonium iodide (4.20 g, 19.1 mmol) in anhydrous DMF (173 mL) under nitrogen was added sodium hydride (60% dispersion in oil) (81.5 mg, 2.04 mmol) at 0° C. The mixture was stirred for 15 minutes after which (E)-3-(3-fluoro-4-isocyanobenzylidene)-5-methoxyindolin-2-one (5.10 g, 17.3 mmol) was added to the solution and the reaction was stirred for 1 h at rt. The solution was quenched with satd. aq. ammonium chloride solution and extracted with EtOAc. The organic layer was then washed with brine, dried with anhyd. Na₂SO₄, filtered and concentrated under vacuum. The crude mixture was purified by column chromatography (10% to 65% EtOAc/heptanes, gradient elution), affording the title compound (1.50 g, 28%) as an orange solid. NOESY NMR experiment confirmed relative stereochemistry. m/z (ESI, +ve ion)=309.0 [M+H]⁺. 1H NMR (400 MHz, CDCl₃) δ 8.14 (s, 1H), 7.63-7.54 (m, 1H), 7.13-7.08 (m, 2H), 6.85 (d, J=8.5 Hz, 1H), 6.67 (dd, J=8.5, 2.5 Hz, 1H), 5.55 (d, J=2.4 Hz, 1H), 3.55 (s, 3H), 3.29 (t, J=8.5 Hz, 1H), 2.26 (dd, J=9.0, 5.0 Hz, 1H), 1.94 (dd, J=8.0, 5.0 Hz, 1H). The corresponding diastereoisomer was found to be less polar and was first to elute in the given conditions. m/z (ESI, +ve ion)=309.0 [M+H]⁺. 1H NMR (400 MHz, CDCl₃) δ 8.08 (s, 1H), 7.52 (dd, J=7.8, 6.9 Hz, 1H), 7.21 (s, 1H), 7.19 (s, 1H), 6.78 (d, J=1.5 Hz, 2H), 6.54 (s, 1H), 3.81 (s, 3H), 3.07 (t, J=8.7 Hz, 1H), 2.34 (dd, J=8.5, 5.3 Hz, 1H), 2.12 (dd, J=8.9, 5.3 Hz, 1H).

### Step C. (1R,2S)-2-(3-Amino-1H-indazol-6-yl)-5′-methoxyspiro[cyclopropane-1,3′-indolin]-2′-one

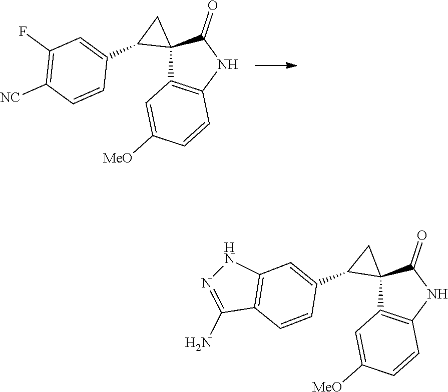 [SMILES: COc1ccc2c(c1)[C@]1(C[C@H]1c1ccc(C#N)c(F)c1)C(=O)N2 ; COc1ccc2c(c1)[C@]1(C[C@H]1c1ccc3c(N)n[nH]c3c1)C(=O)N2]

In a 20 mL vial was dissolved 2-fluoro-4-((1R,2S)-5′-methoxy-2′-oxospiro[cyclopropane-1,3′-indolin]-2-yl)benzonitrile (20.0 mg, 64.9 μmol) in tert-amyl alcohol (10.0 mL) and hydrazine hydrate solution (50.0 μL, 1.58 mmol) was subsequently added. The reaction was refluxed for 16 h. The reaction was cooled to rt and silica was directly added to the mixture and concentrated. The product was purified by column chromatography (0 to 20% MeOH/DCM, gradient elution), affording the title compound (60.0 mg, 58%) as colorless oil. m/z (ESI, +ve ion)=321.1 [M+H]⁺.

### Intermediate 2. (1R,2S)-2-(1H-Indazol-6-yl)-5′-methoxyspiro[cyclopropane-1,3′-indolin]-2′-one

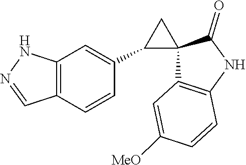 [SMILES: COc1ccc2c(c1)[C@]1(C[C@H]1c1ccc3cn[nH]c3c1)C(=O)N2]

### Step A. 1-Benzyl-5-methoxyindoline-2,3-dione

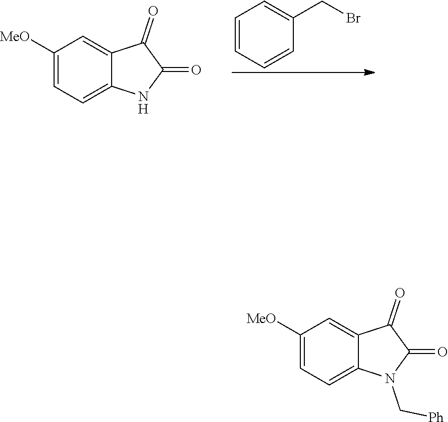

Benzyl bromide (9.65 mL, 79.7 mmol) was added to a mixture of 5-methoxyisatin (12.0 g, 66.4 mmol) and potassium carbonate (27.5 g, 199 mmol) in acetonitrile (250 mL). The mixture was stirred for 15 h at 80° C. then cooled to rt. The mixture was filtered and the filtrate concentrated. It was diluted with water (300 mL) and extracted with EtOAc (3×80 mL). The combined organic layers were washed with brine, then dried (Na₂SO₄), filtered and concentrated. The resulting solid was triturated with heptane, filtered, and washed with heptane, affording the title compound (18.2 g, quantitative yield) as a solid. m/z (ESI, +ve ion)=268.1 [M+H]⁺. 1H NMR (400 MHz, CDCl₃) δ 7.38-7.27 (m, 5H), 7.15 (d, J=2.7 Hz, 1H), 7.02 (dd, J=8.6, 2.7 Hz, 1H), 6.67 (d, J=8.6 Hz, 1H), 4.90 (s, 2H), 3.77 (s, 3H).

### Step B. 1-Benzyl-5-methoxyindolin-2-one

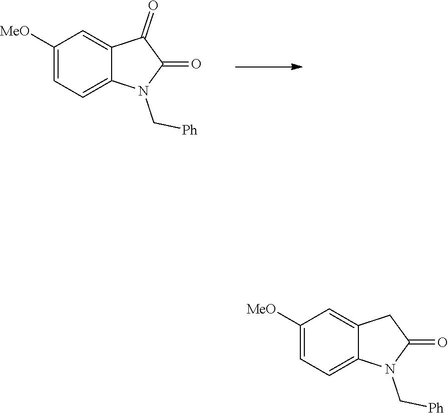 [SMILES: COc1ccc2c(c1)C(=O)C(=O)N2Cc1ccccc1 ; COc1ccc2c(c1)CC(=O)N2Cc1ccccc1]

Hydrazine monohydrate (8.64 mL, 107 mmol) was added to a mixture of 1-benzyl-5-methoxyindoline-2,3-dione (18.2 g, 68.1 mmol) in DMSO (44.1 mL). The mixture was stirred for 5 h at 140° C. then cooled to rt. The mixture was diluted with water (300 mL) and extracted with EtOAc (3×200 mL). The combined organic layers were washed with 1 M H₂SO₄, brine (twice), then dried (Na₂SO₄), filtered and concentrated, affording the title compound (14.0 g, 81%) as a dark oil. m/z (ESI, +ve ion)=254.1 [M+H]⁺. 1H NMR (400 MHz, CDCl₃) δ 7.38-7.22 (m, 5H), 6.90-6.86 (m, 1H), 6.68 (dd, J=8.5, 2.6 Hz, 1H), 6.60 (d, J=8.5 Hz, 1H), 4.89 (s, 2H), 3.75 (s, 3H), 3.61 (s, 2H).

### Step C. 1-Benzyl-6-bromo-1H-indazole

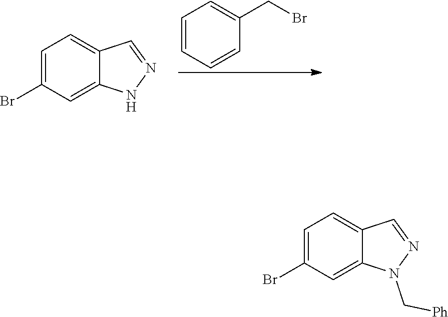

Potassium tert-butoxide (20.5 g, 179 mmol) was added to a mixture of 6-bromo-1H-indazole (30.0 g, 152 mmol) in DMSO (149 mL). The mixture was stirred for 10 min then benzyl chloride (20.8 mL, 179 mmol) was slowly added at 0° C. The mixture was stirred at rt for 3 h then diluted with saturated aqueous NH₄Cl (400 mL) and extracted with MTBE (3×200 mL). The combined organic layers were washed with brine twice, then dried (Na₂SO₄), filtered and concentrated to give crude material as a mixture of 1-benzyl-6-bromo-1H-indazole and 2-benzyl-6-bromo-2H-indazole. Benzyl bromide (37.7 mL, 311 mmol) was added to a mixture of 1-benzyl-6-bromo-1H-indazole and 2-benzyl-6-bromo-2H-indazole (31 g, 108 mmol). The mixture was stirred neat at 150° C. After 6 h, benzyl bromide was removed by distillation under high vacuum (vacuum pump) at 130° C. The residue was triturated in heptanes, then filtered and washed with heptanes. The crude material was put on the high vacuum overnight, affording the tittle compound (20.6 g, 67%) as a solid. m/z (ESI, +ve ion)=287.0 [M+H]⁺.

### Step D. 1-Benzyl-6-vinyl-1H-indazole

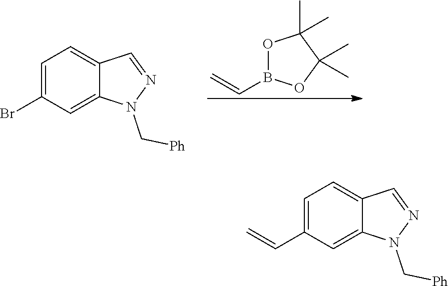 [SMILES: Brc1ccc2cnn(Cc3ccccc3)c2c1 ; C=CB1OC(C)(C)C(C)(C)O1 ; C=Cc1ccc2cnn(Cc3ccccc3)c2c1]

A mixture of 1-benzyl-6-bromo-1H-indazole (6.33 g, 22.0 mmol) and potassium carbonate (9.14 g, 66.1 mmol) in previously degassed (nitrogen bubbled through) DME/water (3:1) (70.0 mL) was purged with nitrogen and nitrogen was further bubbled through the reaction mixture. Vinylboronic acid pinacol ester (4.82 mL, 27.6 mmol) was added, followed by dichlorobis(triphenylphosphine)palladium (II) (774 mg, 1.10 mmol) and the mixture was heated to 80° C. overnight. The mixture was diluted with heptanes and washed with water (3×) and brine. The organic phase was dried with Na₂SO₄, filtered and concentrated. The crude product was purified by column chromatography (0 to 10% EtOAc/hexanes, gradient elution) to afford the title compound (3.80 g, 74%). m/z (ESI, +ve ion)=235.4 [M+H]⁺. 1H NMR (400 MHz, CDCl3) δ 8.01 (d, J=0.9 Hz, 1H), 7.73-7.64 (m, 1H), 7.36-7.23 (m, 5H), 7.23-7.16 (m, 2H), 6.80 (dd, J=17.6, 10.9 Hz, 1H), 5.80 (dd, J=17.5, 0.7 Hz, 1H), 5.60 (s, 2H), 5.30 (dd, J=10.9, 0.6 Hz, 1H).

### Step E. (S)-1-(1-Benzyl-1H-indazol-6-yl)ethane-1,2-diol

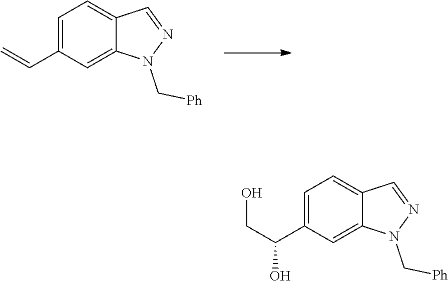 [SMILES: C=Cc1ccc2cnn(Cc3ccccc3)c2c1 ; OC[C@@H](O)c1ccc2cnn(Cc3ccccc3)c2c1]

To a 500 mL flask was added AD-mix-alpha (83.7 g, 59.8 mmol) and t-BuOH/water (1:1) (598 mL) forming a clear biphasic mixture on stirring. The reaction mixture was cooled with an ice bath to 0° C. before adding 1-benzyl-6-vinyl-1H-indazole (14.0 g, 59.8 mmol). The resulting mixture was vigorously stirred at 0° C. and let warm to room temperature with the ice bath slowly warming up. The reaction mixture was stirred for 9 h. The reaction was quench by the portion-wise addition of 92 g of sodium sulfite. The reaction mixture was stirred overnight. The reaction mixture was diluted with brine and DCM and filtered through a pad of Celite. The filtrate was extracted with DCM (4×) and the combined organic layers were dried over MgSO₄, filtered and concentrated. The crude product was recrystallized from toluene (80 mL) to afford the title compound (12.2 g, 76%) as a white solid. m/z (ESI, +ve ion)=269.2 [M+H]⁺. 99.1% ee.

### Step F. (S)-1-(1-Benzyl-1H-indazol-6-yl)ethane-1,2-diyl dimethanesulfonate

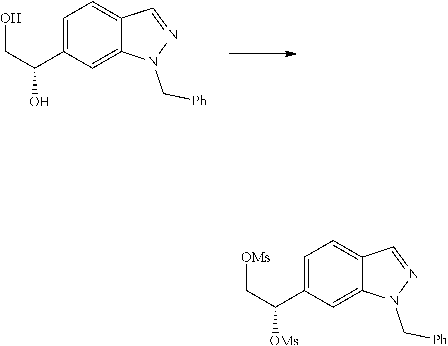 [SMILES: OC[C@@H](O)c1ccc2cnn(Cc3ccccc3)c2c1 ; CS(=O)(=O)OC[C@@H](OS(C)(=O)=O)c1ccc2cnn(Cc3ccccc3)c2c1]

A solution of (S)-1-(1-Benzyl-1H-indazol-6-yl)ethane-1,2-diol (12.2 g, 45.5 mmol) and triethylamine (16.0 mL, 114 mmol) in DCM (227 mL) was cooled in an ice bath and treated by slow addition of methanesulfonyl chloride (7.77 mL, 100 mmol) over 15 minutes. The internal temperature increased to a maximum of 11° C. The resulting mixture was stirred at 0° C. After 6 h, LCMS showed 10% mono mesylated product. 0.400 mL of methanesulfonyl chloride and 0.600 mL of triethylamine were added. The mixture was stirred 1 h and upon completion, diluted with DCM (500 mL) and 1 M aqueous HCl (200 mL) at 0° C. The layers were separated, and the organic layer was washed with saturated aqueous NaHCO₃ (2×200 mL), brine (200 mL), then dried (Na₂SO₄), filtered and concentrated. The crude material was passed through a small pad of Celite eluting with a mixture of DCM/Et₂O (1:1). Removal of the solvents gave a white solid. The solid was triturated in Et₂O (40 mL) and the precipitate was collected by filtration, affording the title compound (17.5 g, 91%) as white crystalline solid. m/z (ESI, +ve ion)=425.0 [M+H]⁺. 1H NMR (400 MHz, CDCl₃) δ 8.08 (s, 1H), 7.81 (d, J=8.3 Hz, 1H), 7.41 (s, 1H), 7.35-7.27 (m, 3H), 7.18 (dd, J=17.3, 7.5 Hz, 3H), 5.89 (dd, J=8.6, 3.2 Hz, 1H), 5.66 (d, J=15.8 Hz, 1H), 5.60 (d, J=15.8 Hz, 1H), 4.53 (dd, J=11.9, 8.6 Hz, 1H), 4.40 (dd, J=11.9, 3.3 Hz, 1H), 3.05 (s, 3H), 2.75 (s, 3H).

### Step G. (1R,2S)-1′-Benzyl-2-(1-benzyl-1H-indazol-6-yl)-5′-methoxyspiro[cyclopropane-1,3′-indolin]-2′-one

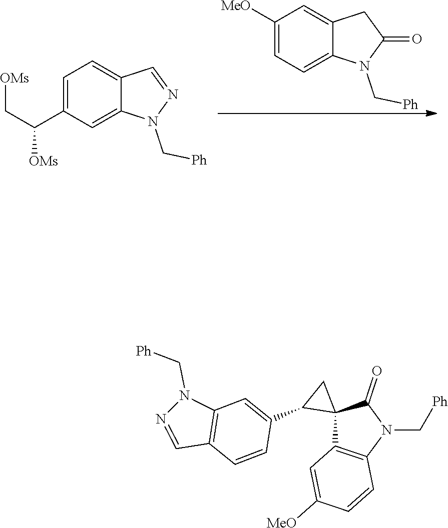 [SMILES: CS(=O)(=O)OC[C@@H](OS(C)(=O)=O)c1ccc2cnn(Cc3ccccc3)c2c1 ; COc1ccc2c(c1)CC(=O)N2Cc1ccccc1 ; COc1ccc2c(c1)[C@]1(C[C@H]1c1ccc3cnn(Cc4ccccc4)c3c1)C(=O)N2Cc1ccccc1]

A solution of (S)-1-(1-benzyl-H-indazol-6-yl)ethane-1,2-diyl dimethanesulfonate (2.03 g, 8.01 mmol) in dry THF (80 mL) under nitrogen was cooled in an ice bath. Sodium hydride (673 mg, 16.8 mmol) was added portion-wise and the mixture was stirred at 0° C. for 15 min. A solution of 1-benzyl-5-methoxyindolin-2-one (3.40 g, 8.01 mmol) in dry THF (50 mL) was added dropwise with an addition funnel. The reaction mixture was stirred for 3 h at 0° C. The reaction was quenched with satd. NH₄Cl solution, diluted with water, and extracted with EtOAc (3×). The organic layer was dried with anhyd. MgSO₄ and concentrated to a crude product. The crude product was triturated with 3:1 hexanes/EtOAc, affording the title compound (2.10 g, 54%) as an orange solid. m/z (ESI, +ve ion)=486.2 [M+H]⁺.

### Step H. (1R,2S)-2-(1H-Indazol-6-yl)-5′-methoxyspiro[cyclopropane-1,3′-indolin]-2′-one

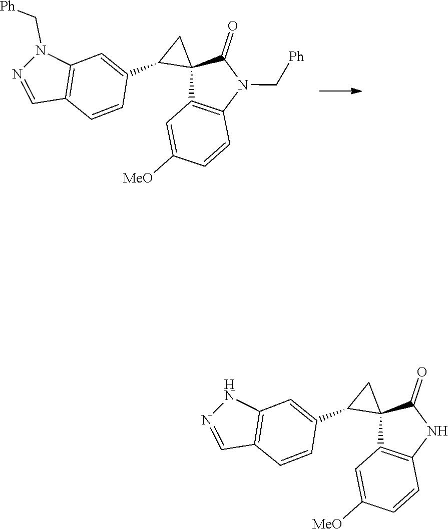 [SMILES: COc1ccc2c(c1)[C@]1(C[C@H]1c1ccc3cnn(Cc4ccccc4)c3c1)C(=O)N2Cc1ccccc1 ; COc1ccc2c(c1)[C@]1(C[C@H]1c1ccc3cn[nH]c3c1)C(=O)N2]

To a round-bottom flask charged with a stir bar was added (1R,2S)-1′-Benzyl-2-(1-benzyl-1H-indazol-6-yl)-5′-methoxyspiro[cyclopropane-1,3′-indolin]-2′-one (4.00 g, 8.24 mmol) in THF (118 mL). The solution was cooled to 0° C. and potassium tert-butoxide (23.0 mL, 165 mmol) was added portion-wise over 20 min and then DMSO (10.7 mL) was added. Oxygen was bubbled through the solution at 0° C. for 1 h. The reaction was quenched with satd. aqueous NH₄Cl at 0° C. and diluted with EtOAc (50 mL). The mixture was washed with satd. aqueous NH₄Cl (1×) and extracted with EtOAc (2×). The organic phase was dried over Na₂SO₄, filtered, and concentrated under vacuum. The crude product was triturated in Et₂O and recrystallized from ethanol, affording the title compound (2.56 g, 56%). m/z (ESI, +ve ion)=306.4 [M+H]⁺.

### Intermediate 3a: (1R,2S)-2-(3-Bromo-1H-indazol-6-yl)-5′-methoxyspiro[cyclopropane-1,3′-indolin]-2′-one

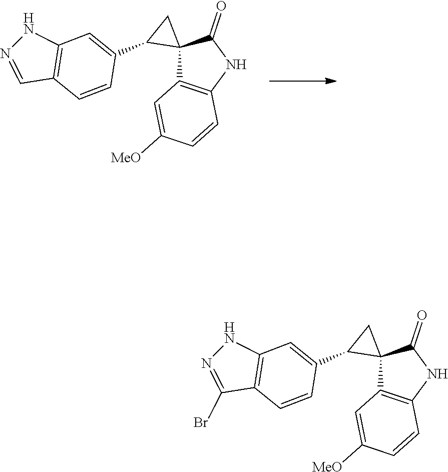 [SMILES: COc1ccc2c(c1)[C@]1(C[C@H]1c1ccc3cn[nH]c3c1)C(=O)N2 ; COc1ccc2c(c1)[C@]1(C[C@H]1c1ccc3c(Br)n[nH]c3c1)C(=O)N2]

In a flask, (1R,2S)-2-(1H-indazol-6-yl)-5′-methoxyspiro[cyclopropane-1,3′-indolin]-2′-one (4.49 g, 12.5 mmol) was dissolved in DMF (16.7 mL) and NBS (2.70 g, 15.0 mmol) dissolved in DMF (8.33 mL) was added dropwise at 0° C. The reaction was stirred for 2 h at rt. The reaction was quenched with an aqueous solution of Na₂S₂O₃ and extracted with EtOAc (3×100 mL). The combined organic layers were washed with brine, dried (Na₂SO₄), filtered and concentrated. The crude product was purified by column chromatography (40 to 100% EtOAc/hexanes, gradient elution), affording the title compound (3.12 g, 65%). m/z (ESI, +ve ion)=384.0, 386.0 [M+H]⁺.

### Intermediate 3b: (1R,2S)-2-(3-Iodo-1H-indazol-6-yl)-5′-methoxyspiro[cyclopropane-1,3′-indolin]-2′-one

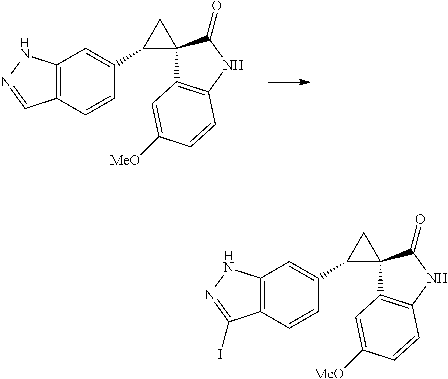 [SMILES: COc1ccc2c(c1)[C@]1(C[C@H]1c1ccc3cn[nH]c3c1)C(=O)N2 ; COc1ccc2c(c1)[C@]1(C[C@H]1c1ccc3c(I)n[nH]c3c1)C(=O)N2]

To an oven-dried flask was added (1R,2S)-2-(1H-indazol-6-yl)-5′-methoxyspiro[cyclopropane-1,3′-indolin]-2′-one (4.00 g, 13.1 mmol) followed by DMF (8 mL and Methanol (8 mL). To this suspension was added K2CO3 (3.62 g, 26.2 mmol). Finally, molecular iodine (4.32 g, 17.0 mmol) dissolved in DMF (8 mL) and was added dropwise and allowed to stir at rt. After 4 h, the reaction was complete. The mixture was quenched with Na2S2O3 in water and stirred for 2 h. Solid was collected by filtration and washed with water. Wet solid was frozen and lyophilized, affording the title compound (4.4 g, 78% yield).

### Intermediate 4a: Tert-butyl 3-bromo-6-((1R,2S)-1′-(tert-butoxycarbonyl)-5′-methoxy-2′-oxospiro[cyclopropane-1,3′-indolin]-2-yl)-1H-indazole-1-carboxylate

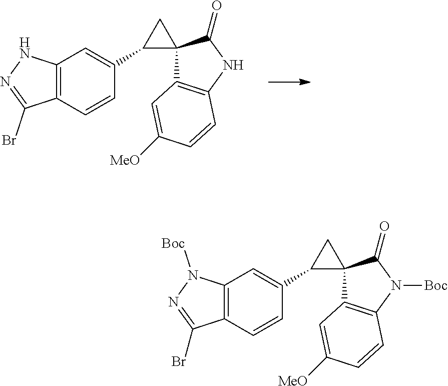 [SMILES: COc1ccc2c(c1)[C@]1(C[C@H]1c1ccc3c(Br)n[nH]c3c1)C(=O)N2 ; COc1ccc2c(c1)[C@]1(C[C@H]1c1ccc3c(Br)nn(C(=O)OC(C)(C)C)c3c1)C(=O)N2C(=O)OC(C)(C)C]

4-Dimethylaminopyridine (79.8 mg, 640 mol) was added to a solution of triethylamine (3.61 mL, 25.6 mmol), di-tert-butyl dicarbonate (4.0 mL, 17.3 mmol) and (1R,2S)-2-(3-bromo-1H-indazol-6-yl)-5′-methoxyspiro[cyclopropane-1,3′-indolin]-2′-one (2.46 g, 6.40 mmol) in DCM (24 mL). The solution was stirred at room temperature for 16 h. LCMS showed incomplete conversion. Di-tert-butyl dicarbonate (0.75 mL, 3.2 mmol, 0.5 equiv.) was added and the reaction was stirred another hour. The crude product was purified by column chromatography (0 to 20% EtOAc/heptanes, gradient elution), affording the title compound (3.06 g, 82%) as a yellow foamy solid. m/z (ESI, +ve ion)=384.0, 386.0 [M+H-Boc]⁺.

### Intermediate 4b: tert-butyl (1R,2S)-2-(1-(tert-butoxycarbonyl)-3-iodo-1H-indazol-6-yl)-5′-methoxy-2′-oxospiro[cyclopropane-1,3′-indoline]-1′-carboxylate

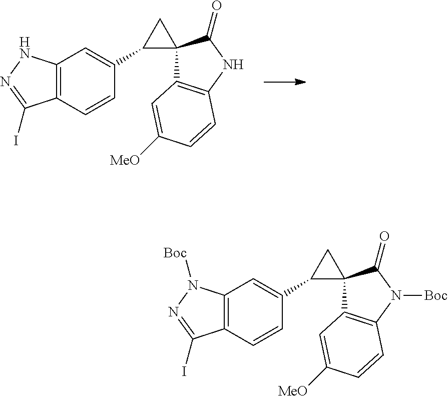 [SMILES: COc1ccc2c(c1)[C@]1(C[C@H]1c1ccc3c(I)n[nH]c3c1)C(=O)N2 ; COc1ccc2c(c1)[C@]1(C[C@H]1c1ccc3c(I)nn(C(=O)OC(C)(C)C)c3c1)C(=O)N2C(=O)OC(C)(C)C]

To an oven-dried flask was added 4-dimethylaminopyridine (9.0 mg, 0.07 mmol) followed by (1R,2S)-2-(3-iodo-1H-indazol-6-yl)-5′-methoxyspiro[cyclopropane-1,3′-indolin]-2′-one (637 mg, 1.48 mmol), N-ethyl-N-isopropyl-propan-2-amine (1.0 mL, 5.9 mmol) and MeCN (5.0 mL). The mixture was stirred at room temperature and di-tert-butyl dicarbonate (967 mg, 4.43 mmol) was added, resulted in light yellow homogeneous solution. After 2 h, the reaction mixture was concentrated and the resulting residue was purified by column chromatography (0% to 25%, EtOAc/hexanes, gradient elution) to afford the product as a white foam (822 mg, 88%).

### Intermediate 5: Tert-butyl 3-amino-6-((1R,2S)-1′-(tert-butoxycarbonyl)-5′-methoxy-2′-oxospiro[cyclopropane-1,3′-indolin]-2-yl)-1H-indazole-1-carboxylate

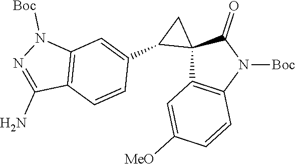 [SMILES: COc1ccc2c(c1)[C@]1(C[C@H]1c1ccc3c(N)nn(C(=O)OC(C)(C)C)c3c1)C(=O)N2C(=O)OC(C)(C)C]

### Step A. Tert-butyl 6-((1R,2S)-1′-(tert-butoxycarbonyl)-5′-methoxy-2′-oxospiro[cyclopropane-1,3′-indolin]-2-yl)-3-((diphenylmethylene)amino)-1H-indazole-1-carboxylate

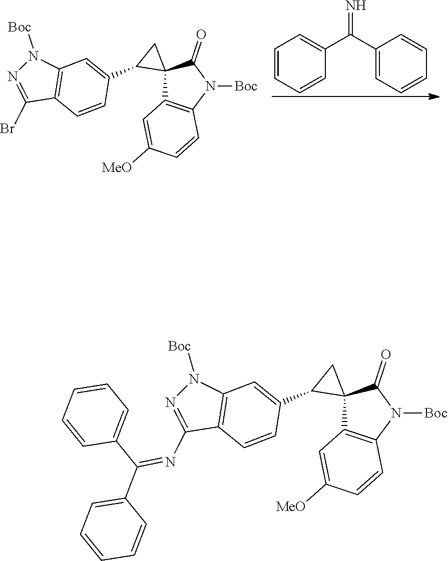

A microwave vial was charged with tert-butyl 3-bromo-6-((1R,2S)-1′-(tert-butoxycarbonyl)-5′-methoxy-2′-oxospiro[cyclopropane-1,3′-indolin]-2-yl)-1H-indazole-1-carboxylate (1.00 g, 1.71 mmol), cesium carbonate (1.14 g, 3.42 mmol), Pd₂(dba)₃ (157 mg, 171 mol) and XantPhos (101 mg, 171 mol). Dry dioxane (17.1 mL) followed by benzophenone imine (310 L, 1.83 mmol) were added and nitrogen was bubbled through the reaction mixture for 5 min. The vial was sealed, and the reaction mixture was heated to 90° C. for 2 h in an oil bath. A satd. aqueous solution of NaHCO₃ was added and the reaction mixture was extracted with EtOAc (3×). The combined extracts were then washed with brine, dried with anhyd. Na₂SO₄, filtered and concentrated under vacuum. The crude product was purified by column chromatography (0 to 30% EtOAc/heptanes, gradient elution), affording the title compound (1.03 g, 88%) as yellow oil. m/z (ESI, +ve ion)=685.4 [M+H]⁺.

### Step B. Tert-butyl 3-amino-6-((1R,2S)-1′-(tert-butoxycarbonyl)-5′-methoxy-2′-oxospiro[cyclopropane-1,3′-indolin]-2-yl)-1H-indazole-1-carboxylate

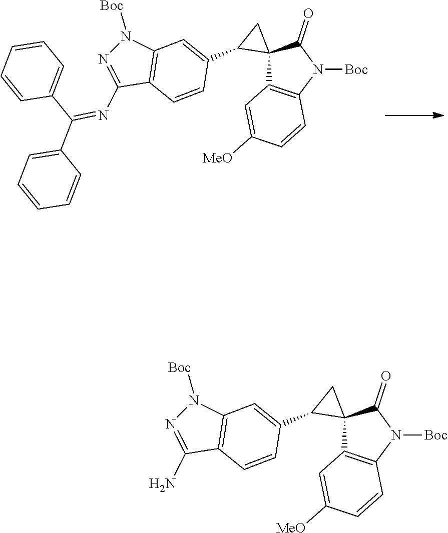 [SMILES: COc1ccc2c(c1)[C@]1(C[C@H]1c1ccc3c(N=C(c4ccccc4)c4ccccc4)nn(C(=O)OC(C)(C)C)c3c1)C(=O)N2C(=O)OC(C)(C)C ; COc1ccc2c(c1)[C@]1(C[C@H]1c1ccc3c(N)nn(C(=O)OC(C)(C)C)c3c1)C(=O)N2C(=O)OC(C)(C)C]

Hydroxylamine hydrochloride (101 mg, 1.46 mmol) and sodium acetate (120 mg, 1.46 mmol) were added to tert-butyl 6-((1R,2S)-1′-(tert-butoxycarbonyl)-5′-methoxy-2′-oxospiro[cyclopropane-1,3′-indolin]-2-yl)-3-((diphenylmethylene)amino)-1H-indazole-1-carboxylate (1.00 g, 1.46 mmol) in dry MeOH (14.6 mL) at room temperature and the reaction was stirred for 16 h. The solvent was removed under reduced pressure. The crude product was purified by column chromatography (0 to 60% EtOAc/heptanes, gradient elution), affording the title compound (640 mg, 84%) as yellow solid. m/z (ESI, +ve ion)=521.0 [M+H]+. 1H NMR (400 MHz, CDCl3) δ8.06 (s, 1H), 7.78 (d, J=8.9 Hz, 1H), 7.41 (d, J=8.2 Hz, 1H), 7.01 (d, J=8.2 Hz, 1H), 6.66 (dd, J=8.9, 2.6 Hz, 1H), 5.55 (d, J=2.3 Hz, 1H), 4.44 (s, 2H), 3.49 (t, J=8.6 Hz, 1H), 3.37 (s, 3H), 2.34 (dd, J=9.2, 4.8 Hz, 1H), 2.14-2.06 (m, 1H), 1.67 (d, J=2.4 Hz, 18H).

### Example 2. (1R,2S)-2-{3-[4-(Methanesulfonyl)-2-methoxyanilino]-1H-indazol-6-yl}-5′-methoxyspiro[cyclopropane-1,3′-indol]-2′(1′H)-one

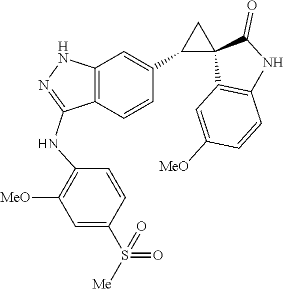 [SMILES: COc1ccc2c(c1)[C@]1(C[C@H]1c1ccc3c(Nc4ccc(S(C)(=O)=O)cc4OC)n[nH]c3c1)C(=O)N2]

### Step A. Tert-butyl (1R,2S)-2-(1-(tert-butoxycarbonyl)-3-((2-methoxy-4-(methylsulfonyl)phenyl)amino)-1H-indazol-6-yl)-5′-methoxy-2′-oxospiro[cyclopropane-1,3′-indoline]-1′-carboxylate

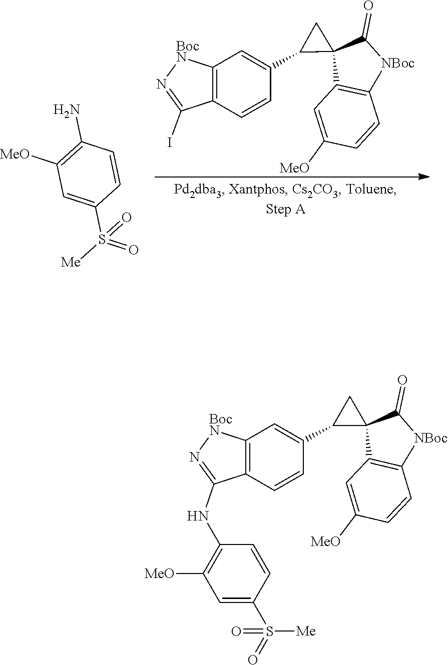 [SMILES: COc1cc(S(C)(=O)=O)ccc1N ; COc1ccc2c(c1)[C@]1(C[C@H]1c1ccc3c(I)nn(C(=O)OC(C)(C)C)c3c1)C(=O)N2C(=O)OC(C)(C)C ; COc1ccc2c(c1)[C@]1(C[C@H]1c1ccc3c(Nc4ccc(S(C)(=O)=O)cc4OC)nn(C(=O)OC(C)(C)C)c3c1)C(=O)N2C(=O)OC(C)(C)C]

Into a 40 mL vial were added 4-methanesulfonyl-2-methoxyaniline (35.06 mg, 0.174 mmol, 1.1 equiv), tert-butyl (1R,2S)-2-(1-(tert-butoxycarbonyl)-3-iodo-1H-indazol-6-yl)-5′-methoxy-2′-oxospiro[cyclopropane-1,3′-indoline]-1′-carboxylate (100 mg, 0.158 mmol, 1.00 equiv), Pd₂(dba)₃ (19 mg, 0.031 mmol, 0.2 equiv), XantPhos (18 mg, 0.031 mmol, 0.2 equiv), Cs₂CO₃ (103 mg, 0.316 mmol, 2 equiv) and toluene (3 mL) at 25° C. The resulting mixture was stirred for 2 h at 90° C. under nitrogen atmosphere. The resulting mixture was filtered, the filter cake was washed with EtOAc (3×10 mL). The filtrate was concentrated under reduced pressure. The residue was purified by silica gel column chromatography, eluted with PE/EtOAc (1/1) to afford the title compound (110 mg, 90%) as a light yellow solid. m/z (ESI, +ve ion)=705.30 [M+H]⁺.

### Step B. (1R,2S)-2-{3-[(4-Methanesulfonyl-2-methoxyphenyl)amino]-1H-indazol-6-yl}-5′-methoxy-1′H-spiro[cyclopropane-1,3′-indol]-2′-one

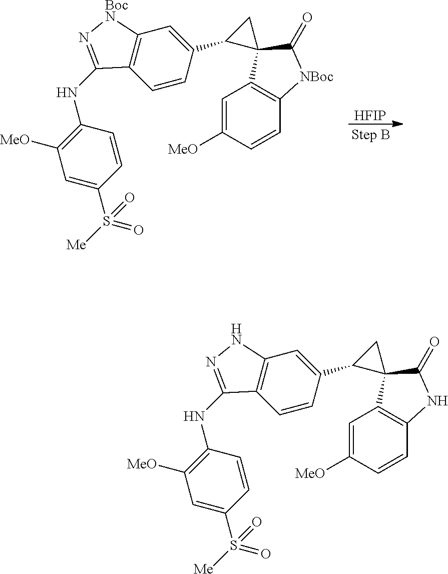 [SMILES: COc1ccc2c(c1)[C@]1(C[C@H]1c1ccc3c(Nc4ccc(S(C)(=O)=O)cc4OC)nn(C(=O)OC(C)(C)C)c3c1)C(=O)N2C(=O)OC(C)(C)C ; "HFIP" ; COc1ccc2c(c1)[C@]1(C[C@H]1c1ccc3c(Nc4ccc(S(C)(=O)=O)cc4OC)n[nH]c3c1)C(=O)N2]

Into a 40 mL vial was added tert-butyl (1R,2S)-2-[1-(tert-butoxycarbonyl)-3-[(4-methanesulfonyl-2-methoxyphenyl)amino]indazol-6-yl]-5′-methoxy-2′-oxospiro[cyclopropane-1,3′-indole]-1′-carboxylate (110 mg, 0.156 mmol, 1 equiv) and HFIP (10 mL) at 25° C. The resulting mixture was stirred for 12 h at 60° C. The mixture was concentrated and the crude product was purified by prep-HPLC with the following conditions: Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 45% B in 10 min, 45% B; wavelength: 254 nm; RT1 (min): 8.5 to afford Example 2 (36.3 mg, 46.10%) as a white solid. m/z (ESI, +ve ion)=505.15 [M+H]⁺. ¹H-NMR (400 MHz, DMSO-d₆) δ 12.39 (s, 1H), 10.43 (s, 1H), 8.35 (s, 1H), 8.09 (d, J=8.5 Hz, 1H), 7.83 (d, J=8.4 Hz, 1H), 7.50-7.34 (m, 3H), 6.92 (d, J=8.3 Hz, 1H), 6.76 (d, J=8.4 Hz, 1H), 6.59 (dd, J=8.4, 2.6 Hz, 1H), 5.71 (d, J=2.5 Hz, 1H), 4.01 (s, 3H), 3.33 (s, 4H), 3.16 (s, 3H), 2.39-2.29 (m, 1H), 2.05-1.94 (m, 1H).

### Example 4. (1R,2S)-5′-Methoxy-2-(3-{[3-methoxy-6-(1,3-oxazol-2-yl)pyridin-2-yl]amino}-1H-indazol-6-yl)spiro[cyclopropane-1,3′-indol]-2′(1′H)-one

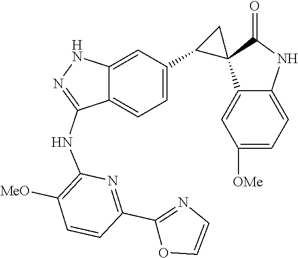 [SMILES: COc1ccc2c(c1)[C@]1(C[C@H]1c1ccc3c(Nc4nc(-c5ncco5)ccc4OC)n[nH]c3c1)C(=O)N2]

### Step A. tert-butyl N-(6-bromo-3-methoxypyridin-2-yl)-N-(tert-butoxycarbonyl) carbamate

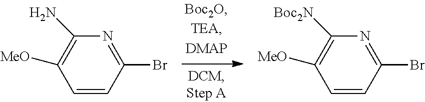

To a solution of 6-bromo-3-methoxypyridin-2-amine (250 mg, 1.231 mmol, 1 equiv) in DCM (4 mL) was added TEA (1121.38 mg, 11.079 mmol, 9 equiv), DMAP (30.09 mg, 0.246 mmol, 0.2 equiv) and (Boc)₂O (2015.46 mg, 9.232 mmol, 7.5 equiv). After stirring for 16 h at room temperature under nitrogen atmosphere, the resulting mixture was concentrated under reduced pressure. The residue was purified on silica gel column chromatography, eluting with 0-100% EtOAc in PE to afford the title compound (350 mg, 70.49%) as a yellow solid. m/z (ESI, +ve ion)=403.15, 405.05 [M+H]⁺.

### Step B. tert-butyl N-(tert-butoxycarbonyl)-N-[3-methoxy-6-(1,3-oxazol-2-yl) pyridin-2-yl]carbamate

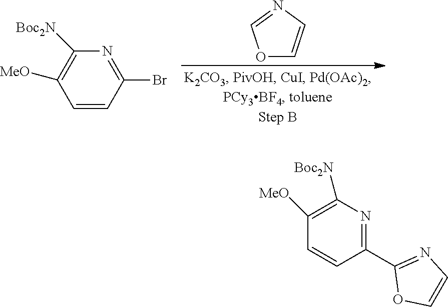

To a solution of tert-butyl N-(6-bromo-3-methoxypyridin-2-yl)-N-(tert-butoxycarbonyl) carbamate (330 mg, 0.818 mmol, 1 equiv) and oxazole (56.51 mg, 0.818 mmol, 1 equiv) in toluene (5 mL) was added Pd(OAc)₂ (18.37 mg, 0.082 mmol, 0.1 equiv), PCy₃·BF₄ (60.27 mg, 0.164 mmol, 0.2 equiv), CuI (171.43 mg, 0.900 mmol, 1.1 equiv), pivalic acid (33.43 mg, 0.327 mmol, 0.4 equiv) and K₂CO₃ (339.28 mg, 2.454 mmol, 3 equiv). After stirring for 16 h at 110° C. under nitrogen atmosphere, the resulting mixture was concentrated under reduced pressure. The residue was purified by silica gel column chromatography, eluting with 0-20% MeOH in DCM to afford the title compound (240 mg, 59.94%) as a yellow solid. m/z (ESI, +ve ion)=392.15 [M+H]⁺.

### Step C. 3-methoxy-6-(1,3-oxazol-2-yl)pyridin-2-amine

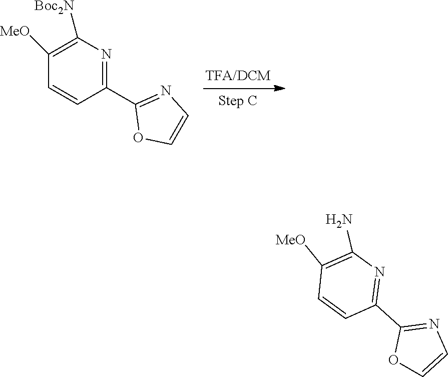

A solution of tert-butyl N-(tert-butoxycarbonyl)-N-[3-methoxy-6-(1,3-oxazol-2-yl)pyridin-2-yl]carbamate (230 mg, 0.588 mmol, 1 equiv) in DCM (1.6 mL) and TFA (0.4 mL) was stirred for 16 h at room temperature. The resulting mixture was concentrated under reduced pressure to afford the title compound (85 mg, crude) as a yellow oil. m/z (ESI, +ve ion)=192.10 [M+H]⁺.

### Step D. tert-butyl (1R, 2S)-2-[1-(tert-butoxycarbonyl)-3-{[3-methoxy-6-(1, 3-oxazol-2-yl) pyridin-2-yl]amino}indazol-6-yl]-5′-methoxy-2′-oxospiro[cyclopropane-1,3′-indole]-1′-carboxylate

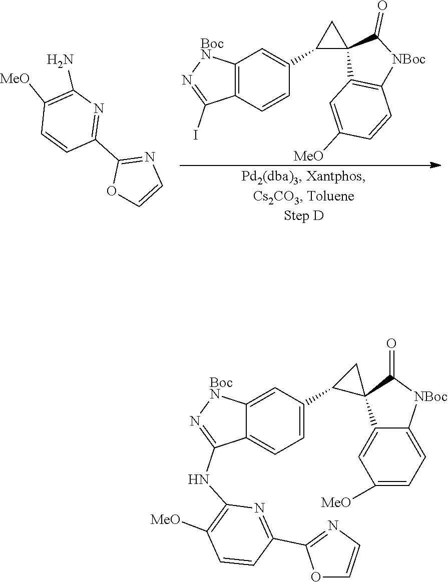 [SMILES: COc1ccc(-c2ncco2)nc1N ; COc1ccc2c(c1)[C@]1(C[C@H]1c1ccc3c(I)nn(C(=O)OC(C)(C)C)c3c1)C(=O)N2C(=O)OC(C)(C)C ; COc1ccc2c(c1)[C@]1(C[C@H]1c1ccc3c(Nc4nc(-c5ncco5)ccc4OC)nn(C(=O)OC(C)(C)C)c3c1)C(=O)N2C(=O)OC(C)(C)C]

To a solution of 3-methoxy-6-(1,3-oxazol-2-yl)pyridin-2-amine (54.50 mg, 0.285 mmol, 1.5 equiv) and tert-butyl (1R,2S)-2-(1-(tert-butoxycarbonyl)-3-iodo-1H-indazol-6-yl)-5′-methoxy-2′-oxospiro[cyclopropane-1,3′-indoline]-1′-carboxylate (120 mg, 0.190 mmol, 1.00 equiv) in toluene (1 mL) was added Pd₂(dba)₃ (34.80 mg, 0.038 mmol, 0.2 equiv), XantPhos (21.99 mg, 0.038 mmol, 0.2 equiv) and Cs₂CO₃ (185.75 mg, 0.570 mmol, 3 equiv). After stirring for 1 h at 90° C. under nitrogen atmosphere, the resulting mixture was concentrated under reduced pressure. The residue was purified by prep-TLC, eluting with EtOAc/PE=2/1 to afford the title compound (100 mg, 75.74%) as a yellow solid. m/z (ESI, +ve ion)=695.50 [M+H]⁺/

### Step E. (1R,2S)-5′-Methoxy-2-(3-{[3-methoxy-6-(1,3-oxazol-2-yl)pyridin-2-yl]amino}-1H-indazol-6-yl)spiro[cyclopropane-1,3′-indol]-2′(1′H)-one

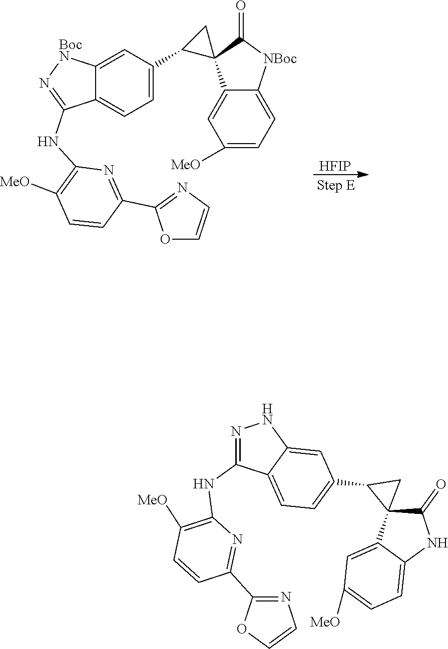 [SMILES: COc1ccc2c(c1)[C@]1(C[C@H]1c1ccc3c(Nc4nc(-c5ncco5)ccc4OC)nn(C(=O)OC(C)(C)C)c3c1)C(=O)N2C(=O)OC(C)(C)C ; "HFIP" ; COc1ccc2c(c1)[C@]1(C[C@H]1c1ccc3c(Nc4nc(-c5ncco5)ccc4OC)n[nH]c3c1)C(=O)N2]

A solution of tert-butyl (1R, 2S)-2-[1-(tert-butoxycarbonyl)-3-{[3-methoxy-6-(1,3-oxazol-2-yl) pyridin-2-yl]amino}indazol-6-yl]-5′-methoxy-2′-oxospiro[cyclopropane-1,3′-indole]-1′-carboxylate (100 mg, 0.144 mmol, 1 equiv) in HFIP (1.5 mL) was stirred for 16 h at 60° C. The mixture was filtered and the filtrate was concentrated under reduced pressure to give the crude product. The crude product was purified by prep-HPLC with the following conditions: Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 31% B to 39% B in 8 min, 39% B; wavelength: 220 nm; RT1 (min): 7.92; The fractions were concentrated under reduced pressure and then lyophilized overnight to afford Example 4 (30 mg, 42.15%) as a light yellow solid. m/z (ESI, +ve ion)=495.20 [M+H]⁺. ¹H-NMR (400 MHz, DMSO-d₆) δ 12.46 (s, 1H), 10.41 (s, 1H), 8.45 (s, 1H), 7.97 (s, 1H), 7.81 (d, J=8.4 Hz, 1H), 7.52 (d, J=8.0 Hz, 1H), 7.39-7.32 (m, 2H), 7.26 (s, 1H), 6.87 (d, J=8.5 Hz, 1H), 6.75 (d, J=8.4 Hz, 1H), 6.59 (dd, J=8.4, 2.6 Hz, 1H), 5.74 (d, J=2.5 Hz, 1H), 3.96 (s, 3H), 3.30 (s, 3H), 3.19 (t, J=8.4 Hz, 1H), 2.32 (dd, J=8.1, 4.8 Hz, 1H), 1.99 (dd, J=9.0, 4.6 Hz, 1H).

### Example 5. (1R,2S)-2-{3-[4-(Ethanesulfonyl)-2-methoxyanilino]-1H-indazol-6-yl}-5′-methoxyspiro[cyclopropane-1,3′-indol]-2′(1′H)-one

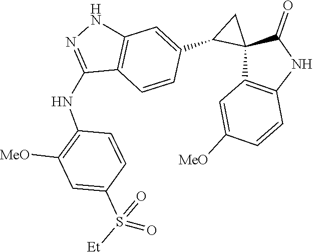 [SMILES: CCS(=O)(=O)c1ccc(Nc2n[nH]c3cc([C@@H]4C[C@@]45C(=O)Nc4ccc(OC)cc45)ccc23)c(OC)c1]

### Step A. 4-(ethanesulfonyl)-2-methoxyaniline

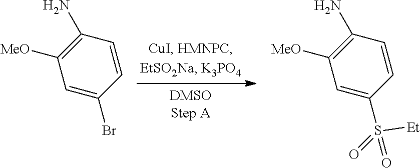 [SMILES: COc1cc(Br)ccc1N ; CCS(=O)(=O)c1ccc(N)c(OC)c1]

To a stirred mixture of 4-bromo-2-methoxyaniline (200 mg, 0.990 mmol, 1 equiv) and (2S, 4R)-4-hydroxy-N-(2-methylnaphthalen-1-yl)pyrrolidine-2-carboxamide (25 mg, 0.092 mmol, 0.09 equiv) in DMSO (5 mL) was added CuI (20 mg, 0.105 mmol, 0.11 equiv), sodium ethanesulfinate (170 mg, 1.464 mmol, 1.48 equiv) and K₃PO₄ (210 mg, 0.989 mmol, 1.00 equiv) at room temperature under nitrogen atmosphere. The resulting mixture was stirred for 16 h at 120° C. under nitrogen atmosphere. The mixture was allowed to cool down to room temperature. The resulting mixture was diluted with water (10 mL). The mixture was extracted with EtOAc (3×20 mL). The combined organic layers were washed with brine (3×5 mL) and dried over anhydrous Na₂SO₄. After filtration, the filtrate was concentrated under reduced pressure. The residue was purified by silica gel column chromatography, eluting with PE/EtOAc (1/1) to afford the title compound (140 mg, 65.70%) as a light yellow solid. m/z (ESI, +ve ion)=216.05 [M+H]⁺.

### Step B. tert-butyl (1R,2S)-2-[1-(tert-butoxycarbonyl)-3-{[4-(ethanesulfonyl)-2-methoxyphenyl]amino}indazol-6-yl]-5′-methoxy-2′-oxospiro[cyclopropane-1,3′-indole]-1′-carboxylate

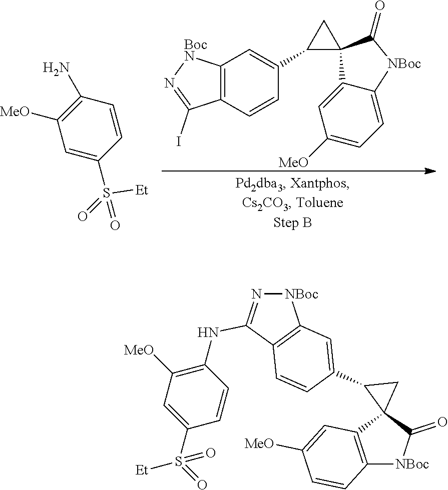 [SMILES: CCS(=O)(=O)c1ccc(N)c(OC)c1 ; COc1ccc2c(c1)[C@]1(C[C@H]1c1ccc3c(I)nn(C(=O)OC(C)(C)C)c3c1)C(=O)N2C(=O)OC(C)(C)C ; CCS(=O)(=O)c1ccc(Nc2nn(C(=O)OC(C)(C)C)c3cc([C@@H]4C[C@@]45C(=O)N(C(=O)OC(C)(C)C)c4ccc(OC)cc45)ccc23)c(OC)c1]

To a solution of 4-(ethanesulfonyl)-2-methoxyaniline (18 mg, 0.084 mmol, 1.06 equiv) and tert-butyl (1R,2S)-2-[1-(tert-butoxycarbonyl)-3-iodoindazol-6-yl]-5′-methoxy-2′-oxospiro[cyclopropane-1,3′-indole]-1′-carboxylate (50 mg, 0.079 mmol, 1.00 equiv) in toluene (2 mL) was added Cs₂CO₃ (55 mg, 0.169 mmol, 2.13 equiv), Pd₂(dba)₃ (15 mg, 0.016 mmol, 0.21 equiv) and XantPhos (10 mg, 0.017 mmol, 0.22 equiv). After stirring for 2 h at 90° C. under a nitrogen atmosphere, the mixture was allowed to cool down to room temperature. The resulting mixture was concentrated under reduced pressure. The residue was purified by silica gel column chromatography, eluting with PE/EtOAc (1/1) to afford the title compound (30 mg, 52.71%) as a light yellow solid. m/z (ESI, +ve ion)=719.25 [M+H]⁺.

### Step C. (1R,2S)-2-{3-[4-(Ethanesulfonyl)-2-methoxyanilino]-1H-indazol-6-yl}-5′-methoxyspiro[cyclopropane-1,3′-indol]-2′(1′H)-one

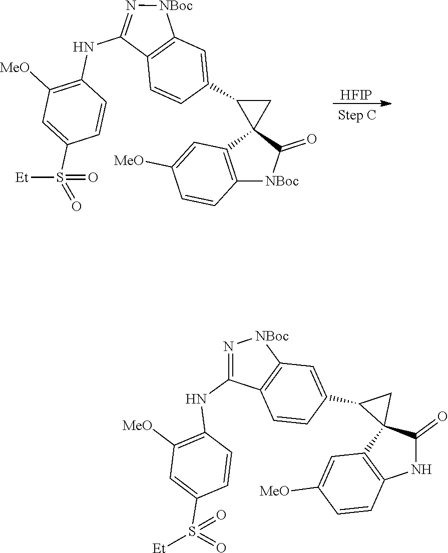 [SMILES: CCS(=O)(=O)c1ccc(Nc2nn(C(=O)OC(C)(C)C)c3cc([C@@H]4C[C@@]45C(=O)N(C(=O)OC(C)(C)C)c4ccc(OC)cc45)ccc23)c(OC)c1 ; "HFIP" ; CCS(=O)(=O)c1ccc(Nc2nn(C(=O)OC(C)(C)C)c3cc([C@@H]4C[C@@]45C(=O)Nc4ccc(OC)cc45)ccc23)c(OC)c1]

Into an 8 mL vial was added tert-butyl (1R,2S)-2-[1-(tert-butoxycarbonyl)-3-{[4-(ethanesulfonyl)-2-methoxyphenyl]amino}indazol-6-yl]-5′-methoxy-2′-oxospiro[cyclopropane-1,3′-indole]-1′-carboxylate (80 mg, 0.111 mmol, 1 equiv) and HFIP (1 mL) at room temperature. The mixture was stirred for 16 h at 60° C. under nitrogen atmosphere. The mixture was allowed to cool down to room temperature. The resulting mixture was concentrated under reduced pressure. The crude product was purified by prep-HPLC with the following conditions (Column: XBridge Prep Phenyl OBD Column, 19×150 mm, 5 μm; Mobile Phase A: Water (10 mmol/L NH₄HCO₃), Mobile Phase B: ACN; Flow rate: 60 mL/min; Gradient: 32% B to 42% B in 9 min, 42% B; Wavelength: 254 nm; RT1 (min): 8) to afford Example 5 (18.4 mg, 31.88%) as a white solid. m/z (ESI, +ve ion)=519.20 [M+H]⁺. ¹H-NMR (400 MHz, Methanol-d₄) δ 7.92 (d, J=8.4 Hz, 1H), 7.67 (d, J=8.3 Hz, 1H), 7.46-7.37 (m, 3H), 6.95 (d, J=8.4 Hz, 1H), 6.84 (d, J=8.4 Hz, 1H), 6.63 (dd, J=8.5, 2.5 Hz, 1H), 5.62 (d, J=2.6 Hz, 1H), 4.07 (s, 3H), 3.38 (d, J=8.6 Hz, 1H), 3.31 (s, 3H), 3.20 (q, J=7.4 Hz, 2H), 2.25-2.18 (m, 2H), 1.25 (t, J=7.4 Hz, 3H).

### Example 6. 6-Methoxy-5-({6-[(1R,2S)-5′-methoxy-2′-oxo-1′,2′-dihydrospiro[cyclopropane-1,3′-indol]-2-yl]-1H-indazol-3-yl}amino)-1H-1-benzothiophene-1,1-dione

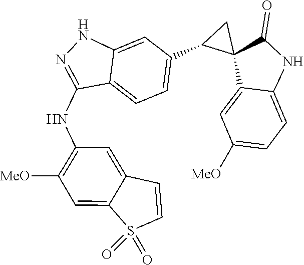 [SMILES: COc1ccc2c(c1)[C@]1(C[C@H]1c1ccc3c(Nc4cc5c(cc4OC)S(=O)(=O)C=C5)n[nH]c3c1)C(=O)N2]

### Step A. methyl 6-methoxy-5-nitro-1-benzothiophene-2-carboxylate

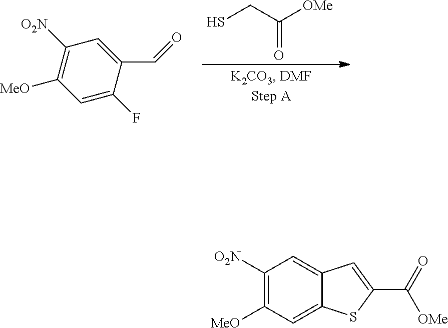

To a stirred mixture of 2-fluoro-4-methoxy-5-nitrobenzaldehyde (1200 mg, 6.026 mmol, 1 equiv) and methyl thioglycolate methyl thioglycolate (767.52 mg, 7.231 mmol, 1.2 equiv) in DMF (15 mL) was added K₂CO₃ (1665.65 mg, 12.052 mmol, 2 equiv) at room temperature under nitrogen atmosphere. The resulting mixture was stirred for 16 h at room temperature under nitrogen atmosphere. The mixture was extracted with EtOAc (3×30 mL). The combined organic layers were washed with water (3×10 mL) and dried over anhydrous Na₂SO₄. After filtration, the filtrate was concentrated under reduced pressure. The residue was purified by silica gel column chromatography, eluted with PE/EtOAc (5/1) to afford title the compound (850 mg, 52.78%) as a yellow solid. m/z (ESI, +ve ion)=268.10 [M+H]⁺.

### Step B. 6-methoxy-5-nitro-1-benzothiophene-2-carboxylic acid

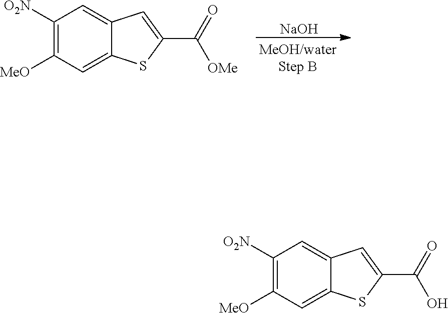 [SMILES: COC(=O)c1cc2cc([N+](=O)[O-])c(OC)cc2s1 ; "NaOH" ; COc1cc2sc(C(=O)O)cc2cc1[N+](=O)[O-]]

To a stirred mixture of methyl 6-methoxy-5-nitro-1-benzothiophene-2-carboxylate (800 mg, 2.993 mmol, 1 equiv) in H₂O (5 mL) and MeOH (5 mL) was added NaOH (478.90 mg, 11.972 mmol, 4 equiv) at room temperature under nitrogen atmosphere. The resulting mixture was stirred for 2 h at room temperature under nitrogen atmosphere. The mixture was basified to pH 7 with HCl (2 mol/L in water). The precipitated solids were collected by filtration and washed with water (3×10 mL). The residue was purified by silica gel column chromatography, eluting with DCM/MeOH (10/1) to afford the title compound (480 mg, 63.32%) as a white solid. ¹H NMR (400 MHz, DMSO-d₆) δ 13.61 (s, 1H), 8.59 (s, 1H), 8.14 (s, 1H), 8.06 (s, 1H), 4.00 (s, 3H).

### Step C. 6-methoxy-5-nitro-1-benzothiophene

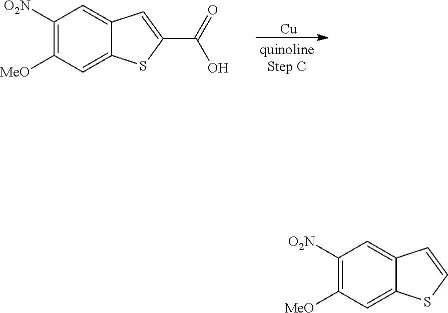

Into a 40 mL vial was added 6-methoxy-5-nitro-1-benzothiophene-2-carboxylic acid (300 mg, 1.185 mmol, 1 equiv), Cu (75.28 mg, 1.185 mmol, 1 equiv) and quinoline (5 mL) at room temperature. The resulting mixture was stirred for 2 h at 170° C. under nitrogen atmosphere. The mixture was allowed to cool down to room temperature. The mixture was acidified to pH 7 with HCl (2 mol/L). The resulting mixture was extracted with EtOAc (3×10 mL). The combined organic layers were dried over anhydrous Na₂SO₄. After filtration, the filtrate was concentrated under reduced pressure. The residue was purified by silica gel column chromatography, eluting with PE/EtOAc (1/1) to afford the title compound (210 mg, 84.72%) as a yellow solid. ¹H NMR (400 MHz, Chloroform-d) δ 8.32 (s, 1H), 7.53 (s, 1H), 7.45 (d, J=5.5 Hz, TH), 7.35 (d, J=5.5 Hz, 1H), 4.04 (s, 3H).

### Step D. 6-methoxy-5-nitrobenzo[b]thiophene 1,1-dioxide

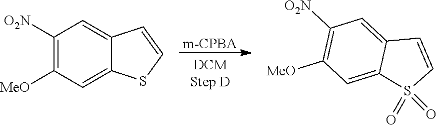

A mixture of 6-methoxy-5-nitro-1-benzothiophene (280 mg, 1.338 mmol, 1 equiv) and mCPBA (461.88 mg, 2.676 mmol, 2 equiv) in DCM (8 mL) was stirred for 2 h at room temperature under nitrogen atmosphere. The resulting mixture was concentrated under reduced pressure. The residue was purified by prep-TLC (DCM/MeOH 10/1) to afford the title compound (290 mg, 89.83%) as a white solid. ¹H NMR (400 MHz, DMSO-d₆) δ 8.14 (s, 1H), 8.05 (s, 1H), 7.63 (dd, J=6.9, 0.9 Hz, 1H), 7.45 (d, J=6.9 Hz, 1H), 4.07 (s, 3H).

### Step E. 5-amino-6-methoxybenzo[b]thiophene 1,1-dioxide

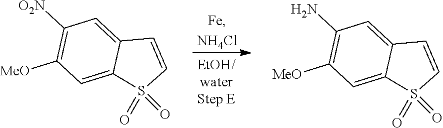

To a stirred solution of 6-methoxy-5-nitrobenzo[b]thiophene 1,1-dioxide (100 mg, 0.415 mmol, 1 equiv) and Fe (115.76 mg, 2.075 mmol, 5 equiv) in EtOH (4 mL) and H₂O (1 mL) was added NH₄Cl (110.87 mg, 2.075 mmol, 5 equiv) at room temperature under nitrogen atmosphere. The resulting mixture was stirred for 2 h at room temperature under nitrogen atmosphere. The mixture was filtered and the filter cake was washed with EtOAc (3×10 mL). The filtrate was concentrated under reduced pressure. The residue was purified by silica gel column chromatography, eluting with DCM/MeOH (10/1) to afford the title compound (60 mg, 68%). m/z (ESI+ve ion)=212.05 [M+H]⁺.

### Step F. tert-butyl (1R, 2S)-2-[1-(tert-butoxycarbonyl)-3-[(6-methoxy-1,1-dioxo-1lambda6-benzothiophen-5-yl)amino]indazol-6-yl]-5′-methoxy-2′-oxospiro[cyclopropane-1,3′-indole]-1′-carboxylate

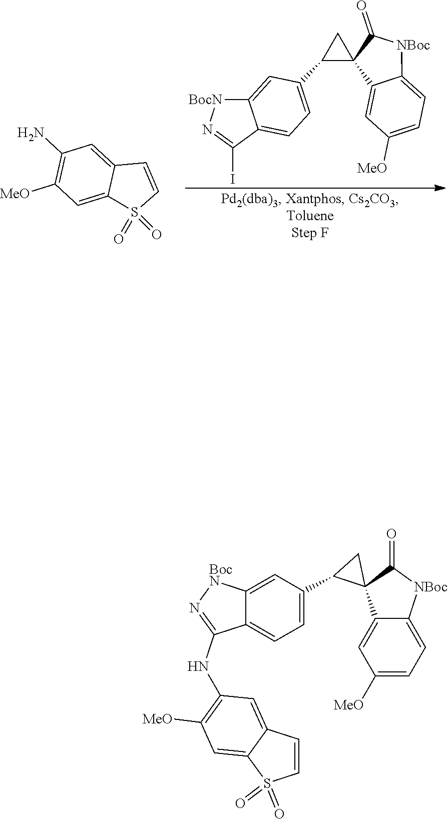 [SMILES: COc1cc2c(cc1N)C=CS2(=O)=O ; COc1ccc2c(c1)[C@]1(C[C@H]1c1ccc3c(I)nn(C(=O)OC(C)(C)C)c3c1)C(=O)N2C(=O)OC(C)(C)C ; "Pd2(dba)3, Xantphos, Cs2CO3,TolueneStep F" ; COc1ccc2c(c1)[C@]1(C[C@H]1c1ccc3c(Nc4cc5c(cc4OC)S(=O)(=O)C=C5)nn(C(=O)OC(C)(C)C)c3c1)C(=O)N2C(=O)OC(C)(C)C]

To a stirred solution of 5-amino-6-methoxy-1lambda6-benzothiophene-1,1-dione (40.14 mg, 0.190 mmol, 1.2 equiv) and tert-butyl (1R,2S)-2-[1-(tert-butoxycarbonyl)-3-iodoindazol-6-yl]-5′-methoxy-2′-oxospiro[cyclopropane-1,3′-indole]-1′-carboxylate (100 mg, 0.16 mmol, 1.00 equiv) in toluene (5 mL) was added Pd₂(dba)₃ (29.00 mg, 0.032 mmol, 0.2 equiv), XantPhos (18.33 mg, 0.032 mmol, 0.2 equiv) and Cs₂CO₃ (103.19 mg, 0.316 mmol, 2 equiv) at room temperature under nitrogen atmosphere. The resulting mixture was stirred for 2 h at 90° C. under nitrogen atmosphere. The residue was purified by prep-TLC (PE/EtOAc=1/1) to afford the title compound (95 mg, 83.93%) as a yellow solid. m/z (ESI, +ve ion)=715.25 [M+H]⁺.

### Step G. 6-Methoxy-5-({6-[(1R,2S)-5′-methoxy-2′-oxo-1′,2′-dihydrospiro[cyclopropane-1,3′-indol]-2-yl]-1H-indazol-3-yl}amino)-1H-1-benzothiophene-1,1-dione

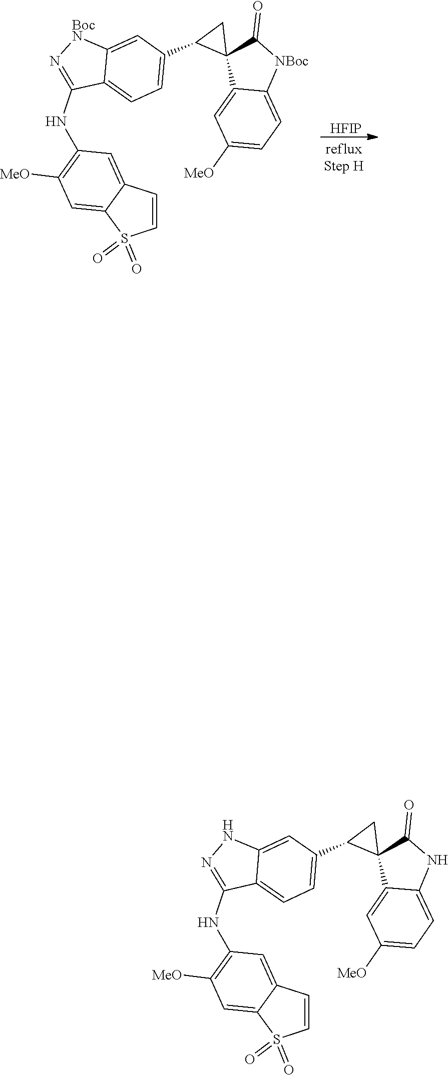

Into an 8 mL vial was added tert-butyl (1R,2S)-2-[1-(tert-butoxycarbonyl)-3-[(6-methoxy-1,1-dioxo-1lambda6-benzothiophen-5-yl)amino]indazol-6-yl]-5′-methoxy-2′-oxospiro[cyclopropane-1,3′-indole]-1′-carboxylate (95 mg, 0.133 mmol, 1 equiv) and HFIP (5 mL) at room temperature. The resulting mixture was stirred for 16 h at 60° C. under nitrogen atmosphere. The mixture was allowed to cool down to room temperature. The resulting mixture was concentrated under reduced pressure. The crude product was purified by prep-HPLC with the following conditions: Column: XBridge Shield RP18 OBD Column, 19×250 mm, 10 μm; Mobile Phase A: Water (10 mmol/L NH₄HCO₃), Mobile Phase B: ACN; Flow rate: 25 mL/min; Gradient: 35% B to 50% B in 8 min, 50% B; Wavelength: 254 nm; RT1 (min): 7.8 to afford Example 6 (30.5 mg, 44.38%) as a white solid. m/z (ESI, +ve ion)=515.15 [M+H]⁺. ¹H-NMR (400 MHz, DMSO-d₆) δ 12.38 (s, 1H), 10.42 (s, 1H), 8.34 (s, 1H), 8.09 (s, 1H), 7.84 (d, J=8.4 Hz, 1H), 7.54-7.46 (m, 2H), 7.37 (s, 1H), 7.15 (d, J=6.8 Hz, 1H), 6.93 (dd, J=8.4, 1.4 Hz, 1H), 6.75 (d, J=8.4 Hz, 1H), 6.59 (dd, J=8.4, 2.6 Hz, 1H), 5.70 (d, J=2.6 Hz, 1H), 4.02 (s, 3H), 3.30 (s, 3H), 3.19 (t, J=8.4 Hz, 1H), 2.33 (dd, J=7.9, 4.7 Hz, 1H), 1.99 (dd, J=9.0, 4.7 Hz, 1H).

### Example 14. (1R,2S)-5′-Methoxy-2-{3-[2-methoxy-5-(1,3-thiazol-2-yl)anilino]-1H-indazol-6-yl}spiro[cyclopropane-1,3′-indol]-2′(1′H)-one

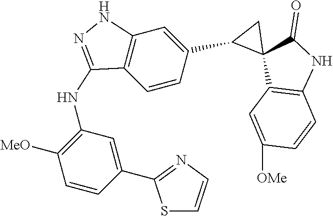 [SMILES: COc1ccc2c(c1)[C@]1(C[C@H]1c1ccc3c(Nc4cc(-c5nccs5)ccc4OC)n[nH]c3c1)C(=O)N2]

### Step A. 5-bromo-2-methoxyaniline

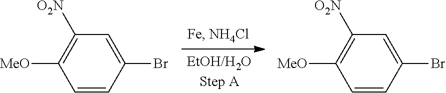 [SMILES: COc1ccc(Br)cc1[N+](=O)[O-] ; COc1ccc(Br)cc1[N+](=O)[O-]]

Into a 50 mL round-bottom flask was added 4-bromo-1-methoxy-2-nitrobenzene (2 g, 8.619 mmol, 1 equiv), Fe (2.4 g, 42.976 mmol, 4.99 equiv), NH₄Cl (2.30 g, 43.009 mmol, 4.99 equiv), EtOH (10 mL) and water (2.5 mL) at room temperature. The mixture was stirred for 3 h at room temperature under nitrogen atmosphere. The resulting mixture was filtered and the filter cake was washed with EtOH (3×10 mL). The filtrate was concentrated under reduced pressure. The residue was purified by silica gel column chromatography, eluting with DCM/MeOH (10/1) to afford the title compound (1.45 g, 83.26%) as a yellow solid. m/z (ESI, +ve ion)=201.90, 203.90 [M+H]⁺.

### Step B. 2-methoxy-5-(1,3-thiazol-2-yl)aniline

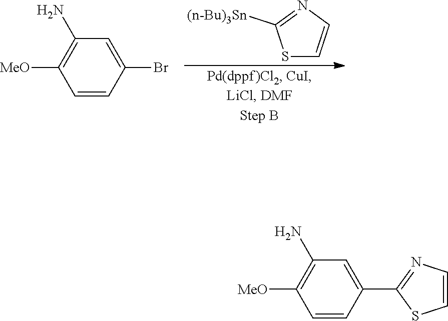

To a stirred mixture of 5-bromo-2-methoxyaniline (200 mg, 0.990 mmol, 1.00 equiv), Pd(dppf)Cl₂·CH₂Cl₂ (81 mg, 0.099 mmol, 0.10 equiv), CuI (100 mg, 0.525 mmol, 0.53 equiv) and LiCl (90 mg, 2.123 mmol, 2.14 equiv) in DMF (5 mL) was added 2-(tributylstannyl)-1,3-thiazole (380 mg, 1.016 mmol, 1.03 equiv) at room temperature under nitrogen atmosphere. The mixture was stirred for 2 h at 100° C. under nitrogen atmosphere. The mixture was allowed to cool down to room temperature. The resulting mixture was extracted with EtOAc (3×10 mL). The combined organic layers were washed with brine (3×10 mL) and dried over anhydrous Na₂SO₄. After filtration, the filtrate was concentrated under reduced pressure. The residue was purified by silica gel column chromatography, eluting with PE/EtOAc (1/1) to afford the title compound (130 mg, 63.67%) as a light yellow solid. m/z (ESI, +ve ion)=207.00 [M+H]⁺.

### Step C. tert-butyl (1R, 2S)-2-[1-(tert-butoxycarbonyl)-3-{[2-methoxy-5-(1,3-thiazol-2-yl)phenyl]amino}indazol-6-yl]-5′-methoxy-2′-oxospiro[cyclopropane-1,3′-indole]-1′-carboxylate

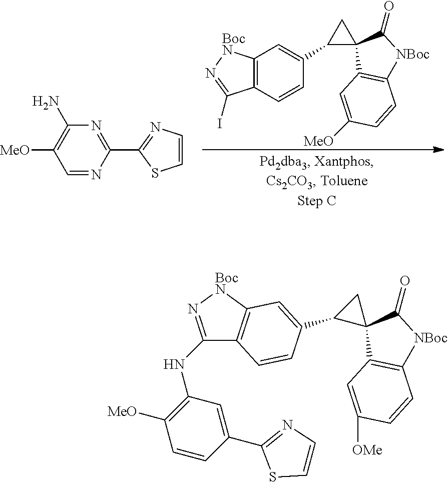

To a solution of tert-butyl (1R, 2S)-2-[1-(tert-butoxycarbonyl)-3-iodoindazol-6-yl]-5′-methoxy-2′-oxospiro[cyclopropane-1,3′-indole]-1′-carboxylate (100 mg, 0.158 mmol, 1 equiv) and 2-methoxy-5-(1,3-thiazol-2-yl)aniline (40 mg, 0.194 mmol, 1.22 equiv) in toluene (2.5 mL) was added Cs₂CO₃ (100 mg, 0.307 mmol, 1.94 equiv), Pd₂(dba)₃ (30 mg, 0.033 mmol, 0.21 equiv) and XantPhos (20 mg, 0.035 mmol, 0.22 equiv). The mixture was stirred for 2 h at 90° C. under nitrogen atmosphere. The mixture was allowed to cool down to room temperature. The resulting mixture was concentrated under reduced pressure. The residue was purified by silica gel column chromatography, eluting with PE/EtOAc (1/1) to afford the title compound (80 mg, 71.17%) as a light yellow solid. m/z (ESI, +ve ion)=710.20 [M+H]⁺.

### Step D. (1R,2S)-5′-Methoxy-2-{3-[2-methoxy-5-(1,3-thiazol-2-yl)anilino]-1H-indazol-6-yl}spiro[cyclopropane-1,3′-indol]-2′(1′H)-one

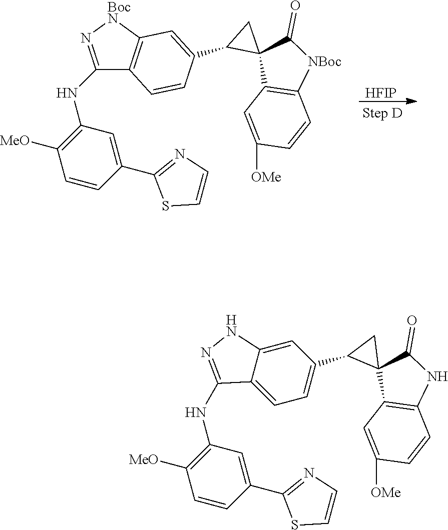

Into an 8 mL vial was added tert-butyl (1R, 2S)-2-[1-(tert-butoxycarbonyl)-3-{[2-methoxy-5-(1,3-thiazol-2-yl)phenyl]amino}indazol-6-yl]-5′-methoxy-2′-oxospiro[cyclopropane-1,3′-indole]-1′-carboxylate (80 mg, 0.113 mmol, 1 equiv) and HFIP (2 mL) at room temperature. The mixture was stirred for 12 h at 60° C. under nitrogen atmosphere. The mixture was allowed to cool down to room temperature. The resulting mixture was concentrated under reduced pressure. The crude product was purified by prep-HPLC with the following conditions (Column: XSelect CSH Prep C18 OBD Column, 19×250 mm, 5 μm; Mobile Phase A: Water (10 mmol/L NH₄HCO₃), Mobile Phase B: MeOH—HPLC; Flow rate: 20 mL/min; Gradient: 70% B to 75% B in 8 min, 75% B; wavelength: 254 nm; RT1 (min): 10) to afford Example 14 (28.1 mg, 48.93%) as a white solid. m/z (ESI, +ve ion)=510.15 [M+H]⁺. ¹H-NMR (300 MHz, DMSO-d₆) δ 12.26 (s, 1H), 10.44 (s, 1H), 8.82 (d, J=2.2 Hz, 1H), 7.95-7.81 (m, 3H), 7.67 (d, J=3.3 Hz, 1H), 7.43 (dd, J=8.3, 2.2 Hz, 1H), 7.35 (s, 1H), 7.10 (d, J=8.4 Hz, 1H), 6.94-6.85 (m, 1H), 6.75 (d, J=8.4 Hz, 1H), 6.59 (dd, J=8.4, 2.5 Hz, 1H), 5.71 (d, J=2.5 Hz, 1H), 3.97 (s, 3H), 3.32 (s, 3H), 3.19 (t, J=8.4 Hz, 1H), 2.35 (dd, J=7.9, 4.7 Hz, 1H), 1.98 (dd, J=9.0, 4.6 Hz, 1H).

### Example 15. (1R,2S)-5′-Methoxy-2-{3-[2-methoxy-5-(1,3-thiazol-4-yl)anilino]-1H-indazol-6-yl}spiro[cyclopropane-1,3′-indol]-2′(1′H)-one

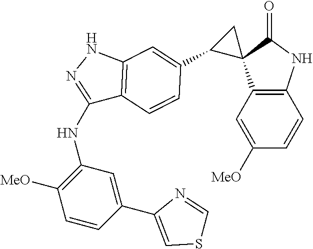 [SMILES: COc1ccc2c(c1)[C@]1(C[C@H]1c1ccc3c(Nc4cc(-c5cscn5)ccc4OC)n[nH]c3c1)C(=O)N2]

### Step A. 2-methoxy-5-(1,3-thiazol-4-yl)aniline

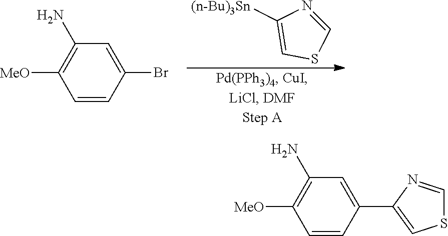

To a solution of 5-bromo-2-methoxyaniline (200 mg, 0.990 mmol, 1 equiv) and 4-(tributylstannyl)-1,3-thiazole (400.00 mg, 1.069 mmol, 1.08 equiv) in DMF (5 mL) was added LiCl (90 mg, 2.123 mmol, 2.14 equiv), CuI (100 mg, 0.525 mmol, 0.53 equiv) and Pd(PPh₃)₄ (125 mg, 0.108 mmol, 0.11 equiv). The mixture was stirred for 2 h at 100° C. under nitrogen atmosphere. The mixture was allowed to cool down to room temperature. The resulting mixture was extracted with EtOAc (3×10 mL). The combined organic layers were washed with brine (3×10 mL) and dried over anhydrous Na₂SO₄. After filtration, the filtrate was concentrated under reduced pressure. The residue was purified by silica gel column chromatography, eluting with DCM/MeOH (10/1) to afford the title compound (90 mg, 44.08%) as a light brown solid. m/z (ESI, +ve ion)=207.05 [M+H]⁺.

### Step B. tert-butyl (1R, 2S)-2-[1-(tert-butoxycarbonyl)-3-{[2-methoxy-5-(1,3-thiazol-4-yl)phenyl]amino}indazol-6-yl]-5′-methoxy-2′-oxospiro[cyclopropane-1,3′-indole]-1′-carboxylate

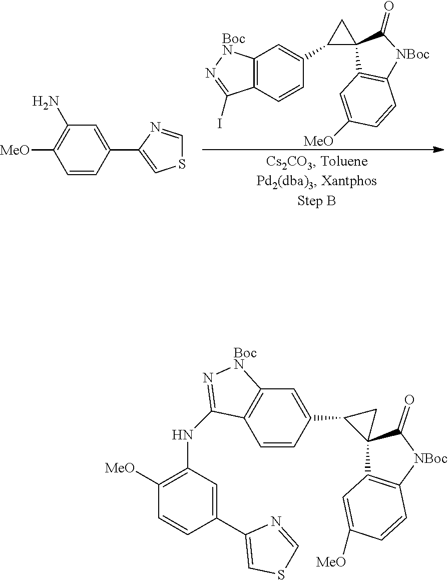

To a solution of tert-butyl (1R, 2S)-2-[1-(tert-butoxycarbonyl)-3-iodoindazol-6-yl]-5′-methoxy-2′-oxospiro[cyclopropane-1,3′-indole]-1′-carboxylate (100 mg, 0.16 mmol, 1 equiv) and 2-methoxy-5-(1,3-thiazol-4-yl)aniline (50 mg, 0.242 mmol, 1.53 equiv) in toluene (2.5 mL) was added Cs₂CO₃ (100 mg, 0.307 mmol, 1.94 equiv), Pd₂(dba)₃ (60 mg, 0.066 mmol, 0.41 equiv) and XantPhos (40 mg, 0.069 mmol, 0.44 equiv). The resulting mixture was stirred for 2 h at 90° C. under nitrogen atmosphere. The mixture was allowed to cool down to room temperature. The resulting mixture was concentrated under reduced pressure. The residue was purified by silica gel column chromatography, eluting with PE/EtOAc (1/1) to afford the title compound (60 mg, 53.38%) as a light yellow solid. m/z (ESI, +ve ion)=710.35 [M+H]⁺.

### Step C. (1R,2S)-5′-Methoxy-2-{3-[2-methoxy-5-(1,3-thiazol-4-yl)anilino]-1H-indazol-6-yl}spiro[cyclopropane-1,3′-indol]-2′(1′H)-one

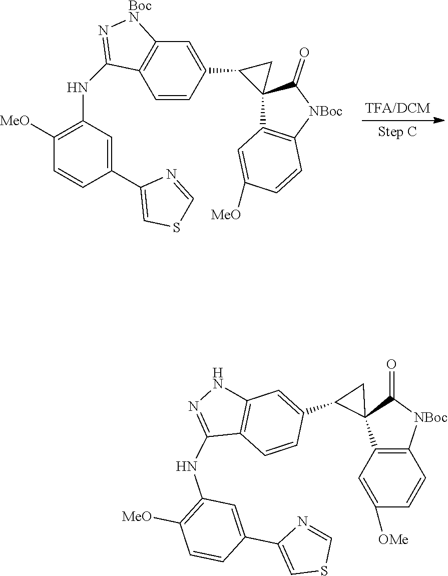 [SMILES: COc1ccc2c(c1)[C@]1(C[C@H]1c1ccc3c(Nc4cc(-c5cscn5)ccc4OC)nn(C(=O)OC(C)(C)C)c3c1)C(=O)N2C(=O)OC(C)(C)C ; COc1ccc2c(c1)[C@]1(C[C@H]1c1ccc3c(Nc4cc(-c5cscn5)ccc4OC)n[nH]c3c1)C(=O)N2C(=O)OC(C)(C)C]

Into an 8 mL vial was added tert-butyl (1R,2S)-2-[1-(tert-butoxycarbonyl)-3-{[2-methoxy-5-(1,3-thiazol-4-yl)phenyl]amino}indazol-6-yl]-5′-methoxy-2′-oxospiro[cyclopropane-1,3′-indole]-1′-carboxylate (60 mg, 0.085 mmol, 1 equiv), TFA (1 mL) and DCM (3 mL) at room temperature. The mixture was stirred for 1 h at room temperature under nitrogen atmosphere. The resulting mixture was azeotroped with toluene (3×5 mL). The mixture was concentrated under reduced pressure. The crude product was purified by prep-HPLC with the following conditions (Column: XSelect CSH Prep C18 OBD Column, 19×250 mm, 5 μm; Mobile Phase A: Water (10 mmol/L NH₄HCO₃), Mobile Phase B: MeOH—HPLC; Flow rate: 20 mL/min; Gradient: 70% B to 75% B in 8 min, 75% B; wavelength: 254 nm; RT1 (min): 8) to afford Example 15 (21.1 mg, 48.99%) as a white solid. m/z (ESI, +ve ion)=510.15 [M+H]⁺. ¹H-NMR (400 MHz, Methanol-d₄) δ 9.00 (d, J=2.0 Hz, 1H), 8.33 (d, J=2.2 Hz, 1H), 7.66 (d, J=8.2 Hz, 1H), 7.60 (d, J=2.0 Hz, 1H), 7.47 (dd, J=8.4, 2.2 Hz, 1H), 7.39 (s, 1H), 7.07 (d, J=8.4 Hz, 1H), 6.91 (d, J=8.5 Hz, 1H), 6.85 (d, J=8.5 Hz, 1H), 6.64 (dd, J=8.5, 2.6 Hz, 1H), 5.63 (d, J=2.5 Hz, 1H), 4.02 (s, 3H), 3.38 (d, J=8.5 Hz, 1H), 3.29 (s, 3H), 2.28-2.16 (m, 2H).

### Example 25: (1R,2S)-5′-Methoxy-2-{3-[(5-methoxy[2,5′-bipyrimidin]-4-yl)amino]-1H-indazol-6-yl}spiro[cyclopropane-1,3′-indol]-2′(1′H)-one

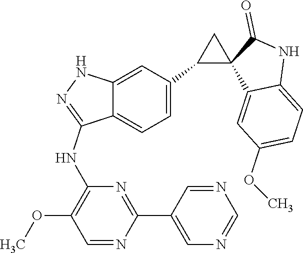 [SMILES: COc1ccc2c(c1)[C@]1(C[C@H]1c1ccc3c(Nc4nc(-c5cncnc5)ncc4OC)n[nH]c3c1)C(=O)N2]

### Step A: 5-Methoxy-2-pyrimidin-5-yl-pyrimidin-4-amine

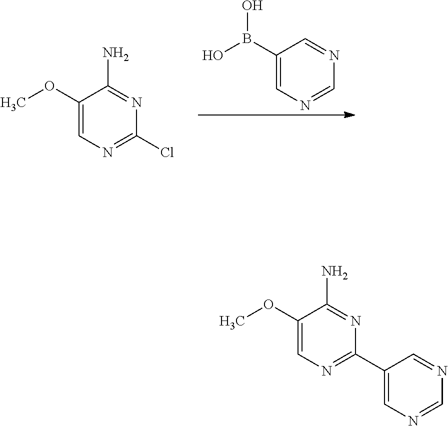

To a mixture of 2-chloro-5-methoxy-4-pyrimidinamine (100 mg, 0.63 mmol), 5-pyrimidinylboronic acid (85 mg, 0.69 mmol), bis(di-tert-butyl(4-dimethylaminophenyl)-phosphine)dichloropalladium (36 mg, 0.05 mmol), potassium acetate (123 mg, 1.25 mmol) and sodium carbonate (199 mg, 1.88 mmol) was added MeCN (3 mL) and water (1.5 mL). Argon was bubbled through the reaction mixture and the reaction mixture was stirred at 80° C. for 16 h. The reaction mixture was diluted with DCM and filtered through celite, eluting with DCM and the filtrate was concentrated in vacuo. The crude residue was purified by flash chromatography (0-100% acetone in hexanes) to afford the product as a light yellow solid (37.2 mg, 29%). m/z (ESI, +ve ion)=204.2 [M+H]+.

### Step B: Tert-butyl (1R,2S)-2-[1-tert-butoxycarbonyl-3-[(5-methoxy-2-pyrimidin-5-yl-pyrimidin-4-yl)amino]indazol-6-yl]-5′-methoxy-2′-oxo-spiro[cyclopropane-1,3′-indoline]-1′-carboxylate

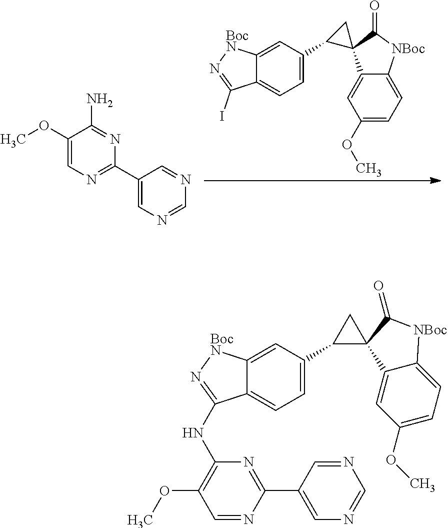 [SMILES: COc1cnc(-c2cncnc2)nc1N ; COc1ccc2c(c1)[C@]1(C[C@H]1c1ccc3c(I)nn(C(=O)OC(C)(C)C)c3c1)C(=O)N2C(=O)OC(C)(C)C ; COc1ccc2c(c1)[C@]1(C[C@H]1c1ccc3c(Nc4nc(-c5cncnc5)ncc4OC)nn(C(=O)OC(C)(C)C)c3c1)C(=O)N2C(=O)OC(C)(C)C]

To a vial was added tert-butyl (1R,2S)-2-(1-tert-butoxycarbonyl-3-iodo-indazol-6-yl)-5′-methoxy-2′-oxo-spiro[cyclopropane-1,3′-indoline]-1′-carboxylate (105 mg, 0.17 mmol), 5-methoxy-2-pyrimidin-5-yl-pyrimidin-4-amine (37 mg, 0.18 mmol), Xantphos Pd G4 (32 mg, 0.03 mmol), Xantphos (19 mg, 0.03 mmol) and cesium carbonate (217 mg, 0.67 mmol) followed by 1,4-dioxane (1.6 mL). Argon was bubbled through the solution for 3 min then the reaction mixture was heated to 100° C. for 2 h, then 110° C. for an additional 2 h. The reaction mixture was diluted with DCM, filtered through celite, eluting with DCM and the filtrate was concentrated in vacuo. The crude residue was purified by flash chromatography (0-100% acetone in hexanes) to afford the product as a brown solid (7.2 mg, 6%). m/z (ESI, +ve ion)=708.4 [M+H]+.

### Step C: (1R,2S)-5′-Methoxy-2-[3-[(5-methoxy-2-pyrimidin-5-yl-pyrimidin-4-yl)amino]-1H-indazol-6-yl]spiro[cyclopropane-1,3′-indoline]-2′-one

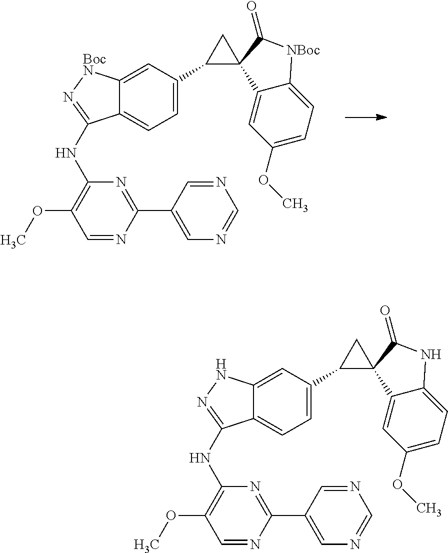 [SMILES: COc1ccc2c(c1)[C@]1(C[C@H]1c1ccc3c(Nc4nc(-c5cncnc5)ncc4OC)nn(C(=O)OC(C)(C)C)c3c1)C(=O)N2C(=O)OC(C)(C)C ; COc1ccc2c(c1)[C@]1(C[C@H]1c1ccc3c(Nc4nc(-c5cncnc5)ncc4OC)n[nH]c3c1)C(=O)N2]

A solution of tert-butyl (1R,2S)-2-[1-tert-butoxycarbonyl-3-[(5-methoxy-2-pyrimidin-5-yl-pyrimidin-4-yl)amino]indazol-6-yl]-5′-methoxy-2′-oxo-spiro[cyclopropane-1,3′-indoline]-1′-carboxylate (7.2 mg, 0.01 mmol) in hexafluoroisopropanol (0.5 mL) was heated to 50° C. for 16 h. The reaction mixture was concentrated in vacuo and the crude residue was purified by RP-HPLC using 10-90% ACN/water (10 mmol/L ammonium bicarbonate) to give the title compound as a beige lyophilized solid (2.2 mg, 43%). m/z (ESI, +ve ion)=507.2 [M+H]⁺. 1H NMR (400 MHz, DMSO) δ 12.75 (s, 1H), 10.42 (s, 1H), 9.53 (s, 1H), 9.20-9.09 (m, 3H), 8.24 (s, 1H), 7.52 (d, J=8.4 Hz, 1H), 7.43 (s, 1H), 6.94 (d, J=8.4 Hz, 1H), 6.74 (d, J=8.4 Hz, 1H), 6.58 (dd, J=8.5, 2.6 Hz, 1H), 5.68 (d, J=2.6 Hz, 1H), 4.02 (s, 3H), 3.28 (s, 3H), 3.25-3.18 (m, 1H), 2.34-2.27 (m, 1H), 2.04-1.96 (m, 1H).

### Example 28. (1R,2S)-2-(3-{[3-Ethoxy-6-(1,3-thiazol-2-yl)pyridin-2-yl]amino}-1H-indazol-6-yl)-5′-methoxyspiro[cyclopropane-1,3′-indol]-2′(1′H)-one

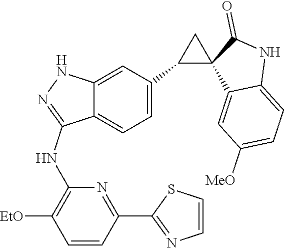 [SMILES: CCOc1ccc(-c2nccs2)nc1Nc1n[nH]c2cc([C@@H]3C[C@@]34C(=O)Nc3ccc(OC)cc34)ccc12]

### Step A. 6-Bromo-3-ethoxypyridin-2-amine

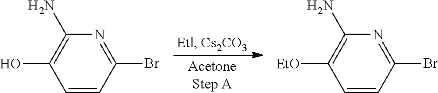 [SMILES: Nc1nc(Br)ccc1O ; CCOc1ccc(Br)nc1N]

To a stirred mixture of 2-amino-6-bromopyridin-3-ol (300 mg, 1.59 mmol, 1.00 equiv) and iodoethane (248 mg, 1.59 mmol, 1.00 equiv) in acetone (5 mL) was added Cs₂CO₃ (1.03 g, 3.17 mmol, 2.00 equiv) at room temperature under nitrogen atmosphere. The resulting mixture was stirred for 16 h at 50° C. under nitrogen atmosphere. The resulting mixture was concentrated under reduced pressure. The residue was purified by silica gel column chromatography, eluted with PE/EtOAc (1/1) to afford the title compound (280 mg, 81.3%) as a white solid. m/z (ESI, +ve ion)=217.15, 219.15 [M+H]⁺.

### Step B. 3-Ethoxy-6-(thiazol-2-yl)pyridin-2-amine

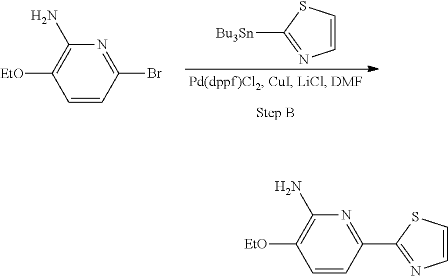

To a stirred mixture of 6-bromo-3-ethoxypyridin-2-amine (250 mg, 1.15 mmol, 1.00 equiv) and 2-(tributylstannyl)-1,3-thiazole (517 mg, 1.38 mmol, 1.20 equiv) in DMF (5 mL) were added Pd(dppf)Cl₂·CH₂Cl₂ (93.8 mg, 0.115 mmol, 0.100 equiv), CuI (110 mg, 0.576 mmol, 0.500 equiv) and LiCl (97.6 mg, 2.30 mmol, 2.00 equiv) at room temperature under nitrogen atmosphere. The resulting mixture was stirred for 3 h at 90° C. under nitrogen atmosphere. The resulting mixture was concentrated under reduced pressure. The residue was purified by silica gel column chromatography, eluted with PE/EtOAc (1/1) to afford the title compound (125 mg, 49.10%) as a white solid. m/z (ESI, +ve ion)=222.05 [M+H]⁺.

### Step C. Tert-butyl (1R, 2S)-2-(1-(tert-butoxycarbonyl)-3-((3-ethoxy-6-(thiazol-2-yl)pyridin-2-yl)amino)-1H-indazol-6-yl)-5′-methoxy-2′-oxospiro[cyclopropane-1,3′-indoline]-1′-carboxylate

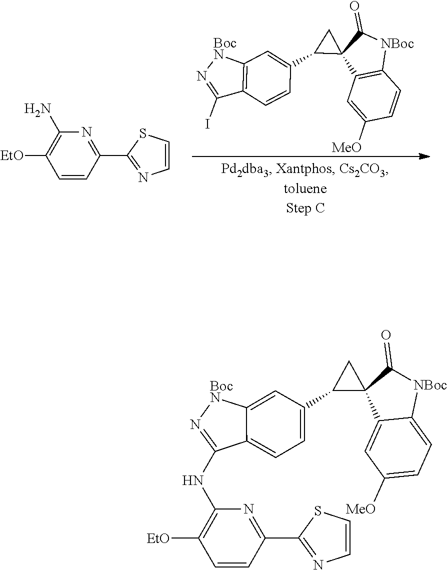 [SMILES: CCOc1ccc(-c2nccs2)nc1N ; COc1ccc2c(c1)[C@]1(C[C@H]1c1ccc3c(I)nn(C(=O)OC(C)(C)C)c3c1)C(=O)N2C(=O)OC(C)(C)C ; CCOc1ccc(-c2nccs2)nc1Nc1nn(C(=O)OC(C)(C)C)c2cc([C@@H]3C[C@@]34C(=O)N(C(=O)OC(C)(C)C)c3ccc(OC)cc34)ccc12]

To a stirred mixture of 3-ethoxy-6-(1,3-thiazol-2-yl)pyridin-2-amine (42.1 mg, 0.190 mmol, 1.20 equiv) and tert-butyl (1R, 2S)-2-[1-(tert-butoxycarbonyl)-3-iodoindazol-6-yl]-5′-methoxy-2′-oxospiro[cyclopropane-1,3′-indole]-1′-carboxylate (100 mg, 0.158 mmol, 1.00 equiv) in toluene (5 mL) were added Pd₂(dba)₃ (29.0 mg, 0.032 mmol, 0.20 equiv), XantPhos (18.3 mg, 0.032 mmol, 0.200 equiv) and Cs₂CO₃ (103 mg, 0.316 mmol, 2.00 equiv) at room temperature under nitrogen atmosphere. The resulting mixture was stirred for 1 h at 90° C. under nitrogen atmosphere. The resulting mixture was concentrated under reduced pressure. The residue was purified by prep-TLC (PE/EtOAc 1/1) to afford the title compound (79 mg, 69%) as a yellow solid. m/z (ESI, +ve ion)=725.25 [M+H]⁺.

### Step D. (1R, 2S)-2-(3-((3-Ethoxy-6-(thiazol-2-yl)pyridin-2-yl)amino)-1H-indazol-6-yl)-5′-methoxyspiro[cyclopropane-1,3′-indolin]-2′-one

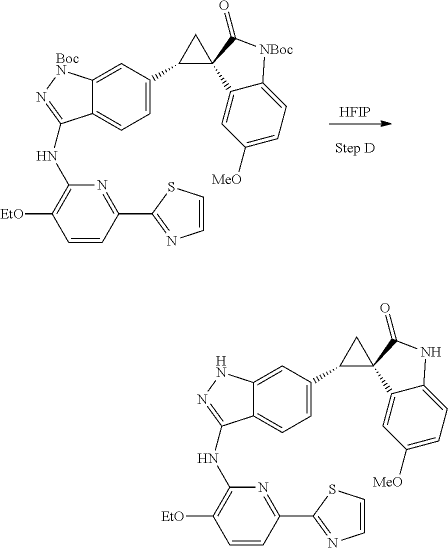 [SMILES: CCOc1ccc(-c2nccs2)nc1Nc1nn(C(=O)OC(C)(C)C)c2cc([C@@H]3C[C@@]34C(=O)N(C(=O)OC(C)(C)C)c3ccc(OC)cc34)ccc12 ; "HFIP" ; CCOc1ccc(-c2nccs2)nc1Nc1n[nH]c2cc([C@@H]3C[C@@]34C(=O)Nc3ccc(OC)cc34)ccc12]

Into a 8 mL vial were added tert-butyl (1R, 2S)-2-[1-(tert-butoxycarbonyl)-3-{[3-ethoxy-6-(1,3-thiazol-2-yl)pyridin-2-yl]amino}indazol-6-yl]-5′-methoxy-2′-oxospiro[cyclopropane-1,3′-indole]-1′-carboxylate (74 mg, 0.10 mmol, 1.0 equiv) and HFIP (5 mL) at room temperature. The resulting mixture was stirred for 16 h at 60° C. under nitrogen atmosphere. The resulting mixture was concentrated under reduced pressure. The crude product was purified by prep-HPLC (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 30% B to 50% B in 11 min, 50% B; Wavelength: 220 nm; RT1 (min): 9.20 to afford Example 28 (14 mg, 25.8%) as a white solid. m/z (ESI, +ve ion)=525.25 [M+H]⁺. ¹H-NMR (400 MHz, DMSO-d₆) δ 12.50 (s, 1H), 10.41 (s, 1H), 8.49 (s, 1H), 7.76 (s, 1H), 7.53-7.47 (m, 2H), 7.40 (s, 1H), 7.33-7.32 (m, 2H), 6.83-6.77 (m, 2H), 6.66-6.63 (m, 1H), 5.74 (s, 1H), 4.24-4.21 (m, 2H), 3.30 (s, 3H), 3.22-3.20 (m, 1H), 2.32-2.31 (m, 1H), 1.99-1.98 (m, 1H), 1.45 (t, J=7.2 Hz, 3H).

### Example 33. (1R,2S)-2-{3-[2-Ethoxy-5-(1,3-thiazol-2-yl)anilino]-1H-indazol-6-yl}-5′-methoxyspiro[cyclopropane-1,3′-indol]-2′(1′H)-one

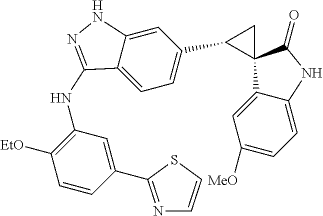 [SMILES: CCOc1ccc(-c2nccs2)cc1Nc1n[nH]c2cc([C@@H]3C[C@@]34C(=O)Nc3ccc(OC)cc34)ccc12]

### Step A. 2-Ethoxy-5-(1,3-thiazol-2-yl)aniline

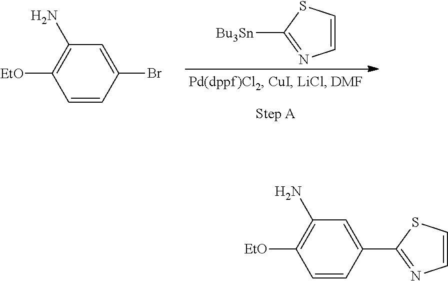

To a stirred solution of 5-bromo-2-ethoxyaniline (220 mg, 1.02 mmol, 1.00 equiv), Pd(dppf)Cl₂·CH₂Cl₂ (82.9 mg, 0.102 mmol, 0.100 equiv) and 2-(tributylstannyl)-1,3-thiazole (571 mg, 1.53 mmol, 1.50 equiv) in DMF (5 mL) were added CuI (97.0 mg, 0.509 mmol, 0.500 equiv) and LiCl (86.3 mg, 2.04 mmol, 2.00 equiv) at room temperature under nitrogen atmosphere. The resulting mixture was stirred for 2 h at 90° C. under nitrogen atmosphere. The mixture was allowed to cool down to room temperature. The reaction was quenched by the addition of water (5 mL) at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EtOAc (1/1) to afford the title compound (65 mg, 29%) as an off-white solid. m/z (ESI, +ve ion)=221.10 [M+H]⁺.

### Step B. Tert-butyl (1R, 2S)-2-[1-(tert-butoxycarbonyl)-3-{[2-ethoxy-5-(1,3-thiazol-2-yl)phenyl]amino}indazol-6-yl]-5′-methoxy-2′-oxospiro[cyclopropane-1,3′-indole]-1′-carboxylate

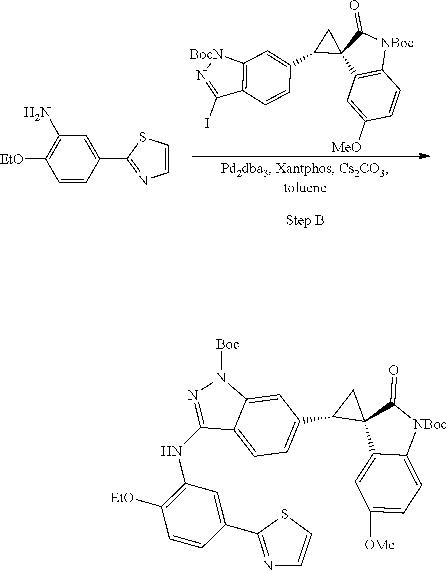

To a stirred solution of tert-butyl (1R,2S)-2-[1-(tert-butoxycarbonyl)-3-iodoindazol-6-yl]-5′-methoxy-2′-oxospiro[cyclopropane-1,3′-indole]-1′-carboxylate (100 mg, 0.158 mmol, 1.00 equiv), Cs₂CO₃ (103 mg, 0.316 mmol, 2.00 equiv) and 2-ethoxy-5-(1,3-thiazol-2-yl)aniline (52.3 mg, 0.237 mmol, 1.50 equiv) in toluene (3 mL) were added XantPhos (18.3 mg, 0.032 mmol, 0.20 equiv) and Pd₂(dba)₃ (29.0 mg, 0.032 mmol, 0.2.00 equiv) at room temperature under nitrogen atmosphere. The resulting mixture was stirred for 2 h at 90° C. under nitrogen atmosphere. The mixture was allowed to cool down to room temperature. The reaction was quenched by the addition of Water (5 mL) at room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EtOAc (1/1) to afford the title compound (110 mg, 97%) as an off-white solid. m/z (ESI, +ve ion)=724.30 [M+H]⁺.

### Step C. (1R, 2S)-2-(3-{[2-ethoxy-5-(1,3-thiazol-2-yl)phenyl]amino}-1H-indazol-6-yl)-5′-methoxy-1′H-spiro[cyclopropane-1,3′-indol]-2′-one

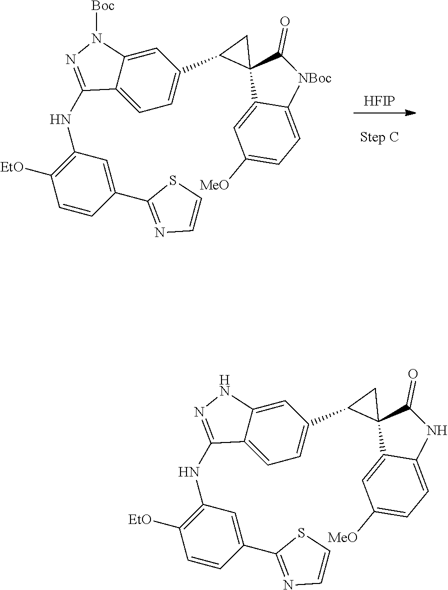 [SMILES: CCOc1ccc(-c2nccs2)cc1Nc1nn(C(=O)OC(C)(C)C)c2cc([C@@H]3C[C@@]34C(=O)N(C(=O)OC(C)(C)C)c3ccc(OC)cc34)ccc12 ; "HFIP" ; CCOc1ccc(-c2nccs2)cc1Nc1n[nH]c2cc([C@@H]3C[C@@]34C(=O)Nc3ccc(OC)cc34)ccc12]

A solution of tert-butyl (1R, 2S)-2-[1-(tert-butoxycarbonyl)-3-{[2-ethoxy-5-(1,3-thiazol-2-yl)phenyl]amino}indazol-6-yl]-5′-methoxy-2′-oxospiro[cyclopropane-1,3′-indole]-1′-carboxylate (110 mg, 0.152 mmol, 1.00 equiv) in HFIP (3 mL) was stirred for 16 h at 60° C. The mixture was allowed to cool down to room temperature. The resulting mixture was concentrated under reduced pressure. The crude product (70 mg) was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 42% B to 52% B in 11 min, 52% B; Wavelength: 220 nm; RT1 (min): 10.23 to afford Example 33 (22.2 mg, 27.5%) as an off-white solid. m/z (ESI, +ve ion)=524.15 [M+H]⁺. ¹H-NMR (400 MHz, DMSO-d₆) δ 12.27 (s, 1H), 10.42 (s, 1H), 8.75 (d, J=2.0 Hz, 1H), 7.84 (d, J=3.2 Hz, 1H), 7.80-7.71 (m, 2H), 7.65 (d, J=3.2 Hz, 1H), 7.49-7.41 (m, 1H), 7.37 (s, 1H), 7.09 (d, J=8.4 Hz, 1H), 6.90 (d, J=8.4 Hz, 1H), 6.76 (d, J=8.4 Hz, 1H), 6.63-6.59 (m, 1H), 5.70 (d, J=2.4 Hz, 1H), 4.24 (q, J=7.2 Hz, 2H), 3.32 (s, 3H), 3.20 (t, J=8.4 Hz, 1H), 2.39-2.33 (m, 1H), 2.05-1.99 (m, 1H), 1.47 (t, J=6.8 Hz, 3H).

### Example 44. (1R,2S)-2-(3-{[5-Ethoxy-2-(1,3-thiazol-2-yl)pyrimidin-4-yl]amino}-1H-indazol-6-yl)-5′-methoxyspiro[cyclopropane-1,3′-indol]-2′(1′H)-one

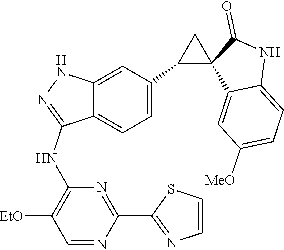 [SMILES: CCOc1cnc(-c2nccs2)nc1Nc1n[nH]c2cc([C@@H]3C[C@@]34C(=O)Nc3ccc(OC)cc34)ccc12]

### Step A. 4-Amino-2-chloropyrimidin-5-ol

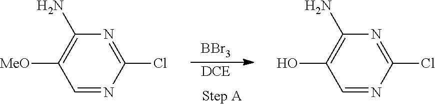 [SMILES: COc1cnc(Cl)nc1N ; "BBr3" ; "DCE" ; Nc1nc(Cl)ncc1O ; "Step A"]

Into a 100 mL vial were added 2-chloro-5-methoxypyrimidin-4-amine (500 mg, 3.13 mmol, 1.00 equiv), BBr₃ (7.8 mg, 31 mmol, 10 equiv) and DCE (15 mL) at room temperature. The resulting mixture was stirred for 16 h at room temperature under nitrogen atmosphere. The resulting mixture was concentrated under reduced pressure. This resulted in 4-amino-2-chloropyrimidin-5-ol (420 mg, 92%) as a yellow solid. The crude product was used in the next step directly without further purification. m/z (ESI, +ve ion)=146.05 [M+H]⁺.

### Step B. 2-Chloro-5-ethoxypyrimidin-4-amine

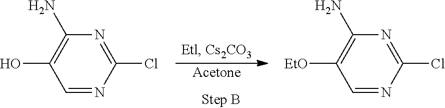 [SMILES: Nc1nc(Cl)ncc1O ; "Acetone" ; "Etl, Cs2CO3" ; CCOc1cnc(Cl)nc1N]

Into an 8 mL vial were added 4-amino-2-chloropyrimidin-5-ol (400 mg, 2.75 mmol, 1.00 equiv), iodoethane (343 mg, 2.20 mmol, 0.800 equiv), Cs₂CO₃ (2695 mg, 8.271 mmol, 3.01 equiv) and acetone (10 mL) at room temperature. The resulting mixture was stirred for 16 h at 60° C. under nitrogen atmosphere. The resulting mixture was extracted with EtOAc (5×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (1/1) to afford the title compound (230 mg, 48.2%) as a white solid. m/z (ESI, +ve ion)=174.05 [M+H]⁺.

### Step C. 5-Ethoxy-2-(1,3-thiazol-2-yl)pyrimidin-4-amine

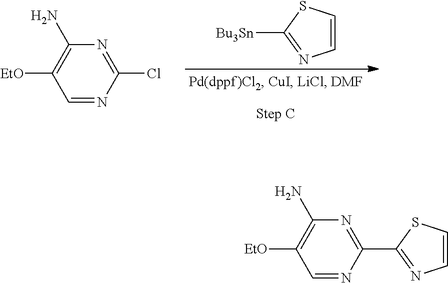

To a stirred mixture of 2-chloro-5-ethoxypyrimidin-4-amine (120 mg, 0.691 mmol, 1.00 equiv) and 2-(tributylstannyl)-1,3-thiazole (388 mg, 1.04 mmol, 1.50 equiv) in DMF (0.5 mL) were added Pd(dppf)Cl₂ (101 mg, 0.138 mmol, 0.200 equiv), CuI (66 mg, 0.35 mmol, 0.50 equiv) and LiCl (58.60 mg, 1.382 mmol, 2 equiv) at room temperature under argon atmosphere. The resulting mixture was stirred for additional 2 h at 110° C. The resulting mixture was extracted with EtOAc (3×30 mL). The combined organic layers were washed with brine (3×20 mL), dried over anhydrous Na₂SO₄. After filtration, the filtrate was concentrated under reduced pressure. The residue was purified by prep-TLC (EtOAc) to afford the title compound (65 mg, 42%) as a white solid. m/z (ESI, +ve ion)=223.00 [M+H]⁺.

### Step D. Tert-butyl (1R, 2S)-2-[1-(tert-butoxycarbonyl)-3-{[5-ethoxy-2-(1,3-thiazol-2-yl)pyrimidin-4-yl]amino}indazol-6-yl]-5′-methoxy-2′-oxospiro[cyclopropane-1,3′-indole]-1′-carboxylate

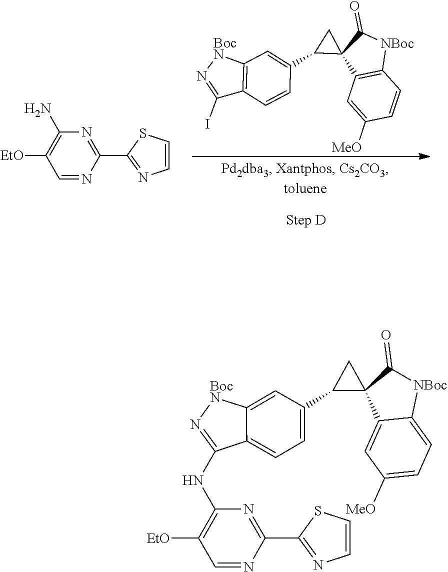 [SMILES: CCOc1cnc(-c2nccs2)nc1N ; COc1ccc2c(c1)[C@]1(C[C@H]1c1ccc3c(I)nn(C(=O)OC(C)(C)C)c3c1)C(=O)N2C(=O)OC(C)(C)C ; CCOc1cnc(-c2nccs2)nc1Nc1nn(C(=O)OC(C)(C)C)c2cc([C@@H]3C[C@@]34C(=O)N(C(=O)OC(C)(C)C)c3ccc(OC)cc34)ccc12]

To a stirred mixture of 5-ethoxy-2-(1,3-thiazol-2-yl)pyrimidin-4-amine (33.6 mg, 0.151 mmol, 1.20 equiv) and tert-butyl (1R, 2S)-2-[1-(tert-butoxycarbonyl)-3-iodoindazol-6-yl]-5′-methoxy-2′-oxospiro[cyclopropane-1,3′-indole]-1′-carboxylate (79.5 mg, 0.126 mmol, 1.00 equiv) in toluene (4 mL) added Pd₂(dba)₃ (34.6 mg, 0.038 mmol, 0.30 equiv), Xantphos (22.0 mg, 0.038 mmol, 0.300 equiv) and Cs₂CO₃ (82.1 mg, 0.252 mmol, 2.00 equiv) at room temperature under argon atmosphere. The resulting mixture was stirred for additional 2 h at 60° C. The resulting mixture was extracted with EtOAc (3×30 mL). The combined organic layers were washed with brine (3×20 mL), dried over anhydrous Na₂SO₄. After filtration, the filtrate was concentrated under reduced pressure. The residue was purified by prep-TLC (CH₂Cl₂/MeOH 15/1) to afford the title compound (37 mg, 40%) as a white solid. m/z (ESI, +ve ion)=726.30 [M+H]⁺.

### Step E. (1R, 2S)-2-(3-{[5-Ethoxy-2-(1,3-thiazol-2-yl)pyrimidin-4-yl]amino}-1H-indazol-6-yl)-5′-methoxy-1′H-spiro[cyclopropane-1,3′-indol]-2′-one

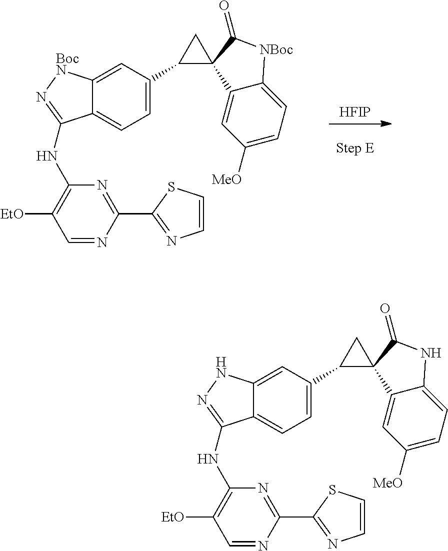 [SMILES: CCOc1cnc(-c2nccs2)nc1Nc1nn(C(=O)OC(C)(C)C)c2cc([C@@H]3C[C@@]34C(=O)N(C(=O)OC(C)(C)C)c3ccc(OC)cc34)ccc12 ; "HFIP" ; CCOc1cnc(-c2nccs2)nc1Nc1n[nH]c2cc([C@@H]3C[C@@]34C(=O)Nc3ccc(OC)cc34)ccc12]

To a stirred mixture of tert-butyl (1R,2S)-2-[1-(tert-butoxycarbonyl)-3-{[5-ethoxy-2-(1,3-thiazol-2-yl)pyrimidin-4-yl]amino}indazol-6-yl]-5′-methoxy-2′-oxospiro[cyclopropane-1,3′-indole]-1′-carboxylate (35 mg, 0.048 mmol, 1.0 equiv) in HFIP (2 mL) at room temperature under argon atmosphere. The resulting mixture was stirred for additional 5 h at 60° C. The resulting mixture was concentrated under reduced pressure. The crude product (50 mg) was purified by prep-HPLC with the following conditions (Column: SunFire C18 OBD Prep Column 19×150 mm, 5 μm; Mobile Phase A: Water (0.1% FA), Mobile Phase B: ACN; Flow rate: 20 mL/min mL/min; Gradient: 30% B to 40% B in 9 min; Wavelength: 254 nm/220 nm; RT₁ (min): 4.92) to afford Example 44 (16.2 mg, 62%) as a white solid. m/z (ESI, +ve ion)=525.95 [M+H]⁺. ¹H NMR (300 MHz, Methanol-d₄) δ 8.08 (s, 1H), 7.81 (dd, J=3.2, 1.1 Hz, 1H), 7.64 (d, J=8.5 Hz, 1H), 7.49 (s, 1H), 7.36 (dd, J=3.2, 1.1 Hz, 1H), 6.88-6.84 (m, 2H), 6.67-6.64 (m, 1H), 5.70 (d, J=2.5 Hz, 1H), 4.37-4.30 (m, 2H), 3.38 (d, J=8.5 Hz, 1H), 3.27 (d, J=1.2 Hz, 3H), 2.29 (dd, J=7.9, 4.9 Hz, 1H), 2.18 (dd, J=9.0, 4.8 Hz, 1H), 1.55 (t, J=9.2 Hz, 3H).

The compounds in Table 1B were prepared using materials and methods analogous to those disclosed herein and methods known to those having ordinary skill in the art.

TABLE 1B

| Example | | |
|---|---|---|
| No. | Name | MS/¹H NMR |
| 1 | 6-Methoxy-5-({6-[(1R,2S)-5′-methoxy-2′- | m/z (ESI, +ve ion) = 517.15 [M + H]⁺. ¹H-NMR |
| | oxo-1′,2′-dihydrospiro[cyclopropane-1,3′- | (400 MHz, DMSO-d₆) δ 12.35 (s, 1H), 10.42 |
| | indol]-2-yl]-1H-indazol-3-yl}amino)-2,3- | (s, 1H), 8.28 (s, 1H), 8.00 (s, 1H), 7.84 (d, J = |
| | dihydro-1H-1-benzothiophene-1,1-dione | 8.4 Hz, 1H), 7.38 (d, J = 1.4 Hz, 1H), 7.23 (s, |
| | | 1H), 6.92 (dd, J = 8.5, 1.4 Hz, 1H), 6.76 (d, J = |
| | | 8.4 Hz, 1H), 6.59 (dd, J = 8.5, 2.6 Hz, 1H), |
| | | 5.70 (d, J = 2.6 Hz, 1H), 3.99 (s, 3H), 3.56- |
| | | 3.46 (m, 2H), 3.30 (s, 3H), 3.27-3.15 (m, |
| | | 3H), 2.37-2.29 (m, 1H), 2.03-1.95 (m, 1H) |
| 3 | (1R,2S)-2-[3-(4-acetyl-2-methoxyanilino)- | m/z (ESI, +ve ion) = 469.15 [M + H]⁺. ¹H-NMR |
| | 1H-indazol-6-yl]-5′- | (400 MHz, DMSO-d₆) δ 12.35 (s, 1H), 10.41 |
| | methoxyspiro[cyclopropane-1,3′-indol]- | (s, 1H), 8.26 (s, 1H), 8.00 (d, J = 8.5 Hz, 1H), |
| | 2′(1′H)-one | 7.82 (d, J = 8.4 Hz, 1H), 7.61 (dd, J = 8.5, 1.9 |
| | | Hz, 1H), 7.48 (d, J = 1.9 Hz, 1H), 7.37 (s, |
| | | 1H), 6.92 (d, J = 8.5 Hz, 1H), 6.75 (d, J = 8.4 |
| | | Hz, 1H), 6.59 (dd, J = 8.5, 2.6 Hz, 1H), 5.70 |
| | | (d, J = 2.6 Hz, 1H), 3.98 (s, 3H), 3.32 (s, 3H), |
| | | 3.19 (t, J = 8.5 Hz, 1H), 2.50 (s, 3H), 2.33 (dd, |
| | | J = 7.9, 4.7 Hz, 1H), 1.99 (dd, J = 9.0, 4.6 Hz, |
| | | 1H) |
| 7 | (1R,2S)-2-{3-[2-Ethoxy-4-(pyrazin-2- | /z (ESI, +ve ion) = 519.25 [M + H]⁺. ¹H-NMR |
| | yl)anilino]-1H-indazol-6-yl}-5′- | (400 MHz, DMSO-d₆) δ 12.26 (s, 1H), 10.42 |
| | methoxyspiro[cyclopropane-1,3′-indol]- | (s, 1H), 9.23 (d, J = 1.6 Hz, 1H), 8.66-8.60 |
| | 2′(1′H)-one | (m, 1H), 8.48 (d, J = 2.5 Hz, 1H), 8.10 (d, J = |
| | | 8.4 Hz, 1H), 7.88 (s, 1H), 7.79-7.71 (m, 3H), |
| | | 7.36 (s, 1H), 6.91 (d, J = 8.4 Hz, 1H), 6.75 (d, |
| | | J = 8.4 Hz, 1H), 6.58 (dd, J = 8.4, 2.6 Hz, |
| | | 1H), 5.69 (d, J = 2.6 Hz, 1H), 4.28 (q, J = 7.0 |
| | | Hz, 2H), 3.33 (s, 3H), 3.19 (t, J = 8.5 Hz, 1H), |
| | | 2.34-2.32 (m, 1H), 1.99 (dd, J = 9.0, 4.6 Hz, |
| | | 1H), 1.49 (t, J = 7.0 Hz, 3H) |
| 8 | (1R,2S)-2-{3-[(3-Ethoxyquinolin-2- | m/z (ESI, +ve ion) = 492.25 [M + H]+. 1H- |
| | yl)amino]-1H-indazol-6-yl}-5′- | NMR (400 MHz, Methanol-d4) δ 7.75 (d, J = |
| | methoxyspiro[cyclopropane-1,3′-indol]- | 8.3 Hz, 1H), 7.66 (d, J = 7.9 Hz, 1H), 7.52 (d, |
| | 2′(1′H)-one | J = 8.1 Hz, 1H), 7.44 (d, J = 6.9 Hz, 2H), 7.37 |
| | | (t, J = 7.7 Hz, 1H), 7.26 (t, J = 7.7 Hz, 1H), |
| | | 6.92 (d, J = 8.4 Hz, 1H), 6.85 (d, J = 8.1 Hz, |
| | | 1H), 6.64 (d, J = 8.5 Hz, 1H), 5.72 (s, 1H), |
| | | 4.34 (d, J = 7.6 Hz, 2H), 3.33-3.30 (m, 4H), |
| | | 2.22 (d, J = 24.4 Hz, 2H), 1.59 (t, J = 6.7 Hz, |
| | | 3H) |
| 9 | (1R,2S)-2-(3-{4-[3- | m/z (ESI, +ve ion) = 481.2 [M − N(CH₃)₂]⁺. ¹H |
| | (Dimethylamino)oxetan-3-yl]-2- | NMR (400 MHz, DMSO) δ 12.05 (s, 1H), |
| | methoxyanilino}-1H-indazol-6-yl)-5′- | 10.42 (s, 1H), 8.05 (d, J = 8.2 Hz, 1H), 7.82 |
| | methoxyspiro[cyclopropane-1,3′-indol]- | (d, J = 8.3 Hz, 1H), 7.69 (s, 1H), 7.31 (s, 1H), |
| | 2′(1′H)-one | 6.87 (dd, J = 8.4, 1.4 Hz, 1H), 6.75 (d, J = 6.8 |
| | | Hz, 1H), 6.73 (s, 1H), 6.68 (dd, J = 8.2, 1.9 |
| | | Hz, 1H), 6.58 (dd, J = 8.4, 2.6 Hz, 1H), 5.69 |
| | | (d, J = 2.6 Hz, 1H), 4.76 (d, J = 6.0 Hz, 2H), |
| | | 4.68 (d, J = 6.0 Hz, 2H), 3.90 (s, 3H), 3.32 (s, |
| | | 3H), 3.18 (t, J = 8.4 Hz, 1H), 2.31 (dd, J = 7.9, |
| | | 4.5 Hz, 1H), 1.98 (dd, J = 9.0, 4.7 Hz, 1H), |
| | | 1.95 (s, 6H) |
| 10 | (1R,2S)-2-{3-[2-Ethoxy-4-(1H-1,2,4- | m/z (ESI, +ve ion) = 508.15 [M + H]⁺. ¹H-NMR |
| | triazol-1-yl)anilino]-1H-indazol-6-yl}-5′- | (400 MHz, DMSO-d₆) δ 12.19 (s, 1H), 10.42 |
| | methoxyspiro[cyclopropane-1,3′-indol]- | (s, 1H), 9.18 (s, 1H), 8.20-8.10 (m, 2H), 7.77 |
| | 2′(1′H)-one | (t, J = 4.2 Hz, 2H), 7.45 (d, J = 2.4 Hz, 1H), |
| | | 7.39-7.32 (m, 2H), 6.90 (d, J = 8.4 Hz, 1H), |
| | | 6.76 (d, J = 8.4 Hz, 1H), 6.59 (dd, J = 8.5, 2.6 |
| | | Hz, 1H), 5.70 (d, J = 2.5 Hz, 1H), 4.27 (q, J = |
| | | 6.9 Hz, 2H), 3.33 (s, 3H), 3.20 (t, J = 8.4 Hz, |
| | | 1H), 2.32 (dd, J = 8.0, 4.7 Hz, 1H), 2.00 (dd, J = |
| | | 8.9, 4.7 Hz, 1H), 1.48 (t, J = 6.9 Hz, 3H) |
| 11 | (1R,2S)-5′-Methoxy-2-{3-[2-methoxy-5- | m/z (ESI, +ve ion) = 507.15 [M + H]⁺. ¹H-NMR |
| | (1-methyl-1H-pyrazol-4-yl)anilino]-1H- | (400 MHz, DMSO-d₆) δ 12.10 (s, 1H), 10.42 |
| | indazol-6-yl}spiro[cyclopropane-1,3′- | (s, 1H), 8.25 (d, J = 1.7 Hz, 1H), 7.89 (s, 1H), |
| | indol]-2′(1′H)-one | 7.83 (d, J = 8.4 Hz, 1H), 7.68 (s, 1H), 7.63 (s, |
| | | 1H), 7.32 (s, 1H), 6.99 (d, J = 2.4 Hz, 2H), |
| | | 6.88 (d, J = 8.6 Hz, 1H), 6.75 (d, J = 8.4 Hz, |
| | | 1H), 6.59 (dd, J = 8.4, 2.6 Hz, 1H), 5.70 (d, J = |
| | | 2.5 Hz, 1H), 3.90 (s, 3H), 3.85 (s, 3H), 3.33 |
| | | (s, 3H), 3.19 (t, J = 8.4 Hz, 1H), 2.33 (dd, J = |
| | | 8.0, 4.7 Hz, 1H), 1.98 (dd, J = 9.0, 4.6 Hz, |
| | | 1H) |
| 12 | (1R,2S)-2-{3-[(7-Ethoxy-1-oxo-1?5- | m/z (ESI, +ve ion) = 508.3 [M + H]⁺. ¹H NMR |
| | quinolin-6-yl)amino]-1H-indazol-6-yl}-5′- | (400 MHz, DMSO) δ 12.35 (s, 1H), 10.36 (s, |
| | methoxyspiro[cyclopropane-1,3′-indol]- | 1H), 8.31 (s, 1H), 8.27 (d, J = 6.1 Hz, 1H), |
| | 2′(1′H)-one | 8.21 (s, 1H), 7.85 (s, 1H), 7.73 (d, J = 8.4 Hz, |
| | | 1H), 7.59 (d, J = 8.4 Hz, 1H), 7.33 (s, 1H), |
| | | 7.17 (dd, J = 8.4, 6.1 Hz, 1H), 6.87 (d, J = 8.4 |
| | | Hz, 1H), 6.68 (d, J = 8.4 Hz, 1H), 6.52 (dd, J = |
| | | 8.4, 2.6 Hz, 1H), 5.62 (d, J = 2.6 Hz, 1H), 4.32 |
| | | (q, J = 6.9 Hz, 2H), 3.14 (t, J = 8.4 Hz, 1H), |
| | | 2.31-2.23 (m, 1H), 1.97-1.89 (m, 1H), 1.49 |
| | | (t, J = 6.9 Hz, 3H) |
| 13 | (1R,2S)-5′-Methoxy-2-(3-((2-methoxy-5- | m/z (ESI, +ve ion) = 494.10 [M + H]⁺. ¹H-NMR |
| | (oxazol-2-yl)phenyl)amino)-1H-indazol-6- | (300 MHz, DMSO-d₆) δ 12.25 (s, 1H), 10.44 |
| | yl)spiro[cyclopropane-1,3′-indolin]-2′-one | (s, 1H), 8.85 (d, J = 2.1 Hz, 1H), 8.14 (d, J = |
| | | 0.8 Hz, 1H), 7.94 (s, 1H), 7.88 (d, J = 8.3 Hz, |
| | | 1H), 7.49 (dd, J = 8.3, 2.2 Hz, 1H), 7.36 (s, |
| | | 1H), 7.31 (d, J = 0.9 Hz, 1H), 7.13 (d, J = 8.5 |
| | | Hz, 1H), 6.89 (d, J = 8.5 Hz, 1H), 6.75 (d, J = |
| | | 8.4 Hz, 1H), 6.58 (dd, J = 8.4, 2.6 Hz, 1H), |
| | | 5.72 (d, J = 2.6 Hz, 1H), 3.98 (s, 3H), 3.32 (s, |
| | | 3H), 3.19 (t, J = 8.4 Hz, 1H), 2.35 (dd, J = 8.0, |
| | | 4.6 Hz, 1H), 1.98 (dd, J = 9.0, 4.6 Hz, 1H) |
| 16 | (1R,2S)-5′-Methoxy-2-{3-[2-methoxy-5- | m/z (ESI, +ve ion) = 494.20 [M + H]⁺. ¹H-NMR |
| | (1,3-oxazol-4-yl)anilino]-1H-indazol-6- | (400 MHz, DMSO-d₆) δ 12.16 (s, 1H), 10.43 |
| | yl}spiro[cyclopropane-1,3′-indol]-2′(1′H)- | (s, 1H), 8.54 (d, J = 2.0 Hz, 1H), 8.41-8.36 |
| | one | (m, 2H), 7.83 (d, J = 8.4 Hz, 1H), 7.74 (s, 1H), |
| | | 7.33 (s, 1H), 7.24 (dd, J = 8.3, 2.1 Hz, 1H), |
| | | 7.05 (d, J = 8.4 Hz, 1H), 6.91-6.84 (m, 1H), |
| | | 6.75 (d, J = 8.4 Hz, 1H), 6.58 (dd, J = 8.4, 2.6 |
| | | Hz, 1H), 5.71 (d, J = 2.5 Hz, 1H), 3.93 (s, |
| | | 3H), 3.32 (s, 3H), 3.19 (t, J = 8.4 Hz, 1H), |
| | | 2.37-2.29 (m, 1H), 1.98 (dd, J = 8.9, 4.7 Hz, |
| | | 1H) |
| 17 | (1R,2S)-5′-Methoxy-2-{3-[2-methoxy-4- | m/z (ESI, +ve ion) = 509.1 [M + H]⁺. ¹H NMR |
| | (2-methyl-2H-tetrazol-5-yl)anilino]-1H- | (400 MHz, DMSO) δ 12.22 (s, 1H), 10.42 (s, |
| | indazol-6-yl}spiro[cyclopropane-1,3′- | 1H), 8.18 (d, J = 8.4 Hz, 1H), 8.04 (s, 1H), |
| | indol]-2′(1′H)-one | 7.85 (d, J = 8.4 Hz, 1H), 7.62 (dd, J = 8.3, 1.9 |
| | | Hz, 1H), 7.58 (d, J = 1.8 Hz, 1H), 7.35 (s, 1H), |
| | | 6.90 (dd, J = 8.4, 1.4 Hz, 1H), 6.75 (d, J = 8.4 |
| | | Hz, 1H), 6.58 (dd, J = 8.4, 2.6 Hz, 1H), 5.70 |
| | | (d, J = 2.6 Hz, 1H), 4.40 (s, 3H), 4.00 (s, 3H), |
| | | 3.32 (s, 3H), 3.19 (t, J = 8.4 Hz, 1H), 2.32 (dd, |
| | | J = 7.8, 4.8 Hz, 1H), 1.98 (dd, J = 9.0, 4.7 Hz, |
| | | 1H) |
| 18 | (1R,2S)-5′-Methoxy-2-{3-[2-methoxy-5- | m/z (ESI, +ve ion) = 494.20 [M + H]⁺. ¹H-NMR |
| | (1,3-oxazol-5-yl)anilino]-1H-indazol-6- | (400 MHz, DMSO-d₆) δ 12.20 (s, 1H), 10.41 |
| | yl}spiro[cyclopropane-1,3′-indol]-2′(1′H)- | (s, 1H), 8.50 (d, J = 2.1 Hz, 1H), 8.36 (s, 1H), |
| | one | 7.85 (t, J = 4.2 Hz, 2H), 7.42 (s, 1H), 7.34 (s, |
| | | 1H), 7.22 (dd, J = 8.3, 2.2 Hz, 1H), 7.10 (d, J = |
| | | 8.4 Hz, 1H), 6.89 (d, J = 8.4 Hz, 1H), 6.75 |
| | | (d, J = 8.4 Hz, 1H), 6.59 (dd, J = 8.5, 2.6 Hz, |
| | | 1H), 5.71 (d, J = 2.5 Hz, 1H), 3.95 (s, 3H), |
| | | 3.30 (s, 3H), 3.19 (t, J = 8.5 Hz, 1H), 2.33 (dd, |
| | | J = 8.0, 4.8 Hz, 1H), 1.99 (dd, J = 9.0, 4.6 Hz, |
| | | 1H) |
| 19 | (1R,2S)-5′-methoxy-2-(3-((3-methoxy-6- | m/z (ESI, +ve ion) = 496.30 [M + H]⁺. ¹H-NMR |
| | (oxazol-2-yl)pyrazin-2-yl)amino)-1H- | (400 MHz, DMSO-d₆) δ 12.65 (s, 1H), 10.41 |
| | indazol-6-yl)spiro[cyclopropane-1,3′- | (s, 1H), 9.17 (s, 1H), 8.19 (s, 1H), 8.04 (s, |
| | indolin]-2′-one | 1H), 7.65 (d, J = 8.4 Hz, 1H), 7.41 (s, 1H), |
| | | 7.29 (s, 1H), 6.89 (dd, J = 8.6, 1.4 Hz, 1H), |
| | | 6.75 (d, J = 8.4 Hz, 1H), 6.59 (dd, J = 8.4, 2.6 |
| | | Hz, 1H), 5.73 (d, J = 2.6 Hz, 1H), 4.07 (s, |
| | | 3H), 3.30 (s, 3H), 3.19 (t, J = 8.4 Hz, 1H), |
| | | 2.32 (dd, J = 8.0, 4.6 Hz, 1H), 1.99 (dd, J = |
| | | 9.0, 4.7 Hz, 1H) |
| 20 | (1R,2S)-2-(3-{[2-(Dimethylamino)-5- | m/z (ESI, +ve ion) = 472.30 [M + H]⁺. ¹H-NMR |
| | methoxypyrimidin-4-yl]amino}-1H- | (300 MHz, DMSO-d₆) δ 12.53 (s, 1H), 10.39 |
| | indazol-6-yl)-5′- | (s, 1H), 8.79 (s, 1H), 7.75 (s, 1H), 7.48 (d, J = |
| | methoxyspiro[cyclopropane-1,3′-indol]- | 8.4 Hz, 1H), 7.37 (s, 1H), 6.90-6.81 (m, 1H), |
| | 2′(1′H)-one | 6.75 (d, J = 8.4 Hz, 1H), 6.58 (dd, J = 8.5, 2.6 |
| | | Hz, 1H), 5.64 (d, J = 2.5 Hz, 1H), 3.79 (s, |
| | | 3H), 3.32 (s, 3H), 3.18 (t, J = 8.4 Hz, 1H), |
| | | 2.73 (s, 6H), 2.29 (dd, J = 7.9, 4.7 Hz, 1H), |
| | | 1.98 (dd, J = 9.0, 4.6 Hz, 1H) |
| 21 | (1R,2S)-5′-Methoxy-2-{3-[2-methoxy-5- | m/z (ESI, +ve ion) = 509.1 [M + H]⁺. ¹H NMR |
| | (2-methyl-2H-tetrazol-5-yl)anilino]-1H- | (400 MHz, DMSO) δ 12.23 (s, 1H), 10.42 (s, |
| | indazol-6-yl}spiro[cyclopropane-1,3′- | 1H), 8.88 (d, J = 2.1 Hz, 1H), 7.89 (s, 1H), |
| | indol]-2′(1′H)-one | 7.86 (d, J = 8.4 Hz, 1H), 7.55 (dd, J = 8.3, 2.1 |
| | | Hz, 1H), 7.34 (s, 1H), 7.14 (d, J = 8.4 Hz, 1H), |
| | | 6.89 (dd, J = 8.5, 1.4 Hz, 1H), 6.75 (d, J = 8.4 |
| | | Hz, 1H), 6.58 (dd, J = 8.4, 2.6 Hz, 1H), 5.71 |
| | | (d, J = 2.6 Hz, 1H), 4.39 (s, 3H), 3.97 (s, 3H), |
| | | 3.31 (s, 3H), 3.18 (t, J = 8.5 Hz, 1H), 2.33 (dd, |
| | | J = 7.9, 4.7 Hz, 1H), 1.98 (dd, J = 9.0, 4.7 Hz, |
| | | 1H) |
| 22 | (1R,2S)-2-(3-{5-[3- | /z (ESI, +ve ion) = 526.2 [M + H]⁺. ¹H NMR |
| | (Dimethylamino)oxetan-3-yl]-2- | (400 MHz, DMSO) δ 12.11 (s, 1H), 10.41 (s, |
| | methoxyanilino}-1H-indazol-6-yl)-5′- | 1H), 7.82 (d, J = 2.2 Hz, 1H), 7.79 (d, J = 8.3 |
| | methoxyspiro[cyclopropane-1,3′-indol]- | Hz, 1H), 7.67 (s, 1H), 7.29 (s, 1H), 6.99 (d, J = |
| | 2′(1′H)-one | 8.3 Hz, 1H), 6.85 (dd, J = 8.4, 1.4 Hz, 1H), |
| | | 6.74 (d, J = 8.4 Hz, 1H), 6.61 (dd, J = 8.2, 2.2 |
| | | Hz, 1H), 6.58 (dd, J = 8.5, 2.6 Hz, 1H), 5.68 |
| | | (d, J = 2.6 Hz, 1H), 4.70 (dd, J = 5.9, 1.3 Hz, |
| | | 2H), 4.66 (dd, J = 5.9, 2.7 Hz, 2H), 3.91 (s, |
| | | 3H), 3.17 (t, J = 8.5 Hz, 1H), 2.31 (dd, J = 8.0, |
| | | 4.7 Hz, 1H), 1.96 (dd, J = 9.0, 4.7 Hz, 1H), |
| | | 1.93 (s, 6H) |
| 23 | (1R,2S)-2-{3-[2-ethoxy-4- | m/z (ESI, +ve ion) = 533.15 [M + H]⁺. ¹H- |
| | (methanesulfonyl)anilino]-1H-indazol-6- | NMR (300 MHz, Methanol-d₄) δ 7.86 (d, J = |
| | yl}-5′-methoxy-1′- | 8.3 Hz, 1H), 7.60 (d, J = 8.4 Hz, 1H), 7.50- |
| | methylspiro[cyclopropane-1,3′-indol]- | 7.37 (m, 3H), 6.94-6.90 (m, 2H), 6.73-6.69 |
| | 2′(1′H)-one | (m, 1H), 5.66 (d, J = 2.5 Hz, 1H), 4.34-4.27 |
| | | (m, 2H), 3.42-3.35 (m, 1H), 3.10 (s, 3H), |
| | | 2.28-2.18 (m, 2H), 1.58-1.54 (m, 3H) |
| 24 | (1R,2S)-5′-methoxy-2-(3-{[3-methoxy-6- | m/z (ESI, +ve ion) = 509.20 [M + H]+. 1H |
| | (1-methyl-1H-pyrazol-4-yl)pyrazin-2- | NMR (400 MHz, DMSO-d6) δ 12.56 (s, 1H), |
| | yl]amino}-1H-indazol-6- | 10.43 (s, 1H), 8.90 (s, 1H), 7.81 (s, 1H), 7.71 |
| | yl)spiro[cyclopropane-1,3′-indol]-2′(1′H)- | (s, 1H), 7.59-7.50 (m, 2H), 7.39 (s, 1H), 6.92 |
| | one | (d, J = 8.5 Hz, 1H), 6.76 (d, J = 8.4 Hz, 1H), |
| | | 6.60 (d, J = 8.5, 2.6 Hz, 1H), 5.69 (d, J = 2.5 |
| | | Hz, 1H), 3.99 (s, 3H), 3.73 (s, 3H), 3.30 (d, J = |
| | | 8.4 Hz, 3H), 3.22 (t, J = 8.4 Hz, 1H), 2.33- |
| | | 2.30 (m, 1H), 1.99-1.95 (m, 1H) |
| 26 | (1R,2S)-5′-methoxy-2-(3-{[2-methoxy-5- | m/z (ESI, +ve ion) = 508.20 [M + H]⁺. ¹H |
| | (1-methyl-1H-pyrazol-4-yl)pyridin-3- | NMR (400 MHz, DMSO-d₆) δ 12.19 (s, 1H), |
| | yl]amino}-1H-indazol-6- | 10.41 (s, 1H), 8.53 (d, J = 2.1 Hz, 1H), 8.04 |
| | yl)spiro[cyclopropane-1,3′-indol]-2′(1′H)- | (s, 1H), 7.98 (s, 1H), 7.92 (d, J = 8.4 Hz, 1H), |
| | one | 7.83 (d, J = 2.1 Hz, 1H), 7.70 (s, 1H), 7.33 (s, |
| | | 1H), 6.90 (d, J = 8.3 Hz, 1H), 6.75 (d, J = 8.4 |
| | | Hz, 1H), 6.59 (dd, J = 8.4, 2.6 Hz, 1H), 5.71 |
| | | (d, J = 2.6 Hz, 1H), 4.00 (s, 3H), 3.87 (s, 3H), |
| | | 3.30 (s, 3H), 3.19 (t, J = 8.4 Hz, 1H), 2.33 (dd, |
| | | J = 8.0, 4.7 Hz, 1H), 1.98 (dd, J = 9.1, 4.7 Hz, |
| | | 1H) |
| 27 | (1R,2S)-5′-methoxy-2-(3-{[5-methoxy-2- | m/z (ESI, +ve ion) = 508.20 [M + H]⁺. ¹H |
| | (1-methyl-1H-pyrazol-4-yl)pyridin-4- | NMR (400 MHz, DMSO-d₆) δ 12.43 (s, 1H), |
| | yl]amino}-1H-indazol-6- | 10.42 (s, 1H), 8.36 (s, 1H), 8.07 (d, J = 9.4 |
| | yl)spiro[cyclopropane-1,3′-indol]-2′(1′H)- | Hz, 2H), 7.99 (s, 1H), 7.84 (d, J = 8.4 Hz, |
| | one | 1H), 7.69 (d, J = 0.8 Hz, 1H), 7.38 (d, J = 1.2 |
| | | Hz, 1H), 6.93 (d, J = 8.5, 1.4 Hz, 1H), 6.76 (d, |
| | | J = 8.4 Hz, 1H), 6.59 (d, J = 8.4, 2.6 Hz, 1H), |
| | | 5.71 (d, J = 2.6 Hz, 1H), 3.98 (s, 3H), 3.86 (s, |
| | | 3H), 3.30 (d, J = 8.4 Hz, 3H), 3.20 (t, J = 8.4 |
| | | Hz, 1H), 2.34 (d, J = 7.9, 4.8 Hz, 1H), 1.99 (d, |
| | | J = 9.0, 4.7 Hz, 1H) |
| 29 | (1R,2S)-2-{3-[2-ethoxy-4-(1-methyl-1H- | m/z (ESI, +ve ion) = 521.25 [M + H]⁺. ¹H NMR |
| | imidazol-4-yl)anilino]-1H-indazol-6-yl}- | (400 MHz, DMSO-d₆) δ 12.05 (s, 1H), 10.41 |
| | 5′-methoxyspiro[cyclopropane-1,3′-indol]- | (s, 1H), 7.99 (d, J = 8.2 Hz, 1H), 7.73 (d, J = |
| | 2′(1′H)-one | 8.3 Hz, 1H), 7.57 (s, 1H), 7.52-7.45 (m, 2H), |
| | | 7.35 (d, J = 16.0 Hz, 2H), 7.25 (d, J = 8.7 Hz, |
| | | 1H), 6.88 (d, J = 8.3 Hz, 1H), 6.76 (d, J = 8.4 |
| | | Hz, 1H), 6.59 (dd, J = 8.7, 2.5 Hz, 1H), 5.69 |
| | | (d, J = 2.6 Hz, 1H), 4.20 (q, J = 6.9 Hz, 2H), |
| | | 3.68 (s, 3H), 3.19 (t, J = 8.4 Hz, 1H), 2.31 (dd, |
| | | J = 7.9, 4.9 Hz, 1H), 1.99 (dd, J = 9.0, 4.6 Hz, |
| | | 1H), 1.46 (t, J = 6.9 Hz, 3H) |
| 30 | (1R,2S)-5′-methoxy-2-{3-[2-methoxy-5- | m/z (ESI, +ve ion) = 510.20 [M + H]⁺. ¹H NMR |
| | (1,2-thiazol-3-yl)anilino]-1H-indazol-6- | (400 MHz, Methanol-d₄) δ 8.89 (d, J = 4.7 Hz, |
| | yl}spiro[cyclopropane-1,3′-indol]-2′(1′H)- | 1H), 8.47 (d, J = 2.2 Hz, 1H), 7.70-7.61 (m, |
| | one | 2H), 7.52 (dd, J = 8.4, 2.2 Hz, 1H), 7.39 (s, |
| | | 1H), 7.09 (d, J = 8.4 Hz, 1H), 6.92 (dd, J = |
| | | 8.4, 1.2 Hz, 1H), 6.85 (d, J = 8.4 Hz, 1H), |
| | | 6.63 (dd, J = 8.5, 2.6 Hz, 1H), 5.63 (d, J = 2.5 |
| | | Hz, 1H), 4.04 (s, 3H), 3.38 (d, J = 8.4 Hz, |
| | | 1H), 3.29 (s, 3H), 2.25 (dd, J = 7.9, 4.8 Hz, |
| | | 1H), 2.19 (dd, J = 9.1, 4.7 Hz, 1H) |
| 31 | (1R,2S)-5′-methoxy-2-(3-{[5-methoxy-2- | m/z (ESI, +ve ion) = 509.20 [M + H]⁺. ¹H- |
| | (1-methyl-1H-pyrazol-4-yl)pyrimidin-4- | NMR (400 MHz, DMSO-d₆) δ 12.63 (s, 1H), |
| | yl]amino}-1H-indazol-6- | 10.41 (s, 1H), 9.11 (s, 1H), 8.03 (s, 1H), 7. 72 |
| | yl)spiro[cyclopropane-1,3′-indol]-2′(1′H)- | (s, 1H), 7.56 (d, J = 8.4 Hz, 1H), 7.50 (s, 1H), |
| | one | 7.40 (s, 1H), 6.94 (d, J = 8.8 Hz, 1H), 6.76 (d, |
| | | J = 8.4 Hz, 1H), 6.61-6.58 (m, 1H), 5.65 (d, J = |
| | | 2.4 Hz, 1H), 3.94 (s, 3H), 3.72 (s, 3H), 3.31 |
| | | (s, 3H), 3.22 (s, 1H), 2.34-2.31 (m, 1H), 2.01- |
| | | 1.98 (m, 1H) |
| 32 | (1R,2S)-5′-methoxy-2-{3-[2-methoxy-5- | m/z (ESI, +ve ion) = 494.15 [M + H]⁺. ¹H- |
| | (1,2-oxazol-3-yl)anilino]-1H-indazol-6- | NMR (400 MHz, Methanol-d₄) δ 8.64 (d, J = |
| | yl}spiro[cyclopropane-1,3′-indol]-2′(1′H)- | 1.7 Hz, 1H), 8.36 (d, J = 2.1 Hz, 1H), 7.68 (d, |
| | one | J = 8.5 Hz, 1H), 7.41-7.33 (m, 2H), 7.10 (d, J = |
| | | 8.4 Hz, 1H), 6.92 (dd, J = 8.5, 1.3 Hz, 1H), |
| | | 6.84 (d, J = 8.5 Hz, 1H), 6.78 (d, J = 1.8 Hz, |
| | | 1H), 6.63 (dd, J = 8.5, 2.6 Hz, 1H), 5.63 (d, J = |
| | | 2.5 Hz, 1H), 4.04 (s, 3H), 3.38 (d, J = 8.5 |
| | | Hz, 1H), 3.29 (s, 3H), 2.25 (dd, J = 7.9, 4.8 |
| | | Hz, 1H), 2.19 (dd, J = 9.0, 4.8 Hz, 1H) |
| 34 | (1R,2S)-2-(3-{[5-ethoxy-2-(1,3-thiazol-2- | m/z (ESI, +ve ion) = 525.25 [M + H]⁺. ¹H- |
| | yl)pyridin-4-yl]amino}-1H-indazol-6-yl)- | NMR (300 MHz, DMSO-d₆) δ 12.57 (s, 1H), |
| | 5′-methoxyspiro[cyclopropane-1,3′-indol]- | 10.41 (s, 1H), 8.64 (s, 1H), 8.43 (s, 1H), 8.18 |
| | 2′(1′H)-one | (s, 1H), 7.86 (d, J = 3.2 Hz, 1H), 7.79 (d, J = |
| | | 8.4 Hz, 1H), 7.72 (d, J = 3.2 Hz, 1H), 7.43 (s, |
| | | 1H), 6.98-6.89 (m, 1H), 6.76 (d, J = 8.4 Hz, |
| | | 1H), 6.59 (dd, J = 8.5, 2.6 Hz, 1H), 5.70 (d, J = |
| | | 2.5 Hz, 1H), 4.32 (q, J = 6.9 Hz, 2H), 3.30 |
| | | (s, 3H), 3.21 (t, J = 8.4 Hz, 1H), 2.34 (dd, J = |
| | | 7.9, 4.7 Hz, 1H), 2.00 (dd, J = 8.9, 4.6 Hz, |
| | | 1H), 1.49 (t, J = 6.9 Hz, 3H) |
| 35 | (1R,2S)-2-(3-{[3-ethoxy-6-(1,3-thiazol-2- | m/z (ESI, +ve ion) = 526.20 [M + H]⁺. ¹H- |
| | yl)pyrazin-2-yl]amino}-1H-indazol-6-yl)- | NMR (300 MHz, DMSO-d₆) δ 12.66 (s, 1H), |
| | 5′-methoxyspiro[cyclopropane-1,3′-indol]- | 10.41 (s, 1H), 9.26 (s, 1H), 8.20 (s, 1H), 7.81 |
| | 2′(1′H)-one | (d, J = 3.2 Hz, 1H), 7.51-7.41 (m, 3H), 6.84 |
| | | (d, J = 8.5 Hz, 1H), 6.78 (d, J = 8.4 Hz, 1H), |
| | | 6.64 (dd, J = 8.4, 2.6 Hz, 1H), 5.72 (d, J = 2.7 |
| | | Hz, 1H), 4.51 (q, J = 7.0 Hz, 2H), 3.30 (s, |
| | | 3H), 3.21 (t, J = 8.4 Hz, 1H), 2.33 (dd, J = 7.9, |
| | | 4.6 Hz, 1H), 1.99 (dd, J = 8.9, 4.6 Hz, 1H), |
| | | 1.45 (t, J = 7.0 Hz, 3H) |
| 36 | (1R,2S)-5′-methoxy-2-{3-[2-methoxy-5- | m/z (ESI, +ve ion) = 537.20 [M + H]⁺. ¹H |
| | (3-methoxy-1-methyl-1H-pyrazol-4- | NMR (300 MHz, DMSO-d₆) δ 12.05 (s, 1H), |
| | yl)anilino]-1H-indazol-6- | 10.40 (s, 1H), 8.24 (d, J = 2.1 Hz, 1H), 7.80 |
| | yl}spiro[cyclopropane-1,3′-indol]-2′(1′H)- | (d, J = 8.3 Hz, 1H), 7.70 (s, 1H), 7.56 (s, 1H), |
| | one | 7.31 (s, 1H), 7.05 (dd, J = 8.3, 2.0 Hz, 1H), |
| | | 6.95 (d, J = 8.4 Hz, 1H), 6.88 (d, J = 8.3 Hz, |
| | | 1H), 6.76 (d, J = 8.4 Hz, 1H), 6.59 (dd, J = |
| | | 8.4, 2.6 Hz, 1H), 5.71 (d, J = 2.5 Hz, 1H), |
| | | 3.88 (s, 3H), 3.81 (s, 3H), 3.70 (s, 3H), 3.33 |
| | | (s, 3H), 3.17 (d, J = 8.4 Hz, 1H), 2.33-2.28 |
| | | (m, 1H), 2.01-1.96 (m, 1H) |
| 37 | (1R,2S)-2-{3-[2-ethoxy-5-(1-methyl-1H- | m/z (ESI, +ve ion) = 521.25 [M + H]⁺. ¹H |
| | pyrazol-4-yl)anilino]-1H-indazol-6-yl}-5′- | NMR (300 MHz, DMSO-d₆) δ 12.11 (s, 1H), |
| | methoxyspiro[cyclopropane-1,3′-indol]- | 10.40 (s, 1H), 8.19 (d, J = 1.4 Hz, 1H), 7.87 |
| | 2′(1′H)-one | (s, 1H), 7.73 (d, J = 8.3 Hz, 1H), 7.62 (d, J = |
| | | 0.8 Hz, 1H), 7.52 (s, 1H), 7.34 (s, 1H), 6.97 |
| | | (d, J = 1.2 Hz, 2H), 6.89 (d, J = 8.3 Hz, 1H), |
| | | 6.76 (d, J = 8.5 Hz, 1H), 6.59 (dd, J = 8.4, 2.6 |
| | | Hz, 1H), 5.70 (d, J = 2.5 Hz, 1H), 4.15 (q, J = |
| | | 6.9 Hz, 2H), 3.85 (s, 3H), 3.32 (s, 3H), 3.19 (t, |
| | | J = 8.4 Hz, 1H), 2.31 (dd, J = 7.9, 4.7 Hz, |
| | | 1H), 1.99 (dd, J = 9.0, 4.7 Hz, 1H), 1.43 (t, J = |
| | | 6.9 Hz, 3H) |
| 38 | (1R,2S)-2-(3-{[3-ethoxy-5-(1H-1,2,4- | m/z (ESI, +ve ion) = 509.25 [M + H]+. 1H |
| | triazol-1-yl)pyridin-2-yl]amino}-1H- | NMR (300 MHz, DMSO-d6) δ 12.51 (s, 1H), |
| | indazol-6-yl)-5′- | 10.40 (s, 1H), 9.13 (s, 1H), 8.47 (s, 1H), 8.20 |
| | methoxyspiro[cyclopropane-1,3′-indol]- | (s, 1H), 7.99 (d, J = 2.2 Hz, 1H), 7.62 (d, J = |
| | 2′(1′H)-one | 2.2 Hz, 1H), 7.46 (d, J = 8.4 Hz, 1H), 7.39 (s, |
| | | 1H), 6.87 (d, J = 8.4 Hz, 1H), 6.75 (d, J = 8.4 |
| | | Hz, 1H), 6.59 (dd, J = 8.4, 2.6 Hz, 1H), 5.73 |
| | | (d, J = 2.6 Hz, 1H), 4.26 (q, J = 7.0 Hz, 2H), |
| | | 3.35 (s, 3H), 3.19 (t, J = 8.5 Hz, 1H), 2.32 (dd, |
| | | J = 7.9, 4.8 Hz, 1H), 1.99 (dd, J = 9.0, 4.6 Hz, |
| | | 1H), 1.46 (t, J = 6.9 Hz, 3H) |
| 39 | (1R,2S)-2-(3-{5-[1-(2,2-difluoroethyl)- | m/z (ESI, +ve ion) = 557.20 [M + H]⁺. ¹H |
| | 1H-pyrazol-4-yl]-2-methoxyanilino}-1H- | NMR (400 MHz, DMSO-d₆) δ 12.09 (s, 1H), |
| | indazol-6-yl)-5′- | 10.41 (s, 1H), 8.27 (d, J = 2.0 Hz, 1H), 7.99 |
| | methoxyspiro[cyclopropane-1,3′-indol]- | (d, J = 0.9 Hz, 1H), 7.83 (d, J = 8.3 Hz, 1H), |
| | 2′(1′H)-one | 7.77 (d, J = 0.8 Hz, 1H), 7.69 (s, 1H), 7.32 (s, |
| | | 1H), 7.07-6.96 (m, 2H), 6.89 (dd, J = 8.5, 1.4 |
| | | Hz, 1H), 6.75 (d, J = 8.4 Hz, 1H), 6.59 (dd, J = |
| | | 8.5, 2.6 Hz, 1H), 6.39 (tt, J = 55.0, 3.8 Hz, |
| | | 1H), 5.70 (d, J = 2.6 Hz, 1H), 4.64-4.60 (m, |
| | | 2H), 3.91 (s, 3H), 3.33 (s, 3H), 3.19 (t, J = 8.4 |
| | | Hz, 1H), 2.32 (dd, J = 7.9, 4.7 Hz, 1H), 1.99 |
| | | (dd, J = 9.0, 4.7 Hz, 1H) |
| 40 | (1R,2S)-5′-methoxy-2-(3-{[5-methoxy-2- | m/z (ESI, +ve ion) = 496.30 [M + H]⁺. ¹H |
| | (1,3-oxazol-5-yl)pyrimidin-4-yl]amino}- | NMR (400 MHz, DMSO-d₆) δ 12.70 (s, 1H), |
| | 1H-indazol-6-yl)spiro[cyclopropane-1,3′- | 10.41 (s, 1H), 9.38 (s, 1H), 8.32 (s, 1H), 8.13 |
| | indol]-2′(1′H)-one | (s, 1H), 7.54 (d, J = 8.4 Hz, 1H), 7.42 (s, 1H), |
| | | 7.15 (s, 1H), 6.90 (d, J = 8.4 Hz, 1H), 6.75 (d, |
| | | J = 8.4 Hz, 1H), 6.61-6.65 (m, 1H), 5.68 (d, J = |
| | | 2.4 Hz, 1H), 3.99 (s, 3H), 3.31 (s, 3H), 3.23- |
| | | 3.19 (m, 1H), 2.33 (d, J = 4.4 Hz, 1H), 2.01- |
| | | 1.98 (m, 1H) |
| 41 | (1R,2S)-2-(3-{[2-ethoxy-5-(1,3-thiazol-2- | m/z (ESI, +ve ion) = 525.85 [M + H]⁺. ¹H |
| | yl)pyridin-3-yl]amino}-1H-indazol-6-yl)- | NMR (400 MHz, DMSO-d₆) δ 8.72 (d, J = 2.2 |
| | 5′-methoxyspiro[cyclopropane-1,3′-indol]- | Hz, 1H), 8.18 (d, J = 2.2 Hz, 1H), 7.81 (d, J = |
| | 2′(1′H)-one | 3.3 Hz, 1H), 7.68 (d, J = 8.7 Hz, 1H), 7.54 (d, |
| | | J = 3.3 Hz, 1H), 7.38 (d, J = 1.1 Hz, 1H), 6.91 |
| | | (dd, J = 8.5, 1.3 Hz, 1H), 6.82 (d, J = 8.5 Hz, |
| | | 1H), 6.60 (dd, J = 8.5, 2.5 Hz, 1H), 5.61 (d, J = |
| | | 2.5 Hz, 1H), 4.58 (q, J = 7.1 Hz, 2H), 3.36 |
| | | (d, J = 8.5 Hz, 1H), 3.27 (s, 3H), 2.24-2.20 |
| | | (m, 2H), 1.51 (t, J = 7.1 Hz, 3H) |
| 42 | (1R,2S)-5′-methoxy-2-(3-{[2-methoxy-5- | m/z (ESI, +ve ion) = 495.15 [M + H]⁺. ¹H |
| | (1,3-oxazol-4-yl)pyridin-3-yl]amino}-1H- | NMR (300 MHz, DMSO-d₆) δ 12.25 (s, 1H), |
| | indazol-6-yl)spiro[cyclopropane-1,3′- | 10.42 (s, 1H), 8.80 (d, J = 2.1 Hz, 1H), 8.48 |
| | indol]-2′(1′H)-one | (dd, J = 12.3, 1.0 Hz, 2H), 8.13 (s, 1H), 8.05 |
| | | (d, J = 2.1 Hz, 1H), 7.93 (d, J = 8.4 Hz, 1H), |
| | | 7.34 (s, 1H), 6.94-6.85 (m, 1H), 6.75 (d, J = |
| | | 8.4 Hz, 1H), 6.58 (dd, J = 8.4, 2.6 Hz, 1H), |
| | | 5.71 (d, J = 2.5 Hz, 1H), 4.03 (s, 3H), 3.32 (s, |
| | | 3H), 3.18 (t, J = 8.4 Hz, 1H), 2.33 (dd, J = 7.9, |
| | | 4.8 Hz, 1H), 1.98 (dd, J = 9.1, 4.6 Hz, 1H) |
| 43 | (1R,2S)-2-{3-[(5-ethoxy-2- | m/z (ESI, +ve ion) = 460.15 [M + H]⁺. ¹H |
| | methylpyrimidin-4-yl)amino]-1H-indazol- | NMR (300 MHz, Methanol-d₄) δ 7.82 (s, 1H), |
| | 6-yl}-5′- | 7.59 (d, J = 8.4 Hz, 1H), 7.42 (s, 1H), 6.92 (d, |
| | [(2H3)methyloxy]spiro[cyclopropane-1,3′- | J = 8.5 Hz, 1H), 6.82 (d, J = 8.5 Hz, 1H), 6.60 |
| | indol]-2′(1′H)-one | (dd, J = 8.4, 2.6 Hz, 1H), 5.61 (d, J = 2.5 Hz, |
| | | 1H), 4.22 (q, J = 7.0 Hz, 2H), 3.36 (d, J = 8.4 |
| | | Hz, 1H), 2.29 (s, 3H), 2.24-2.20 (m, 2H), |
| | | 1.50 (t, J = 7.0 Hz, 3H) |
| 45 | (1R,2S)-2-(3-((5-ethoxy-2-(3-hydroxy-3- | m/z (ESI, +ve ion) = 525.10 [M + H]⁺. ¹H |
| | methylbut-1-yn-1-yl)pyrimidin-4- | NMR (400 MHz, Methanol-d₄) δ 7.89 (s, 1H), |
| | yl)amino)-1H-indazol-6-yl)-5′- | 7.66 (d, J = 8.5 Hz, 1H), 7.41 (s, 1H), 6.96 |
| | methoxyspiro[cyclopropane-1,3′-indolin]- | (dd, J = 8.5, 1.4 Hz, 1H), 6.81 (d, J = 8.5 Hz, |
| | 2′-one | 1H), 6.60 (dd, J = 8.5, 2.5 Hz, 1H), 5.60 (d, J = |
| | | 2.5 Hz, 1H), 4.27 (q, J = 7.0 Hz, 2H), 3.36 |
| | | (d, J = 8.5 Hz, 1H), 3.28 (s, 3H), 2.23 (dd, J = |
| | | 8.0, 4.8 Hz, 1H), 2.17 (dd, J = 9.1, 4.8 Hz, |
| | | 1H), 1.51 (t, J = 6.9 Hz, 3H), 1.45 (d, J = 3.2 |
| | | Hz, 6H) |
| 46 | (1R,2S)-2-(3-((5-(1-(2,2-difluoroethyl)- | m/z (ESI, +ve ion) = 558.15 [M + H]⁺. ¹H |
| | 1H-pyrazol-4-yl)-2-methoxypyridin-3- | NMR (400 MHz, DMSO-d₆) δ 12.20 (s, 1H), |
| | yl)amino)-1H-indazol-6-yl)-5′- | 10.43 (s, 1H), 8.56 (d, J = 2.1 Hz, 1H), 8.09 |
| | methoxyspiro[cyclopropane-1,3′-indolin]- | (d, J = 2.7 Hz, 2H), 7.94 (d, J = 8.4 Hz, 1H), |
| | 2′-one | 7.86 (dd, J = 6.4, 1.4 Hz, 2H), 7.33 (s, 1H), |
| | | 6.94-6.87 (m, 1H), 6.75 (d, J = 8.4 Hz, 1H), |
| | | 6.59 (dd, J = 8.5, 2.6 Hz, 1H), 6.57-6.20 (m, |
| | | 1H), 5.71 (d, J = 2.6 Hz, 1H), 4.67 (td, J = |
| | | 15.1, 3.8 Hz, 2H), 4.01 (s, 3H), 3.30 (s, 3H), |
| | | 3.19 (t, J = 8.5 Hz, 1H), 2.33 (t, J = 6.3 Hz, |
| | | 1H), 1.98 (dd, J = 9.0, 4.6 Hz, 1H) |

### BIOLOGICAL ACTIVITY EXAMPLES

### Biological Activity Example No. 1: PLK4 Biochemical Assay

Activity of human recombinant PLK4 (ThermoFisher, cat #PV6396) was measured by quantification of adenosine diphosphate (ADP) using the ADP-Glo Kinase Assay Kit (Promega, cat #V9102). Test compounds were solubilized in dimethyl sulfoxide (DMSO) and dispensed into 384-well white polystyrene nonbinding plates (Greiner, cat #781094) using the Echo acoustic dispenser (Labcyte Inc.) in a 11-point 3-fold titration in duplicates. 5 μL of 1.0 nM PLK4 protein in assay buffer (50 mM HEPES, pH 7.5, 0.01% Brij-35, 0.01% BSA, 10 mM MgCl₂, 1 mM EGTA, 1 mM DTT) was added to the plates. Test compounds and PLK4 were incubated for 15 minutes at room temperature (RT). Then 5 μL of a 16 μM adenosine triphosphate (ATP) (Promega, cat #V915B) and 9.3 μM Myelin Basic Protein (MBP) (SignalChem, cat #M42-51N) substrate solution in assay buffer was added and the reaction mixture was incubated for 6 hours at RT. The final concentration of PLK4, ATP and MBP in the reactions were 0.5 nM, 8.0 μM and 4.7 μM, respectively. Reactions were stopped and the remaining ATP depleted by adding 10 μL of ADP-Glo reagent (Promega, cat #V912B) and incubating for 40 minutes at RT. The simultaneous conversion of the remaining ADP to ATP and measurement of the newly synthesized ATP was achieved by addition of 20 μL Kinase Detection reagent (Promega, cat #V914B), incubation for 30 min at RT, and luminescence detection using the EnVision plate reader (PerkinElmer). Reactions lacking PLK4 were used as 100% inhibition controls. Reactions containing DMSO alone were used as 0% inhibition controls. The IC₅₀ values reported in Table 2 were determined using four parameter non-linear regression curve fit.

### Biological Activity Example No. 2: CHP134 CellTiter-Glo (CTG) Assay

CIP-134 cells (DSMZ-German Collection of Microorganisms and Cell Cultures, Braunschweig Germany) were cultured in RPMI 1640 supplemented with 10% fetal bovine serum, penicillin (100 U/ml), 1% L-Glutamine and streptomycin (100 mg/ml). Cells were seeded (200 cells/well) in 384-well plates for 16 hours. On day two, nine serial 1:3 compound dilutions were made in DMSO in a 96-well plate. The compounds were then further diluted into growth media using a BRAVO robot (Agilent, Santa Clara, CA). The diluted compounds were then added to quadruplicate wells in the 384-well cell plate and incubated at 37° C. and 5% CO2. After 5 days, relative numbers of viable cells were measured by luminescence using CellTiter-Glo® (Promega) according to the manufacturer's instructions and read on a SPARK Multimode Microplate Reader (Tecan, Mannedorf Switzerland). The IC₅₀ calculations for the values reported in Table 2 were carried out using Prism 6.0 software (GraphPad, San Diego).

### Biological Activity Example No. 3: Aurora a Kinase Biochemical Assay

Activity of human recombinant Aurora A (ThermoFisher, cat #PR5935A) was measured by quantification of adenosine diphosphate (ADP) using the ADP-Glo Kinase Assay Kit (Promega, cat #V9102). Test compounds were solubilized in dimethyl sulfoxide (DMSO) and dispensed into 384-well white polystyrene nonbinding plates (Greiner, cat #781094) using the Echo acoustic dispenser (Labcyte Inc.) in a 11-point 3-fold titration in duplicates. 5 μL of 5.0 nM Aurora A in assay buffer (50 mM HEPES, pH 7.5, 0.01% Brij-35, 0.01% BSA, 10 mM MgCl₂, 1 mM EGTA, 1 mM DTT) was added to the plates. Test compounds and Aurora A were incubated for 15 minutes at room temperature (RT). Then 5 μL of a 40 μM adenosine triphosphate (ATP) (Promega, cat #V915B) and 9.3 μM Myelin Basic Protein (MBP) (SignalChem, cat #M42-51N) substrate solution in assay buffer was added and the reaction mixture was incubated for 2 hours at RT. The final concentration of Aurora A, ATP and MBP in the reactions were 2.5 nM, 20 μM and 4.7 μM, respectively. Reactions were stopped and the remaining ATP depleted by adding 10 μL of ADP-Glo reagent (Promega, cat #V912B) and incubating for 40 minutes at RT. The simultaneous conversion of the remaining ADP to ATP and measurement of the newly synthesized ATP was achieved by addition of 20 μL Kinase Detection reagent (Promega, cat #V914B), incubation for 30 min at RT, and luminescence detection using the EnVision plate reader (PerkinElmer). Reactions lacking Aurora A were used as 100% inhibition controls. Reactions containing DMSO alone were used as 0% inhibition controls. The IC₅₀ values reported in Table 2 were determined using four parameter non-linear regression curve fit.

### Biological Activity Example No. 4: Aurora B Kinase Biochemical Assay

Activity of human recombinant Aurora B (ThermoFisher, cat #PR9210B) was measured by quantification of adenosine diphosphate (ADP) using the ADP-Glo Kinase Assay Kit (Promega, cat #V9102). Test compounds were solubilized in dimethyl sulfoxide (DMSO) and dispensed into 384-well white polystyrene nonbinding plates (Greiner, cat #781094) using the Echo acoustic dispenser (Labcyte Inc.) in a 11-point 3-fold titration in duplicates. 5 μL of 20 nM Aurora B in assay buffer (50 mM HEPES, pH 7.5, 0.01% Brij-35, 0.01% BSA, 10 mM MgCl₂, 1 mM EGTA, 1 mM DTT) was added to the plates. Test compounds and Aurora B were incubated for 15 minutes at room temperature (RT). Then 5 μL of a 228 μM adenosine triphosphate (ATP) (Promega, cat #V915B) and 9.3 μM Myelin Basic Protein (MBP) (SignalChem, cat #M42-51N) substrate solution in assay buffer was added and the reaction mixture was incubated for 2 hours at RT. The final concentration of Aurora b, ATP and MBP in the reactions were 10 nM, 114 μM and 4.7 μM, respectively. Reactions were stopped and the remaining ATP depleted by adding 10 μL of ADP-Glo reagent (Promega, cat #V912B) and incubating for 40 minutes at RT. The simultaneous conversion of the remaining ADP to ATP and measurement of the newly synthesized ATP was achieved by addition of 20 μL Kinase Detection reagent (Promega, cat #V9141B), incubation for 30 min at RT, and luminescence detection using the EnVision plate reader (PerkinElmer). Reactions lacking Aurora B were used as 10000 inhibition controls. Reactions containing DMSO alone were used as 000 inhibition controls. The IC₅₀ values reported in Table 2 were determined using four parameter non-linear regression curve fit.

As shown in Table 2, many of the compounds of Formula (I), (Ia), (Ib), (II), and (III) demonstrated potent inhibition of PLK4 and less potent inhibition of Aurora A kinase and Aurora B kinase. As such, the compounds of Formula (I), (Ia), (Ib), (II), and (III) demonstrated selective inhibition of PLK4. As also set forth in Table 2, many of the compounds of Formula (I), (Ia), (Ib), (II), and (III) demonstrated, surprisingly and unexpectedly, greater selectivity in the inhibition of PLK4 versus inhibition of Aurora A kinase and/or Aurora B kinase than the CFI-400495 compound. In Table 2, ND means not determined.

TABLE 2

| | | | [Aurora A | [Aurora B |
|---|---|---|---|---|
| | PLK4 | CTG | IC₅₀ (nM)]/ | IC₅₀ (nM)]/ |
| | IC₅₀ | IC₅₀ | [PLK4 | [PLK4 |
| Ex. No. | (nM) | (nM) | IC₅₀ (nM)] | IC₅₀ (nM)] |
| 1 | 0.7971 | 241.6 | 2164.09484 | 791.6196 |
| 2 | 0.5758 | 88.91 | 801.1462 | 229.2462 |
| 3 | 2.76 | 307.9 | 1671.3768 | 256.2681 |
| 4 | 0.682 | 53.09 | 538.2697 | 549.2668 |
| 5 | 0.7076 | 39.14 | 567.6936 | 238.2702 |
| 6 | 0.9522 | 39.41 | 581.0754 | 427.6412 |
| 7 | 1.705 | 166.4 | 10956.01173 | 702.6392 |
| 8 | 1.826 | 490.2 | 27382.2563 | 1829.1347 |
| 9 | 0.862 | 166.5 | 421.2296 | 293.3874 |
| 10 | 0.557 | 119.9 | 2540.3949 | 396.7684 |
| 11 | 0.52 | 110.3 | 897.6923 | 1051.5384 |
| 12 | 3.1 | 1100 | 630 | 810 |
| 13 | 0.65 | 348.1 | 414.76923 | 1120.7692 |
| 14 | 0.639 | 88.72 | 187.4804 | 897.1830 |
| 15 | 0.65 | 93.76 | 146.5230 | 511.8461 |
| 16 | 0.894 | 160 | 259.06040 | 730.6487 |
| 17 | 2 | 370 | 1000 | 190 |
| 18 | 0.71 | 170 | 500 | 520 |
| 19 | 0.99 | 170 | 360 | 460 |
| 20 | 2.8 | 440 | 170 | 120 |
| 21 | 2.6 | 400 | 1900 | 990 |
| 22 | ND | ND | ND | ND |
| 23 | 1.405 | 111.3 | 2258.3629 | 1017.08185 |
| 24 | 0.408 | 49.87 | 842.4019 | 263.2352 |
| 25 | 2.409 | | 934.8277 | 608.5512 |
| 26 | 0.8403 | 171.4 | 992.1456 | 921.6946 |
| 27 | 1.057 | 463 | 360.5487 | 954.5884 |
| 28 | 0.3407 | 16.66 | 1135.01614 | 1526.5629 |
| 29 | 1.955 | 465.5 | 15611.2532 | 211.2020 |
| 30 | 0.6943 | 97.37 | 916.8947 | 1970.3298 |
| 31 | 0.2592 | 135.1 | 743.0555 | 429.3981 |
| 32 | 0.827 | 106.1 | 325.6348 | 1218.8633 |
| 33 | 0.753 | 41.44 | 1496.6799 | 45644.0903 |
| 34 | 1.073 | 138.8 | 14827.5862 | 46598.3224 |
| 35 | 0.338 | 13.83 | 727.2189 | 2740.5325 |
| 36 | 2.288 | | 4350.9615 | 12202.7972 |
| 37 | 1.796 | 442 | 27839.6436 | 27839.6436 |
| 38 | 0.8166 | 265.9 | 2756.5515 | 559.14768 |
| 39 | 1.933 | 202.1 | 1647.6978 | 764.09725 |
| 40 | 0.2795 | 237.2 | 288.40787 | 410.7334 |
| 41 | 0.722 | 131.1 | 4711.9113 | 8707.7562 |
| 42 | 1.309 | 280.7 | 157.8304 | 465.6990 |
| 43 | 1.894 | 411.3 | 4818.3738 | 341.1826 |
| 44 | 0.16 | 22 | 702.5 | 844.375 |
| 45 | 0.139 | 890 | 42971.2230 | 8798.5611 |
| 46 | 1.249 | 370 | 469.9759 | 469.6557 |
| CFI-400495 | 0.4962 | 9.082 | 86.3160 | 12.0435 |

### EMBODIMENTS

Embodiment 1. A compound of Formula (IV), or a pharmaceutically acceptable salt, solvate, or stereoisomer thereof

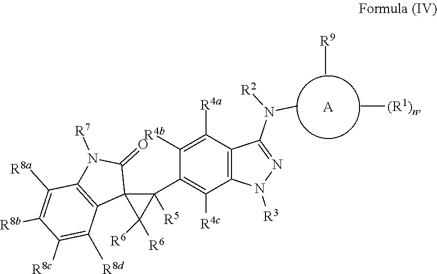

wherein:
Ring A is C₆-C₁₀aryl or heteroaryl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, —P(O)(R^{a})₂, —P(O)₂(R^{a})₂, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3, 4, 5, 6, 7, or 8;
R² is hydrogen, C₁-C₆alkyl, C₁-C₆haloalkyl, or C₁-C₆deuteroalkyl;
R³ is hydrogen, C₁-C₆alkyl, C₁-C₆haloalkyl, or C₁-C₆deuteroalkyl;
each of R^{4a}, R^{4b}, and R^{4c} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁵ is hydrogen, deuterium, halogen, —CN, —OH, —OR^{a}, —NR^{C}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R⁶ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁷ is hydrogen, C₁-C₆alkyl, C₁-C₆haloalkyl, C₁-C₆deuteroalkyl, C₁-C₆hydroxyalkyl, or C₁-C₆aminoalkyl;
each of R^{8a}, R^{8b}, R^{8c}, and R^{8d} is independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
R⁹ is heteroaryl optionally substituted with one or more R^{1a}, or oxetanyl substituted with one or more R^{1a};
each R^{a} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆alkoxy, C₁-C₆aminoalkyl, C₁-C₆alkylamino, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
provided the compound of Formula (IV) is not

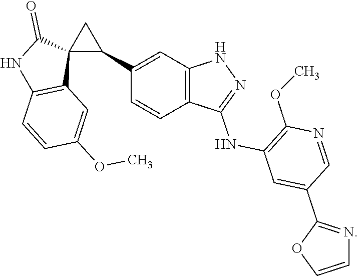 [SMILES: COc1ccc2c(c1)[C@]1(C[C@H]1c1ccc3c(Nc4cc(-c5ncco5)cnc4OC)n[nH]c3c1)C(=O)N2]

Embodiment 2. The compound according to embodiment 1, or a pharmaceutically acceptable salt, solvate, or stereoisomer thereof, wherein Ring A is C₆-C₁₀aryl.
Embodiment 3. The compound according to embodiment 2, or a pharmaceutically acceptable salt, solvate, or stereoisomer thereof, wherein Ring A is phenyl.
Embodiment 4. The compound according to embodiment 1, or a pharmaceutically acceptable salt, solvate, or stereoisomer thereof, wherein Ring A is heteroaryl.
Embodiment 5. The compound according to embodiment 4, or a pharmaceutically acceptable salt, solvate, or stereoisomer thereof, wherein Ring A is furanyl, pyrrolyl, thiophenyl, oxazolyl, imidazolyl, thiazolyl, pyrazolyl, isoxazolyl, isothiazolyl, triazolyl, pyridinyl, pyrazinyl, pyrimidinyl, or pyridazinyl.
Embodiment 6. The compound according to embodiment 5, or a pharmaceutically acceptable salt, solvate, or stereoisomer thereof, wherein Ring A is pyrazolyl, pyridinyl, pyrazinyl, pyrimidinyl, or pyridazinyl.
Embodiment 7. The compound according to embodiment 6, or a pharmaceutically acceptable salt, solvate, or stereoisomer thereof, wherein Ring A is pyrazolyl, pyridinyl, pyrazinyl, or pyrimidinyl.
Embodiment 8. The compound according to embodiment 7, or a pharmaceutically acceptable salt, solvate, or stereoisomer thereof, wherein Ring A is pyrazolyl.
Embodiment 9. The compound according to embodiment 8, or a pharmaceutically acceptable salt, solvate, or stereoisomer thereof, wherein Ring A is 1-pyrazolyl, 3-pyrazolyl, 4-pyrazolyl, or 5-pyrazolyl.
Embodiment 10. The compound according to embodiment 9, or a pharmaceutically acceptable salt, solvate, or stereoisomer thereof, wherein Ring A is 1-pyrazolyl.
Embodiment 11. The compound according to embodiment 9, or a pharmaceutically acceptable salt, solvate, or stereoisomer thereof, wherein Ring A is 3-pyrazolyl.
Embodiment 12. The compound according to embodiment 9, or a pharmaceutically acceptable salt, solvate, or stereoisomer thereof, wherein Ring A is 4-pyrazolyl.
Embodiment 13. The compound according to embodiment 9, or a pharmaceutically acceptable salt, solvate, or stereoisomer thereof, wherein Ring A is 5-pyrazolyl.
Embodiment 14. The compound according to embodiment 7, or a pharmaceutically acceptable salt, solvate, or stereoisomer thereof, wherein Ring A is pyridinyl.
Embodiment 15. The compound according to embodiment 14, or a pharmaceutically acceptable salt, solvate, or stereoisomer thereof, wherein Ring A is 2-pyridinyl, 3-pyridinyl, 4-pyridinyl, 5-pyridinyl, or 6-pyridinyl.
Embodiment 16. The compound according to embodiment 15, or a pharmaceutically acceptable salt, solvate, or stereoisomer thereof, wherein Ring A is 2-pyridinyl.
Embodiment 17. The compound according to embodiment 15, or a pharmaceutically acceptable salt, solvate, or stereoisomer thereof, wherein Ring A is 3-pyridinyl.
Embodiment 18. The compound according to embodiment 15, or a pharmaceutically acceptable salt, solvate, or stereoisomer thereof, wherein Ring A is 4-pyridinyl.
Embodiment 19. The compound according to embodiment 15, or a pharmaceutically acceptable salt, solvate, or stereoisomer thereof, wherein Ring A is 5-pyridinyl.
Embodiment 20. The compound according to embodiment 15, or a pharmaceutically acceptable salt, solvate, or stereoisomer thereof, wherein Ring A is 6-pyridinyl.
Embodiment 21. The compound according to embodiment 7, or a pharmaceutically acceptable salt, solvate, or stereoisomer thereof, wherein Ring A is pyrazinyl.
Embodiment 22. The compound according to embodiment 21, or a pharmaceutically acceptable salt, solvate, or stereoisomer thereof, wherein Ring A is 2-pyrazinyl, 3-pyrazinyl, 5-pyrazinyl, or 6-pyrazinyl.
Embodiment 23. The compound according to embodiment 22, or a pharmaceutically acceptable salt, solvate, or stereoisomer thereof, wherein Ring A is 2-pyrazinyl.
Embodiment 24. The compound according to embodiment 22, or a pharmaceutically acceptable salt, solvate, or stereoisomer thereof, wherein Ring A is 3-pyrazinyl.
Embodiment 25. The compound according to embodiment 22, or a pharmaceutically acceptable salt, solvate, or stereoisomer thereof, wherein Ring A is 5-pyrazinyl.
Embodiment 26. The compound according to embodiment 22, or a pharmaceutically acceptable salt, solvate, or stereoisomer thereof, wherein Ring A is 6-pyrazinyl.
Embodiment 27. The compound according to embodiment 7, or a pharmaceutically acceptable salt, solvate, or stereoisomer thereof, wherein Ring A is pyrimidinyl.
Embodiment 28. The compound according to embodiment 27, or a pharmaceutically acceptable salt, solvate, or stereoisomer thereof, wherein Ring A is 2-pyrimidinyl, 4-pyrimidinyl, 5-pyrimidinyl, or 6-pyrimidinyl.
Embodiment 29. The compound according to embodiment 28, or a pharmaceutically acceptable salt, solvate, or stereoisomer thereof, wherein Ring A is 2-pyrimidinyl.
Embodiment 30. The compound according to embodiment 28, or a pharmaceutically acceptable salt, solvate, or stereoisomer thereof, wherein Ring A is 4-pyrimidinyl.
Embodiment 31. The compound according to embodiment 28, or a pharmaceutically acceptable salt, solvate, or stereoisomer thereof, wherein Ring A is 5-pyrimidinyl.
Embodiment 32. The compound according to embodiment 28, or a pharmaceutically acceptable salt, solvate, or stereoisomer thereof, wherein Ring A is 6-pyrimidinyl.
Embodiment 33. The compound according to embodiment 6, or a pharmaceutically acceptable salt, solvate, or stereoisomer thereof, wherein Ring A is pyridazinyl.
Embodiment 34. The compound according to embodiment 33, or a pharmaceutically acceptable salt, solvate, or stereoisomer thereof, wherein Ring A is 3-pyridazinyl, 4-pyridazinyl, 5-pyridazinyl, or 6-pyridazinyl.
Embodiment 35. The compound according to embodiment 34, or a pharmaceutically acceptable salt, solvate, or stereoisomer thereof, wherein Ring A is 3-pyridazinyl.
Embodiment 36. The compound according to embodiment 34, or a pharmaceutically acceptable salt, solvate, or stereoisomer thereof, wherein Ring A is 4-pyridazinyl.
Embodiment 37. The compound according to embodiment 34, or a pharmaceutically acceptable salt, solvate, or stereoisomer thereof, wherein Ring A is 5-pyridazinyl.
Embodiment 38. The compound according to embodiment 34, or a pharmaceutically acceptable salt, solvate, or stereoisomer thereof, wherein Ring A is 6-pyridazinyl.
Embodiment 39. The compound according to any one of embodiments 1 to 38, or a pharmaceutically acceptable salt, solvate, or stereoisomer thereof, wherein each R1 is independently methyl, ethyl, trifluoromethyl, methoxy, ethoxy, methanesulfonyl, ethanesulfonyl, acetyl, or dimethylamino.
Embodiment 40. The compound according to any one of embodiments 1 to 39, or a pharmaceutically acceptable salt, solvate, or stereoisomer thereof, wherein n is 1, 2, or 3.
Embodiment 41. The compound according to embodiment 40, or a pharmaceutically acceptable salt, solvate, or stereoisomer thereof, wherein n is 1.
Embodiment 42. The compound according to embodiment 40, or a pharmaceutically acceptable salt, solvate, or stereoisomer thereof, wherein n is 2.
Embodiment 43. The compound according to embodiment 40, or a pharmaceutically acceptable salt, solvate, or stereoisomer thereof, wherein n is 3.
Embodiment 44. The compound according to any one of embodiments 1 to 43, or a pharmaceutically acceptable salt, solvate, or stereoisomer thereof, wherein R² is hydrogen.
Embodiment 45. The compound according to any one of embodiments 1 to 44, or a pharmaceutically acceptable salt, solvate, or stereoisomer thereof, wherein R³ is hydrogen.
Embodiment 46. The compound according to any one of embodiments 1 to 45, or a pharmaceutically acceptable salt, solvate, or stereoisomer thereof, wherein R^{4a}, R^{4b}, and R^{4c} are independently hydrogen or halogen.
Embodiment 47. The compound according to embodiment 46, or a pharmaceutically acceptable salt, solvate, or stereoisomer thereof, wherein R^{4a} is halogen and R^{4b} and R^{4c} are hydrogen.
Embodiment 48. The compound according to embodiment 46, or a pharmaceutically acceptable salt, solvate, or stereoisomer thereof, wherein R^{4a} and R^{4c} are hydrogen and R^{4b} is halogen.
Embodiment 49. The compound according to embodiment 46, or a pharmaceutically acceptable salt, solvate, or stereoisomer thereof, wherein R^{4a} and R^{4b} are hydrogen and R^{4c} is halogen.
Embodiment 50. The compound according to embodiment 46, or a pharmaceutically acceptable salt, solvate, or stereoisomer thereof, wherein R^{4a} and R^{4b} are halogen and R^{4c} is hydrogen.
Embodiment 51. The compound according to embodiment 46, or a pharmaceutically acceptable salt, solvate, or stereoisomer thereof, wherein R^{4a} and R^{4c} are halogen and R^{4b} is hydrogen.
Embodiment 52. The compound according to embodiment 46, or a pharmaceutically acceptable salt, solvate, or stereoisomer thereof, wherein R^{4a}, R^{4b} and R^{4c} are halogen.
Embodiment 53. The compound according to embodiment 46, or a pharmaceutically acceptable salt, solvate, or stereoisomer thereof, wherein R^{4a}, R^{4b}, and R^{4c} are hydrogen.
Embodiment 54. The compound according to any one of embodiments 1 to 53, or a pharmaceutically acceptable salt, solvate, or stereoisomer thereof, wherein R⁵ is hydrogen.
Embodiment 55. The compound according to any one of embodiments 1 to 54, or a pharmaceutically acceptable salt, solvate, or stereoisomer thereof, wherein each R⁶ is hydrogen.
Embodiment 56. The compound according to any one of embodiments 1 to 55, or a pharmaceutically acceptable salt, solvate, or stereoisomer thereof, wherein R⁷ is hydrogen or C₁-C₆alkyl.
Embodiment 57. The compound according to embodiment 56, or a pharmaceutically acceptable salt, solvate, or stereoisomer thereof, wherein R⁷ is hydrogen.
Embodiment 58. The compound according to embodiment 56, or a pharmaceutically acceptable salt, solvate, or stereoisomer thereof, wherein R⁷ is C₁-C₆alkyl.
Embodiment 59. The compound according to any one of embodiments 1 to 58, or a pharmaceutically acceptable salt, solvate, or stereoisomer thereof, wherein each of R^{8a}, R^{8b}, R^{8c}, and R^{8d} is independently hydrogen, halogen, or —OR^{a}.
Embodiment 60. The compound according to embodiment 59, or a pharmaceutically acceptable salt, solvate, or stereoisomer thereof, wherein each of R^{8a}, R^{8b}, and R^{8d} are hydrogen and R^{8c} is hydrogen, halogen, or —OR^{a}.
Embodiment 61. The compound according to embodiment 60, or a pharmaceutically acceptable salt, solvate, or stereoisomer thereof, wherein R^{8c} is halogen or —OR^{a}.
Embodiment 62. The compound according to embodiment 61, or a pharmaceutically acceptable salt, solvate, or stereoisomer thereof, wherein R^{8c} is halogen.
Embodiment 63. The compound according to embodiment 62, or a pharmaceutically acceptable salt, solvate, or stereoisomer thereof, wherein RIC is fluoro, chloro, bromo, or iodo.
Embodiment 64. The compound according to embodiment 61, or a pharmaceutically acceptable salt, solvate, or stereoisomer thereof, wherein RIC is —OR^{a}.
Embodiment 65. The compound according to embodiment 64, or a pharmaceutically acceptable salt, solvate, or stereoisomer thereof, wherein R^{a} is C₁-C₆alkyl.
Embodiment 66. The compound according to embodiment 65, or a pharmaceutically acceptable salt, solvate, or stereoisomer thereof, wherein R^{a} is —CH₃.
Embodiment 67. The compound according to any one of embodiments 1 to 66, or a pharmaceutically acceptable salt, solvate, or stereoisomer thereof, wherein R⁹ is furanyl, pyrrolyl, thiophenyl, oxazolyl, imidazolyl, thiazolyl, pyrazolyl, isoxazolyl, isothiazolyl, triazolyl, pyridinyl, pyrazinyl, pyrimidinyl, pyridazinyl, or tetrazolyl, each of which is optionally substituted with one or more R^{1a}.
Embodiment 68. The compound according to embodiment 67, or a pharmaceutically acceptable salt, solvate, or stereoisomer thereof, wherein R⁹ is furanyl optionally substituted with one or more R^{1a}.
Embodiment 69. The compound according to embodiment 67, or a pharmaceutically acceptable salt, solvate, or stereoisomer thereof, wherein R⁹ is pyrrolyl optionally substituted with one or more R^{1a}.
Embodiment 70. The compound according to embodiment 67, or a pharmaceutically acceptable salt, solvate, or stereoisomer thereof, wherein R⁹ is thiophenyl optionally substituted with one or more R^{1a}.
Embodiment 71. The compound according to embodiment 67, or a pharmaceutically acceptable salt, solvate, or stereoisomer thereof, wherein R⁹ is oxazolyl optionally substituted with one or more R^{1a}.
Embodiment 72. The compound according to embodiment 67, or a pharmaceutically acceptable salt, solvate, or stereoisomer thereof, wherein R⁹ is imidazolyl optionally substituted with one or more R^{1a}.
Embodiment 73. The compound according to embodiment 67, or a pharmaceutically acceptable salt, solvate, or stereoisomer thereof, wherein R⁹ is thiazolyl optionally substituted with one or more R^{1a}.
Embodiment 74. The compound according to embodiment 67, or a pharmaceutically acceptable salt, solvate, or stereoisomer thereof, wherein R⁹ is pyrazolyl optionally substituted with one or more R^{1a}.
Embodiment 75. The compound according to embodiment 67, or a pharmaceutically acceptable salt, solvate, or stereoisomer thereof, wherein R⁹ is isoxazolyl optionally substituted with one or more R^{1a}.
Embodiment 76. The compound according to embodiment 67, or a pharmaceutically acceptable salt, solvate, or stereoisomer thereof, wherein R⁹ is isothiazolyl optionally substituted with one or more R^{1a}.
Embodiment 77. The compound according to embodiment 67, or a pharmaceutically acceptable salt, solvate, or stereoisomer thereof, wherein R⁹ is triazolyl optionally substituted with one or more R^{1a}.
Embodiment 78. The compound according to embodiment 67, or a pharmaceutically acceptable salt, solvate, or stereoisomer thereof, wherein R⁹ is pyridinyl optionally substituted with one or more R^{1a}.
Embodiment 79. The compound according to embodiment 67, or a pharmaceutically acceptable salt, solvate, or stereoisomer thereof, wherein R⁹ is pyrazinyl optionally substituted with one or more R^{1a}.
Embodiment 80. The compound according to embodiment 67, or a pharmaceutically acceptable salt, solvate, or stereoisomer thereof, wherein R⁹ is pyrimidinyl optionally substituted with one or more R^{1a}.
Embodiment 81. The compound according to embodiment 67, or a pharmaceutically acceptable salt, solvate, or stereoisomer thereof, wherein R⁹ is pyridazinyl optionally substituted with one or more R^{1a}.
Embodiment 82. The compound according to embodiment 67, or a pharmaceutically acceptable salt, solvate, or stereoisomer thereof, wherein R⁹ is tetrazolyl optionally substituted with one or more R^{1a}.
Embodiment 83. The compound according to any one of embodiments 1 to 66, or a pharmaceutically acceptable salt, solvate, or stereoisomer thereof, wherein R⁹ is oxetanyl substituted with one or more R^{1a}.
Embodiment 84. A compound according to any one of embodiments 1 to 83, or a pharmaceutically acceptable salt, solvate, or stereoisomer thereof, wherein the compound of Formula (IV) has the structure of Formula (IVa):

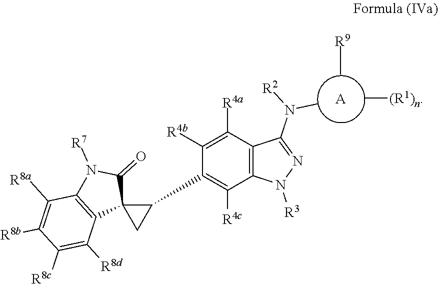

Embodiment 85. A compound according to any one of embodiments 1 to 83, or a pharmaceutically acceptable salt, solvate, or stereoisomer thereof, wherein the compound of Formula (IV) has the structure of Formula (IVb):

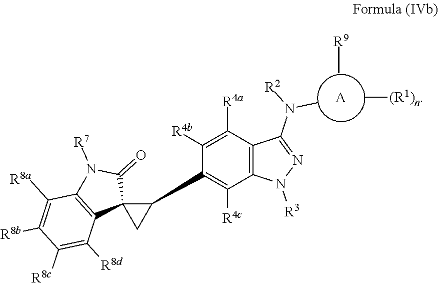

Embodiment 86. A compound of Formula (V), or a pharmaceutically acceptable salt, solvate, or stereoisomer thereof

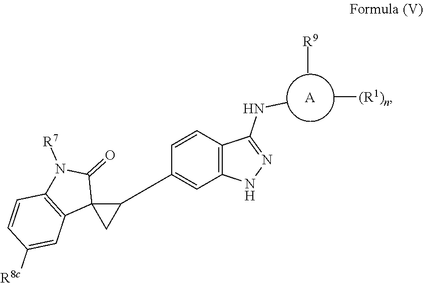

wherein:
Ring A is C₆-C₁₀aryl or heteroaryl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, —P(O)(R^{a})₂, —P(O)₂(R^{a})₂, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3, 4, 5, 6, 7, or 8;
R⁷ is hydrogen, C₁-C₆alkyl, C₁-C₆haloalkyl, C₁-C₆deuteroalkyl, C₁-C₆hydroxyalkyl, or C₁-C₆aminoalkyl;
R^{8c} is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
R⁹ is heteroaryl optionally substituted with one or more R^{1a}, or oxetanyl substituted with one or more R^{a};
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆alkoxy, C₁-C₆aminoalkyl, C₁-C₆alkylamino, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
provided the compound of Formula (V) is not

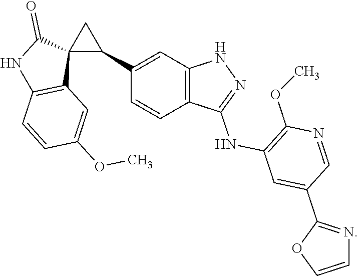 [SMILES: COc1ccc2c(c1)[C@]1(C[C@H]1c1ccc3c(Nc4cc(-c5ncco5)cnc4OC)n[nH]c3c1)C(=O)N2]

Embodiment 87. The compound according to embodiment 86, or a pharmaceutically acceptable salt, solvate, or stereoisomer thereof, wherein Ring A is C₆-C₁₀aryl.
Embodiment 88. The compound according to embodiment 87, or a pharmaceutically acceptable salt, solvate, or stereoisomer thereof, wherein Ring A is phenyl.
Embodiment 89. The compound according to embodiment 86, or a pharmaceutically acceptable salt, solvate, or stereoisomer thereof, wherein Ring A is heteroaryl.
Embodiment 90. The compound according to embodiment 89, or a pharmaceutically acceptable salt, solvate, or stereoisomer thereof, wherein Ring A is pyrazolyl, pyridinyl, pyrazinyl, pyrimidinyl, or pyridazinyl.
Embodiment 91. The compound according to embodiment 90, or a pharmaceutically acceptable salt, solvate, or stereoisomer thereof, wherein Ring A is pyrazolyl.
Embodiment 92. The compound according to embodiment 91, or a pharmaceutically acceptable salt, solvate, or stereoisomer thereof, wherein Ring A is 1-pyrazolyl, 3-pyrazolyl, 4-pyrazolyl, or 5-pyrazolyl.
Embodiment 93. The compound according to embodiment 92, or a pharmaceutically acceptable salt, solvate, or stereoisomer thereof, wherein Ring A is 1-pyrazolyl.
Embodiment 94. The compound according to embodiment 92, or a pharmaceutically acceptable salt, solvate, or stereoisomer thereof, wherein Ring A is 3-pyrazolyl.
Embodiment 95. The compound according to embodiment 92, or a pharmaceutically acceptable salt, solvate, or stereoisomer thereof, wherein Ring A is 4-pyrazolyl.
Embodiment 96. The compound according to embodiment 92, or a pharmaceutically acceptable salt, solvate, or stereoisomer thereof, wherein Ring A is 5-pyrazolyl.
Embodiment 97. The compound according to embodiment 90, or a pharmaceutically acceptable salt, solvate, or stereoisomer thereof, wherein Ring A is pyridinyl.
Embodiment 98. The compound according to embodiment 97, or a pharmaceutically acceptable salt, solvate, or stereoisomer thereof, wherein Ring A is 2-pyridinyl, 3-pyridinyl, 4-pyridinyl, 5-pyridinyl, or 6-pyridinyl.
Embodiment 99. The compound according to embodiment 98, or a pharmaceutically acceptable salt, solvate, or stereoisomer thereof, wherein Ring A is 2-pyridinyl.
Embodiment 100. The compound according to embodiment 98, or a pharmaceutically acceptable salt, solvate, or stereoisomer thereof, wherein Ring A is 3-pyridinyl.
Embodiment 101. The compound according to embodiment 98, or a pharmaceutically acceptable salt, solvate, or stereoisomer thereof, wherein Ring A is 4-pyridinyl.
Embodiment 102. The compound according to embodiment 98, or a pharmaceutically acceptable salt, solvate, or stereoisomer thereof, wherein Ring A is 5-pyridinyl.
Embodiment 103. The compound according to embodiment 98, or a pharmaceutically acceptable salt, solvate, or stereoisomer thereof, wherein Ring A is 6-pyridinyl.
Embodiment 104. The compound according to embodiment 90, or a pharmaceutically acceptable salt, solvate, or stereoisomer thereof, wherein Ring A is pyrazinyl.
Embodiment 105. The compound according to embodiment 104, or a pharmaceutically acceptable salt, solvate, or stereoisomer thereof, wherein Ring A is 2-pyrazinyl, 3-pyrazinyl, 5-pyrazinyl, or 6-pyrazinyl.
Embodiment 106. The compound according to embodiment 105, or a pharmaceutically acceptable salt, solvate, or stereoisomer thereof, wherein Ring A is 2-pyrazinyl.
Embodiment 107. The compound according to embodiment 105, or a pharmaceutically acceptable salt, solvate, or stereoisomer thereof, wherein Ring A is 3-pyrazinyl.
Embodiment 108. The compound according to embodiment 105, or a pharmaceutically acceptable salt, solvate, or stereoisomer thereof, wherein Ring A is 5-pyrazinyl.
Embodiment 109. The compound according to embodiment 105, or a pharmaceutically acceptable salt, solvate, or stereoisomer thereof, wherein Ring A is 6-pyrazinyl.
Embodiment 110. The compound according to embodiment 90, or a pharmaceutically acceptable salt, solvate, or stereoisomer thereof, wherein Ring A is pyrimidinyl.
Embodiment 111. The compound according to embodiment 110, or a pharmaceutically acceptable salt, solvate, or stereoisomer thereof, wherein Ring A is 2-pyrimidinyl, 4-pyrimidinyl, 5-pyrimidinyl, or 6-pyrimidinyl.
Embodiment 112. The compound according to embodiment 111, or a pharmaceutically acceptable salt, solvate, or stereoisomer thereof, wherein Ring A is 2-pyrimidinyl.
Embodiment 113. The compound according to embodiment 111, or a pharmaceutically acceptable salt, solvate, or stereoisomer thereof, wherein Ring A is 4-pyrimidinyl.
Embodiment 114. The compound according to embodiment 111, or a pharmaceutically acceptable salt, solvate, or stereoisomer thereof, wherein Ring A is 5-pyrimidinyl.
Embodiment 115. The compound according to embodiment 111, or a pharmaceutically acceptable salt, solvate, or stereoisomer thereof, wherein Ring A is 6-pyrimidinyl.
Embodiment 116. The compound according to embodiment 90, or a pharmaceutically acceptable salt, solvate, or stereoisomer thereof, wherein Ring A is pyridazinyl.
Embodiment 117. The compound according to embodiment 116, or a pharmaceutically acceptable salt, solvate, or stereoisomer thereof, wherein Ring A is 3-pyridazinyl, 4-pyridazinyl, 5-pyridazinyl, or 6-pyridazinyl.
Embodiment 118. The compound according to embodiment 117, or a pharmaceutically acceptable salt, solvate, or stereoisomer thereof, wherein Ring A is 3-pyridazinyl.
Embodiment 119. The compound according to embodiment 117, or a pharmaceutically acceptable salt, solvate, or stereoisomer thereof, wherein Ring A is 4-pyridazinyl.
Embodiment 120. The compound according to embodiment 117, or a pharmaceutically acceptable salt, solvate, or stereoisomer thereof, wherein Ring A is 5-pyridazinyl.
Embodiment 121. The compound according to embodiment 117 or a pharmaceutically acceptable salt, solvate, or stereoisomer thereof, wherein Ring A is 6-pyridazinyl.
Embodiment 122. The compound according to any one of embodiments 86 to 121, or a pharmaceutically acceptable salt, solvate, or stereoisomer thereof, wherein R⁹ is furanyl, pyrrolyl, thiophenyl, oxazolyl, imidazolyl, thiazolyl, pyrazolyl, isoxazolyl, isothiazolyl, triazolyl, pyridinyl, pyrazinyl, pyrimidinyl, pyridazinyl, or tetrazolyl, each of which is optionally substituted with one or more R^{1a}.
Embodiment 123. The compound according to embodiment 122, or a pharmaceutically acceptable salt, solvate, or stereoisomer thereof, wherein R⁹ is furanyl optionally substituted with one or more R^{1a}.
Embodiment 124. The compound according to embodiment 122, or a pharmaceutically acceptable salt, solvate, or stereoisomer thereof, wherein R⁹ is pyrrolyl optionally substituted with one or more R^{1a}.
Embodiment 125. The compound according to embodiment 122, or a pharmaceutically acceptable salt, solvate, or stereoisomer thereof, wherein R⁹ is thiophenyl optionally substituted with one or more R^{1a}.
Embodiment 126. The compound according to embodiment 122, or a pharmaceutically acceptable salt, solvate, or stereoisomer thereof, wherein R⁹ is oxazolyl optionally substituted with one or more R^{1a}.
Embodiment 127. The compound according to embodiment 122, or a pharmaceutically acceptable salt, solvate, or stereoisomer thereof, wherein R⁹ is imidazolyl optionally substituted with one or more R^{1a}.
Embodiment 128. The compound according to embodiment 122, or a pharmaceutically acceptable salt, solvate, or stereoisomer thereof, wherein R⁹ is thiazolyl optionally substituted with one or more R^{1a}.
Embodiment 129. The compound according to embodiment 122, or a pharmaceutically acceptable salt, solvate, or stereoisomer thereof, wherein R⁹ is pyrazolyl optionally substituted with one or more R^{1a}.
Embodiment 130. The compound according to embodiment 122, or a pharmaceutically acceptable salt, solvate, or stereoisomer thereof, wherein R⁹ is isoxazolyl optionally substituted with one or more R^{1a}.
Embodiment 131. The compound according to embodiment 122, or a pharmaceutically acceptable salt, solvate, or stereoisomer thereof, wherein R⁹ is isothiazolyl optionally substituted with one or more R^{1a}.
Embodiment 132. The compound according to embodiment 122, or a pharmaceutically acceptable salt, solvate, or stereoisomer thereof, wherein R⁹ is triazolyl optionally substituted with one or more R^{1a}.
Embodiment 133. The compound according to embodiment 122, or a pharmaceutically acceptable salt, solvate, or stereoisomer thereof, wherein R⁹ is pyridinyl optionally substituted with one or more R^{1a}.
Embodiment 134. The compound according to embodiment 122, or a pharmaceutically acceptable salt, solvate, or stereoisomer thereof, wherein R⁹ is pyrazinyl optionally substituted with one or more R^{1a}.
Embodiment 135. The compound according to embodiment 122, or a pharmaceutically acceptable salt, solvate, or stereoisomer thereof, wherein R⁹ is pyrimidinyl optionally substituted with one or more R^{1a}.
Embodiment 136. The compound according to embodiment 122, or a pharmaceutically acceptable salt, solvate, or stereoisomer thereof, wherein R⁹ is pyridazinyl optionally substituted with one or more R^{1a}.
Embodiment 137. The compound according to embodiment 122, or a pharmaceutically acceptable salt, solvate, or stereoisomer thereof, wherein R⁹ is tetrazolyl optionally substituted with one or more R^{1a}.
Embodiment 138. The compound according to any one of embodiments 86 to 121, or a pharmaceutically acceptable salt, solvate, or stereoisomer thereof, wherein R⁹ is oxetanyl substituted with one or more Rⁱ.
Embodiment 139. The compound according to any one of embodiments 86 to 138, or a pharmaceutically acceptable salt, solvate, or stereoisomer thereof, wherein R⁷ is hydrogen.
Embodiment 140. The compound according to any one of embodiments 86 to 139, or a pharmaceutically acceptable salt, solvate, or stereoisomer thereof, wherein R^{8c} is halogen or —OR^{a}.
Embodiment 141. The compound according to embodiment 140, or a pharmaceutically acceptable salt, solvate, or stereoisomer thereof, wherein R^{8c} is halogen.
Embodiment 142. The compound according to embodiment 141, or a pharmaceutically acceptable salt, solvate, or stereoisomer thereof, wherein R^{8c} is fluoro, chloro, bromo, or iodo.
Embodiment 143. The compound according to embodiment 140, or a pharmaceutically acceptable salt, solvate, or stereoisomer thereof, wherein R^{8c} is —OR^{a}.
Embodiment 144. The compound according to embodiment 143, or a pharmaceutically acceptable salt, solvate, or stereoisomer thereof, wherein R^{a} is C₁-C₆alkyl.
Embodiment 145. The compound according to embodiment 144, or a pharmaceutically acceptable salt, solvate, or stereoisomer thereof, wherein R^{a} is —CH₃.
Embodiment 146. The compound according to any one of embodiments 86 to 144, or a pharmaceutically acceptable salt, solvate, or stereoisomer thereof, wherein n is 0, 1, or 2.
Embodiment 147. The compound according to embodiment 146, or a pharmaceutically acceptable salt, solvate, or stereoisomer thereof, wherein n is 0.
Embodiment 148. The compound according to embodiment 146, or a pharmaceutically acceptable salt, solvate, or stereoisomer thereof, wherein n is 1.
Embodiment 149. The compound according to embodiment 146, or a pharmaceutically acceptable salt, solvate, or stereoisomer thereof, wherein n is 2.
Embodiment 150. The compound according to any one of embodiments 86 to 149, or a pharmaceutically acceptable salt, solvate, or stereoisomer thereof, wherein each R¹ is independently methyl, ethyl, trifluoromethyl, methoxy, ethoxy, methanesulfonyl, ethanesulfonyl, acetyl, or dimethylamino.
Embodiment 151. The compound according to any one of embodiments 86 to 150, or a pharmaceutically acceptable salt, solvate, or stereoisomer thereof, wherein the compound of Formula (V) has the structure of Formula (Va):

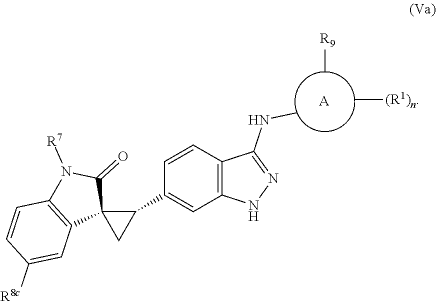

Embodiment 152. The compound according to any one of embodiments 86 to 150, or a pharmaceutically acceptable salt, solvate, or stereoisomer thereof, wherein the compound of Formula (V) has the structure of Formula (Vb):

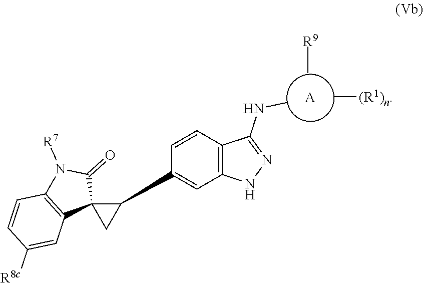

Embodiment 153. A compound, or a pharmaceutically acceptable salt, solvate, or stereoisomer thereof, selected from the group consisting of 6-methoxy-5-({6-[(1R,2S)-5′-methoxy-2′-oxo-1′,2′-dihydrospiro[cyclopropane-1,3′-indol]-2-yl]-1H-indazol-3-yl}amino)-2,3-dihydro-1H-1-benzothiophene-1,1-dione; (1R,2S)-2-{3-[4-(methanesulfonyl)-2-methoxyanilino]-1H-indazol-6-yl}-5′-methoxyspiro[cyclopropane-1,3′-indol]-2′(1′H)-one; (1R,2S)-2-[3-(4-acetyl-2-methoxyanilino)-1H-indazol-6-yl]-5′-methoxyspiro[cyclopropane-1,3′-indol]-2′(1′H)-one; (1R,2S)-5′-methoxy-2-(3-{[3-methoxy-6-(1,3-oxazol-2-yl)pyridin-2-yl]amino}-1H-indazol-6-yl)spiro[cyclopropane-1,3′-indol]-2′(1′H)-one; (1R,2S)-2-{3-[4-(ethanesulfonyl)-2-methoxyanilino]-1H-indazol-6-yl}-5′-methoxyspiro[cyclopropane-1,3′-indol]-2′(1′H)-one; 6-methoxy-5-({6-[(1R,2S)-5′-methoxy-2′-oxo-1′,2′-dihydrospiro[cyclopropane-1,3′-indol]-2-yl]-1H-indazol-3-yl}amino)-1H-1-benzothiophene-1,1-dione; (1R,2S)-2-{3-[2-ethoxy-4-(pyrazin-2-yl)anilino]-1H-indazol-6-yl}-5′-methoxyspiro[cyclopropane-1,3′-indol]-2′(1′H)-one; (1R,2S)-2-{3-[(3-ethoxyquinolin-2-yl)amino]-1H-indazol-6-yl}-5′-methoxyspiro[cyclopropane-1,3′-indol]-2′(1′H)-one; (1R,2S)-2-(3-{4-[3-(dimethylamino)oxetan-3-yl]-2-methoxyanilino}-1H-indazol-6-yl)-5′-methoxyspiro[cyclopropane-1,3′-indol]-2′(1′H)-one; (1R,2S)-2-{3-[2-ethoxy-4-(1H-1,2,4-triazol-1-yl)anilino]-1H-indazol-6-yl}-5′-methoxyspiro[cyclopropane-1,3′-indol]-2′(1′H)-one; (1R,2S)-5′-methoxy-2-{3-[2-methoxy-5-(1-methyl-1H-pyrazol-4-yl)anilino]-1H-indazol-6-yl}spiro[cyclopropane-1,3′-indol]-2′(1′H)-one; 7-ethoxy-6-((6-((1R,2S)-5′-methoxy-2′-oxospiro[cyclopropane-1,3′-indolin]-2-yl)-1H-indazol-3-yl)amino)quinoline 1-oxide; (1R,2S)-5′-methoxy-2-{3-[2-methoxy-5-(1,3-oxazol-2-yl)anilino]-1H-indazol-6-yl}spiro[cyclopropane-1,3′-indol]-2′(1′H)-one; (1R,2S)-5′-methoxy-2-{3-[2-methoxy-5-(1,3-thiazol-2-yl)anilino]-1H-indazol-6-yl}spiro[cyclopropane-1,3′-indol]-2′(1′H)-one; (1R,2S)-5′-methoxy-2-{3-[2-methoxy-5-(1,3-thiazol-4-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-4-yl)anilino]-1H-indazol-6-yl}spiro[cyclopropane-1,3′-indol]-2′(1′H)-one; (1R,2S)-5′-methoxy-2-{3-[2-methoxy-4-(2-methyl-2H-tetrazol-5-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-5-yl)anilino]-1H-indazol-6-yl}spiro[cyclopropane-1,3′-indol]-2′(1′H)-one; (1R,2S)-5′-methoxy-2-(3-{[3-methoxy-6-(1,3-oxazol-2-yl)pyrazin-2-yl]amino}-1H-indazol-6-yl)spiro[cyclopropane-1,3′-indol]-2′(1′H)-one; (1R,2S)-2-(3-{[2-(dimethylamino)-5-methoxypyrimidin-4-yl]amino}-1H-indazol-6-yl)-5′-methoxyspiro[cyclopropane-1,3′-indol]-2′(1′H)-one; (1R,2S)-5′-methoxy-2-{3-[2-methoxy-5-(2-methyl-2H-tetrazol-5-yl)anilino]-1H-indazol-6-yl}spiro[cyclopropane-1,3′-indol]-2′(1′H)-one; (1R,2S)-2-(3-{5-[3-(dimethylamino)oxetan-3-yl]-2-methoxyanilino}-1H-indazol-6-yl)-5′-methoxyspiro[cyclopropane-1,3′-indol]-2′(1′H)-one; (1R,2S)-2-{3-[2-ethoxy-4-(methanesulfonyl)anilino]-1H-indazol-6-yl}-5′-methoxy-1′-methylspiro[cyclopropane-1,3′-indol]-2′(1′H)-one; (1R,2S)-5′-methoxy-2-(3-{[3-methoxy-6-(1-methyl-1H-pyrazol-4-yl)pyrazin-2-yl]amino}-1H-indazol-6-yl)spiro[cyclopropane-1,3′-indol]-2′(1′H)-one; (1R,2S)-5′-methoxy-2-{3-[(5-methoxy[2,5′-bipyrimidin]-4-yl)amino]-1H-indazol-6-yl}spiro[cyclopropane-1,3′-indol]-2′(1′H)-one; (1R,2S)-5′-methoxy-2-(3-{[2-methoxy-5-(1-methyl-1H-pyrazol-4-yl)pyridin-3-yl]amino}-1H-indazol-6-yl)spiro[cyclopropane-1,3′-indol]-2′(1′H)-one; (1R,2S)-5′-methoxy-2-(3-{[5-methoxy-2-(1-methyl-1H-pyrazol-4-yl)pyridin-4-yl]amino}-1H-indazol-6-yl)spiro[cyclopropane-1,3′-indol]-2′(1′H)-one; (1R,2S)-2-(3-{[3-ethoxy-6-(1,3-thiazol-2-yl)pyridin-2-yl]amino}-1H-indazol-6-yl)-5′-methoxyspiro[cyclopropane-1,3′-indol]-2′(1′H)-one; (1R,2S)-2-{3-[2-ethoxy-4-(1-methyl-1H-imidazol-4-yl)anilino]-1H-indazol-6-yl}-5′-methoxyspiro[cyclopropane-1,3′-indol]-2′(1′H)-one; (1R,2S)-5′-methoxy-2-{3-[2-methoxy-5-(1,2-thiazol-3-yl)anilino]-1H-indazol-6-yl}spiro[cyclopropane-1,3′-indol]-2′(1′H)-one; (1R,2S)-5′-methoxy-2-(3-{[5-methoxy-2-(1-methyl-1H-pyrazol-4-yl)pyrimidin-4-yl]amino}-1H-indazol-6-yl)spiro[cyclopropane-1,3′-indol]-2′(1′H)-one; (1R,2S)-5′-methoxy-2-{3-[2-methoxy-5-(1,2-oxazol-3-yl)anilino]-1H-indazol-6-yl}spiro[cyclopropane-1,3′-indol]-2′(1′H)-one; (1R,2S)-2-{3-[2-ethoxy-5-(1,3-thiazol-2-yl)anilino]-1H-indazol-6-yl}-5′-methoxyspiro[cyclopropane-1,3′-indol]-2′(1′H)-one; (1R,2S)-2-(3-{[5-ethoxy-2-(1,3-thiazol-2-yl)pyridin-4-yl]amino}-1H-indazol-6-yl)-5′-methoxyspiro[cyclopropane-1,3′-indol]-2′(1′H)-one; (1R,2S)-2-(3-{[3-ethoxy-6-(1,3-thiazol-2-yl)pyrazin-2-yl]amino}-1H-indazol-6-yl)-5′-methoxyspiro[cyclopropane-1,3′-indol]-2′(1′H)-one; (1R,2S)-5′-methoxy-2-{3-[2-methoxy-5-(3-methoxy-1-methyl-1H-pyrazol-4-yl)anilino]-1H-indazol-6-yl}spiro[cyclopropane-1,3′-indol]-2′(1′H)-one; (1R,2S)-2-{3-[2-ethoxy-5-(1-methyl-1H-pyrazol-4-yl)anilino]-1H-indazol-6-yl}-5′-methoxyspiro[cyclopropane-1,3′-indol]-2′(1′H)-one; (1R,2S)-2-(3-{[3-ethoxy-5-(1H-1,2,4-triazol-1-yl)pyridin-2-yl]amino}-1H-indazol-6-yl)-5′-methoxyspiro[cyclopropane-1,3′-indol]-2′(1′H)-one; (1R,2S)-2-(3-{5-[1-(2,2-difluoroethyl)-1H-pyrazol-4-yl]-2-methoxyanilino}-1H-indazol-6-yl)-5′-methoxyspiro[cyclopropane-1,3′-indol]-2′(1′H)-one; (1R,2S)-5′-methoxy-2-(3-{[5-methoxy-2-(1,3-oxazol-5-yl)pyrimidin-4-yl]amino}-1H-indazol-6-yl)spiro[cyclopropane-1,3′-indol]-2′(1′H)-one; (1R,2S)-2-(3-{[2-ethoxy-5-(1,3-thiazol-2-yl)pyridin-3-yl]amino}-1H-indazol-6-yl)-5′-methoxyspiro[cyclopropane-1,3′-indol]-2′(1′H)-one; (1R, 2S)-5′-methoxy-2-(3-{[2-methoxy-5-(1,3-oxazol-4-yl)pyridin-3-yl]amino}-1H-indazol-6-yl)spiro[cyclopropane-1,3′-indol]-2′(1′H)-one; (1R,2S)-2-{3-[(5-ethoxy-2-methylpyrimidin-4-yl)amino]-1H-indazol-6-yl}-5′-[(2H3)methyloxy]spiro[cyclopropane-1,3′-indol]-2′(1′H)-one; (1R,2S)-2-(3-{[5-ethoxy-2-(1,3-thiazol-2-yl)pyrimidin-4-yl]amino}-1H-indazol-6-yl)-5′-methoxyspiro[cyclopropane-1,3′-indol]-2′(1′H)-one; (1R,2S)-2-(3-((5-ethoxy-2-(3-hydroxy-3-methylbut-1-yn-1-yl)pyrimidin-4-yl)amino)-1H-indazol-6-yl)-5′-methoxyspiro[cyclopropane-1,3′-indolin]-2′-one; (1R,2S)-2-(3-((5-(1-(2,2-difluoroethyl)-1H-pyrazol-4-yl)-2-methoxypyridin-3-yl)amino)-1H-indazol-6-yl)-5′-methoxyspiro[cyclopropane-1,3′-indolin]-2′-one; (1R,2S)-2-(3-((6-(1-(2,2-difluoroethyl)-1H-pyrazol-4-yl)-3-methoxypyrazin-2-yl)amino)-1H-indazol-6-yl)-5′-methoxyspiro[cyclopropane-1,3′-indolin]-2′-one; (1R)-2-(3-((2-ethoxy-5-(oxazol-4-yl)pyridin-3-yl)amino)-1H-indazol-6-yl)-5′-methoxyspiro[cyclopropane-1,3′-indolin]-2′-one; (1R,2S)-2-(3-((5-ethoxy-2-ethynylpyrimidin-4-yl)amino)-1H-indazol-6-yl)-5′-methoxyspiro[cyclopropane-1,3′-indolin]-2′-one; (1R,2S)-2-(3-((2-ethoxy-5-(1H-imidazol-1-yl)pyridin-3-yl)amino)-1H-indazol-6-yl)-5′-methoxyspiro[cyclopropane-1,3′-indolin]-2′-one; (1R,2S)-5′-methoxy-2-(3-((1-methyl-1H-1,2,4-triazol-5-yl)amino)-1H-indazol-6-yl)spiro[cyclopropane-1,3′-indolin]-2′-one; (1R,2S)-2-(3-{[6-(1H-imidazol-1-yl)-3-methoxypyrazin-2-yl]amino}-1H-indazol-6-yl)-5′-methoxyspiro[cyclopropane-1,3′-indol]-2′(1′H)-one; (1R,2S)-5′-methoxy-2-(3-{[3-methoxy-6-(1H-pyrazol-1-yl)pyrazin-2-yl]amino}-1H-indazol-6-yl)spiro[cyclopropane-1,3′-indol]-2′(1′H)-one; 1R,2S)-5′-methoxy-2-(3-{[5-methoxy-2-(1,3-oxazol-4-yl)pyrimidin-4-yl]amino}-1H-indazol-6-yl)spiro[cyclopropane-1,3′-indol]-2′(1′H)-one; 2-[4-({6-[(1R,2S)-5′-methoxy-2′-oxo-1′,2′-dihydrospiro[cyclopropane-1,3′-indol]-2-yl]-1H-indazol-3-yl}amino)-5-methyl-1H-pyrazol-1-yl]-2-methylpropanenitrile; (1R,2S)-5′-methoxy-2-(3-{[3-methyl-1-(trifluoromethyl)-1H-pyrazol-5-yl]amino}-1H-indazol-6-yl)spiro[cyclopropane-1,3′-indol]-2′(1′H)-one; (1R, 2S)-5′-methoxy-2-(3-{[2-methoxy-5-(morpholin-4-yl)pyridin-3-yl]amino}-1H-indazol-6-yl)spiro[cyclopropane-1,3′-indol]-2′(1′H)-one; and 5-({6-[(1R,2S)-5′-methoxy-2′-oxo-1′,2′-dihydrospiro[cyclopropane-1,3′-indol]-2-yl]-1H-indazol-3-yl}amino)-1-methyl-1H-pyrazole-3-carbonitrile.
Embodiment 154. A pharmaceutical composition comprising an amount of a compound according to any one of embodiments 1 to 153, or a pharmaceutically acceptable salt, solvate, or stereoisomer thereof, and one or more pharmaceutically acceptable excipients.
Embodiment 155. A method of treating cancer in a subject in need thereof, comprising administering to the subject a therapeutically effective amount of a compound according to any one of embodiments 1 to 153, or a pharmaceutically acceptable salt, solvate, or stereoisomer thereof.
Embodiment 156. A method of treating cancer in a subject in need thereof, comprising administering to the subject a therapeutically effective amount of a pharmaceutical composition according to embodiment 154.
Embodiment 157. The method according to embodiment 155 or 156, wherein the cancer in the subject is a solid tumor.
Embodiment 158. The method according to any one of embodiments 155 to 157, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma.
Embodiment 159. The method according to any one of embodiments 155 to 158, wherein the cancer in the subject expresses polo-like kinase 4 (PLK4).
Embodiment 160. The method according to any one of embodiments 155 to 158, wherein the cancer in the subject has been determined to express polo-like kinase 4 (PLK4) prior to administering the compound according to any one of embodiments 1 to 153, or a pharmaceutically acceptable salt, solvate, or stereoisomer thereof, or the pharmaceutical composition according to embodiment 154, to the subject.
Embodiment 161. The method according to any one of embodiments 155 to 160, wherein the cancer in the subject exhibits an overexpression of the E3 ubiquitin-protein ligase (TRIM37) protein.
Embodiment 162. The method according to embodiment 161, wherein the cancer in the subject exhibits an overexpression of the gene that encodes the tripartite motif-containing protein 37 (TRIM37).
Embodiment 163. The method according to embodiment 161, wherein the cancer in the subject exhibits an amplification of the gene that encodes the tripartite motif-containing protein 37 (TRIM37).
Embodiment 164. A method of treating cancer in a subject comprising administering to the subject a therapeutically effective amount of a compound according to any one of embodiments 1 to 153, or a pharmaceutically acceptable salt, solvate, or stereoisomer thereof, wherein the cancer in the subject has been determined to overexpress the gene that encodes the tripartite motif-containing protein 37 (TRIM37) prior to administration of the compound to the subject.
Embodiment 165. A method of treating cancer in a subject, wherein the cancer in the subject has been determined to overexpress the gene that encodes the tripartite motif-containing protein 37 (TRIM37), comprising administering to the subject a therapeutically effective amount of a compound according to any one of embodiments 1 to 153, or a pharmaceutically acceptable salt, solvate, or stereoisomer thereof.
Embodiment 166. A method of treating cancer in a subject, comprising:
   a. obtaining a biological sample of the cancer from the subject;
   b. determining whether the biological sample of the cancer overexpresses the gene that encodes the tripartite motif-containing protein 37 (TRIM37); and
   c. administering to the subject a therapeutically effective amount of a compound according to any one of embodiments 1 to 153, or a pharmaceutically acceptable salt, solvate, or stereoisomer thereof, if the biological sample of the cancer is determined to overexpress the gene that encodes the tripartite motif-containing protein 37 (TRIM37).
Embodiment 167. The method according to any one of embodiments 155 to 166, wherein the cancer is neuroblastoma or breast cancer.
Embodiment 168. The method according to embodiment 167, wherein the cancer is neuroblastoma.
Embodiment 169. The method according to embodiment 167, wherein the cancer is breast cancer.
Embodiment 170. The method according to any one of embodiments 155 to 169, wherein the compound according to any one of embodiments 1 to 153, or a pharmaceutically acceptable salt, solvate, or stereoisomer thereof, or the pharmaceutical composition according to embodiment 154 is administered to the subject with one or more additional therapeutic agents.
Embodiment 171. The method according to embodiment 170, wherein the one or more additional therapeutic agents is selected from one or more mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, and immuno-oncology agents.
Embodiment 172. A method of inhibiting polo-like kinase 4 (PLK4) in a subject in need thereof, comprising administering to the subject a therapeutically effective amount of a compound according to any one of embodiments 1 to 153, or a pharmaceutically acceptable salt, solvate, or stereoisomer thereof.
Embodiment 173. A method of inhibiting polo-like kinase 4 (PLK4) in a subject in need thereof, comprising administering to the subject a therapeutically effective amount of a pharmaceutical composition according to embodiment 154.
Embodiment 174. A method of inhibiting polo-like kinase 4 (PLK4) in a subject having cancer, comprising administering to the subject a therapeutically effective amount of a compound according to any one of embodiments 1 to 153, or a pharmaceutically acceptable salt, solvate, or stereoisomer thereof, wherein the cancer in the subject has been determined to express polo-like kinase 4 (PLK4) prior to administering the compound, or a pharmaceutically acceptable salt, solvate, or stereoisomer thereof, to the subject.
Embodiment 175. A method of inhibiting polo-like kinase 4 (PLK4) in a subject having cancer, comprising administering to the subject a therapeutically effective amount of a pharmaceutical composition according to embodiment 154, wherein the cancer in the subject has been determined to express polo-like kinase 4 (PLK4) prior to administering the pharmaceutical composition to the subject.
Embodiment 176. A compound according to any one of embodiments 1 to 153, or a pharmaceutically acceptable salt, solvate, or stereoisomer thereof, for use in a method of treating cancer in a subject in need thereof.
Embodiment 177. A pharmaceutical composition according to embodiment 154 for use in a method of treating cancer in a subject in need thereof.
Embodiment 178. A compound for use according to embodiment 176, wherein the cancer in the subject is a solid tumor.
Embodiment 179. A compound for use according to embodiment 176 or 178, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma.
Embodiment 180. A compound for use according to any one of embodiments 176, 178, or 179 wherein the cancer in the subject expresses polo-like kinase 4 (PLK4).
Embodiment 181. A compound for use according to any one of embodiments 176, 178, or 179, wherein the cancer in the subject has been determined to express polo-like kinase 4 (PLK4) prior to administering the compound to the subject.
Embodiment 182. A compound for use according to any one of embodiments 176, 178, or 179, wherein the cancer in the subject exhibits an overexpression of the E3 ubiquitin-protein ligase (TRIM37) protein.
Embodiment 183. A compound for use according to any one of embodiments 176, 178, or 179, wherein the cancer in the subject exhibits an overexpression of the gene that encodes the tripartite motif-containing protein 37 (TRIM37).
Embodiment 184. A compound for use according to any one of embodiments 176, 178, or 179, wherein the cancer in the subject exhibits an amplification of the gene that encodes the tripartite motif-containing protein 37 (TRIM37).
Embodiment 185. A compound for use according to any one of embodiments 176, 178, or 179, wherein the cancer in the subject has been determined to overexpress the gene that encodes the tripartite motif-containing protein 37 (TRIM37) prior to administration of the compound to the subject.
Embodiment 186. A compound for use according to any one of embodiments 176, 178, or 179, wherein the cancer is neuroblastoma or breast cancer.
Embodiment 187. A compound for use according to 186, wherein the cancer is neuroblastoma.
Embodiment 188. A compound for use according to 186, wherein the cancer is breast cancer.
Embodiment 189. A compound according to any one of embodiments 1 to 153, or a pharmaceutically acceptable salt, solvate, or stereoisomer thereof, for use in inhibiting polo-like kinase 4 (PLK4) in a subject having cancer.
Embodiment 190. Use of a compound according to any one of embodiments 1 to 153, or a pharmaceutically acceptable salt, solvate, or stereoisomer thereof, in the manufacture of a medicament for the treatment of cancer in a subject in need thereof.
Embodiment 191. Use according to embodiment 190, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma.
Embodiment 192. Use according to embodiment 190 or 191, wherein the cancer in the subject expresses polo-like kinase 4 (PLK4).
Embodiment 193. Use according to embodiment 190 or 191, wherein the cancer in the subject has been determined to express polo-like kinase 4 (PLK4) prior to administering the compound to the subject.
Embodiment 194. Use according to any one of embodiments 190 to 193, wherein the cancer in the subject exhibits an overexpression of the E3 ubiquitin-protein ligase (TRIM37) protein.
Embodiment 195. Use according to any one of embodiments 190 to 194, wherein the cancer in the subject exhibits an overexpression of the gene that encodes the tripartite motif-containing protein 37 (TRIM37).
Embodiment 196. Use according to any one of embodiments 190 to 194, wherein the cancer in the subject exhibits an amplification of the gene that encodes the tripartite motif-containing protein 37 (TRIM37).
Embodiment 197. Use according to any one of embodiments 190 to 194, wherein the cancer in the subject has been determined to overexpress the gene that encodes the tripartite motif-containing protein 37 (TRIM37) prior to administration of the compound to the subject.
Embodiment 198. Use according to any one of embodiments 190 to 197, wherein the cancer is neuroblastoma or breast cancer.
Embodiment 199. Use according to embodiment 198, wherein the cancer is neuroblastoma.
Embodiment 200. Use according to embodiment 198, wherein the cancer is breast cancer.

What is claimed is:

1. A compound of Formula (IV), or a pharmaceutically acceptable salt, solvate, or stereoisomer thereof 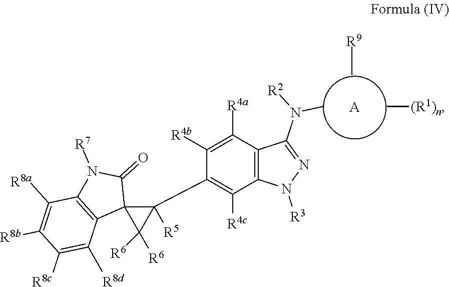
wherein:
Ring A is C₆-C₁₀aryl or heteroaryl;
each R¹ is independently deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, —P(O)(R^{a})₂, —P(O)₂(R^{a})₂, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
or two R¹ on adjacent atoms are taken together to form a C₃-C₁₀cycloalkyl or heterocycloalkyl; each optionally substituted with one or more R^{1b};
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1a} on the same atom are taken together to form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
or two R^{1b} on the same atom are taken together to form an oxo;
n is 0, 1, 2, 3, 4, 5, 6, 7, or 8;
R² is hydrogen, C₁-C₆alkyl, C₁-C₆haloalkyl, or C₁-C₆deuteroalkyl;
R³ is hydrogen, C₁-C₆alkyl, C₁-C₆haloalkyl, or C₁-C₆deuteroalkyl;
each of R^{4a}, R^{4b}, and R^{4c} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R⁶ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R⁷ is hydrogen, C₁-C₆alkyl, C₁-C₆haloalkyl, C₁-C₆deuteroalkyl, C₁-C₆hydroxyalkyl, or C₁-C₆aminoalkyl;
each of R^{8a}, R^{8b}, R^{8c}, and R^{8d} is independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, or heteroaryl;
R⁹ is heteroaryl optionally substituted with one or more Rⁱ, or oxetanyl substituted with one or more R^{1a};
each R^{a} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆alkoxy, C₁-C₆aminoalkyl, C₁-C₆alkylamino, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), or C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
provided the compound of Formula (IV) is not

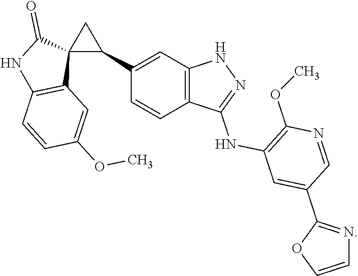 [SMILES: COc1ccc2c(c1)[C@]1(C[C@H]1c1ccc3c(Nc4cc(-c5ncco5)cnc4OC)n[nH]c3c1)C(=O)N2]

2. The compound according to claim 1, or a pharmaceutically acceptable salt, solvate, or stereoisomer thereof, wherein Ring A is C₆-C₁₀aryl.

3. The compound according to claim 1, or a pharmaceutically acceptable salt, solvate, or stereoisomer thereof, wherein Ring A is heteroaryl.

4. The compound according to any one of claims 1 to 3, or a pharmaceutically acceptable salt, solvate, or stereoisomer thereof, wherein n is 1, 2, or 3.

5. The compound according to any one of claims 1 to 4, or a pharmaceutically acceptable salt, solvate, or stereoisomer thereof, wherein R² is hydrogen, and R³ is hydrogen.

6. The compound according to any one of claims 1 to 5, or a pharmaceutically acceptable salt, solvate, or stereoisomer thereof, wherein R^{4a}, R^{4b}, and R^{4c} are hydrogen.

7. The compound according to any one of claims 1 to 6, or a pharmaceutically acceptable salt, solvate, or stereoisomer thereof, wherein R⁵ is hydrogen.

8. The compound according to any one of claims 1 to 7, or a pharmaceutically acceptable salt, solvate, or stereoisomer thereof, wherein each R⁶ is hydrogen.

9. The compound according to any one of claims 1 to 8, or a pharmaceutically acceptable salt, solvate, or stereoisomer thereof, wherein R⁷ is hydrogen.

10. The compound according to any one of claims 1 to 9, or a pharmaceutically acceptable salt, solvate, or stereoisomer thereof, wherein each of R^{8a}, R^{8b}, and R^{8d} are hydrogen and R^{8c} is hydrogen, halogen, or —OR^{a}.

11. The compound according to claim 10, or a pharmaceutically acceptable salt, solvate, or stereoisomer thereof, wherein R^{8c} is —OR^{a}, and R^{a} is C₁-C₆alkyl.

12. A compound, or a pharmaceutically acceptable salt, solvate, or stereoisomer thereof, selected from the group consisting of 6-methoxy-5-({6-[(1R,2S)-5′-methoxy-2′-oxo-1′,2′-dihydrospiro[cyclopropane-1,3′-indol]-2-yl]-1H-indazol-3-yl}amino)-2,3-dihydro-1H-1-benzothiophene-1,1-dione; (1R,2S)-2-{3-[4-(methanesulfonyl)-2-methoxyanilino]-1H-indazol-6-yl}-5′-methoxyspiro[cyclopropane-1,3′-indol]-2′(1′H)-one; (1R,2S)-2-[3-(4-acetyl-2-methoxyanilino)-1H-indazol-6-yl]-5′-methoxyspiro[cyclopropane-1,3′-indol]-2′(1′H)-one; (1R,2S)-5′-methoxy-2-(3-{[3-methoxy-6-(1,3-oxazol-2-yl)pyridin-2-yl]amino}-1H-indazol-6-yl)spiro[cyclopropane-1,3′-indol]-2′(1′H)-one; (1R,2S)-2-{3-[4-(ethanesulfonyl)-2-methoxyanilino]-1H-indazol-6-yl}-5′-methoxyspiro[cyclopropane-1,3′-indol]-2′(1′H)-one; 6-methoxy-5-({6-[(1R,2S)-5′-methoxy-2′-oxo-1′,2′-dihydrospiro[cyclopropane-1,3′-indol]-2-yl]-1H-indazol-3-yl}amino)-1H-1-benzothiophene-1,1-dione; (1R,2S)-2-{3-[2-ethoxy-4-(pyrazin-2-yl)anilino]-1H-indazol-6-yl}-5′-methoxyspiro[cyclopropane-1,3′-indol]-2′(1′H)-one; (1R,2S)-2-{3-[(3-ethoxyquinolin-2-yl)amino]-1H-indazol-6-yl}-5′-methoxyspiro[cyclopropane-1,3′-indol]-2′(1′H)-one; (1R,2S)-2-(3-{4-[3-(dimethylamino)oxetan-3-yl]-2-methoxyanilino}-1H-indazol-6-yl)-5′-methoxyspiro[cyclopropane-1,3′-indol]-2′(1′H)-one; (1R,2S)-2-{3-[2-ethoxy-4-(1H-1,2,4-triazol-1-yl)anilino]-1H-indazol-6-yl}-5′-methoxyspiro[cyclopropane-1,3′-indol]-2′(1′H)-one; (1R,2S)-5′-methoxy-2-{3-[2-methoxy-5-(1-methyl-1H-pyrazol-4-yl)anilino]-1H-indazol-6-yl}spiro[cyclopropane-1,3′-indol]-2′(1′H)-one; 7-ethoxy-6-((6-((1R,2S)-5′-methoxy-2′-oxospiro[cyclopropane-1,3′-indolin]-2-yl)-1H-indazol-3-yl)amino)quinoline 1-oxide; (1R,2S)-5′-methoxy-2-{3-[2-methoxy-5-(1,3-oxazol-2-yl)anilino]-1H-indazol-6-yl}spiro[cyclopropane-1,3′-indol]-2′(1′H)-one; (1R,2S)-5′-methoxy-2-{3-[2-methoxy-5-(1,3-thiazol-2-yl)anilino]-1H-indazol-6-yl}spiro[cyclopropane-1,3′-indol]-2′(1′H)-one; (1R,2S)-5′-methoxy-2-{3-[2-methoxy-5-(1,3-thiazol-4-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-4-yl)anilino]-1H-indazol-6-yl}spiro[cyclopropane-1,3′-indol]-2′(1′H)-one; (1R,2S)-5′-methoxy-2-{3-[2-methoxy-4-(2-methyl-2H-tetrazol-5-yl)anilino]-1H-indazol-6-yl}spiro[cyclopropane-1,3′-indol]-2′(1′H)-one; (1R,2S)-5′-methoxy-2-{3-[2-methoxy-5-(1,3-oxazol-5-yl)anilino]-1H-indazol-6-yl}spiro[cyclopropane-1,3′-indol]-2′(1′H)-one; (1R,2S)-5′-methoxy-2-(3-{[3-methoxy-6-(1,3-oxazol-2-yl)pyrazin-2-yl]amino}-1H-indazol-6-yl)spiro[cyclopropane-1,3′-indol]-2′(1′H)-one; (1R,2S)-2-(3-{[2-(dimethylamino)-5-methoxypyrimidin-4-yl]amino}-1H-indazol-6-yl)-5′-methoxyspiro[cyclopropane-1,3′-indol]-2′(1′H)-one; (1R,2S)-5′-methoxy-2-{3-[2-methoxy-5-(2-methyl-2H-tetrazol-5-yl)anilino]-1H-indazol-6-yl}spiro[cyclopropane-1,3′-indol]-2′(1′H)-one; (1R,2S)-2-(3-{5-[3-(dimethylamino)oxetan-3-yl]-2-methoxyanilino}-1H-indazol-6-yl)-5′-methoxyspiro[cyclopropane-1,3′-indol]-2′(1′H)-one; (1R,2S)-2-{3-[2-ethoxy-4-(methanesulfonyl)anilino]-1H-indazol-6-yl}-5‘-methoxy-l’-methylspiro[cyclopropane-1,3′-indol]-2′(1′H)-one; (1R,2S)-5′-methoxy-2-(3-{[3-methoxy-6-(1-methyl-1H-pyrazol-4-yl)pyrazin-2-yl]amino}-1H-indazol-6-yl)spiro[cyclopropane-1,3′-indol]-2′(1′H)-one; (1R,2S)-5′-methoxy-2-{3-[(5-methoxy[2,5′-bipyrimidin]-4-yl)amino]-1H-indazol-6-yl}spiro[cyclopropane-1,3′-indol]-2′(1′H)-one; (1R,2S)-5′-methoxy-2-(3-{[2-methoxy-5-(1-methyl-1H-pyrazol-4-yl)pyridin-3-yl]amino}-1H-indazol-6-yl)spiro[cyclopropane-1,3′-indol]-2′(1′H)-one; (1R,2S)-5′-methoxy-2-(3-{[5-methoxy-2-(1-methyl-1H-pyrazol-4-yl)pyridin-4-yl]amino}-1H-indazol-6-yl)spiro[cyclopropane-1,3′-indol]-2′(1′H)-one; (1R,2S)-2-(3-{[3-ethoxy-6-(1,3-thiazol-2-yl)pyridin-2-yl]amino}-1H-indazol-6-yl)-5′-methoxyspiro[cyclopropane-1,3′-indol]-2′(1′H)-one; (1R,2S)-2-{3-[2-ethoxy-4-(1-methyl-1H-imidazol-4-yl)anilino]-1H-indazol-6-yl}-5′-methoxyspiro[cyclopropane-1,3′-indol]-2′(1′H)-one; (1R,2S)-5′-methoxy-2-{3-[2-methoxy-5-(1,2-thiazol-3-yl)anilino]-1H-indazol-6-yl}spiro[cyclopropane-1,3′-indol]-2′(1′H)-one; (1R,2S)-5′-methoxy-2-(3-{[5-methoxy-2-(1-methyl-1H-pyrazol-4-yl)pyrimidin-4-yl]amino}-1H-indazol-6-yl)spiro[cyclopropane-1,3′-indol]-2′(1′H)-one; (1R,2S)-5′-methoxy-2-{3-[2-methoxy-5-(1,2-oxazol-3-yl)anilino]-1H-indazol-6-yl}spiro[cyclopropane-1,3′-indol]-2′(1′H)-one; (1R,2S)-2-{3-[2-ethoxy-5-(1,3-thiazol-2-yl)anilino]-1H-indazol-6-yl}-5′-methoxyspiro[cyclopropane-1,3′-indol]-2′(1′H)-one; (1R,2S)-2-(3-{[5-ethoxy-2-(1,3-thiazol-2-yl)pyridin-4-yl]amino}-1H-indazol-6-yl)-5′-methoxyspiro[cyclopropane-1,3′-indol]-2′(1′H)-one; (1R,2S)-2-(3-{[3-ethoxy-6-(1,3-thiazol-2-yl)pyrazin-2-yl]amino}-1H-indazol-6-yl)-5′-methoxyspiro[cyclopropane-1,3′-indol]-2′(1′H)-one; (1R,2S)-5′-methoxy-2-{3-[2-methoxy-5-(3-methoxy-1-methyl-1H-pyrazol-4-yl)anilino]-1H-indazol-6-yl}spiro[cyclopropane-1,3′-indol]-2′(1′H)-one; (1R,2S)-2-{3-[2-ethoxy-5-(1-methyl-1H-pyrazol-4-yl)anilino]-1H-indazol-6-yl}-5′-methoxyspiro[cyclopropane-1,3′-indol]-2′(1′H)-one; (1R,2S)-2-(3-{[3-ethoxy-5-(1H-1,2,4-triazol-1-yl)pyridin-2-yl]amino}-1H-indazol-6-yl)-5′-methoxyspiro[cyclopropane-1,3′-indol]-2′(1′H)-one; (1R,2S)-2-(3-{5-[1-(2,2-difluoroethyl)-1H-pyrazol-4-yl]-2-methoxyanilino}-1H-indazol-6-yl)-5′-methoxyspiro[cyclopropane-1,3′-indol]-2′(1′H)-one; (1R,2S)-5′-methoxy-2-(3-{[5-methoxy-2-(1,3-oxazol-5-yl)pyrimidin-4-yl]amino}-1H-indazol-6-yl)spiro[cyclopropane-1,3′-indol]-2′(1′H)-one; (1R,2S)-2-(3-{[2-ethoxy-5-(1,3-thiazol-2-yl)pyridin-3-yl]amino}-1H-indazol-6-yl)-5′-methoxyspiro[cyclopropane-1,3′-indol]-2′(1′H)-one; (1R, 2S)-5′-methoxy-2-(3-{[2-methoxy-5-(1,3-oxazol-4-yl)pyridin-3-yl]amino}-1H-indazol-6-yl)spiro[cyclopropane-1,3′-indol]-2′(1′H)-one; (1R,2S)-2-{3-[(5-ethoxy-2-methylpyrimidin-4-yl)amino]-1H-indazol-6-yl}-5′-[(2H3)methyloxy]spiro[cyclopropane-1,3′-indol]-2′(1′H)-one; (1R,2S)-2-(3-{[5-ethoxy-2-(1,3-thiazol-2-yl)pyrimidin-4-yl]amino}-1H-indazol-6-yl)-5′-methoxyspiro[cyclopropane-1,3′-indol]-2′(1′H)-one; (1R,2S)-2-(3-((5-ethoxy-2-(3-hydroxy-3-methylbut-1-yn-1-yl)pyrimidin-4-yl)amino)-1H-indazol-6-yl)-5′-methoxyspiro[cyclopropane-1,3′-indolin]-2′-one; (1R,2S)-2-(3-((5-(1-(2,2-difluoroethyl)-1H-pyrazol-4-yl)-2-methoxypyridin-3-yl)amino)-1H-indazol-6-yl)-5′-methoxyspiro[cyclopropane-1,3′-indolin]-2′-one; (1R,2S)-2-(3-((6-(1-(2,2-difluoroethyl)-1H-pyrazol-4-yl)-3-methoxypyrazin-2-yl)amino)-1H-indazol-6-yl)-5′-methoxyspiro[cyclopropane-1,3′-indolin]-2′-one; (1R)-2-(3-((2-ethoxy-5-(oxazol-4-yl)pyridin-3-yl)amino)-1H-indazol-6-yl)-5′-methoxyspiro[cyclopropane-1,3′-indolin]-2′-one; (1R,2S)-2-(3-((5-ethoxy-2-ethynylpyrimidin-4-yl)amino)-1H-indazol-6-yl)-5′-methoxyspiro[cyclopropane-1,3′-indolin]-2-one; (1R,2S)-2-(3-((2-ethoxy-5-(1H-imidazol-1-yl)pyridin-3-yl)amino)-1H-indazol-6-yl)-5′-methoxyspiro[cyclopropane-1,3′-indolin]-2′-one; (1R,2S)-5′-methoxy-2-(3-((1-methyl-1H-1,2,4-triazol-5-yl)amino)-1H-indazol-6-yl)spiro[cyclopropane-1,3′-indolin]-2′-one; (1R,2S)-2-(3-{[6-(1H-imidazol-1-yl)-3-methoxypyrazin-2-yl]amino}-1H-indazol-6-yl)-5′-methoxyspiro[cyclopropane-1,3′-indol]-2′(1′H)-one; (1R,2S)-5′-methoxy-2-(3-{[3-methoxy-6-(1H-pyrazol-1-yl)pyrazin-2-yl]amino}-1H-indazol-6-yl)spiro[cyclopropane-1,3′-indol]-2′(1′H)-one; 1R,2S)-5′-methoxy-2-(3-{[5-methoxy-2-(1,3-oxazol-4-yl)pyrimidin-4-yl]amino}-1H-indazol-6-yl)spiro[cyclopropane-1,3′-indol]-2′(1′H)-one; 2-[4-({6-[(1R,2S)-5′-methoxy-2′-oxo-1′,2′-dihydrospiro[cyclopropane-1,3′-indol]-2-yl]-1H-indazol-3-yl}amino)-5-methyl-1H-pyrazol-1-yl]-2-methylpropanenitrile; (1R,2S)-5′-methoxy-2-(3-{[3-methyl-1-(trifluoromethyl)-1H-pyrazol-5-yl]amino}-1H-indazol-6-yl)spiro[cyclopropane-1,3′-indol]-2′(1′H)-one; (1R, 2S)-5′-methoxy-2-(3-{[2-methoxy-5-(morpholin-4-yl)pyridin-3-yl]amino}-1H-indazol-6-yl)spiro[cyclopropane-1,3′-indol]-2′(1′H)-one; and 5-({6-[(1R,2S)-5′-methoxy-2′-oxo-1′,2′-dihydrospiro[cyclopropane-1,3′-indol]-2-yl]-1H-indazol-3-yl}amino)-1-methyl-TH-pyrazole-3-carbonitrile.

13. A pharmaceutical composition comprising an amount of a compound according to any one of claims 1 to 12, or a pharmaceutically acceptable salt, solvate, or stereoisomer thereof, and one or more pharmaceutically acceptable excipients.

14. A method of treating cancer in a subject in need thereof, comprising administering to the subject a therapeutically effective amount of a compound according to any one of claims 1 to 12, or a pharmaceutically acceptable salt, solvate, or stereoisomer thereof.

15. The method according to claim 14, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma.
